(19) United States
(12) Patent Application Publication
(10) Pub. No.: US 2005/0042624 A1
(43) Pub. Date: Feb. 24, 2005

(54) Tumor antigen

(75) Inventors: Kyogo Itoh, Kiyama-cho (JP); Shigeki Shichijo, Kurume-shi (JP)

Correspondence Address: SUGHRUE MION, PLLC, 2100 PENNSYLVANIA AVENUE, N.W., SUITE 800, WASHINGTON, DC, 20037, US

(73) Assignee: Kyogo ITOH

(21) Appl. No.: 10/734,049

(22) Filed: Dec. 12, 2003

Related U.S. Application Data
(63) Continuation-in-part of application No. PCT/JP02/05799, filed on Jun. 11, 2002.

(30) Foreign Application Priority Data
Jun. 12, 2001 (JP) JP2001-177058
Aug. 21, 2001 (JP) JP2001-250728

(51) Int. Cl. C12Q 1/68; C07H 21/04; A61K 39/395; C07K 14/74

(52) U.S. Cl. 435/6; 435/69.1; 435/320.1; 435/325; 530/350; 536/23.5

(57) ABSTRACT

A gene encoding a tumor antigen that is capable of being recognized by and/or inducing cytotoxic T lymphocyte (CTL) in an HLA-A2 restricted manner was identified from cDNA library of human colon cancer cell line by using a gene expression cloning method, and further found was a peptide having an epitope of the tumor antigen, which is capable of being recognized by and/or inducing cytotoxic T lymphocyte (CTL) in an HLA-A2 restricted manner. In addition, a gene encoding a tumor antigen that is capable of being recognized by and/or inducing cytotoxic T lymphocyte (CTL) in an HLA-A26 restricted manner was identified from a cDNA library of human esophageal cancer cell line, and further found was a peptide having an epitope of the tumor antigen. Further provided are a polypeptide and a peptide encoded by the above-described gene, a polynucleotide encoding any one of those or the complementary strand thereof, a recombinant vector, a transformant, an antibody against the polypeptide or the peptide, a compound that interacts with the polypeptide or the peptide and/or HLA-A2 or HLA-A26, a compound that interacts with the polynucleotide, a CTL inducer consisting of the peptide and/or polypeptide, a pharmaceutical composition comprising any one of those, a method for inducing CTL that comprises using the peptide and/or polypeptide, a method for producing the peptide and/or polypeptide, a method for screening for the compound, a method for measuring the same, and a reagent kit for use in the screening method or the measuring method.

### FIELD OF THE INVENTION

The present invention relates to a tumor antigen, and more particularly to a polypeptide or a peptide recognized by tumor-specific cytotoxic T lymphocytes, a polynucleotide encoding the polypeptide or the peptide and a complementary strand thereto, a recombinant vector comprising the polynucleotide, a transformant comprising the recombinant vector, an antibody against the polypeptide or the peptide, a compound having any interaction with the polypeptide or the peptide and/or an HLA or with the polynucleotide, a cytotoxic T lymphocyte inducer consisting of the peptide and/or the polypeptide, and a pharmaceutical composition comprising the same, and a method for producing the polypeptide and the peptide, a method for screening for the compound, a method for inducing cytotoxic T lymphocytes using the polypeptide and/or the peptide, a method for measuring the polypeptide or the peptide or the polynucleotide encoding the same, and a reagent kit used for the screening method or the measuring method.

### BACKGROUND OF THE INVENTION

The immune system, particularly cytotoxic T lymphocytes that participate in a cellular immunity play an important role in the exclusion of cancer in vivo. Infiltration of cytotoxic T lymphocytes exhibiting cytotoxicity against tumor cells has been detected at the tumor site of a cancer patient (Arch. Surg., 126:200-205, 1990.) A target molecule (tumor antigen) of the tumor-specific cytotoxic T lymphocytes was first discovered in a melanoma. A tumor antigen generated in a tumor cell is degraded in the cell into a peptide (tumor antigen peptide) consisting of 8 to 11 amino acids, which binds to a human leukocyte antigen (HLA) molecule that is the major histocompatibility complex antigen to be presented on the surface of the tumor cell. The cytotoxic T lymphocytes recognize a complex consisting of an HLA molecule and an HLA-restricted manner.

HLA is a cell membrane antigen, and is expressed on almost all eukaryotic cells. HLA is mainly classified into class I antigen and class II antigen. The HLA recognized by the cytotoxic T lymphocytes together with an antigen peptide belongs to class I antigens. HIA class I antigens are further classified into HLA-A, HLA-B, HLA-C and so on. HLA-A, HLA-B, and HLA-C are expressed on each human eukaryotic cell in various amounts. It has been reported that HLA has genetic polymorphism. For example, HLA-A has diversity such as A1, A2, A24, A26, A31, and so on. HLA-B has diversity such as B8, B27, B46, and so on. HLA-C has diversity such as Cw3, Cw6, and so on. In this context, the type of HLA is not same in each individual. The HLA-A2 allele is found in approximately 40% of Japanese, approximately 53% of Chinese, approximately 49% of Northern Caucasians, approximately 38% of Southern Caucasians, and approximately 23% of African Blacks. The HLA-A26 allele is found in approximately 22% of Japanese, approximately 16% of Koreans, and approximately 8% of Northern Caucasians (Imanishi, T. et al., HLA 1991, 1:1065-1220, 1992, Oxford, Oxford Scientific publications.)

When a complex consisting of an HLA class I antigen and a tumor antigen peptide is recognized by cytotoxic T lymphocytes, the type of HLA is also recognized thereby. The amino acid sequence of the tumor antigen peptide capable of binding to HLA molecule is well-known to have a motif (a specific sequence) that differs depending on each type of HLA.

In recent years, molecules, such as a tumor rejection antigen gene, a T cell receptor (TCR), and so, which are involved in specific immunity, have been identified in melanoma, esophageal cancer, and other cancers, and a specific immunotherapy of advanced cancer or metastatic cancer has been studied using the peptide (Science, 254:1643-1647, 1991; J. Exp. Med, 183:1185-1192, 1996; J. Immunol., 163:4994-5004, 1999; Proc. Natl. Acad. Sci. USA, 92:432-436, 1995; Science, 269:1281-1284, 1995; J. Exp. Med, 186:785-793, 1997.)

Now, in Europe and in the United States, cancer vaccine therapy has been developed in which cytotoxic T lymphocytes are activated by an administration of a tumor antigen in a cancer patient. Results from a clinical test of a melanoma specific tumor antigen have been reported. For example, administration of a melanoma antigen gp-100 peptide subcutaneously to melanoma patients along with administering interleukin-2 (IL-2) intravenously gave a tumor regression in 42% of the patients (Nature Medicine, 4:321, 1998.) In this way, by utilizing a tumor antigen as a vaccine, an effective treatment against cancer can be achieved.

However, almost all of the identified tumor antigens are derived from melanomas. Tumor antigens derived from epithelial cancers and adenocarcinomas, which occur at high incidence rates, have been reported for such specific immunotherapy only in a few papers. In addition, in view of the diversity of cancer, an identical tumor antigen may not be expressed in the same degree in all cancer cells. Variations of the type or the tissue of cancer cells gives variations of the type or the amount of a tumor antigen being expressed in the same. In addition, the type of HLA varies in each individual because of the polymorphism of HLA gene, so that the type of a tumor antigen peptide capable of working in each individual is considered to be different. Naturally, cancer vaccine therapy by activating the cytotoxic T lymphocytes using one kind of tumor antigen has a therapeutic effect on cancer having the tumor antigen. However, in order to induce and/or activate specific cytotoxic T lymphocytes in cancer therapy and obtain a high therapeutic effect corresponding to the diversity of cancer, it is important to discover and use many novel tumor antigens.

### SUMMARY OF THE INVENTION

An embodiment of the present invention is an isolated peptide consisting of an amino acid sequence selected from the group consisting of any one of SEQ ID NO:1 to SEQ ID NO:213, SEQ ID NO:358 to SEQ ID NO:381, and SEQ ID NO:388 to SEQ ID NO:408.

Another embodiment of the present invention is an isolated polypeptide consisting of an amino acid sequence selected from the group consisting of any one of SEQ ID NO:215 to SEQ ID NO:288 (excluding SEQ ID NO:228, SEQ ID NO:236, SEQ ID NO:261, and SEQ ID NO:269), SEQ ID NO:356, SEQ ID NO:357, SEQ ID NO:385, and SEQ ID NO:386.

Still another embodiment of the present invention is an isolated peptide consisting of an amino acid sequence selected from the group consisting of any one of SEQ ID NO:1 to SEQ ID NO:213, SEQ ID NO:358 to SEQ ID NO:381, and SEQ ID NO:388 to SEQ ID NO:408, wherein the peptide is recognized by a cytotoxic T lymphocyte and/or induces a cytotoxic T lymphocyte.

Yet another embodiment of the present invention is the peptide, wherein said peptide is recognized by a cytotoxic T lymphocyte in an HLA-A2-restricted manner or HLA-A26-restricted manner and/or induces a cytotoxic T lymphocyte in an HLA-A2-restricted manner or HLA-A26-restricted manner.

An additionally, embodiment of the present invention is an isolated polypeptide consisting of an amino acid sequence selected from the group consisting of anyone of SEQ ID NO:214 to SEQ ID NO:288, SEQ ID NO:356, SEQ ID NO:357, and SEQ ID NO:385 to SEQ ID NO:387, wherein the polypeptide is recognized by a cytotoxic T lymphocyte and/or induces a cytotoxic T lymphocyte.

An embodiment of the present invention is the polypeptide, wherein said polypeptide is recognized by the cytotoxic T lymphocyte in an HLA-A2-restricted manner or HLA-A26-restricted manner and/or induces the cytotoxic T lymphocyte in an HLA-A2-restricted manner or HLA-A26-restricted manner.

Another embodiment of the present invention is a pharmaceutical composition comprising one or more of peptides that consist of an amino acid sequence selected from the group consisting of any of one of SEQ ID NO:1 to SEQ ID NO:213, SEQ ID NO:358 to SEQ ID NO:381, and SEQ ID NO:388 to SEQ ID NO:408, and/or, one or more of polypeptides that consist of an amino acid sequence selected from the group consisting of any one of SEQ ID NO:214 to SEQ ID NO:288, SEQ ID NO:356, SEQ ID NO:357, and SEQ ID NO:385 to SEQ ID NO:387; and a pharmaceutically acceptable carrier.

Yet another embodiment of the present invention is a cancer vaccine comprising an immunoprotective effective amount of one or more of peptides that consist of an amino acid sequence selected from the group consisting of any one of SEQ ID NO:1 to SEQ ID NO:213, SEQ ID NO:358 to SEQ ID NO:381, and SEQ ID NO:388 to SEQ ID NO:408, and/or, one or more of polypeptides that consist of an amino acid sequence selected from the group consisting of any one of SEQ ID NO:214 to SEQ ID NO:288, SEQ ID NO:356, SEQ ID NO:357, and SEQ ID NO:385 to SEQ ID NO:387; and a pharmaceutically acceptable carrier.

Still another embodiment of the present invention is the cancer vaccine, wherein the vaccine is used for treating one or more of cancers selected from the group consisting of colon cancer, esophageal cancer, oral squamous cell cancer, renal cancer, pulmonary cancer, gynecological cancer, and prostate cancer.

An additional embodiment of the present invention is a method for inducing a cytotoxic T lymphocyte, wherein the method comprises contacting peripheral blood mononuclear cells with one or more of peptides that consist of an amino acid sequence selected from the group consisting of any one of SEQ ID NO:1 to SEQ ID NO:213, SEQ ID NO:358 to SEQ ID NO:381, and SEQ ID NO:388 to SEQ ID NO:408, and/or, one or more of polypeptides that consist of an amino acid sequence selected from the group consisting of any one of SEQ ID NO:214 to SEQ ID NO:288, SEQ ID NO:356, SEQ ID NO:357, and SEQ ID NO:385 to SEQ ID NO:387.

An embodiment of the present invention is an isolated polynucleotide encoding a peptide or polypeptide consisting of an amino acid sequence selected from the group consisting of any one of SEQ ID NO:1 to SEQ ID NO:288, SEQ ID NO:356 to SEQ ID NO:381, and SEQ ID NO:385 to SEQ ID NO:408, or a complementary strand thereof.

Another embodiment of the present invention is an isolated polynucleotide consisting of a nucleotide sequence selected from the group consisting of any one of SEQ ID NO:290 to SEQ ID NO:355 (excluding SEQ ID NO:299 and SEQ ID NO:332) and SEQ ID NO:382 to SEQ ID NO:384, or a complementary strand thereof.

Still another embodiment of the present invention is an isolated polynucleotide consisting of a nucleotide sequence selected from the group consisting of any one of SEQ ID NO:289 to SEQ ID NO:355 and SEQ ID NO:382 to SEQ ID NO:384, or a complementary strand thereof, wherein a polypeptide encoded by the polynucleotide induces a cytotoxic T lymphocyte and/or is recognized by a cytotoxic T lymphocyte.

Yet another embodiment of the present invention is the polynucleotide or the complementary strand thereof, wherein said polypeptide induces a cytotoxic T lymphocyte in an HLA-A2-restrcited manner or HLA-A26-restricted manner and/or is recognized by a cytotoxic T lymphocyte in an HLA-A2-restrcited manner or HLA-A26-restricted manner.

An additional embodiment of the present invention is an isolated polynucleotide that hybridizes to the polynucleotide or the complementary strand thereof under stringent conditions.

An embodiment of the present invention is a recombinant vector comprising the polynucleotide or the complementary strand thereof, or a polynucleotide that hybridizes to said polynucleotide or the complementary strand thereof under stringent conditions.

Another embodiment of the present invention is the recombinant vector, wherein the recombinant vector is a recombinant expression vector.

Still another embodiment of the present invention is a transformant transformed with a recombinant vector or a recombinant expression vector, wherein the recombinant vector or the recombinant expression vector comprises the polynucleotide or the complementary strand thereof, or a polynucleotide that hybridizes to said polynucleotide or the complementary strand thereof under stringent conditions.

Yet another embodiment of the present invention is a method for producing a polypeptide, wherein the method comprises culturing a transformant transformed with the recombinant vector.

An additional embodiment of the present invention is an antibody immunologically recognizing the peptide or the polypeptide.

An embodiment of the present invention is a method for screening for a compound that enhances recognition of the peptide or the polypeptide at least by an HLA-A2-restricted or HLA-A26-restricted cytotoxic T lymphocyte, wherein the method comprises contacting said peptide or said polypeptide, with a compound; and determining whether said compound enhances said recognition by measuring IFN-γ production from said cytotoxic T lymphocytes.

Another embodiment of the present invention is a method for screening for a compound that enhances recognition of the peptide at least by an HLA-A2-restricted or HLA-A26-restricted cytotoxic T lymphocyte, wherein said method comprises contacting HLA-A2⁺ cells or HLA-A26⁺ cells which have been pulsed with said peptide, with said cytotoxic T lymphocytes which recognize a complex of the peptide and HLA-A2 molecule or a complex of the peptide and HLA-A26 molecule in the presence or absence of a compound; and determining whether said compound enhances said recognition by measuring IFN-γ production from said cytotoxic T lymphocytes.

Still another embodiment of the present invention is a method for screening for a compound that enhances recognition of the peptide at least by an HLA-A2-restricted or HLA-A26-restricted cytotoxic T lymphocyte, wherein said method comprises contacting HLA-A2⁺ cells or HLA-A26⁺ cells into which the polynucleotide have been transfected, with said cytotoxic T lymphocytes which recognize a complex of the peptide and HLA-A2 molecule or a complex of the peptide and HLA-A26 molecule in the presence or absence of a compound; and determining whether said compound enhances said recognition by measuring IFN-γ production from said cytotoxic T lymphocytes.

Yet another embodiment of the present invention is a compound that is obtained by the screening method.

An additional embodiment of the present invention is a compound that enhances recognition of at least one of the peptide and/or the polypeptide by an HLA-A2-restricted or HLA-A26-restricted cytotoxic T lymphocyte through interaction with the same.

An embodiment of the present invention is a compound that enhances the expression of the polynucleotide or the complementary strand thereof through interaction with the same.

Another embodiment of the present invention is a pharmaceutical composition used for treating cancer, the composition comprising at least one member selected from the group consisting of the peptides, the polypeptides, the polynucleotide or the complementary strand thereof, or a polynucleotide that hybridizes to said polynucleotide or the complementary strand thereof under stringent conditions, a recombinant vector or recombinant expression vector comprising said polynucleotide or the complementary strand thereof, a transformant comprising said recombinant vector or recombinant expression vector, and an antibody that immunologically recognizes said peptide or polypeptide; and a pharmaceutically acceptable carrier.

Still another embodiment of the present invention is a method for measuring quantitatively or qualitatively the peptide, or the polypeptide, or the polynucleotide or the complementary strand thereof, or a polynucleotide that hybridizes to said polynucleotide or the complementary strand thereof under stringent conditions, wherein said method comprises detecting the presence of or determining the amount of said peptide or said polypeptide or said polynucleotide in a sample.

Yet another embodiment of the present invention is the measuring method, wherein the method is used in an examination for a cancer disease.

An additional embodiment of the present invention is a reagent kit comprising at least one member selected from a group consisting of the peptide, the polypeptide, an antibody that immunologically recognizes said peptide or polypeptide, the polynucleotide or the complementary strand thereof, or a polynucleotide that hybridizes to said polynucleotide or the complementary strand thereof under stringent conditions, a recombinant vector or recombinant expression vector comprising said polynucleotide or the complementary strand thereof, a transformant comprising said recombinant vector or recombinant expression vector; and a buffered solution.

An embodiment of the present invention is the reagent kit, wherein the reagent kit is used in diagnosing cancer disease.

Another embodiment of the present invention is a method for treating cancer comprising in vivo administering the cancer vaccine to a patient afflicted with cancer.

An additional embodiment of the present invention is a method for treating cancer comprising in vivo administering the cancer vaccine to a patient afflicted with cancer, in an amount sufficient to induce cytotoxic T lymphocytes in said patient which recognize a complex of the peptide and HLA-A2 molecule or a complex of said peptide and HLA-A26 molecule, and thereby to lyse cancer cells in said patient.

An embodiment of the present invention is a method for treating a cancer patient comprising treating peripheral blood mononuclear cells which have been isolated from said patient with the cancer vaccine; and administering the thus treated peripheral blood mononuclear cells to said patient.

Another embodiment of the present invention is a method for treating a cancer patient comprising treating peripheral blood mononuclear cells which have been isolated from said patient with the cancer vaccine in an amount sufficient to induce cytotoxic T lymphocytes in said patient which recognize a complex of the peptide and HLA-A2 molecule or a complex of said peptide and HLA-A26 molecule, and thereby lyse cancer cells in said patient; and administering the thus treated peripheral blood mononuclear cells to said patient.

An additional embodiment of the present invention is a method for diagnosing cancer comprising measuring quantitatively or qualitatively the peptide, or the polypeptide, or the polynucleotide or the complementary strand thereof, or a polynucleotide that hybridizes to said polynucleotide or the complementary strand thereof under stringent conditions.

### BRIEF DESCRIPTION OF THE DRAWINGS

FIG. 1 illustrates that cDNA clone 2 was recognized by an HLA-A2-restricted cytotoxic T lymphocyte OK-CTLd, and enhanced the interferon-γ (IFN-γ) production from OK-CTLd.

FIG. 2 illustrates that cDNA clone 29 was recognized by an HLA-A2-restricted cytotoxic T lymphocyte OK-CTLd, and enhanced IFN-γ production from OK-CTLd.

FIG. 3 illustrates that cDNA clone 40 was recognized by an HLA-A2-restricted cytotoxic T lymphocyte OK-CTLd, and enhanced IFN-γ production from OK-CTLd.

FIG. 4 illustrates that peptides derived from cDNA clone 2 were recognized by an HLA-A2-restricted cytotoxic T lymphocyte OK-CTLd, and enhanced IFN-γ production from OK-CTLd.

FIG. 5 illustrates that peptides derived from cDNA clone 29 were recognized by an HLA-A2-restricted cytotoxic T lymphocyte OK-CTLd, and enhanced IFN-γ production from OK-CTLd.

FIG. 6 illustrates that peptides derived from cDNA clone 40 were recognized by an HLA-A2-restricted cytotoxic T lymphocyte OK-CTLd, and enhanced IFN-γ production from OK-CTLd.

FIG. 7 illustrates the cytotoxicity of a subline of an HLA-A2-restricted cytotoxic T lymphocyte OK-CTLd and T lymphocyte antigen receptor Vβ usages (TCR Vβ usages.) FIG. 7a illustrates the cytotoxicity of a pooled OK-CTLd subline against T2 cells pulsed with each of five peptides or with a peptide derived from human immunodeficiency virus (HIV) used as a negative control (n.c.). In this figure, the Y-axis denotes percentage of ⁵¹Cr released from T2 cells, i.e., specific cytolytic activity; and the X-axis denotes the ratio of effector cells to target cells (E/T ratio.) FIG. 7b illustrates TCR Vβ usages of peripheral blood mononuclear cells (OK-PBMC) derived from a cancer patient OK, OK-CTLd-6, OK-CTLd-9, or sublines that were established from OK-CTLd, which were pooled as shown in the figure. In the figure, “non-reactive sublines” denote sublines that do not respond to the peptides, and numbers shown in the upper side denotes types of TCR Vβ.

FIG. 8 illustrates that peripheral blood mononuclear cells (PBMC), derived from a cancer patient, recognized each cDNA clone or each peptide in an HLA-A2-restricted manner. FIG. 8a illustrates that PBMC (OK-PBMC) derived from a cancer patient recognized COS-7 cells, into which a plasmid carrying each cDNA clone was co-transfected with HLA-A0207, resulting in enhancement of IFN-γ production from the PBMC. FIG. 8b illustrates that PBMC derived from a cancer patient recognized COS-7 cells, into which HLA-A0207 was transfected and pulsed with a peptide, resulting in enhancement of IFN-γ production from the PBMC.

FIG. 9 illustrates the expression of clone 10, clone 30, clone 41, and clone 108 in various cells such as Panc-1 cells, SW620 cells, peripheral blood mononuclear cells (PBMC) and PHA-blast at the mRNA level.

FIG. 10 illustrates that each of cDNA clones, derived from a human colon cancer, such as SW620-cl.48 (FIG. 10a) and SW620-cl.121 (FIG. 10b) was recognized by an HLA-A2-restricted cytotoxic T lymphocyte OK-CTLd, and enhanced IFN-γ production from OK-CTLd.

FIG. 11 illustrates that peptides derived from cDNA clone SW620-cl.48, which is derived from a human colon cancer, were recognized by HLA-A2-restricted cytotoxic T lymphocyte OK-CTLd, and enhanced IFN-γ production from OK-CTLd.

FIG. 12 illustrates that each of cDNA clones, derived from a human esophageal cancer, such as KE4-cl.17, KE4-cl.18, and KE4-cl.21, was recognized by an HLA-A26-restricted cytotoxic T lymphocyte KE4-CTL, and enhanced IFN-γ production from KE4-CTL.

FIG. 13 illustrates that peptides derived from a cDNA clone KE4-cl.21, which is derived from a human esophageal cancer, were recognized by an HLA-A26-restricted cytotoxic T lymphocyte KE4-CTL, and enhanced IFN-γ production from KE4-CTL. FIG. 13a and FIG. 13b show the results of two experiments. In FIG. 13a, ‘-●-’ denotes KE4-21•P28, ‘-▴-’ denotes KE4-21•P29, ‘-▪-’ denotes KE4-21•P39, and ‘-◯-’ denotes KE4-21•P40. In FIG. 13b, ‘-●-’ denotes KE4-21•P28, ‘-▴-’ denotes KE4-21•P38, ‘-◯-’ denotes KE4-21•P40, and ‘-▪-’ denotes KE4-21•P47.

FIG. 14 illustrates that cytotoxic T lymphocytes were induced and/or activated in an HLA-A26-restricted manner from peripheral blood mononuclear cells (PBMC) derived from a cancer patient, by peptides derived from cDNA clones KE4-cl.18 and KE4-cl.21 that are derived from a human esophageal cancer. In the figure, ‘Pt’ denotes a patient. FIG. 14a illustrates that PBMC, which had been previously stimulated with a peptide, recognized the corresponding peptide, resulting in enhancement of IFN-γ production from the PBMC. In FIG. 14a, ‘*’ denotes that the Two-tailed Student's T-test showed a significant difference (P<0.05) in data. FIG. 14b illustrates cytotoxicity against HLA-A26⁺ esophageal cancer (KE4), HLA-A26⁻ esophageal cancer (KE3), and PHA-blast, which were examined by a 6-hour ⁵¹Cr release test after culturing for 21 days of PBMC derived from a cancer patient together with a peptide. The Y-axis denotes percentage of specific lysis while the X-axis denotes ratios of effector cells against target cells (E/T ratio.) In FIG. 14b, ‘*’ denotes that the Two-tailed Student's T-test showed a significant difference (P<0.05) between the percentage of lysis of KE4 tumor cells and those of KE3 tumor cells

### DETAILED DESCRIPTION OF THE INVENTION

Isolation and Identification of Tumor Antigen Gene

OK-CTL is an HLA-A2-restricted tumor-specific cytotoxic T lymphocyte, which is activated by recognizing a tumor antigen peptide together with an HLA-A2 that is the major type for Japanese among HLA-A molecules, has been established in the present invention from tumor infiltrating lymphocytes (which, hereinafter, may be abbreviated to TIL) of a colon cancer patient according to the method described in the literature [Gomi, S. et al., J. Immunol. (1999) 163: 4994-5004]. OK-CTL obtained is a T lymphocyte in which 80% of the cells have a phenotype of CD3⁺CD4⁻CD8⁺ and the remaining have a phenotype of CD3⁺CD4⁺CD8⁻. OK-CTL recognized all of the tested HLA-A2⁺ tumor cells and produced interferon-γ (IFN-γ) showing an adequate cytotoxicity, while it did not lyse HLA-A2⁻tumor cells, Epstein-Barr virus-transformed autologous B cells (EBV-B), and autologous PHA-blastoid T cells. The cytotoxicity of OK-CTL was inhibited by a monoclonal antibody (mAb) against HLA class I, CD8 or HLA-A2. These results revealed that OK-CTL activates by recognizing tumor cells in an HLA-A2 restricted manner to show cytotoxicity, wherein HLA-A2 is an HLA class I antigen. OK-CTLd that is one of sublines of OK-CTL was used in the present invention. Cryopreserved OK-CTLd was incubated with 100 U/ml of interleukin-2 (which, hereinafter, may be abbreviated to IL-2) for 2 weeks or more for use in the experiments described herein.

A tumor antigen that is recognized by OK-CTLd and can activate OK-CTLd was isolated/identified from a cDNA library of human colon cancer cell line SW620 (HLA-A0201/A2402) by using the gene expression cloning method [Shichijo, S. et al., J. Exp. Med. (1998) 187: 277-288]. A gene encoding the tumor antigen was identified by co-transfecting a cDNA of human colon cancer cell line SW620 together with a cDNA of HLA-A0207 into COS-7 cells, followed by selecting a cell that enhances IFN-γ production from OK-CTLd among cells expressing the transfected gene. The method will be described in more detail in the following Examples. As a result, sixty-seven cDNA clones were obtained which encode gene products recognized by OK-CTLd in an HLA-A2-restricted manner. Hereafter, “gene product” denotes a polypeptide comprising an amino acid sequence being coded by each gene. The nucleotide sequences of the obtained cDNA clones were determined by the dideoxynucleotide sequencing method. These nucleotide sequences are shown in the sequence listing as SEQ ID NO:289 to SEQ ID NO:355. Among these, it was revealed that clone 76 (SEQ ID NO:346) is identical to clone 78 (SEQ ID NO:295.) The above genes were registered in DNA Data Bank of Japan (DDBJ) of National Institute of Genetics (Tables 1 to 7.)

A homology search for these clones using the known database resulted in the finding of genes, derived from human, that are highly homologous to fifty-seven clones among the above obtained sixty-seven genes (see Tables 1 to 7 below.) However, no highly homologous gene was found with respect to nine genes, i.e., clone 4, clone 37, clone 58, clone 71, clone 79, clone 87, clone 89, clone 92, and clone 97. With respect to clone 30, a highly homologous gene was found in murine genes but not in human genes. Although the nucleotide sequences highly homologous to the genes according to the present invention and the deduced amino acid sequences thereof are disclosed, it has not been reported and not been disclosed that these genes encode tumor antigens, even in the open database of National Center for Biotechnology Information (NCBI) when it was browsed on Nov. 2, Nov. 6, and Nov. 7; 2001.

Tumor rejection antigen genes that have been cloned so far, such as those of melanoma, include relatively many genes encoding unusual proteins that contain mutant antigens. However, the genes obtained by the present invention include many genes highly homologous to various enzymes and molecules involved in transcription, translation, and so on. The kinds of the proteins having such homology are similar to those of antigens detected by the Serological analysis of recombinant cDNA expression libraries (SEREX.) The SEREX method permits detecting antigens by using antibodies appearing in a patient's serum [Sahin, U., et al., Proc. Natl. Acad. Sci. USA, 92: 11810-11813, 1995]. In such a method, often detected are antigens that induce humoral immunity. Approximately 1,500 tumor antigens have been identified so far using the method.

Amino acid sequences (SEQ ID NO:214, SEQ ID NO:228, SEQ ID NO:269, SEQ ID NO:261, and SEQ ID NO:236) encoded by five genes (clone 12, clone 65, clone 81, clone 86, and clone 100) among the genes obtained by the present invention are identical to those encoded by known genes ID456, ID629, ID1226, ID163, and ID116, respectively, that were identified by the SEREX method and disclosed in the SEREX database (http://www-ludwig.unil.ch/SEREX.html). Moreover, nucleotide sequences (SEQ ID NO:289, SEQ ID NO:299, and SEQ ID NO:332) of clone 12, clone 82, and clone 86, are identical to those of ID456, ID1197, and ID163, respectively. In addition, it was revealed that clone 32, clone 41, clone 74, and clone 87 are partially homologous to ID1072, ID1233, ID979, and ID567, respectively.

Although the SEREX method can be used to detect tumor antigens, only MAGE-1, tyrosinase, and NY-ESO-1, were so far identified as tumor antigens capable of inducing both cellular and humoral immunities among approximately 1,500 tumor antigens or more that were detected by the method. All of those identified as tumor antigens by the SEREX method do not always induce and/or activate cytotoxic T lymphocytes (which, hereinafter, may be abbreviated to CTL) that are involved in the cellular immunity. The SEREX database disclosed the nucleotide sequences of the above genes and the amino acid sequences of their gene products, while it was not disclosed in the database browsed on Jun. 10, 2002 that the gene products are recognized by CTL and, induce and/or activate CTL.

The present invention revealed for the first time that gene products of the genes shown in Tables 1 to 7 including the above genes (clone 12, clone 65, clone 81, clone 82, clone 86, and clone 100) are recognized by CTL in an HLA-A2-restricted manner and can induce and/or activate CTL.

The above genes obtained by the present invention are genes encoding tumor antigens that are recognized by a tumor-specific CTL in an HLA-A2-restricted manner, which are recognized by CTL in an HLA-A2-restricted manner and can induce and/or activate CTL when expressed in a cell. Amino acid sequences encoded by these genes are shown as SEQ ID NO:214 to SEQ ID NO:288, SEQ ID NO:356, and SEQ ID NO:357 of in the sequence listing (see Tables 1 to 7.) Clone 76 was identical to clone 78, so that it was revealed that amino acid sequences (SEQ ID NO:277 and SEQ ID NO:220) encoded by both genes are also identical each other.

TABLE 1

| | | polypeptide | | |
|---|---|---|---|---|
| cDNA clone | SEQ | encoded by | SEQ | |
| (base length, | ID | cDNA (AA | ID | |
| bp) | NO: | length) | NO: | Homologous genes [accession number of GenBank] |
| 12 | 289 | PP 12 | 214 | glyceraldehyde-3-phosphate dehydrogenase |
| (1280) | | (335) | | [XM_006959] |
| [AB062273] | | | | |
| 40 | 290 | PP 40 | 215 | ATP-binding cassette, sub-family E(OABP), |
| (2978) | | (599) | | member1 (ABCE1) [XM_003555] |
| [AB062293] | | | | |
| 43 | 291 | PP 43 | 216 | ubiquitin-homology domain protein PIC1 |
| (1218) | | (101) | | [U61397] |
| [AB062294] | | | | ubiquitin-like1 (sentrin) [BC006462] |
| 2 | 292 | PP 2 | 217 | ribosomal protein S6 [NM_001010] |
| (825) | | (249) | | FLJ23534 fis [AK027187] |
| [AB062123] | | | | |
| 11 | 293 | PP 11 | 218 | member of Ras oncogene family (RAP1B) |
| (1978) | | (184) | | [BC000176] |
| [AB062128] | | | | |
| 8 | 294 | PP 8 | 219 | transcription factor BTF 3. |
| (895) | | (162) | | [X74070] |
| [AB062126] | | | | |
| 78 | 295 | PP 78 | 220 | CGI-37 [AF132971] |
| (1358) | | (180) | | HSPC031 [XM_007837] |
| [AB062479] | | | | |
| 67 | 296 | PP 67 | 221 | NOF1 [U39400] |
| (2033) | | (166) | | chromosome 11 open reading frame 4 |
| [AB062395] | | | | [BC004378] |
| 9 | 297 | PP 9 | 222 | putative oncogene [XM_016246] |
| (1059) | | (194) | | inosine triphosphate pyrophosphatase |
| [AB062127] | | | | [AF219116] |
| 95 | 298 | PP 95 | 223 | Annexin A7 [BC002632] |
| (1769) | | (466) | | |
| [AB062429] | | | | |

TABLE 2

| | | polypeptide | | |
|---|---|---|---|---|
| cDNA clone | SEQ | encoded by | SEQ | Homologous genes |
| (base length, | ID | cDNA (AA | ID | [accession number |
| bp) | NO: | length) | NO: | of GenBank] |
| 82 | 299 | PP 82 | 224 | ribosomal protein S15a |
| (463) | | (130) | | [X84407] [BC001697] |
| [AB062400] | | | | |
| 103 | 300 | PP 103 | 225 | ribosomal protein L9 |
| (703) | | (192) | | [BC004206] |
| [AB062431] | | | | |
| 4 | 301 | PP 4 | 226 | [AL365207] |
| (887) | | (67) | | |
| [AB062435] | | | | |
| 14 | 302 | PP 14 | 227 | caldesmon, 3′UTR |
| (905) | | (66) | | [AJ223812] [AC090497] |
| [AB062484] | | | | |
| 65 | 303 | PP 65 | 228 | Gu protein [U41387] |
| (1832) | | (145) | | DEAD/H box polypeptide |
| | | | | 21(DDX21) |
| [AB062394] | | | | [NM_004728] |
| 69 | 304 | PP 69 | 229 | tumor protein D52-like 2 |
| (1824) | | (49) | | [XM_009688] |
| [AB062396] | | | | |
| 83 | 305 | PP 83 | 230 | ribosomal protein S8 |
| (759) | | (208) | | [NM_001012] |
| [AB062401] | | | | |
| 84 | 306 | PP 84 | 231 | ferritin heavy polypeptide 1 |
| (938) | | (183) | | [M11146] [BC000857] |
| [AB062402] | | | | |
| 32 | 307 | PP 32 | 232 | ribosomal protein L3 |
| (1281) | | (403) | | [NM_000967] |
| [AB062291] | | | | |
| 21 | 308 | PP 21 | 233 | uridine monophosphate |
| (1698) | | (480) | | synthetase |
| [AB062285] | | | | [NM_000373] |
| 33 | 309 | PP 33 | 234 | splicing factor, |
| (1102) | | (86) | | arginine/serine-rich 11 |
| [AB062486] | | | | [XM_001835] |
| 68 | 310 | PP 68 | 235 | glutaminyl-peptide |
| (519) | | (54) | | cyclotransferase |
| [AB062487] | | | | [NM_012413] |

TABLE 3

| | | polypeptide | | |
|---|---|---|---|---|
| cDNA clone | SEQ | encoded by | SEQ | |
| (base length, | ID | cDNA (AA | ID | Homologous genes [accession |
| bp) | NO: | length) | NO: | number of GenBank] |
| 100 | 311 | PP 100 | 236 | FLJ10669 |
| (2335) | | (672) | | [XM_009301] [BC006358] |
| [AB062430] | | | | |
| 73 | 312 | PP 73 | 237 | ubiquitin carrier protein |
| (1027) | | (222) | | (E2-EPF) |
| [AB062397] | | | | [NM_014501] [BC004236] |
| 27 | 313 | PP 27 | 238 | integrin beta 4 binding protein |
| (1068) | | (245) | | [BC001119] |
| [AB062289] | | | | |
| 26 | 314 | PP 26 | 239 | IMR-90 ribosomal protein S3 |
| (810) | | (117) | | [U14992] [BC003577] |
| [AB062288] | | | | |
| 56 | 315 | PP 56 | 240 | β-tublin |
| (2505) | | (444) | | [AF141349] [BC002347] |
| [AB062393] | | | | |
| 5 | 316 | PP 5 | 241 | tropomyosin 4 |
| (1588) | | (92) | | [BC000771] [X04588] |
| [AB062125] | | | | |
| 10 | 317 | PP 10 | 242 | FLJ12118 |
| (1831) | | (453) | | [NM_024537] |
| [AB062436] | | | | |
| 22 | 318 | PP 22 | 243 | peroxisomal farnesylated |
| (3476) | | (209) | | protein [NM_002857] |
| [AB062286] | | | | |
| 30 | 319 | PP 30 | 244 | 8 days embryo of |
| (1665) | | (354) | | MUS musculus |
| [AB062438] | | | | [AK019987] |
| 88 | 320 | PP 88 | 245 | sulfotransferase family, |
| (1571) | | (295) | | cytosolic1A phenol-preferring |
| [AB062428] | | | | member1 [NM_001055] |
| 45 | 321 | PP 45 | 246 | dolichyl- |
| (1549) | | (439) | | diphosphooligosaccharide- |
| [AB062391] | | | | protein glycosyltransferase |
| | | | | [BC002594] |

TABLE 4

| | | polypeptide | | |
|---|---|---|---|---|
| cDNA clone | SEQ | encoded by | SEQ | Homologous genes |
| (base length, | ID | cDNA (AA | ID | [accession |
| bp) | NO: | length) | NO: | number of GenBank] |
| 58 | 322 | PP 58-F3 (56) | 247 | — |
| (2064) | | PP 58-F2 (46) | 248 | |
| 18 | 323 | PP 18-F1 (61) | 249 | methylthioadenosine |
| (1317) | | PP 18-F2 (73) | 250 | phosphorylase |
| [AB062485] | | PP 18-F3 (43) | 251 | [XM_011800] |
| 87 | 324 | PP 87 | 252 | — |
| (1483) | | (30) | | |
| 24 | 325 | PP 24 | 253 | prolyl 4-hydroxylase |
| (1067) | | (87) | | beta-subunit and disulfide |
| [AB062434] | | | | isomerase [M22806] |
| 46 | 326 | PP 46 | 254 | ribosomal protein L13 |
| (915) | | (211) | | [NM_000977] |
| [AB062392] | | | | |
| 110 | 327 | PP 110 | 255 | phosphoglycerate kinase 1 |
| (2338) | | (417) | | (PGK1) [XM_010102] |
| [AB062432] | | | | |
| 20 | 328 | PP 20 | 256 | |
| (2519) | | (568) | | macrophage |
| [AB062284] | | | | protein 2 [AF064484] |
| 6 | 329 | PP 6-F1 (46) | 257 | DKFZp762E1112 |
| (1623) | | PP 6-F2 (36) | 258 | [AL162047] |
| 108 | 330 | PP 108 | 259 | DKFZp434G2226 |
| (3379) | | (898) | | [NM_031217] |
| [AB062483] | | | | |
| 23 | 331 | PP 23 | 260 | heterogeneous nuclear |
| (964) | | (71) | | ribonucleoprotein F |
| [AB062287] | | | | [BC004254] [L28010] |

TABLE 5

| | | polypeptide | | |
|---|---|---|---|---|
| cDNA clone | SEQ | encoded by | SEQ | |
| (base length, | ID | cDNA (AA | ID | |
| bp) | NO: | length) | NO: | Homologous genes [accession number of GenBank] |
| 86 | 332 | PP 86 | 261 | 5-aminoimidazole-4-carboxamide-1-beta-D- |
| (1937) | | (592) | | ribonucleotide transformylase/inosinicase |
| [AB062403] | | | | [D82348] |
| 89 | 333 | PP 89 | 262 | — |
| (2029) | | (62) | | |
| [AB062482] | | | | |
| 92 | 334 | PP 92 | 263 | — |
| (2923) | | (43) | | |
| [AB062488] | | | | |
| 85 | 335 | PP 85-F2 (303) | 264 | KIAA0036 |
| (2283) | | PP 85-F3 (264) | 265 | [NM_014642] [BC005806] |
| [AB062481] | | | | |
| 3 | 336 | PP 3 | 266 | Human SF2p33 [M69040] |
| (2765) | | (248) | | splicing factor, arginine/serine-rich 2 (SC-35) |
| [AB062124] | | | | [BC006181] |
| 29 | 337 | PP 29 | 267 | thymidylate synthetase |
| (1567) | | (313) | | [NM 001071] [XM 008753] |
| [AB062290] | | | | |
| 35 | 338 | PP 35 | 268 | catenin beta 1(CTNNB1) |
| (2224) | | (511) | | [NM_001904] [XM_003222] |
| [AB062292] | | | | |
| 81 | 339 | PP 81 | 269 | RAN, member RAS oncogene family |
| (854) | | (128) | | [BC004272] |
| [AB062399] | | | | |
| 114 | 340 | PP 114 | 270 | FLJ13660, similar to Rattus CDK5 activator- |
| (1816) | | (506) | | binding protein [AK023722] |
| [AB062433] | | | | FLJ20253 [AK000260] |
| 19 | 341 | PP 19 | 271 | protein kinase Njmu-R1 |
| (696) | | (136) | | [XM_015338] |
| [AB062437] | | | | |

TABLE 6

| cDNA clone | SEQ | polypeptide | SEQ | |
|---|---|---|---|---|
| (base length, | ID | encoded by cDNA | ID | |
| bp) | NO: | (AA length) | NO: | Homologous genes [accession number of GenBank] |
| 74 | 342 | PP 74 | 272 | KIAA0795 |
| (4912) | | (509) | | [AB018338] |
| [AB062478] | | | | |
| 41 | 343 | PP 41-F1 (49) | 273 | FLJ20489 |
| (2731) | | PP 41-F3 (109) | 274 | [AK000496] |
| [AB062477] | | | | |
| 79 | 344 | PP 79 | 275 | [AC008088] |
| (561) | | (54) | | |
| [AB062480] | | | | |
| 36 | 345 | PP 36 | 276 | FLJ22245 [AK025898] |
| (3443) | | (66) | | FLJ20489 [AK000496] |
| 76 | 346 | PP 76 | 277 | HSPC031 |
| (1358) | | (180) | | [XM_007837] |
| [AB062398] | | | | |
| 37 | 347 | PP 37 | 278 | Intron of UDP-N-acetylglucosamine 2- |
| (1047) | | (34) | | epimerase gene [AF317635] |
| 38 | 348 | PP 38-F1 | 279 | 3′UTR of tumor protein P53 |
| (1306) | | (168) | | (Li-Fraumenisyndrome) |
| | | PP 38-F3 | 280 | [BC003596] |
| | | (158) | | |
| 70 | 349 | PP 70 | 281 | Phosphoglycerate mutase 1 (PGAM1) |
| (341) | | (43) | | [XM_017950] |
| 71 | 350 | PP 71 | 282 | — |
| (791) | | (61) | | |
| 75 | 351 | PP 75 | 283 | eukaryotic translation initiation factor 4 gamma, |
| (1474) | | (207) | | 2 (E1F4G2) [XM_006326] |
| 97 | 352 | PP 97-F1 (105) | 284 | — |
| (2932) | | PP 97-F2 (91) | 285 | |
| | | PP 97-F3 (75) | 286 | |
| 53 | 353 | PP 53-F1 (83) | 287 | synaptogyrin 2 (SYNGR2) |
| (1254) | | PP 53-F2 (117) | 288 | [NM_004710] |

TABLE 7

| | | polypeptide | | |
|---|---|---|---|---|
| cDNA clone | SEQ | encoded by | SEQ | Homologous genes |
| (base length, | ID | cDNA (AA | ID | [accession number |
| bp) | NO: | length) | NO: | of GenBank] |
| SW620-cl.48 | 354 | SW620-48-PP | 356 | solute carrier family 25 |
| (1324) | | (361) | | (mitochondrial carrier; |
| | | | | phosphate carrier), |
| | | | | member 3 (SLC25A3) |
| | | | | [XM_039619] |
| SW620-cl.121 | 355 | SW620-121-PP | 357 | aminolevulinate, delta-, |
| (2303) | | (640) | | synthase 1 (ALAS1) |
| | | | | [NM_000688] |

In addition, an HLA-A26-restricted tumor-specific cytotoxic T lymphocyte KE4-CTL, from an esophageal cancer patient (HLA-A2601/2402), which is activated by recognizing HLA-A26 and a tumor antigen peptide has been established [Nakao, M. et al., Cancer Res., 55: 4248-4252, 1995]. Moreover, tumor antigens recognized by KE4-CTL were isolated/identified from the cDNA library of human esophageal cancer cell line KE4 (HLA-A2601/2402) in a manner similar to one described above. Three cDNA clones encoding tumor antigens recognized by KE4-CTL in an HLA-A26-restricted manner were obtained by co-transfecting cDNA of a human esophageal cancer cell line KE4 and cDNA of HLA-A2601 into VA13 cells, followed by selecting clones that enhance IFN-γ production from KE4-CTL from cells in which the transfected genes were expressed. These nucleotide sequences and deduced amino acid sequences, were determined in a manner similar to one described above and are shown as SEQ ID NO:382 to SEQ ID NO:384 and SEQ ID NO:385 to SEQ ID NO:387 in the sequence listing, respectively. These genes were registered in DNA Data Bank of Japan (DDBJ) of National Institute of Genetics (Table 8.) In addition, as described in Table 8, genes highly homologous to these genes were found by homology search. Among these, the nucleotide sequence of KE4-cl.21 is identical to that of ribosomal protein L10a except for the 640^{th} nucleotide, and both deduced amino acid sequences were identical each other. Although nucleotide sequences of these highly homologous genes and deduced amino acid sequences thereof are disclosed, it has not been reported that these encode tumor antigens and not been disclosed even in the open database of NCBI when it was browsed on Nov. 7; 2001. The genes thus obtained are those encoding tumor antigens recognized by tumor-specific CTL in an HLA-A26-restricted manner, which are recognized by CTL in an HLA-A26-restricted manner when expressed in a cell and can induce and/or activate CTL.

TABLE 8

| | | polypeptide | | |
|---|---|---|---|---|
| cDNA clone | SEQ | encoded by | SEQ | Homologous genes |
| (base length, | ID | cDNA (AA | ID | [accession number of |
| bp) | NO: | length) | NO: | GenBank] |
| KE4-cl.17 | 382 | KE4-17-PP | 385 | ribosomal protein L13a |
| (974) | | (142) | | [NM_012423] |
| [AB082924] | | | | [XM_027885] |
| KE4-cl.18 | 383 | KE4-18-PP | 386 | ribosomal protein S2 |
| (821) | | (233) | | [BC001795] |
| [AB082925] | | | | |
| KE4-cl.21 | 384 | KE4-21-PP | 387 | ribosomal protein L10a |
| (741) | | (217) | | [BC006791] [AAH06791] |
| [AB082926] | | | | |

In the present specification, “a tumor antigen” means a protein or a peptide that is recognize by tumor-specific cytotoxic T lymphocytes and/or is capable of inducing tumor-specific cytotoxic T lymphocytes, which is present in a tumor cell. Moreover, a “tumor antigen peptide” means a peptide that is generated as a result of degradation of the tumor antigen in a tumor cell, which is recognize by tumor-specific cytotoxic T lymphocytes through being presented on a cell surface by binding to an HLA molecule and/or is capable of inducing tumor-specific cytotoxic T lymphocytes. In addition, the site of the amino acid sequence that is capable of inducing and/or activating tumor-specific cytotoxic T lymphocytes, which is present in a tumor antigen, is called “a tumor antigen epitope (tumor antigen determinant.)”

“Recognize” herein means that a subject distinguishes an object from others and cognates it, for example, binds to the object cognized. Particularly, in the present specification, that CTL recognize the tumor cells or the tumor antigen peptides means that CTL binds through a T cell receptor (which, hereinafter, may be abbreviated to TCR) to the tumor antigen peptides that are presented by HLA molecules. “Activate” herein means to enhance or to make it work further a thing or a state that has an activity or an action. Particularly, in the present specification, activation of CTL means that CTL recognizes an antigen being presented by an HLA molecule to produce IFN-γ or to show cytotoxicity against the target cells recognized thereby.

“Induce” herein means to generate an activity or an action from a thing or a state that are in a phase merely having the activity or the action. Particularly, in the present specification, induction of an antigen-specific CTL means to make CTL, which specifically recognizes a certain antigen, differentiate and/or proliferate in vitro or in vivo. Moreover in the present specification, the inducer of cytotoxic T lymphocytes means an agent which changes the state where CD8-positive T lymphocytes specifically recognizing a certain antigen is absent or present in a very low degree, to the state where the cytotoxic T lymphocytes recognizing the antigen is present in a very degree.

Identification of Tumor Antigen Peptide CTL

In order to obtain a tumor antigen peptide from amino acid sequences encoded by the above genes, an HLA-A2 binding motif (a specific sequence) was searched using the homepage (http://bimas.dcrt.nih.gov/molbio/hla_bind/) of Bioinformatics & Molecular Analysis Section (BIMAS), and an amino acid sequence suitable for the motif was specified based on the amino acid sequences encoded by the above genes and the amino acid sequences of gene products of genes highly homologous to the above genes. Based on the result, various peptides of 9-mer and 10-mer having an HLA-A2-binding motif were designed and synthesized.

Each synthetic peptide was pulsed to T2 cells expressing HLA-A2, followed by culturing the T2 cells with OK-CTLd to measure IFN-γ production from OK-CTLd. Peptides recognized by OK-CTLd were selected using the amount of IFN-γ production as an index. Among 628 peptides synthesized, 237 peptides (SEQ ID NO:1 to SEQ ID NO:213 and SEQ ID NO:358 to SEQ ID NO:381) were recognized by OK-CTLd and enhanced IFN-γ production from OK-CTLd in a dose dependent manner of the peptide. Among these peptides, P77 (SEQ ID NO:20) and P79 (SEQ ID NO:21) derived from clone 78 were identical to P656 (SEQ ID NO:211) and P658 (SEQ ID NO:212) derived from clone 76, respectively. Thus, 235 of tumor antigen peptides were obtained that are recognized by CTL in an HLA-A2-restricted manner and can activate the CTL.

Various peptides of 9-mer and 10-mer capable of binding to HLA-A26 were designed and synthesized based on the amino acid sequences of the above tumor antigen (SEQ ID NO:385 to SEQ ID NO:387) recognized by CTL in an HLA-A26-restricted manner, according to the method described in the literature [J. Exp. Med., 184: 735-740, 1996]. With respect to the gene KE4-cl.17, peptides related to the amino acid sequences encoded by genes highly homologous to the gene were also designed and synthesized. Each synthetic peptide described above was pulsed to VA13 cells that were made to express HLA-A26, followed by culturing the VA13 cells with KE4-CTL to measure IFN-γ production from KE4-CTL for use as an index so as to select peptides recognized by KE4-CTL in an HLA-A26-restricted manner. Twenty-one peptides (SEQ ID NO:388 to SEQ ID NO:408) among the synthesized peptides were recognized by KE4-CTL and enhanced IFN-γ production from KE4-CTL in a dose dependent manner of each peptide. Thus, twenty-one tumor antigen peptides were obtained that are recognized by CTL in an HLA-A26-restricted manner and can activate the CTL.

Specificity of TCR Recognizing Peptide

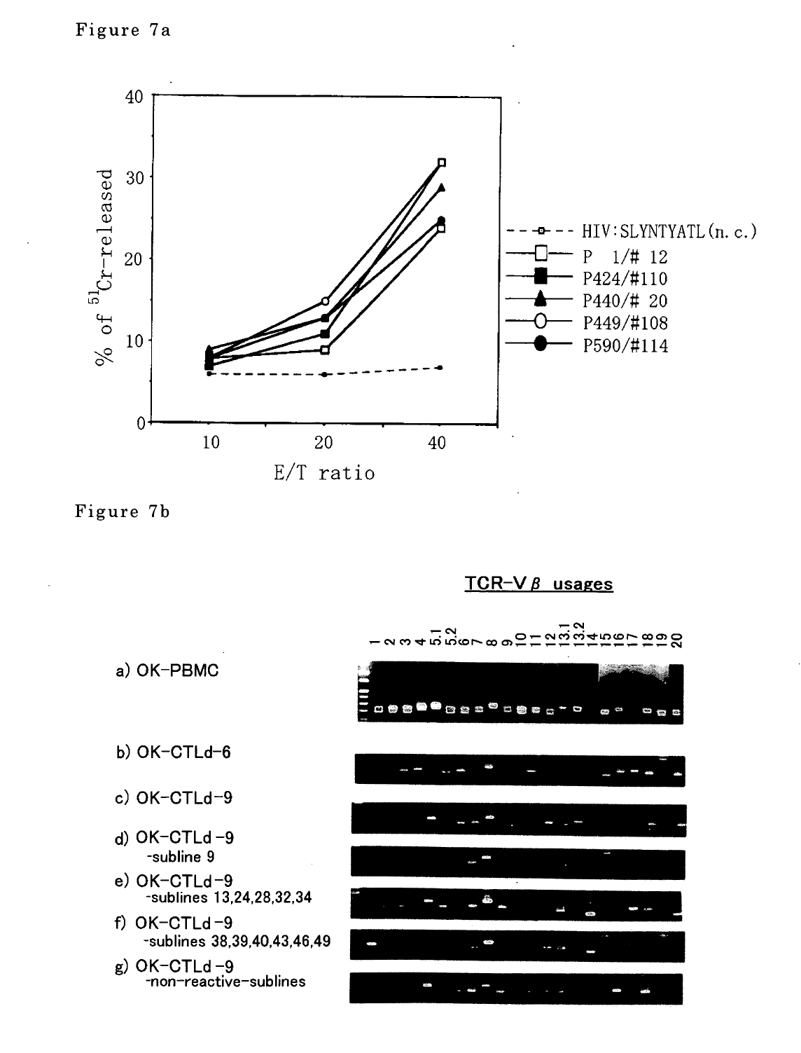

CTL recognize a complex comprising a tumor antigen peptide and an HLA molecule through a TCR that is expressed on the cell surface thereof. It was reported that the diversity of TCR is over 10¹⁷. Investigation using 480 OK-CTLd sublines established from a parent cell line OK-CTLd, revealed that the recognition of peptide by the sublines depends on the kind of subline (see Table 9.) Moreover, TCR Vβ of these OK-CTLd sublines recognizing each peptide was different in each subline (see FIG. 7b.) These results revealed that CTL having different TCR Vβ shows different specificity against each peptide. These results further revealed that even CTL established from a tumor-infiltrating lymphocyte of one patient is a population consisting of cells having different TCR, whence it can recognize various antigens and peptides.

Induction and/or Activation of CTL in Peripheral Blood Mononuclear Cells Derived from a Cancer Patient

It was investigated whether forty-five gene products among the above gene products, which are recognized by OK-CTLd in an HLA-A2-restricted manner, are recognized by a peripheral blood mononuclear cell (which, hereinafter may be abbreviated to PBMC) derived from an HLA-A2⁺ colon cancer patient. As a result, the PBMC derived from one colon cancer patient, who provided TIL for use to establish OK-CTL, recognized six gene products (clone 20, clone 43, clone 56, clone 84, clone 86, and clone 108.) The PBMC derived from another colon cancer patient recognized four gene products (clone 20, clone 84, clone 86, and clone 108.) On the other hand, these clones were not recognized by PBMC derived from a metastatic melanoma patient, PBMC derived from a healthy donor, or a cytotoxic T lymphocyte GK-CTL established from a lung cancer tissue-infiltrating lymphocyte.

In addition, peptides derived from these gene products, P430, P431, P434, and P440 (SEQ ID NO:360, SEQ ID NO:361, SEQ ID NO:144, and SEQ ID NO:146) derived from clone 20, P45 (SEQ ID NO:10) derived from clone 43, P288 (SEQ ID NO:358) and P289 (SEQ ID NO:359) derived from clone 56, P184 (SEQ ID NO:65) derived from clone 84, P485 (SEQ ID NO:166) and P486 (SEQ ID NO:363) derived from clone 86, P449 (SEQ ID NO:153) and P463 (SEQ ID NO:362) derived from clone 108 were recognized by at least one of the PBMC derived from the both colon cancer patients described above and enhanced IFN-γ production from the PBMC. A cell that recognizes the above gene product or the above peptide and produces IFN-γ was an HLA-A2-restricted CD8-positive T lymphocyte in PBMC.

As described above, it was revealed that CTL precursor recognizing the above tumor antigen and peptides derived therefrom in an HLA-A2-restricted manner is present in PBMC of a colon cancer patient. These tumor antigens and peptides derived therefrom would be able to induce and/or activate a tumor-specific CTL in an HLA-A2-restricted manner in this patient. Therefore, tumor antigens shown in Tables 1 to 7 and peptides derived therefrom would be suitable for use in the specific immunotherapy for HLA-A2⁺ colon cancer patients.

Moreover, among the above peptides recognized by KE4-CTL in an HLA-A26-restricted manner, CTL-inducing ability of PBMC derived from HLA-A26⁺ patients (oral squamous cell cancer, four cases; renal cancer, one case; and pulmonary cancer, one case) was investigated with respect to the following six peptides that strongly activated KE4-CTL: peptides KE4-18•P5 (SEQ ID NO:395), KE-4-18•P22 (SEQ ID NO:399), and KE4-18•P25 (SEQ ID NO:400) derived from KE4-cl.18 as well as KE4-21•P28 (SEQ ID NO:403), KE4-21•P29 (SEQ ID NO:404) and KE4-21•P40 (SEQ ID NO:407) derived from KE4-cl.21. As a result, the above six peptides were recognized by PBMC derived from the cancer patients and significantly enhanced IFN-γ production from the PBMC, while PBMC derived from a healthy donor did not enhance IFN-γ production. In addition, PBMC stimulated with KE4-18•P22, KE4-21•P28, or KE4-21•P29 showed strong cytotoxicity against HLA-A26⁺ cancer cells (KE4), while showing a weak cytotoxicity against HLA-A26⁻ cancer cells (KE3) or not responding to an HLA-A26⁺ PHA-blast.

Thus, tumor antigens shown in Table 8 and peptides derived therefrom can induce and/or activate a tumor-specific CTL in an HLA-A26-stricted manner in PBMC of a cancer patient, so that they would be applicable to use in the specific immunotherapy for HLA-A26⁺ cancer patients.

Polypeptide and Peptide

In the present specification, “a polypeptide” means a long chain peptide of arbitrary peptides comprising two or more amino acids bound to each other by a peptide bond or by a modified peptide bond. For example, a protein is included in the definition of polypeptide herein. Moreover, a short chain peptide sometimes called an oligopeptide or an oligomer is simply called “a peptide” herein. Amino acid sequence may be given both in one-letter symbols and three-letter symbols hereafter.

A polypeptide according the present invention is a polypeptide encoded by one of the above genes obtained from human colon cancer cell line SW620 or human esophageal cancer cell line KE4, preferably a polypeptide consisting of an amino acid sequence described in any one of those of SEQ ID NO:214 to SEQ ID NO:288, SEQ ID NO:356, SEQ ID NO:357, and SEQ ID NO:385 to SEQ ID NO:387 in the sequence listing. A polypeptide consisting of an amino acid sequence of SEQ ID NO:214, SEQ ID NO:228, SEQ ID NO:236, SEQ ID NO:261, or SEQ ID NO:269 is identical to a polypeptide already disclosed in SEREX database. Moreover, a polypeptide consisting of the amino acid sequence of SEQ ID NO:387 is identical to a polypeptide disclosed in GenBank.

These polypeptides can be used for tumor antigens that induce and/or activate CTL. For example, a polypeptide consisting of an amino acid sequence described in any one of those of SEQ ID NO:214 to SEQ ID NO:288, SEQ ID NO:356, and SEQ ID NO:357 in the sequence listing is recognized by an HLA-A2-restricted antigen-specific CTL and, hence, can be used for a tumor antigen that induces and/or activates the CTL. Moreover, a polypeptide consisting of an amino acid sequence described in any one of those from SEQ ID NO:385 to SEQ ID NO:387 is recognized by an HLA-A26-restricted antigen-specific CTL and can be used for a tumor antigen that induces and/or activates the CTL. In addition, these polypeptides can be used for materials to specify a tumor antigen epitope and to obtain a tumor antigen peptide.

Tumor antigen peptides according to the present invention can be obtained by selecting ones that are recognized by CTL and/or induce CTL, from peptides designed based on amino acid sequences of the above polypeptides. The selection can be carried out as shown in the Examples described hereinafter, for example, by culturing a peptide-pulsed antigen-presenting cell together with an HLA-A2-restricted or HLA-A26-restricted CTL such as OK-CTLd or KE4-CTL, followed by measuring IFN-γ production from the activated CTL as an index to select ones. A peptide according to the present invention may be the peptide having a property of a tumor antigen epitope presented on the surface of an antigen-presenting cell through binding to HLA-A2 or HLA-A26 and recognized by CTL. The peptide consists of at least about 5 or more, preferably about 7 or more, and more preferably 9 to 10 amino acid residues. Particularly preferably, the peptide is one consisting of an amino acid sequence described in any one of those of SEQ ID NO:1 to SEQ ID NO:213, SEQ ID NO:358 to SEQ ID NO:381, and SEQ ID NO:388 to SEQ ID NO:408 in the sequence listing. These peptides can be used as a tumor antigen peptide for activating and/or inducing CTL. For example, a peptide consisting of an amino acid sequence described in any one of those of SEQ ID NO:1 to SEQ ID NO:213 and SEQ ID NO:358 to SEQ ID NO:381 in the sequence listing is recognized by an HLA-A2-restricted antigen-specific CTL, and hence can be used as a tumor antigen peptide for activating and/or inducing the CTL. Moreover, a peptide consisting of an amino acid sequence described in any one of those from SEQ ID NO:388 to SEQ ID NO:408 in the sequence listing is recognized by an HLA-A26-restricted antigen-specific CTL, and hence can be used as a tumor antigen peptide for activating and/or inducing the CTL.

For inducing and/or activating CTL, one of the above-described polypeptide or peptide may be used or they may be used in combination of two or more. CTL is a cell population consisting of several kinds of peptide-specific CTL, so that it is recommended to use these peptides preferably in combination of two or more.

A polypeptide or peptide, which has one or several amino acid(s) with a mutation such as deletion, substitution, addition, or insertion introduced into the polypeptide or peptide specified as above, and is recognized by or is capable of inducing at least the HLA-A2-restricted or HLA-A26-restricted CTL, is also included within the scope of the present invention. A polypeptide or peptide having such mutation(s) can be a naturally existing polypeptide or peptide, or can be a polypeptide or peptide in which such mutation(s) is introduced. A means for introducing mutations such as a deletion, substitution, addition, or insertion is well-known and, for example, Ulmer's technique (Science, 219:666, 1983) can be employed. When introducing such mutation, in view of preventing a change of the fundamental properties (such as the physical properties, activity, or immunological activity) of the polypeotide or peptide, mutual substitution among, for example, amino acids having similar properties (polar amino acids, non-polar amino acids, hydrophobic amino acids, hydrophilic amino acids, positively charged amino acids, negatively charged amino acids, aromatic amino acids, and so on) can be carried out. In addition, some modification, such as modification of the constitutive amino group or carboxyl group, can be made on these polypeptides or peptides to such an extent that there is no notable change of their function.

Polynucleotide

A polynucleotide according to the present invention is one encoding the above polypeptide or peptide that is recognized by CTL and/or induces CTL in an HLA-A2-restricted or HLA-A26-restricted manner, or a complementary strand thereof. It is preferably a polynucleotide encoding a peptide consisting of an amino acid sequence described in any one of those of SEQ ID NO:1 to SEQ ID NO:213, SEQ ID NO:358 to SEQ ID NO:381, and SEQ ID NO:388 to SEQ ID NO:408, or encoding a polypeptide consisting of an amino acid sequence described in any one of those of SEQ ID NO:214 to SEQ ID NO:288, SEQ ID NO:356, SEQ ID NO:357, and SEQ ID NO:385 to SEQ ID NO:387 in the sequence listing, or a complementary strand thereof. Exemplified is a polynucleotide consisting of a nucleotide sequence described in any one of those of SEQ ID NO:289 to SEQ ID NO:355 and SEQ ID NO:382 to SEQ ID NO:384 in the sequence listing, or its complementary strand. Among these polynucleotides, a polynucleotide consisting of a nucleotide sequence described in any one of those of SEQ ID NO:289, SEQ ID NO:299, and SEQ ID NO:332 is identical to those that have already disclosed in SEREX database.

A polynucleotide according to the present invention may consist of at least about 15 or more, preferably about 21 to 30 or more nucleotides, wherein the nucleotides correspond to a region encoding a tumor antigen epitope in the amino acid sequence of the polypeptides according to the present invention, or a complementary strand thereof. Selection of such a useful polynucleotide and determination of the nucleotide sequence thereof can be carried out, for example, by employing well-known protein expression systems to confirm the ability of the expressed peptide or polypeptide to induce and/or activate CTL.

The above polynucleotide is recognized by CTL in an HLA-A2-restricted or HLA-A26-restricted manner when expressed in a cell having HLA-A2 or HLA-A26, and hence can induce and/or activate CTL. For example, a polynucleotide consisting of a nucleotide sequence described in any one of those from SEQ ID NO:289 to SEQ ID NO:355 in the sequence listing can be recognized by an HLA-A2-restricted antigen-specific CTL and/or can induce the CTL when expressed in a cell having HLA-A2. In addition, a polynucleotide consisting of a nucleotide sequence described in any one of those from SEQ ID NO:382 to SEQ ID NO:384 can be recognized by an HLA-A26-restricted antigen-specific CTL and/or can induce the CTL when expressed in a cell having HLA-A26.

Moreover, a polynucleotide that hybridizes to the above-described polynucleotide under stringent conditions is also included in the scope of the present invention. Preferably such is a polynucleotide that a polypeptide or peptide encoded thereby is recognized by CTL in an HLA-A2-restricted or HLA-A26-restricted manner and/or induces the CTL. In the case where the polynucleotide molecule is a DNA molecule, “a DNA molecule that hybridizes to a DNA molecule under stringent conditions” can be obtained, for example, by the method described in “Molecular Cloning: A Laboratory Manual (edited by Sambrook et al, Cold spring Harbor Laboratory Press, New York, 1989.)” “To hybridize under stringent conditions” herein means that a positive hybridizing signal is still observed even under the condition in which, for example, hybridization is carried out in a solution containing 6×SSC, 0.5% SDS, and 50% formamide at 42° C. and then, washing is carried out in a solution containing 0.1×SSC and 0.5% SDS at 68° C. A polynucleotide that hybridizes to the above polynucleotide under stringent conditions, which encodes a polypeptide or peptide that is recognized by CTL in an HLA-A2-restricted or HLA-A26-restricted manner and/or induces CTL, can be selected, for example, by employing well-known protein expression systems to confirm the ability of the expressed polypeptide or peptide to induce and/or activate CTL. The selection can be carried out as shown in the Examples described hereinafter, for example, by culturing a peptide-pulsed antigen-presenting cell together with an HLA-A2-restricted or HLA-A26-restricted CTL such as OK-CTLd or KE4-CTL and so on, followed by measuring IFN-γ production from the activated CTL for use as an index to select ones.

The above-described polynucleotide may have a poly(A) structure in its 3′-terminal. The number of poly(A) does not have any influence on the site encoding the amino acid acting as a tumor antigen, so that the number of poly(A) of the polynucleotide is not limited.

All of the above-described polynucleotides provide genetic information useful for producing a polypeptide or a peptide according to the present invention or can be also utilized as a reagent and a standard of a nucleic acid.

Recombinant Vector

A recombinant vector can be obtained by inserting the above-described polynucleotide into an adequate DNA vector. The DNA vector used is properly selected in accordance with the kind of host and the purpose of use. The DNA vector may be a naturally existing one and also may be one that lacks a part of its DNA other than that necessary for replication. For example, vectors can be exemplified as those derived from a chromosome, an episome, and a virus, for example, vectors derived from a bacterial plasmid, derived from a bacteriophage, derived from a transposon, derived from an enzyme episome, derived from an inserting element, and derived from an enzyme chromosome element, for example, vectors derived from a virus such as baculovirus, papovavirus, SV40, vacciniavirus, adenovirus, fowlpox virus, pseudorabies virus, and retrovirus, and vectors prepared by combination of them, for example, vectors derived from the genetic element of the plasmid and the bacteriophage, for example, a cosmid and a phagemid. Further, an expression vector and a cloning vector etc. can be used in accordance with the desired purpose.

The recombinant vector, which comprises the constitutional elements of the desired gene sequence and a gene sequence possessing information relating to replication and regulation, such as a promoter, a ribosome-binding site, a terminator, a signal sequence, an enhancer, and so on, can be prepared by combining them using well-known methods. As a method for inserting the polynucleotide according to the present invention into the above-described DNA vector, well-known methods can be employed. For example, a method can be used, wherein an appropriate restriction enzyme is chosen for treating a DNA to cleave it at a specific site, and then, the DNA is mixed with the DNA used as a vector treated in the same way, followed by ligating with a ligase. A desired recombinant vector can also be obtained by ligating an adequate linker to the desired polynucleotide followed by inserting the resultant molecule into a multi-cloning site of a vector suitable for a purpose.

(Transformant)

The DNA vector in which the above-described polynucleotide has been inserted can be used to obtain a transformant by transforming a well-known host such as Escherichia coli, yeast, Bacillus subtillis, an insect cell, or a mammalian cell therewith by well-known methods. In the case of carrying out the transformation, a more preferable system is exemplified by the method for integrating the gene in the chromosome, in view of achieving stability of the gene. However, an autonomous replication system using a plasmid can be conveniently used. Introduction of the DNA vector into the host cell can be carried out by standard methods such that described in “Molecular Cloning: A Laboratory Manual” (ed. by Sambrook et al., Cold Spring Harbor Laboratory Press, Cold Spring Harbor, N.Y., 1989.) Concretely, calcium phosphate transfection, DEAE-dextran-mediated transfection, microinjection, cation lipid-mediated transfection, electroporation, transduction, scrape loading, ballistic introduction and infection can be employed.

Using an expression vector as a DNA vector for the above-described transformant, a polypeptide or a peptide according to the present invention canbe provided. A transformant, transformed with a DNA expression vector comprising the above-described polynucleotide, is cultured under well-known culture conditions suitable for each host. Culturing may be conducted by using indicators, such as a function of the polypeptide or a peptide according to the present invention that is expressed by the transformant, particularly at least the activity to induce and/or activate CTL, or the peptide or the amount of the peptide produced in the host or outside of the host. Subculturing or batch culturing may be also carried out using an amount of the transformant in the culture as an indicator.

Production of the Polypeptide or Peptide

A polypeptide or a peptide according to the present invention can be produced by a genetic engineering technique as above by using the vector or the transformant described above. Moreover, it can also be produced by a general method known in peptide chemistry. For example, “Peptide Synthesis (Maruzen) 1975” and “Peptide Synthesis, Interscience, New York, 1996” are exemplified. However, any widely known method can be used.

A polypeptide or peptide according to the present invention can be purified and collected by a method, such as gel filtration chromatography, ion column chromatography, affinity chromatography, and the like, in combination, or by fractionation means on the basis of a difference in solubility using ammonium sulfate, alcohol, and the like, using CTL-inducing and/or activating ability of the polypeptide or the peptide as an indicator. More preferably used is a method, wherein the polypeptide or the peptides are specifically adsorbed and collected by using polyclonal antibodies or monoclonal antibodies, which are prepared against the polypeptide or the peptides based on the information of their amino acid sequences.

Antibody

An antibody according to the present invention is prepared by using the above-described polypeptide or peptide as an antigen. An antigen may be the above-described polypeptide or peptide itself, or its fragment that is composed of at least 5, more preferably at least 8 to 10 amino acids. In order toprepare the antibody specific to the above-described polypeptide or peptide, a region consisting of the amino acid sequence intrinsic to the above-described polypeptide or peptide is desirably used. The amino acid sequence is not necessarily homologous to the amino acid sequence of the polypeptide or the peptide, but is preferably a site exposed to the outside of a stereo-structure of the polypeptide or the peptide. In such a case, it is sufficient that the amino acid sequence of the exposed site is consecutive in the exposed site, even if it may be discrete in its primary structure. The antibody is not limited as long as it binds or recognizes the polypeptide or the peptide immunologically. The presence or absence of the binding or the recognition can be determined by a well-known antigen-antibody binding reaction.

In order to produce an antibody, a well-known method for antibody production can be employed. For example, the antibody is obtained by administration of the polypeptide or peptide according to the present invention to an animal in the presence or absence of an adjuvant with or without linking such to a carrier so as to induce humoral immunity and/or cell-mediated immunity. Any carrier can be used as long as it is not harmful to the host and is capable of enhancing immunogenicity. For example, cellulose, a polymerized amino acid, albumin, keyholelimpet hemocyanine (KLH), and the like are exemplified. An adjuvant can be exemplified by Freund's complete adjuvant (FCA), Freund's incomplete adjuvant (FIA), Ribi (MPL), Ribi (TDM), Ribi (MPL+TDM), Bordetella pertussis vaccine, muramyl-dipeptide (MDP), aluminum adjuvant (ALUM), and a combination thereof. As an animal used for immunization, a mouse, rat, rabbit, goat, horse, and so on, is preferably used.

A polyclonal antibody can be obtained from serum of an animal subjected to the above-described immunological means by a well-known method for collecting antibodies. A preferable means is exemplified by immunoaffinity chromatography.

A monoclonal antibody can be produced by collecting antibody-producing cells (for example, a lymphocyte derived from a spleen or a lymph node) from the animal subjected to the above-described immunological method, followed by introducing a well-known transformation technique with a permanently proliferating cell (for example, myeloma strain such as P3X63Ag8 cells.) For example, the antibody-producing cells are fused with the permanent proliferating cells by a well-known method to prepare hybridomas. Then, the hybridomas are subjected to cloning, followed by selecting ones producing the antibody that recognizes specifically the above-described polypeptide or peptide to collect the antibody from a culture solution of the hybridoma.

The polyclonal antibody or the monoclonal antibody thus obtained, which recognizes and binds to the above-described polypeptide or peptide, can be utilized as an antibody for purification, a reagent, a labeling marker and so on.

Method for Screening for a Compound

The above-described polypeptide or peptide, the polynucleotide encoding the same or the complementary strand thereof, the cell transformed based on the information concerning the amino acid sequence and nucleotide sequence, or the antibody immunologically recognizing the same, provide an effective means for screening a substance capable of enhancing the recognition of the same by CTL, when using them solely or in combination with each other. The screening method can be constructed utilizing a well-known screening system. For example, there can be a system in which HLA-A2 restricted CTL (such as OK-CTL) is cultured together with an antigen-presenting cell that is pulsed with a tumor antigen peptide followed by measuring an amount of IFN-γ produced from the CTL for using as an indicator to detect the recognition of the peptide by the CTL. Addition of a test substance to the system allows one to select a substance capable of enhancing the recognition of the above-described polypeptide or peptide by HLA-A2 restricted CTL. Using HLA-A26-restricted CTL (such as KE4-CTL) in place of HLA-A2-restricted CTL in the system, allows one to select a substance capable of enhancing the recognition of the above polypeptide or peptide by HLA-A26-restricted CTL. This system describes one screening method, but the screening method according to the present invention is not limited thereto.

A compound obtained by the above-described screening method is also part of the present invention. The compound may be a compound that interacts with the above-described polypeptide or peptide recognized by CTL in an HLA-A2 restricted manner, for example, a polypeptide or peptide consisting of the amino acid sequence of any one of those of SEQ ID NO:1 to SEQ ID NO:213 and SEQ ID NO:358 to SEQ ID NO:381, and/or HLA-A2 molecule, and enhances the recognition of the polypeptide or the peptide by HLA-A2 restricted CTL. Further, it may be a compound that interacts with the polynucleotide encoding the above-described polypeptide or peptide recognized by CTL in an HLA-A2 restricted manner, and enhances the expression of the same.

A compound may be also provided that interacts with the above-described polypeptide or peptide recognized by CTL in an HLA-A26 restricted manner, for example, a polypeptide consisting the amino acid sequence of any one of those of SEQ ID NO:385 to SEQ ID NO:387 or a peptide consisting the amino acid sequence of any one of those of SEQ ID NO:388 to SEQ ID NO:408, and/or HLA-A26 molecule, and enhances the recognition of the polypeptide or the peptide by HLA-A26 restricted CTL. Further, a compound may be provided that enhances the expression of the polynucleotide encoding the above-described polypeptide or peptide.

The compound thus selected can be used in a pharmaceutical composition by further selecting ones in consideration of the balance of biological usefulness and toxicity.

Pharmaceutical Composition

A polypeptide or peptide provided in the present invention can be used as a tumor antigen or tumor antigen peptide to induce and/or activate a tumor-specific CTL. In other words, a medicament consisting of one or more selected from the above polypeptides and peptides, a method for inducing CTL comprising using one or more selected from the above polypeptides and peptides, and an inducer of CTL comprising one or more selected from the above polypeptides and peptides, are also included in the scope of the present invention.

Moreover, the present invention provides a pharmaceutical composition that contains at least one selected from a group consisting of the above-described polypeptide and peptide, the polynucleotide encoding the polypeptide and peptide, or the complementary strand thereof, the recombinant vector prepared based on the information of these amino acid sequence and nucleotide sequence, the cell transformed by the recombinant vector, the antibody immunologically recognizing the polypeptide or peptide, the compound enhancing the recognition of CTL through interaction with the polypeptide and peptide, and the compound enhancing the expression of the polynucleotide through interaction therewith, when using them solely or in combination with each other. The pharmaceutical composition is useful for treating cancers. The above polypeptide or peptide obtained from colon cancer cell line SW620 is useful, for example, for treating colon cancer. The above polypeptide or peptide obtained from esophageal cancer cell line KE4 is useful, for example, for treating esophageal cancer, oral squamous cell cancer, and renal cancer. The above polypeptide or peptide is also applicable to various cancers such as gynecological cancers (e.g., breast cancer, endometrial cancer, cervical cancer, ovarian cancer) and prostate cancer. HLA-A2 allele is found in approximately 40% of Japanese, approximately 53% of Chinese, approximately 49% of North Caucasians, approximately 38% of South Caucasians, and approximately 23% of African Blacks, while HLA-A26 allele is found in approximately 22% of Japanese, approximately 16% of Korean, and approximately 8% of North Caucasians [Imanishi, T. et al., HLA 1991, 1: 1065-1220, 1992, Oxford, Oxford Scientific publications]. Therefore, the above medicament and pharmaceutical composition would be effective for many patients.

Concretely, for example, a medicament consisting of one or more selected from the group consisting of the above polypeptide and peptide, and a pharmaceutical composition consisting of one or more selected from the group consisting of the above polypeptide and peptide, can be used as so-called “cancer vaccine”. “Cancer vaccine”, herein, means a medicament capable of damaging a cancer cell selectively by inducing and/or enhancing a specific immunological response to a cancer cell. The amount of administration thereof can be determined depending on the degree of the recognition of the polypeptide or the peptide by CTL. Generally, as an active ingredient, the amount ranges 0.01 mg to 100 mg/day/adult human body, preferably 0.1 mg to 10 mg/day/adult human body. Administration can be carried out according to well-known methods for administrating a peptide for medical use, preferably subcutaneously, intravenously, or intramuscularly. When administering, in order to induce and/or enhance an immunological response, a polypeptide and/or peptide according to the present invention can be used in the presence or absence of an appropriate adjuvant with or without linking such to a carrier. Any carriers can be used as long as it is not harmful to the human body and can enhance the immunogenicity, such as cellulose, a polymerized amino acid, albumin, and so on. An adjuvant generally used for the peptide vaccination can be used for the present invention, such as Freund's incomplete adjuvant (FIA), aluminum adjuvant (ALUM), Bordetella pertussis vaccine, mineral oil, and so on. Moreover, the formulation can be appropriately selected from well-known methods for formulating peptides.

Alternately, an effective action of a cancer vaccine can also be obtained by inducing and/or activating CTL in a mononuclear cell fraction that is collected from the peripheral blood of a patient, followed by returning the fraction, back into the blood of the patient. Culture conditions, such as the concentration of mononuclear cells and the concentration of the polypeptide or the peptide according to the present invention, and the culture period, can be determined using simple experiments. Further, a substance, such as interleukin 2 (IL-2) having an ability to induce the growth of lymphocytes may be added to the culture.

In the case of using the above-described polypeptide or the peptide as a cancer vaccine, using even only one polypeptide or one peptide is effective as a cancer vaccine. However, plural kinds of the above-described polypeptide or peptide can be used in combination. Recently, it has been reported that multi-peptide based immunotherapy is effective [Miyagi, Y. et al., Clin. Cancer Res., 7: 3950-3962, 2001] [Lee, P. et al., J. Clin. Oncol., 19: 3836-3847, 2001] [Nestle, F. O. et al., Nat. Med., 4: 328-332, 1998]. CTL of a cancer patient is a cell population consisting of plural kinds of peptide-specific CTL, so that using plural kinds of polypeptides or peptides as an anti-cancer vaccine may give a higher effect than using only one kind. Especially in a cancer patient having both HLA-A26 and HLA-A2, a higher effect may be sometimes obtained when using the combination of a polypeptide or peptide recognized by an HLA-A26-restricted CTL and a polypeptide or peptide recognized by an HLA-A2-restricted CTL.

A polynucleotide encoding the above-described polypeptide or peptide, preferably the peptide, or its complementary strand can be used for the gene therapy of a cancer. Both a method in which one of these polynucleotides is carried on a vector and the obtained vector is directly transduced into a body for expression and a method in which a cell is collected from a human followed by introducing the polynucleotide into the cell in vitro for expression, can be utilized. Retrovirus, adenovirus, vaccinia virus, and so on, are known as a vector. Recommended is retrovirus. With respect to these viruses, replication-deficient viruses are used. Although the amount of administration is variable depending on the degree of the recognition of the polypeptide or peptide by CTL, it is generally 0.1 μg to 100 mg/day/adult human body, preferably 1 μg to 50 mg/day/human adult body, as an amount of DNA encoding a polypeptide or peptide according to the present invention. This amount is administered once every several days to several months.

Assay Method and Reagent

The above-described polypeptide or peptide, the polynucleotide encoding the polypeptide or peptide or its complementary strand, and the antibody that immunologically recognizes the polypeptide or the peptide, can be used independently for a diagnostic marker or a reagent, etc. When used as a reagent, the reagent can contain buffer(s), salt(s), stabilizing agent(s), and/or antiseptic(s.) Moreover, the present invention also provides a reagent kit comprising one container or more filled with one of them or more. For the preparation thereof, it is sufficient to use a well-known means for their preparation according to each of peptide, polypeptide, polynucleotide, antibody, and so on.

These reagents and reagent kits can be used for a screening method according to the present invention or can be used for the quantitative or qualitative measurement of a polypeptide or peptide according to the present invention or a polynucleotide encoding any one of these. A method for the measurement can be constructed by using well-known method(s) for those skilled in the art. Available methods include radioimmunoassay, competitive binding assay, Western blot analysis, ELISA, and so on. In addition, nucleic acids can be detected or quantified at an RNA level by using, for example, amplification, PCR, RT-PCR, RNase protection, Northern blotting and other hybridization methods.

The sample subjected to measurement is exemplified by the cells derived from an individual human body, blood, urine, saliva, spinal fluid, tissue biopsy, or autopsy material, and the like. The nucleic acid subjected to measurement is obtained from the each sample described above by a well-known method for nucleic acid preparation. For the nucleic acid, genomic DNA can be directly used for detection, or it may be enzymatically amplified by using PCR or any other amplification method before the analysis. RNA or cDNA may be similarly used.

In comparing with a normal genotype, a deletion or insertion of a gene can be detected by measuring the change in size of the above amplification product. Hybridizing the amplified product with the labeled DNA encoding the above-described polypeptide can permit identifying point mutations.

Approximately 87% of the above genes obtained from colon cancer cell line SW620 are more highly (about twice) expressed in colon cancers compared with normal colon tissues. For example, it was revealed that clone 10, clone 30, and clone 108, are selectively expressed in colon cancer cells (see Example 6.)

Although mRNA of KE4-cl.18 obtained from esophageal cancer cell line KE4 was expressed in all of the tested cancer cells and non-malignant proliferating cells (PHA blastoid T cell and VA13), it was not expressed in normal tissues other than testis, muscle, and peripheral blood mononuclear cells. Moreover, although mRNA of KE4-cl.21 was expressed in all of the tested cancer cells and non-malignant proliferating cells as well as normal tissue, the expression level in the normal tissue was low. It was revealed that these mRNAs are over-expressed especially in cancer cells in the head and the neck, and non-malignant proliferating cells.

Therefore, detecting mutation of, reduction of, and increase in the polypeptide or peptide according to the present invention and the DNA encoding anyone of these, by the above-described measuring method, makes it possible to diagnose diseases, to which the polypeptide or peptide or DNA is associated, such as epithelial cell cancer and adenocarcinoma, and so on.

### EXAMPLES

Although the present invention is described concretely by Examples below, the present invention is not limited to these examples.

### Example 1

Establishment of HLA-A2-restricted CTL

An HLA-A2-restricted tumor-specific CTL was established from TIL of a colon cancer patient (HLA-A0207/3101, -B46/51, -Cw1) according to the method described in the literature [Int. J. Cancer, 81: 459-466, 1999] [J. Immunol., 163: 4997-5004, 1999]. TIL obtained from the colon tumor patient was cultured for a long period up to 50 days or longer with the addition of 100 U/ml of recombinant human interleukin 2 (IL-2.) Every 7 days, a portion of TIL activated by IL-2 was collected and cultured together with various kinds of tumor cells or normal cells to evaluate its CTL activity by measuring IFN-γ production and by measuring ⁵¹Cr released from the cancer cells (Gomi, S. et al., J. Immunol., 163:4997-5004, 1999.) IFN-γ was measured by an enzyme-linked immunosorbent assay (ELISA.) At day 58 after the start of the culture, CTL showing HLA-A2-restricted and tumor-specific CTL activity (which may be called as OK-CTL), was obtained. OK-CTL obtained is a cell population in which 80% of the cells have a phenotype of CD3⁺CD4⁻CD8⁺ and 20% of the cells have a phenotype of CD3⁺CD4⁺CD8⁻. The above-established CTLs were frozen in small portions and preserved until use.

OK-CTL recognized HLA-A0201⁺ pancreatic adenocarcinoma cells, Panc-1, HLA-A0201⁺ colon adenocarcinoma cells SW620, HLA-A0206⁺ squamous cell carcinoma (SCC) cells KE3, and HLA-A0207⁺ oral SCC cells CA9-22, resulting in production of IFN-γ, and showed adequate cytotoxicity against these cells. However, it did not show any cytotoxicity against HLA-A2⁻ tumor cells, Epstein-Barr virus-transformed autologous B cells (EBV-B), and phytohemagglutinin (PHA) blast autologous T cells. Moreover, OK-CTL lysed all of the tested HLA-A2⁺ tumor cells (HLA-A0201⁺ breast adenocarcinoma R27, primary hepatocellular carcinoma HAK-2, melanoma SK-MEL-5, astrocytoma SF126, HLA-A0206⁺ pulmonary adenocarcinoma PC9, HLA-A0207⁺ pulmonary adenocarcinoma 1-87, and cervical SCC cells OMC-4.) The activity was inhibited by anti-HLA class I monoclonal antibody, anti-CD8 monoclonal antibody or anti-HLA-A2 monoclonal antibody (monoclonal antibody may be sometimes abbreviated as “mAb” hereafter), but not inhibited by other mAbs. Thus, it was confirmed that OK-CTL is an HLA-A2-restricted CTL.

The phenotype of an HLA class I allele of the above-described tumor cells has already been reported [Gomi, S. et al., J. Immunol., 163: 4997-5004, 1999]. Moreover, the serotype of HLA class I of the above patient was determined with peripheral blood mononuclear cells (PBMC) according to the conventional method. In addition, the HLA-A2 subtype was determined by the sequence-specific oligonucleotide probe method and direct DNA sequencing. The surface phenotype of OK-CTL was analyzed by the direct immunofluorescence method with fluorescein isothiocyanate (FITC)-labeled anti-CD3 mAb, anti-CD4 mAb, anti-CD8 mAb (Nichirei), oranti-TCRαβ mAb (WT31, Becton Dickinson.) Moreover, the antibodies used for testing the HLA-restriction and specificity of OK-CTL were anti-HLA class I mAb (W6/32, IgG2a), anti-HLA-A2 mAb (BB7.2, IgG2b), anti-CD8 mAb (Nu-Ts/c, IgG2a), anti-HLA class II mAb (H-DR-1, IgG2a), and anti-CD4 (Nu-Th/i, IgG1) mAb. Anti-CD13 mAb (MCS-2, IgG1) and anti-CD14 mAb (JML-H14, IgG2a) were used as isotype-matched control mAbs.

### Example 2

Identification of cDNA Clone Encoding HLA-A2-restricted Tumor Antigen

A gene encoding a tumor antigen recognized by OK-CTLd that is one of sublines of OK-CTL was isolated and identified from a cDNA library of human colon cancer cell line SW620 according to the known method [Shichijo, S. et al., J. Exp. Med., 187: 277-288, 1998]. Cryopreserved OK-CTLd was cultured in the presence of 100 U/ml of IL-2 for 2 weeks or more to use in the experiments.

First of all, a poly (A) ⁺RNA of SW620 cells was converted to cDNA, and ligated with a SalI adapter so as to insert into the expression vector pCMV-SPORT-2 (Invitrogen Corp.). Moreover, cDNA of HLA-A0207 was obtained by the reverse transcription-polymerase chain reaction (RT-PCR) and cloned into the eukaryotic cell expression vector pCR3 (Invitrogen Corp.).

A cDNA library of SW620 cells was pooled so that each pool contained 100 clones, and 200 ng of the cDNA pooled in each well and 200 ng of the HLA-A0207 cDNA were mixed in 100 ul of lipofectoamine (Invitrogen Corp.)/Opti-MEM (Invitrogen Corp.) 1:200 mixture for 30 min. 50 μl of the obtained mixture was added to a simian kidney cell line COS-7 (1×10⁴), and incubated for 6 h for co-transduction. Then, RPMI-1640 culture medium containing 10% FCS was added to and culturing was carried out for 2 days, followed by addition of OK-CTLd (2×10⁵) to each well. After a further 18 h incubation, 100 μl of the supernatant was collected, and IFN-γ production was measured by ELISA. In this case, as a negative control, using COS-7 cells into which the gene had not been transfected as a target, IFN-γ production from OK-CTLd was examined and the value of IFN-γ produced was subtracted as a background from that of each measurement.

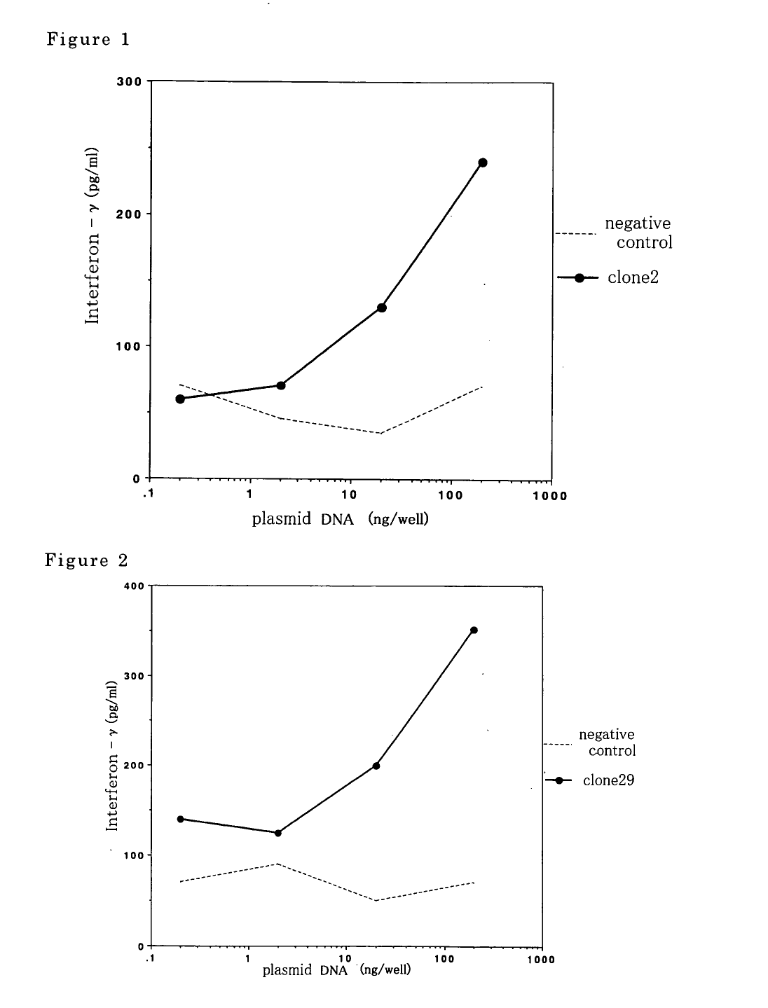

A pool of cDNA library was screened with the criteria in which cDNA capable of enhancing IFN-γ production from OK-CTLd was judged as a positive one having CTL-activating ability. After confirming reproducibility of CTL-activating ability of the pool, individual clones were taken up from the pool with which CTL-activating ability was observed, and screening was further carried out to select positive clones having CTL-activating ability from an independent pool. Dose dependency in OK-CTLd activation of clones obtained was confirmed, resulting to give finally 65 clones. On the other hand, with respect to COS-7 cells into which only expression vector pCMV-SPORT-2 was co-transduced together with HLA-A0207, IFN-γ production from OK-CTLd was not enhanced. As representative data, results with clone 2, clone 29, and clone 40 are illustrated in FIG. 1, FIG. 2, and FIG. 3, respectively. Similar results were obtained also with respect to other clones.

The nucleotide sequences of the cDNA clones obtained were determined by the dideoxynucleotide sequencing method using an ABI PRISM™ 377 DNA Sequencer (Perkin-Elmer, Inc.) and a DNA sequencing kit (Perkin-Elmer, Inc.). In addition, amino acid sequences encoding the cDNA clones were deduced from the nucleotide sequences (see Tables 1 to 6 above.) It was revealed that clone 76 (SEQ ID NO:346) is identical to clone 78 (SEQ ID NO:295) among the clones obtained.

With respect to each of the genes obtained, a homology search was carried out using GenBank/DDBJ, and the results obtained are summarized in Tables 1 to 6 above. Moreover, a homology search using the SEREX database (http://www-ludwig.unil.ch/SEREX.html) revealed that amino acid sequences encoded by clone 12, clone 65, clone 81, clone 86, and clone 100 1422 are identical to ones encoded by known genes ID456, ID629, ID1226, ID163, and ID116, respectively, which are disclosed in SEREX database. Moreover, the nucleotide sequences of clone 12, clone 82, and clone 86 are identical to those of ID456, ID1197, and ID163, respectively. In addition, it was found that clone 32, clone 41, clone 74, and clone 87 are partially homologous to ID1072, ID1233, ID979, and ID567, respectively.

### Example 3

Identification of HLA-A2-restricted Tumor Antigen Peptide

In order to obtain tumor-antigen peptides from tumor antigens encoded by genes obtained in Example 2, an HLA-A2 binding motif (a specific sequence) was searched for amino acid sequences encoded by the genes using a home page (http://bimas.dcrt.nih.gov/molbio/hla_bind/) of Bioinformatics & Molecular Analysis Section (BIMAS.) The type of HLA of OK-CTLd is HLA-A0207, so that motif search was carried out for peptides capable of binding to HLA-A0207 molecule. HLA-A0207 molecule is different from HLA-A0201 molecule only in the 123^{rd} amino acid residue in the amino acid sequence, which is Y in the sequence of the former and is C in the sequence of the latter. This amino acid residue is not located in an α-helix or β-sheet that is related with peptide binding, but is located in a coil region of the secondary structure, so that the difference of this amino acid residue does not affect the peptide binding. Therefore, a peptide that is suitable for an HLA-A0201-binding motif [Rammensee, H.-G. et al., Immunogenetics, 41: 178-228, 1995] would bind to HLA-A0207. Then, peptides capable of binding to an HLA-A0207 molecule were designed based on a result obtained by searching an HLA-A0201-binding motif, and various peptides of 9-mer and 10-mer (whose purity is 70% or higher) were synthesized by a well-known method. Moreover, with respect to clone 5, clone 23, clone 26, clone 35, clone 65, clone 81, and clone 100, peptides were designed and synthesized based on the amino acid sequences encoded by genes highly homologous to each gene.

In order to examine the recognition of the above-described peptide by CTL, T2 cells were used as an antigen-presenting cell. T2 cells express the HLA-A2 molecule on a cell surface without binding to a peptide, because of deficiency of transporters associated with antigen processing (TAP) [Cancer Res., 54: 1071-1076, 1994.] First of all, each synthesized peptide (10 μM) described above was incubated together with T2 cells under 5% CO₂/95% air at 37° C. for 3 h to make the peptide bind to HLA-A2 expressed on the cell surface. T2 cells thus pulsed with a peptide was used as a target cell (T). In addition, OK-CTLd obtained in Example 1 was used as an effector cell (E). Target cells (1×10⁴) and effector cells (2×10⁴) were mixed (E/T ratio=2) and incubated for 18 h, followed by collecting 100 μl of supernatant to measure IFN-γ by ELISA. At this time, the amount of IFN-γ produced from OK-CTLd against T2 cells that had not been pulsed with the peptide was subtracted as a background from each measurement value. As a result, 213 peptides, each consisting of amino acid sequence of SEQ ID NO:1 to 213 in the sequence listing, were recognized by OK-CTLd and enhanced IFN-γ production from OK-CTLd in a dose dependent manner of each peptide. The obtained 213 peptides were P1 to P4 (SEQ ID NO:1 to SEQ ID NO:4) derived from clone 12; P26, P28, P34, P38 and P39 (SEQ ID NO:5 to SEQ ID NO:9) derived from clone 40; P45 (SEQ ID NO:10) derived from clone 43; P46, P48, P53 and P54 (SEQ ID NO:11 to SEQ ID NO:14) derived from clone 2; P57 and P64 (SEQ ID NO:15 and SEQ ID NO:16) derived from clone 11; P71, P73 and P75 (SEQ ID NO:17 to SEQ ID NO:19) derived from clone 8; P77, P79, P80 and P81 (SEQ ID NO:20 to SEQ ID NO:23) derived from clone 78; P85, P86 and P88 (SEQ ID NO:24 to SEQ ID NO:26) derived from clone 67; P90-P93 and P95 (SEQ ID NO:27 to SEQ ID NO:31) derived from clone 9; P98, P100, P102, P103 and P104 (SEQ ID NO:32 to SEQ ID NO:36) derived from clone 95; P110 to P112 (SEQ ID NO:37 to SEQ ID NO:39) derived from clone 82; P118, P120 and P121 (SEQ ID NO:40 to SEQ ID NO:42) derived from clone 103; P122 and P131 (SEQ ID NO:43 and SEQ ID NO:44) derived from clone 4; P132 and P133 (SEQ ID NO:45 and SEQ ID NO:46) derived from clone 14; P138 to P141, P144, P146 , P149 , P150 and P152 (SEQ ID NO:47 to SEQ ID NO:55) derived from clone 65; P153 (SEQ ID NO:56) derived from clone 69; P175 to P177, P179 and P180 (SEQ ID NO:57 to SEQ ID NO:61) derived from clone 83; P181 to P184 (SEQ ID NO:62 to SEQ ID NO:65) derived from clone 84; P193 to P195 and P197 (SEQ ID NO:66 to SEQ ID NO:69) derived from clone 32; P201, P214, P217 and P226 (SEQ ID NO:70 to SEQ ID NO:73) derived from clone 21; P229, P230 and P236 (SEQ ID NO:74 to SEQ ID NO:76) derived from clone 33; P242 and P243 (SEQ ID NO:77 and SEQ ID NO:78) derived from clone 68; P247, P249, P251, P255 and P259 (SEQ ID NO:79 to SEQ ID NO:83) derived from clone 100; P260 and P262 to P265 (SEQ ID NO:84 to SEQ ID NO:88) derived from clone 73; P268, P272 and P273 (SEQ ID NO:89 to SEQ ID NO:91) derived from clone 27; P277 to P280 and P282 (SEQ ID NO:92 to SEQ ID NO:96) derived from clone 26; P294 , P295 and P297 (SEQ ID NO:97 to SEQ ID NO:99) derived from clone 56; P300, P302 and P303 (SEQ ID NO:100 to SEQ ID NO:102) derived from clone 5; P312, P317 and P319 (SEQ ID NO:103 to SEQ ID NO:105) derived from clone 10; P321 to P323 and P330 (SEQ ID NO:106 to SEQ ID NO:109) derived from clone 22; P333, P340, P342, P344, P347 and P348 (SEQ ID NO:110 to SEQ ID NO:115) derived from clone 30; P354, P358 and P360 (SEQ ID NO:116 to SEQ ID NO:118) derived from clone 88; P362, P363, P367, P369, P379 and P380 (SEQ ID NO:119 to SEQ ID NO:124) derived from clone 45; P384 and P385 (SEQ ID NO:125 and SEQ ID NO:126) derived from clone 58; P388 and P389 (SEQ ID NO:127 and SEQ ID NO:128) derived from clone 18; P390 and P391 (SEQ ID NO:129 and SEQ ID NO:130) derived from clone 87; P393 and P394 (SEQ ID NO:131 and SEQ ID NO:132) derived from clone 24; P400 and P401 (SEQ ID NO:133 and SEQ ID NO:134) derived from clone 46; P406, P409, P410, P412, P418, P421 and P424 (SEQ ID NO:135 to SEQ ID NO:141) derived from clone 110; P426, P429, P434, P436, P440 and P441 (SEQ ID NO:142 to SEQ ID NO:147) derived from clone 20; P443 and P445 to P447 (SEQ ID NO:148 to SEQ ID NO:151) derived from clone 6; P448 to P450, P453, P455-P457 and P461 (SEQ ID NO:152 to SEQ ID NO:159) derived from clone 108; P470 (SEQ ID NO:160) derived from clone 23; P479, P480, P482 to P485, P489, P492 and P495 (SEQ ID NO:161 to SEQ ID NO:169) derived from clone 86; P497 (SEQ ID NO:170) derived from clone 89; P511 (SEQ ID NO:171) derived from clone 92; P515, P522 to P524, P528, P529, P532, P533, P536, P525 and P526 (SEQ ID NO:172 to SEQ ID NO:182) derived from clone 85; P538 and P539 (SEQ ID NO:183 and SEQ ID NO:184) derived from clone 3; P542, P543, P546 and P548 (SEQ ID NO:185 to SEQ ID NO:188) derived from clone 29; P550, P555 and P556 (SEQ ID NO:189 to SEQ ID NO:191) derived from clone 35; P570 and P579 (SEQ ID NO:192 and SEQ ID NO:193) derived from clone 81; P583, P587, P589, P590, P596 and P597 (SEQ ID NO:194 to SEQ ID NO:199) derived from clone 114; P603 (SEQ ID NO:200) derived from clone 19; P614, P616, P617 and P623 (SEQ ID NO:201 to SEQ ID NO:204) derived from clone 74n; P634 and P636 (SEQ ID NO:205 and SEQ ID NO:206) derived from clone 41; P642 and P644 (SEQ ID NO:207 and SEQ ID NO:208) derived from clone 79; P649 and P654 (SEQ ID NO:209 and SEQ ID NO:210) derived from clone 36; and P656, P658 and P661 (SEQ ID NO:211 to SEQ ID NO:213) derived from clone 76. Among these, P77 (SEQ ID NO:20) and P79 (SEQ ID NO:21) derived from clone 78 were identical to P656 (SEQ ID NO:211) and P658 (SEQ ID NO:212) derived from clone 76, respectively.

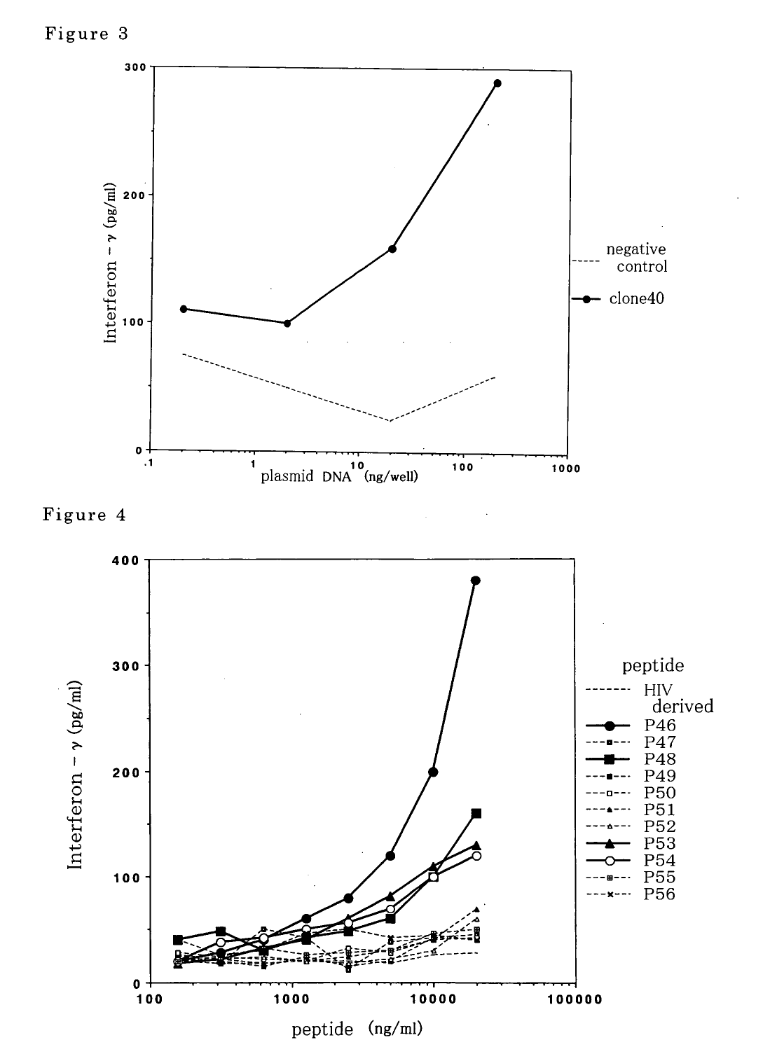

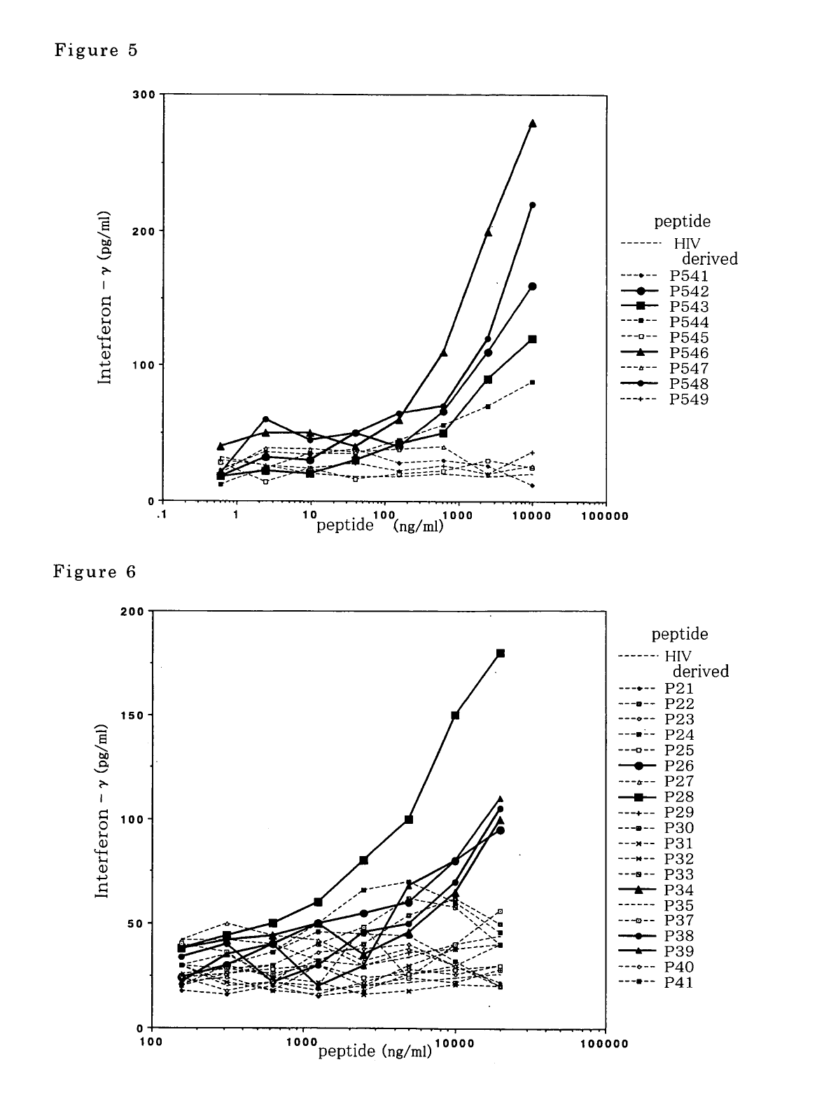

Results with peptides derived from clone 2, clone 29, and clone 40 are representatively illustrated in FIGS. 4, 5, and 6, respectively. In the figures, peptides recognized by OK-CTLd and judged as ones capable of activating OK-CTLd are shown with bold (solid) lines. A peptide (SLYNTVATL) (negative control) derived from HIV (human immunodeficiency virus) that is suitable for an HLA-A2-binding motif was not recognized by OK-CTLd.

### Example 4

Specificity of TCR that Recognizes a Peptide

Tumor antigen peptides obtained in Example 3 are presented on the cell surface by HLA-A2 and recognized by a peptide-specific OK-CTLd clone together with HLA molecule through a T cell receptor (TCR) expressed on each clone. It is reported that the diversity of TCR is larger than 10¹⁷, and the type of HLA molecule recognized and the peptide recognized are totally depending on the TCR.

Therefore, using 480 sublines (which, hereinafter, may be called “OK-CTLd sublines”) established using OK-CTLd as a parent strain, the specificity of each subline in the recognition of a tumor cell and the above peptide was examined. OK-CTLd sublines were established from a parent strain (OK-CTLd) by incubating the strain at a density of 1,000 cells/well according to the method described in the literature [Ito, M. et al., Cancer Res., 61: 2038-2046, 2001]. The recognition by OK-CTLd sublines of tumor cells (SW620, and COS-7 as a negative control) and of T2 cell pulsed with the above-described peptide was examined in a manner similar to Example 3 using IFN-γ production from the subline as an index.

As a result, one hundred one of the OK-CTLd sublines (21%) reacted with SW620 and produced a significantly higher level of IFN-γ than the amount produced in the reaction against COS-7. Results of twenty-seven sublines that showed good growth are summarized in Table 9. The symbol of “−” in Table 9 denotes that the amount of IFN-γ produced is 20 pg/ml or less. These sublines react with at least one peptide (eight sublines), maximally eight peptides (subline #70), with the average number of peptides that reacted with each subline being 2.6. Fifteen peptides shown in Table 9 were recognized by at least one subline (three peptides), maximally twenty-one sublines (P440 derived from clone 20, SEQ ID NO:146), with the average number of sublines that responded to each peptide being 4.7. Moreover, these sublines did not recognize T2 cells that were not pulsed with a peptide (which is designated as “NC” in Table 9) or T2 cells that were pulsed with a peptide derived from HIV suitable for an HLA-A2-binding motif (which is designated as “HIV” in Table 9.)

TABLE 9
IFN-γ produced from OK-CTLd-9 subline (pg/ml)

| | | CTL subline | | | | | | | | | | | | | |
|---|---|---|---|---|---|---|---|---|---|---|---|---|---|---|---|
| peptide | cDNA | 9 | 13 | 24 | 28 | 32 | 33 | 34 | 38 | 39 | 40 | 43 | 46 | 48 | 49 |
| P1 | #012 | — | — | — | — | — | — | — | 100 | — | — | — | — | — | — |
| P46 | #002 | — | — | — | — | — | — | 174 | — | — | — | — | — | — | — |
| P77 | #076 | — | — | — | — | — | — | — | 216 | — | — | — | — | — | — |
| P102 | #095 | — | — | — | — | — | — | 156 | — | — | — | 148 | — | — | — |
| P180 | #083 | — | — | — | — | — | — | — | 276 | — | — | — | — | — | — |
| P264 | #073 | 392 | — | — | 242 | — | — | 340 | 850 | 162 | — | — | 238 | — | — |
| P295 | #056 | 108 | — | — | — | — | — | — | — | — | — | 128 | — | — | — |
| P319 | #010 | — | — | — | — | — | — | 194 | — | — | — | — | 116 | — | — |
| P424 | #110 | — | — | — | — | — | — | 166 | 104 | — | — | — | — | — | — |
| P440 | #020 | 122 | 102 | 224 | 536 | — | 118 | 498 | 492 | 650 | 202 | — | 150 | 156 | — |
| P449 | #108 | — | — | — | — | — | — | — | — | — | — | — | — | — | — |
| P480 | #086 | — | — | — | — | — | — | — | — | — | — | — | — | — | — |
| P533 | #085 | — | — | 386 | — | — | — | — | — | — | — | — | — | — | — |
| P546 | #029 | — | — | — | — | — | — | — | — | — | — | — | — | — | 156 |
| P590 | #114 | 636 | 153 | 328 | 360 | 106 | — | — | — | — | 220 | — | 270 | — | — |
| HIV | | — | — | — | — | — | — | — | — | — | — | — | — | — | — |
| NC | | — | — | — | — | — | — | — | — | — | — | — | — | — | — |
| COS-7 | | — | — | — | 394 | — | — | 172 | 402 | 272 | 294 | — | 296 | 188 | 306 |
| SW620 | | 100 | 168 | 154 | 554 | 184 | 294 | 334 | 568 | 314 | 360 | 284 | 404 | 324 | 432 |

| | | CTL subline | | | | | | | | | | | | |
|---|---|---|---|---|---|---|---|---|---|---|---|---|---|---|
| peptide | cDNA | 51 | 54 | 56 | 61 | 65 | 66 | 68 | 70 | 72 | 81 | 89 | 91 | 96 |
| P1 | #012 | — | — | — | — | — | — | 122 | — | — | — | — | — | — |
| P46 | #002 | — | — | — | — | — | — | — | — | — | — | — | — | — |
| P77 | #076 | — | — | — | — | — | — | — | 116 | — | — | — | — | — |
| P102 | #095 | — | — | — | — | — | — | — | — | 134 | — | — | — | — |
| P180 | #083 | — | — | — | — | — | — | — | 146 | — | — | — | — | 105 |
| P264 | #073 | 310 | — | — | — | — | 112 | — | 354 | — | — | — | — | — |
| P295 | #056 | — | — | — | — | — | 106 | — | 280 | 104 | — | — | 170 | — |
| P319 | #010 | — | — | — | — | — | — | — | 340 | — | — | 112 | — | — |
| P424 | #110 | — | — | — | — | — | — | — | — | — | — | — | — | 130 |
| P440 | #020 | — | 180 | 162 | 180 | 132 | 192 | 306 | 198 | — | 130 | 123 | 196 | — |
| P449 | #108 | — | — | — | — | — | — | — | — | 120 | — | — | — | — |
| P480 | #086 | — | — | — | — | — | — | — | 134 | — | — | — | — | — |
| P533 | #085 | — | — | — | — | — | 110 | — | 344 | — | — | 102 | — | — |
| P546 | #029 | — | — | 230 | — | — | — | — | — | — | — | — | — | — |
| P590 | #114 | — | — | — | 106 | — | — | — | — | — | — | — | — | — |
| HIV | | — | — | — | — | — | — | — | — | — | — | — | — | — |
| NC | | — | — | — | — | — | — | — | — | — | — | — | — | — |
| COS-7 | | 552 | — | 364 | 336 | — | 234 | 246 | 216 | — | 134 | — | 354 | — |
| SW620 | | 782 | 180 | 482 | 434 | 280 | 368 | 420 | 308 | 198 | 230 | 152 | 420 | 248 |

OK-CTLd sublines were pooled to use as effector cells, and cytotoxicity against target cells was examined by the ⁵¹Cr release assay. As a target cell, a T2 cell was used that was pulsed with P1 (SEQ ID NO:1) derived from clone 12, P424 (SEQ ID NO:141) derived from clone 110, P440 (SEQ ID NO:146) derived from clone 20, P449 (SEQ ID NO:153) derived from clone 108, or P590 (SEQ ID NO:197) derived from clone 114, and labeled with ⁵¹Cr. Effector cells and target cells were mixed each other at an E/T ratio of 10, 20, and 40, and then incubated for 6 h, followed by measuring a radioactivity of ⁵¹Cr released in the culture supernatant. As a result, as shown in FIG. 7a, the OK-CTLd subline showed cytotoxicity at a significant level against target cells pulsed with a peptide, but did not show any cytotoxicity against target cells pulsed with a peptide derived from HIV suitable for an HLA-A2-binding motif (negative control.)

Moreover, when TCR Vβ of these OK-CTLd sublines that recognize each peptide was analyzed, it was revealed that the type differs in each subline (FIG. 7b.) TCR Vβ usages were analyzed according to the known method [Ito, M. et al., Cancer Res., 61: 2038-2046, 2001].

These results revealed that the difference of TCR Vβ makes CTL showing non-identical specificity against each peptide. On the other hand, all the tested TCR Vβ are expressed on OK-PBMC (a PBMC of a colon cancer patient who provided TIL used for establishing OK-CTL) or on PBMC of a healthy donor at a similar level.

Thus, it was revealed that even CTL established from a tumor-infiltrating lymphocyte of one patient are not homogeneous but are a cell population consisting of plural kinds of peptide-specific CTL and recognize various antigens and peptides.

### Example 5

Analysis of HLA-A2-restricted CTL in PBMC of a Tumor Patient

Mononuclear cell fractions were prepared from the peripheral blood of three HLA-A2⁺ cancer patients (i.e., a colon cancer patient OK who provided TIL used for establishing OK-CTL, another colon cancer patient TT, and a metastatic melanoma patient) and cultured in the presence of IL-2 for 30 days. Then, their reactivity to the cDNA clone encoding the tumor antigen, which was obtained in Example 2, was examined in a manner similar to Example 2. In addition, their reactivity to the peptide encoded by the cDNA clone was examined in a manner similar to Example 3. At this time, analysis was also carried out in a similar manner using GK-CTL established from a tumor infiltrating lymphocyte of a pulmonary cancer tissue [Gomi, S. et al., J. Immunol., 163: 4994-5004, 1999.]

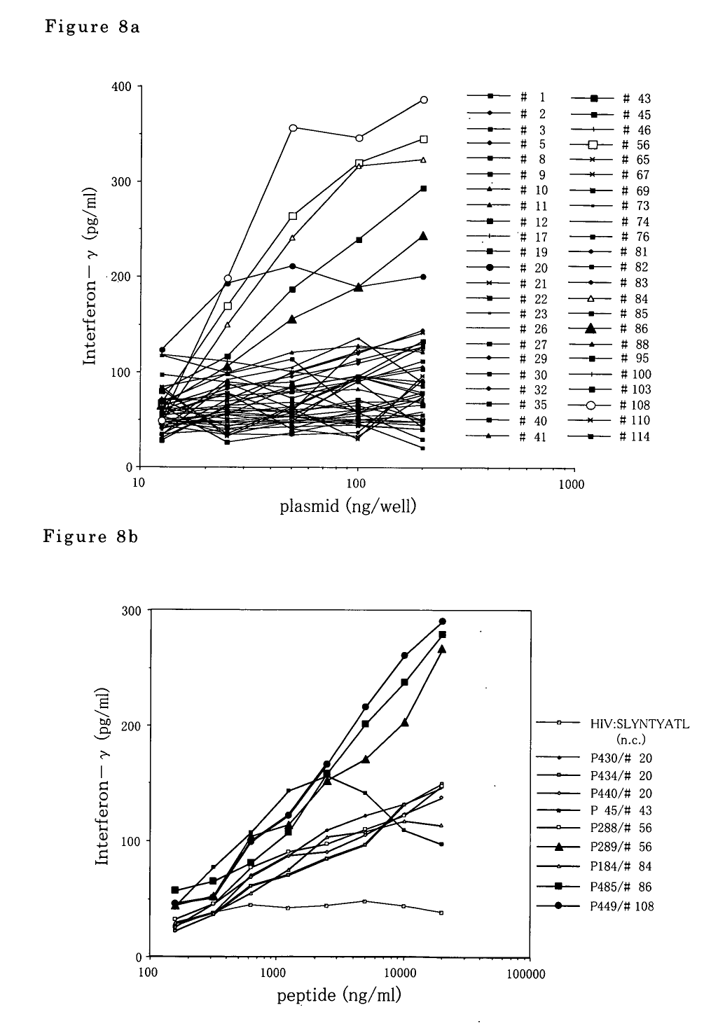

Products of six genes (clone 20, clone 43, clone 56, clone 84, clone 86, and clone 108) were recognized by PBMC derived from colon cancer patient OK (which, hereinafter, may be called OK-PBMC) (FIG. 8a), and products of four genes (i.e., clone 20, clone 84, clone 86, and clone 108) were recognized by activated PBMC (which, hereinafter, may be called TT-PBMC) derived from colon cancer patient TT, and as a result IFN-γ production from each PBMC was enhanced.

Moreover, HLA-A2-binding peptides derived from these six gene products, namely three peptides, one peptide, two peptides, one peptide, one peptide, and one peptide encoded by clone 20, clone 43, clone 56, clone 84, clone 86, and clone 108, respectively, enhanced IFN-γ production from OK-PBMC in a dose dependent manner of each peptide (FIG. 8b.) These peptides are P430, P434, and P440 (SEQ ID NO:360, 144, and 146) derived from clone 20; P45 (SEQ ID NO:10) derived from clone 43; P288 (SEQ ID NO:358) and P289 (SEQ ID NO:359) derived from clone 56; P184 (SEQ ID NO:65) derived from clone 84; P485 (SEQ ID NO:166) derived from clone 86; and P449 (SEQ ID NO:153) derived from clone 108. OK-PBMC strongly reacted to P289 (SEQ ID NO:359) derived from clone 56, P449 (SEQ ID NO:153) derived from clone 108, and P485 (SEQ ID NO:166) derived from clone 86, and produced a greater amount of IFN-γ than that in the reaction to other peptides.

In addition, the IFN-γ production was inhibited bya monoclonal antibody against HLA class I antigen, CD8, or HLA-A2, but not inhibited by an anti-HLA class II antigen mAb, an anti-CD4 mAb, or an anti-CD14 mAb that was used as an isotype-matched control antibody. This revealed that the recognition of these peptides by the activated PBMC described above is restricted by HLA-A2 that is HLA-class I antigen and that PBMC activated by recognizing the peptide is CD8-positive CTL.

TT-PBMC recognized P431 (SEQ ID NO:361) derived from clone 20, P184 (SEQ ID NO:65) derived from clone 84, P486 (SEQ ID NO:363) derived from clone 86, and P463 (SEQ ID NO:362) derived from clone 108.

Among the above peptides, P288 (SEQ ID NO:358) and P289 (SEQ ID NO:359) derived from clone 56, P430 (SEQ ID NO:360) and P431 (SEQ ID NO:361) derived from clone 20, P463 (SEQ ID NO:362) derived from clone 108, and P486 (SEQ ID NO:363) derived from clone 86 did not strongly enhance IFN-γ production from OK-CTLd in the examination of Example 3, but, as described above, were recognized by a PBMC of a colon cancer patient in an HLA-A2-restricted manner and strongly enhanced IFN-γ production from the same.

Thus, it was revealed that at least products of the above genes (hi.e., clone 20, clone 43, clone 56, clone 84, clone 86, and clone 108) and peptides derived from the gene products, namely, the above twelve peptides were recognized by PBMC of a colon cancer patient and can activate CTL in an HLA-2-restricted manner. On the other hand, the above gene products were not recognized by GK-CTL or activated PBMC derived from a metastatic melanoma patient or PBMC derived from a healthy donor.

Thus, it was revealed that a CTL precursor recognizing the above tumor antigens and peptides derived therefrom in an HLA-A2-restricted manner is present in PBMC of a colon cancer patient. Therefore, these tumor antigens and peptides derived therefrom would be able to induce and/or activate a tumor-specific CTL in an HLA-A2-restricted manner in this patient.

### Example 6

Expression Level of Gene

Expression level of genes obtained in Example 2 in colon cancer (four colon cancer cell lines and two primary tumors) and normal colon tissue (two colon epithelial tissues) were examined by the SAGE analysis [Velculescu, V. E., et al. Science, 270: 484-487, 1995.] Comparison of expression level of cDNA between colon cancer and normal colon tissue revealed that approximately 87% of the above genes are more highly expressed in colon cancer than in normal colon tissue with the ratio being about twice. On the contrary, clone 11, clone 35, clone 84, clone 95, and clone 110 were more highly expressed in normal colon tissue.

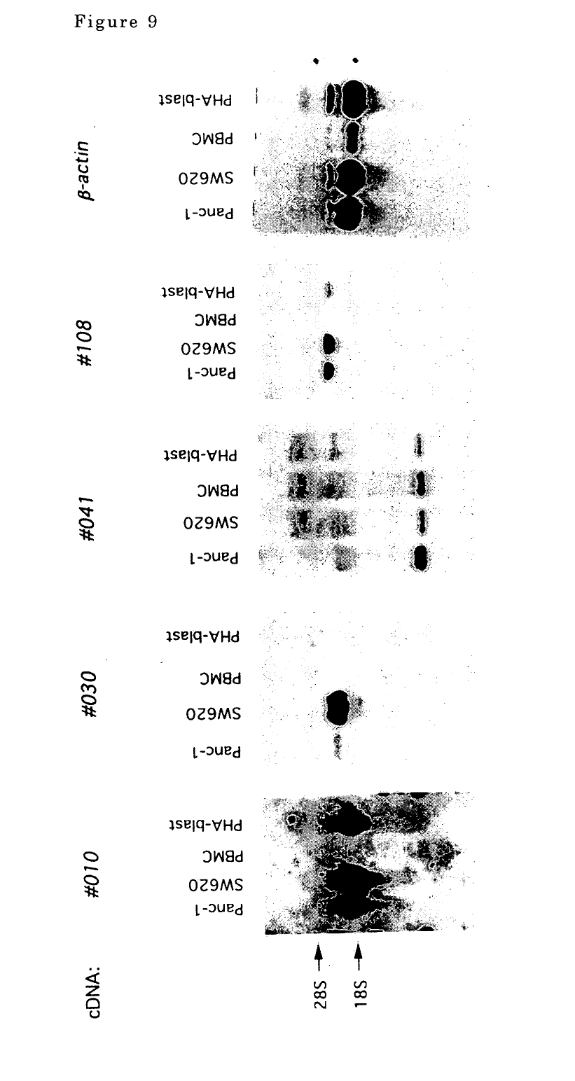

In addition, expression of clone 10, clone 30, clone 41, and clone 108 in various cells was examined by the northern blot analysis. The northern blot analysis was carried out using total RNA (10 μg/lane) extracted from PBMC or PHA-blast derived from a healthy donor, SW620 (colon cancer cell line), or Panc-1 (pancreatic cancer cell line), according to the method described in the paper [Nishizaka, S. et al., Cancer Res., 60: 4830-4837, 2000.] The following ³²P-labelled probe cDNAs were used: a 593 bp cDNA fragment obtained by digesting clone 10 with AccI so as to contain the 2,028^{th} to the 2,260^{th} nucleotide; a 1,020 bp cDNA fragment obtained by digesting clone 30 with DraI and HincII to contain the 885^{th} to the 1,904^{th} nucleotide; an 812 bp cDNA fragment obtained by digesting clone 41 with AccI so as to contain the 2,709^{th} to the 3,520^{th} nucleotide; and a 1,109 bp cDNA fragment obtained by digesting clone 108 with AvaII so as to contain the 2,146th to the 3,254^{th} nucleotide. Moreover, β-actin mRNA was examined similarly as a control concerning the expression degree. As a result, it was revealed that clone 10, clone 30, and clone 108 are expressed selectively in a colon cancer cell (FIG. 9.)

### Example 7

Isolation/identification of cDNA Clone Encoding an HLA-A2 Tumor Antigen

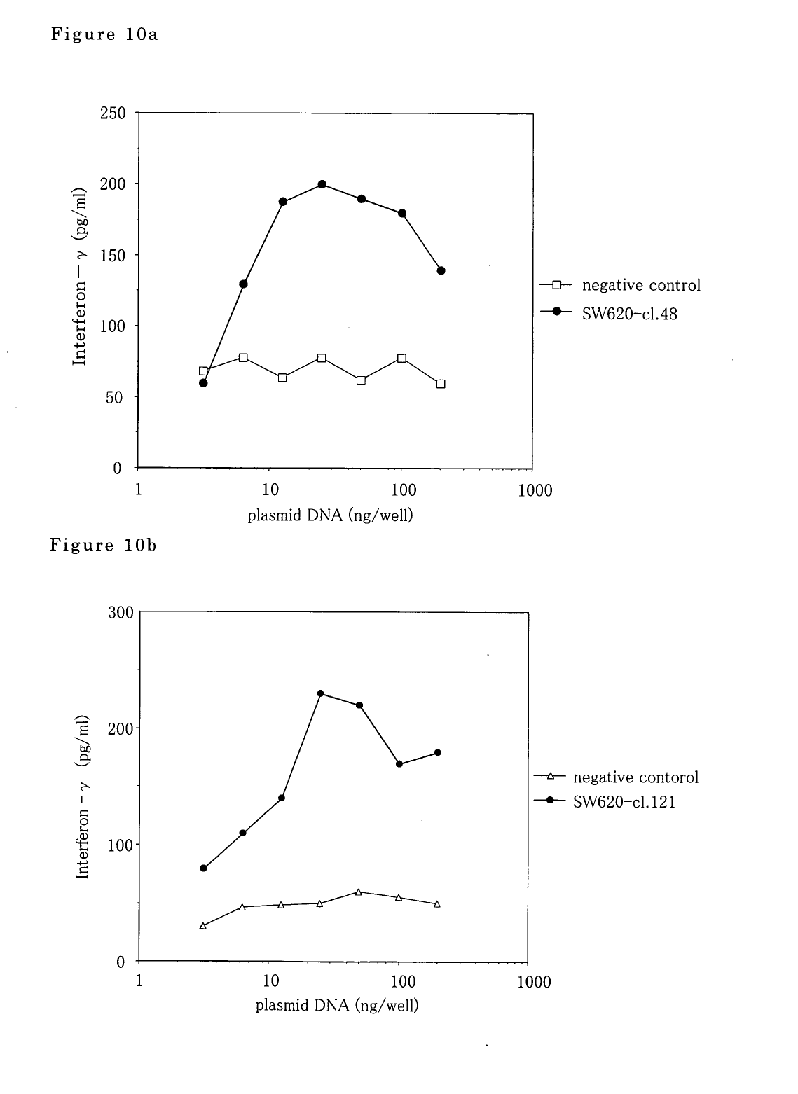

Two new cDNA clones SW620-cl.48 (SEQ ID NO:354) and SW620-cl.121. (SEQ ID NO:355) that are recognized by OK-CTLd were obtained by screening a CDNA library pool of SW620 in a manner similar to Example 2. As illustrated in FIGS. 10a and 10b, each of these cDNA clones was recognized by OK-CTLd in an HLA-A2-restricted manner and in a dose dependent manner of plasmid, and enhanced IFN-γ production from OK-CTLd. On the other hand, in the case where COS-7 cells into which only expression vector pCMV-SPORT-2 was co-transduced together with HLA-A0207, IFN-γ production from OK-CTLd was not enhanced. Thus, two cDNA clones encoding tumor antigens, which are recognized by CTL in an HLA-A2-restricted manner and can activate CTL, were obtained.

Nucleotide sequences of the cDNA clones obtained were determined in a manner similar to Example 2, and an amino acid sequence encoded by each cDNA clone was deduced from each nucleotide sequence. Moreover, a homology search was carried out for GenBank with respect to the nucleotide sequence of each clone obtained. The result is summarized in Table 7 described above.

### Example 8

Identification of an HLA-A2-restricted Tumor Antigen Peptide

Based on amino acid sequences encoded by genes SW620-cl.48 and SW620-cl.121 obtained in Example 7, various peptides of 9-mer and 10-mer suitable for an HLA-A0207 binding motif (a specific sequence) were designed and synthesized by a well-known method in a manner similar to Example 3, and were obtained with a purity of 70% or higher.

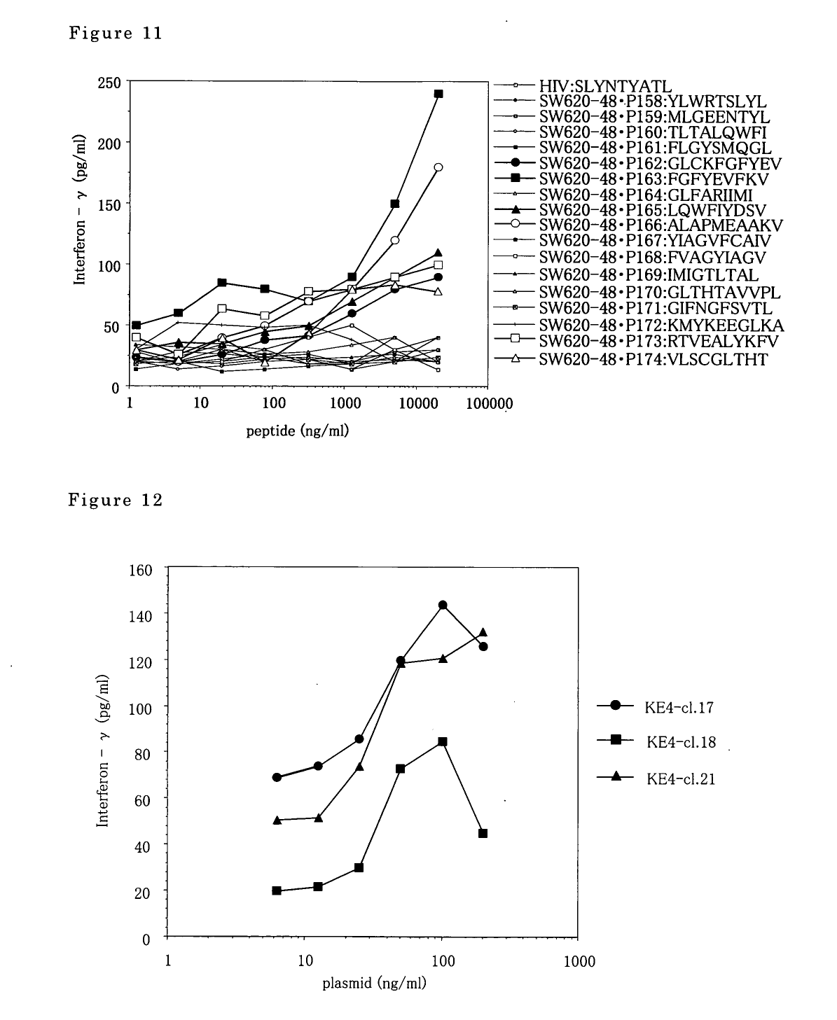

Among the synthesized peptides (1.22 ng/ml to 20 μg/ml), peptides recognized by OK-CTLd in an HLA-A2-restricted manner were selected in a manner similar to Example 3. As a result, eighteen peptides of SEQ ID NO:364 to 381 in the sequence listing were recognized by OK-CTLd in a dose dependent manner and enhanced IFN-γ production from OK-CTLd, while a peptide (SLYNTVATL) derived from HIV that was used in place of the above peptide as a negative control did not enhance IFN-γ production from CTL. The obtained eighteen peptides are SW620-48•P162, SW620-48•P163, SW620-48•P165, SW620-48•P166, SW620-48•P173, and SW620-48•P174 (SEQ ID NO:364 to 369) derived from SW620-cl.48 as well as SW620-121•P665, SW620-121•P666, SW620-121•P667, SW620-121•P668, SW620-121•P669, SW620-121•P676, SW620-121•P677, SW620-121•P678, SW620-121•P679, SW620-121•P685, SW620-121•P686, and SW620-121•P688 (SEQ ID NO:370 to 381) derived from SW620-cl.121. As representative data, FIG. 11 illustrates that six peptides derived from SW620-cl.48 were recognized by OK-CTLd in a dose dependent manner and enhanced IFN-γ production from OK-CTLd.

### Example 9

Establishment of HLA-A26-restricted CTL

HLA-A26-restricted tumor-specific CTL was established from an esophageal cancer patient (HLA-A2601/2402) according to a method described in the literature [Nakao, M., Cancer Res., 55:4248-4252, 1995.] As a result, a cell line showing tumor-specific cytotoxicity in an HLA-A26-restricted manner was obtained and designated as KE4-CTL. The established CTL was cryopreserved in small portions until use.

### Example 10

Isolation/identification of cDNA Clone Encoding an HLA-A26-restricted Tumor Antigen

A gene encoding a tumor antigen recognized by KE4-CTL was isolated/identified from a human esophageal cancer cell line KE4 according to a known method [J. Exp. Med., 187: 277-288, 1998.] First of all, poly (A) ⁺RNA of KE4 cells was converted to cDNA, ligated with a SalI adaptor so as to insert into the expression vector pSV-SPORT-1 (Invitrogen Corp.). Moreover, cDNA of HLA-A2601 was obtained by the reverse transcription-polymerase chain reaction (RT-PCR) and cloned into the eukaryotic cell expression vector pCR3 (Invitrogen Corp.).

A cDNA library of KE4 cells was pooled so that each pool contained 100 clones, and 200 ng of the cDNA pooled in each well and 200 ng of the HLA-A2601cDNA were mixed in 100 μl of lipofectoamine (Invitrogen Corp.)/Opti-MEM (Invitrogen Corp.) 1:200 mixture for 30 min. 50 μl of the obtained mixture was added to VA13 cells (1×10⁴), and incubated for 6 h for co-transduction. Then, RPMI-1640 culture medium containing 10% FCS was added thereto and culturing was carried out for 2 days, followed by addition of KE4-CTL (2×10⁵) to each well. After a further 18 h incubation, 100 μl of the supernatant was collected, and IFN-γ production was measured by ELISA.

A pool of cDNA library was screened with the criteria in which cDNA capable of enhancing IFN-γ production from KE4-CTL was judged as a positive one having CTL-activating ability. After confirming reproducibility of CTL-activating ability of the pool with which the ability was observed, individual clones were taken up from the pool and screening was further carried out to select positive clones having CTL-activating ability from an independent pool. Dose dependency of the obtained clones was confirmed in a similar manner, resulting in giving finally three cDNA clones KE4-cl.17, KE4-cl. 18, and KE4-cl.21. These CDNA clones were recognized by KE4-CTL in an HLA-A26-restricted manner and in a dose dependent manner of plasmid, and enhanced IFN-γ production from KE4-CTL (FIG. 12.) On the other hand, when VA13 cell was used into which only expression vector pSV-SPORT-1 was co-transduced together with HLA-A2601, enhancement of IFN-γ production from KE4-CTL was not observed.

Nucleotide sequences of the cDNA clones obtained were determined in a manner similar to Example 2, and an amino acid sequence encoded by each of the cDNA clones was deduced from each of the nucleotide sequences. Moreover, with respect to the nucleotide sequence of each of the clones obtained, a homology search was carried out from GenBank. The result is summarized in Table 8 described above.

### Example 11

Identification of an HLA-A26-restricted Tumor Antigen Peptide

In order to obtain tumor-antigen peptides from genes KE4-17, KE4-18, and KE4-21 encoding tumor antigens, various peptides of 9-mer and 10-mer were designed and synthesized according to a method described in the literature [J. Exp. Med., 184: 735-740, 1996] based on each amino acid sequence encoded by each gene, and were obtained at a purity of 70% or higher. With respect to gene KE4-17, peptides were designed and synthesized based on amino acid sequences encoded by genes highly homologous to the gene.

After each of the synthesized peptides was pulsed to VA13 cells in which HLA-A2601 was expressed, the resultant cell was cultured together with KE4-CTL, followed by measuring IFN-γ produced from KE4-CTL so as to select the peptides recognized by CTL in an HLA-A26-restricted manner. First of all, HLA-A2601 plasmid at 100 ng/well was added to VA13 cells, and then incubated for two days for gene-transduction, followed by incubating in the presence of each of the synthesized peptides at 1.22 ng/ml to 20 μg/ml under the 5% CO₂/95% air at 37° C. for 4 h so as to make the peptide binding to an HLA-A26 expressed on the cell surface. VA13 cells to which the peptide was pulsed was used as a target cell (T), and KE4-CTL was used as an effector cell (E). Target cells (1×10⁴) and effector cells (2×10⁴) were mixed (E/T ratio=2), and then incubated for 18 h. After incubation, 100 μl of the supernatant was collected to measure IFN-γ by ELISA. At this time, the value of IFN-γ produced from CTL in response to VA13 cells to which a peptide was not pulsed, was subtracted as a background from that of each measurement. This examination was carried out twice for each peptide. In each of the two experiments, cryopreserved KE4-CTL was thawed and cultured using a feeder cell before use. Namely, lots of KE4-CTL used in the two experiments were different from each other.

As a result, twenty-one peptides of SEQ ID NO:388 to 408 in the sequence listing were recognized by KE4-CTL and enhanced IFN-γ production from KE4-CTL. The twenty-one peptides obtained were KE4-17•P12 and KE4-17•P19 (SEQ ID NO:393 and SEQ ID NO:394) derived from KE4-17; KE4-17•P1, KE4-17•P3, KE4-17•P5, KE4-17•P7, and KE4-17•P11 (SEQ ID NO:388 to SEQ ID NO:392) derived from a gene highly homologous to KE4-17; KE4-18•P5, KE4-18•P9, KE4-18•P15, KE4-18•P16, KE4-18•P22, KE4-18•P25, KE4-18•P26, and KE4-18•P27 (SEQ ID NO:395 to SEQ ID NO:402) derived from KE4-18; as well as KE4-21•P28, KE4-21•P29, KE4-21•P38, KE4-21•P39, KE4-21•P40, and KE4-21•P47 (SEQ ID NO:403 to SEQ ID NO:408) derived from KE4-21. Among these, KE4-17•P3 (SEQ ID NO:389), KE4-17•P12 (SEQ ID NO:393), KE4-18•P15 (SEQ ID NO:397), KE4-18•P27 (SEQ ID NO:402), KE4-21•P28 (SEQ ID NO:403), and KE4-21•P40 (SEQ ID NO:407) were recognized by KE4-CTL and enhanced IFN-γ production from KE4-CTL in both of the two experiments. The remaining fifteen peptides were recognized by KE4-CTL and enhanced IFN-γ production from KE4-CTL only in one of the two experiments. The reason why the two experiments gave different results could be explained as follows: CTL is a cell population consisting of various peptide-specific CTL, so that in the case where different feeder cells are used for culturing after being thawed from a frozen state as described above, CTL showing different reactivity to peptides could be obtained.

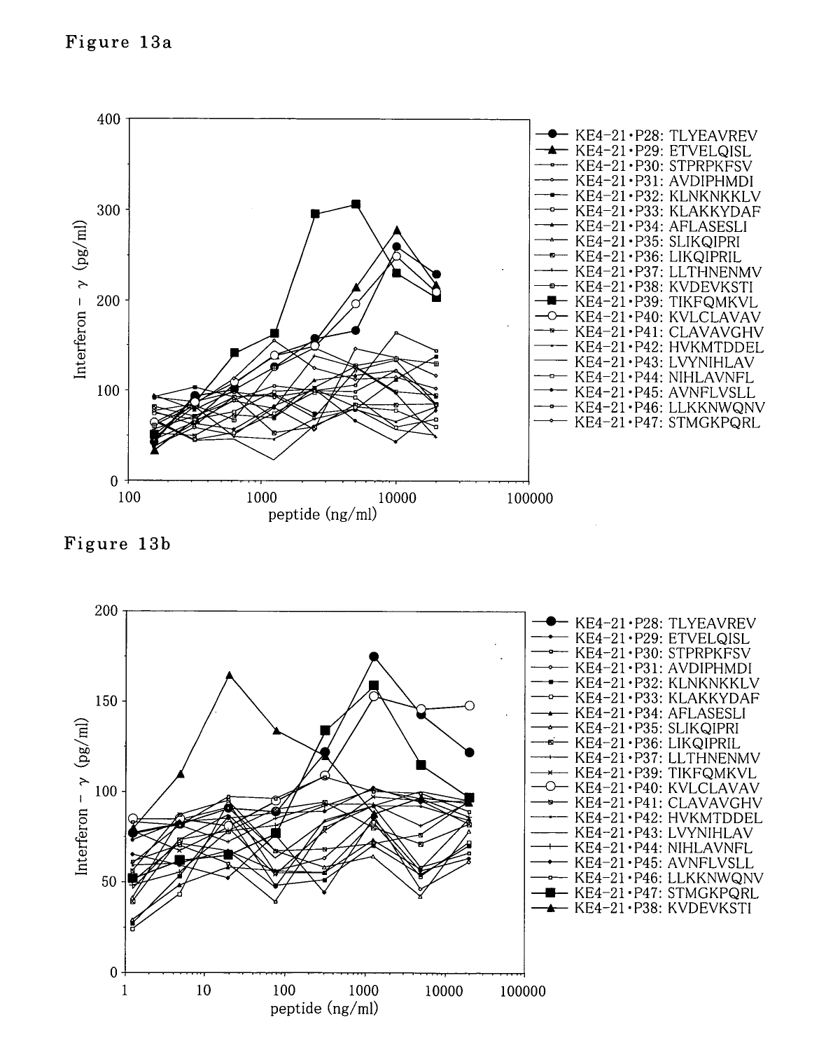

As representative data, FIG. 13 illustrates that peptides derived from KE4-21 enhanced IFN-γ production from KE4-CTL in a dose dependent manner of the peptide. FIGS. 13a and 13b illustrate the results of the two experiments.

### Example 12

Induction of an HLA-A26-restricted CTL in PBMC

PBMC (1×10⁵) obtained from HLA-A26⁺ cancer patients (four cases of oral squamous cell cancer, one case of renal cancer, and one case of pulmonary cancer) and five healthy donors were incubated with a peptide (purity>90%) at 10 μM in the presence of 100 U/ml of IL-2. At day 3, 6, and 9 after starting the culture, a half of the culture medium was replaced with a fresh medium containing a corresponding peptide at 20 μM. After three times of stimulation, added was VA13 cells that were pulsed with a corresponding peptide after being transfected into with HLA-A2601, and incubation was carried out for 18 h, followed by measuring concentration of IFN-γ in the culture supernatant. Statistic analysis was carried out using Two-tailed Student's T-test.

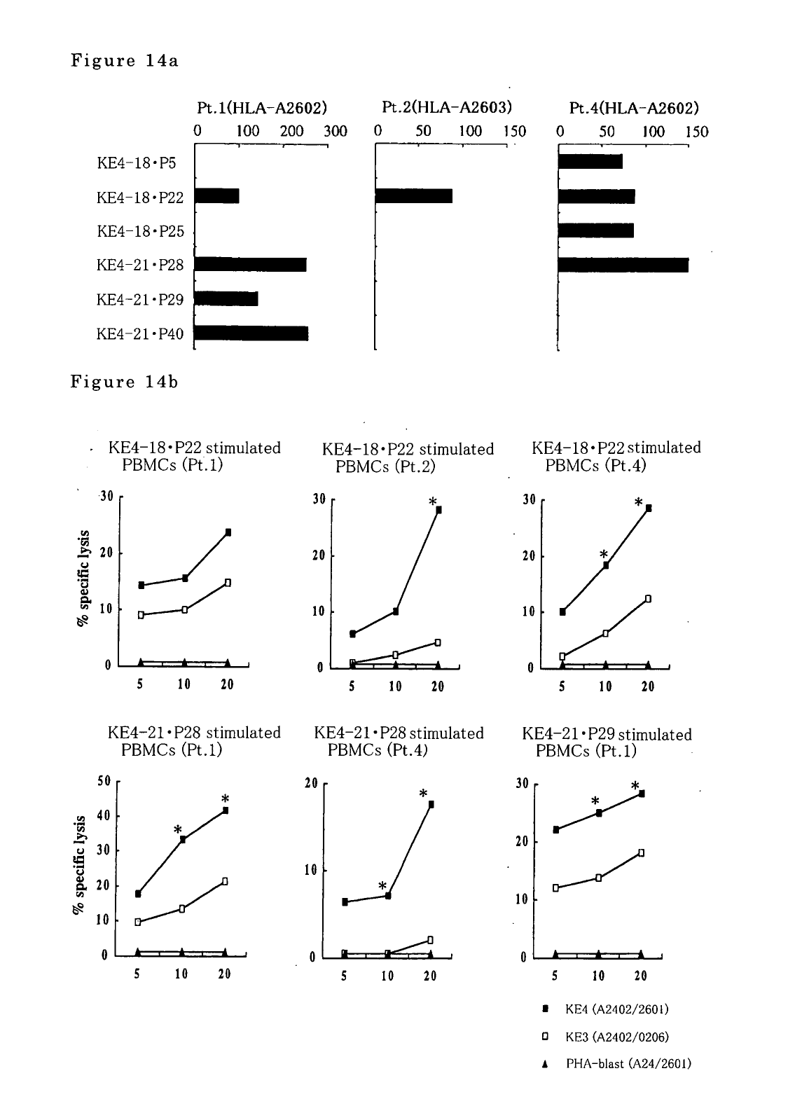

Peptides KE4-18•P5 (SEQ ID NO:395), KE4-18•P22 (SEQ ID NO:399), and KE4-18•P25 (SEQ ID NO:400) derived from KE4-cl.18 as well as peptides KE4-21•P28 (SEQ ID NO:403), KE4-21 P29 (SEQ ID NO:404) and KE4-21•P40 (SEQ ID NO:407) derived from KE4-cl.21 were used for the examination. As a result, four peptides, one peptide and four peptides among the above six peptides were recognized by PBMC of three cancer patients (Pt.1, Pt.2, and Pt.4), respectively, and significantly enhanced IFN-γ production from the PBMC (FIG. 14a.) KE4-18•P22 and KE4-21•P28 were recognized by PBMC of three cancer patients among five cases and by PBMC of two cancer patients among six cases, respectively, and enhanced IFN-γ production from the PBMC. On the other hand, these peptides did not enhance IFN-γ production from PBMC prepared from the rest of the three cancer patients and five healthy donors. These cellular responses were inhibited by an anti-HLA class I mAb and an anti-CD8 mAb, but not inhibited by other antibodies used for the test. These results revealed that a cell that recognizes the above peptide and produces IFN-γ is an HLA class I-restricted CD8-positive T lymphocyte in PBMC.

Cytotoxicity against HLA-A26⁺ or A26⁻ cancer cells was examined using PBMC stimulated with these peptides by carrying out the ⁵¹Cr release test for 6 h at three different E/T ratios. PBMC stimulated with KE4-18•P22, KE4-21•P28, or KE4-21•P29 exhibited strong cytotoxicity against an HLA-A26⁺ cancer cell (KE4), while it exhibited a weak cytotoxicity against an HLA-A26⁻ cancer cell (KE3), and did not react with an HLA-A26⁺ PHA-blast (FIG. 14b.) A significant difference was observed between the cytotoxicity against KE4 cells and that against KE3 cells.

These results revealed that HLA-A26-restricted tumor antigens and peptides derived therefrom according to the present invention can induce and/or activate tumor-specific CTL in an HLA-A26-restricted manner in PBMC of a cancer patient.

### Example 13

With respect to genes KE4-cl.18 and KE4-cl.21 obtained in Example 10, expression of their mRNAs in tissues and various cancer cells was examined by the northern blot analysis. The northern blot analysis was carried out by a method similar to one described in Example 6. As a probe cDNA, ribosomal protein S2 cDNA and L10a cDNA highly homologous to KE4-cl.18 and KE4-cl.21, which were labelled with ³²P, were used. Similarly, β-actin mRNA was examined as a control with respect to the expression degree [Shichijo, S. et al., J. Exp. Med., 187: 277-288, 1998.]

Based on the obtained results, relative expression levels were calculated using the Equation 1 below [Shichijo, S. et al., J. Exp. Med., 187: 277-288, 1998] and summarized in Table 10.

Equation 1
Index=(concentration of S2 or L10a in a specimen/concentration of β-actin in a specimen)×(concentration of β-actin in KE4 cells/concentration of S2 or L10a in KE4 cells)

KE4-cl.18 was expressed in all of the cancer cells tested (i.e., KE4, KE3, Kuma-1, Kuma-3, HSC-2, HSC-3, HSC-4, OSC-20, Ca9-22, QG56, Sq-1, LC99A, LK79, 11-18, LK87, and PC-9) as well as non-malignant proliferating cells (PHA-blast and VA13) but not expressed in normal tissues other than testis, muscle, and peripheral blood mononuclear cells, which are brain, colon, heart, kidney, liver, lung, placenta, small intestine, spleen, and stomach.

Although KE4-cl.21 was expressed in all of the tested cancer cells andnon-malignant proliferating cells as well as normal tissues, the expression level in the normal tissue was low.

It was observed that mRNAs of KE4-cl.18 and KE4-cl.21 are over-expressed especially in cancer cells in the head and neck as well as in non-malignant proliferating cell.

TABLE 10

Relative Index

| Cell lines or Tissues | S2 mRNA | L10a |
|---|---|---|
| Esophageal SCCs | | |
| KE4 (control) | 1.0 | 1.0 |
| KE3 | 1.6 | 0.6 |
| Head and Neck SCCs | | |
| Kuma-1 | 1.6 | 1.2 |
| Kuma-3 | 1.5 | 1.2 |
| Oral SCCs | | |
| HSC-2 | 1.1 | 1.2 |
| HSC-3 | 1.3 | 1.3 |
| HSC-4 | 1.0 | 1.1 |
| OSC-20 | 1.0 | 1.2 |
| Ca9-22 | 1.5 | 1.1 |
| Lung SCCs | | |
| QG56 | 1.0 | 1.0 |
| Sq-1 | 1.6 | 1.0 |
| Lung large cell carcinoma | | |
| LC99A | 1.2 | 2.0 |
| Lung small cell carcinoma | | |
| LK79 | 0.9 | 0.6 |
| Lung adenocarcinomas | | |
| 11-18 | 1.2 | 0.9 |
| LK87 | 0.8 | 1.2 |
| PC-9 | 1.2 | 1.5 |
| Non-malignant proliferationg cells | | |
| PHA-blastoid T cell | 1.5 | 1.1 |
| VA13 | 1.2 | 0.8 |
| Normal tissues | | |
| Brain | <0.2 | 1.0 |
| Colon | <0.2 | <0.2 |
| Heart | <0.2 | <0.2 |
| Kidney | <0.2 | 0.5 |
| Liver | <0.2 | 0.5 |
| Lung | <0.2 | 0.7 |
| Muscle | 0.4 | 1.0 |
| Placenta | <0.2 | <0.2 |
| Small Intestine | <0.2 | 0.3 |
| Spleen | <0.2 | 0.3 |
| Stomach | <0.2 | <0.2 |
| Testis | 0.6 | 0.6 |
| PBMC | 0.6 | 0.8 |

Industrial Applicability

The present invention makes it possible to induce and/or activate CTL in an HLA-A2-restricted manner or HLA-A26-restricted manner so as to conduct a specific immunotherapy for treating epithelial cancers, adenocarcinomas, and the like, such as colon cancer, esophageal cancer, oral cancer, renal cancer, pulmonary cancer, gynecological cancer, and prostate cancer. At present, 160,000 new colon cancer patients are found in the United States per year. Moreover, about 58,000 patients die due to colon cancer per year, which is approximately 18% of the mortality due to cancer. The colon cancer is the third cause of death due to cancer. An HLA-A2 allele is found in approximately 40% of Japanese, approximately 53% of Chinese, approximately 49% of North Caucasians, approximately 38% of South Caucasians, and approximately 23% of African Blacks. Moreover, an HLA-A26 allele is found in approximately 22% of Japanese, approximately 16% of Korean, and approximately 8% of North Caucasians. Therefore, the present invention can make a great contribution to the treatment of cancers. Moreover, the present invention will make a great contribution also to the basic research on molecules involved in the recognition of epithelial cancers, adenocarcinomas, and the like by a T lymphocyte.

Summary of the Sequence Listing

SEQ ID NO:1: Designed peptide recognized by HLA-A2 restricted cytotoxic T lymphocytes
SEQ ID NO:2: Designed peptide recognized by HLA-A2 restricted cytotoxic T lymphocytes
SEQ ID NO:3: Designed peptide recognized by HLA-A2 restricted cytotoxic T lymphocytes
SEQ ID NO:4: Designed peptide recognized by HLA-A2 restricted cytotoxic T lymphocytes
SEQ ID NO:5: Designed peptide recognized by HLA-A2 restricted cytotoxic T lymphocytes
SEQ ID NO:6: Designed peptide recognized by HLA-A2 restricted cytotoxic T lymphocytes
SEQ ID NO:7: Designed peptide recognized by HLA-A2 restricted cytotoxic T lymphocytes
SEQ ID NO:8: Designed peptide recognized by HLA-A2 restricted cytotoxic T lymphocytes
SEQ ID NO:9: Designed peptide recognized by HLA-A2 restricted cytotoxic T lymphocytes
SEQ ID NO:10: Designed peptide recognized by HLA-A2 restricted cytotoxic T lymphocytes
SEQ ID NO:11: Designed peptide recognized by HLA-A2 restricted cytotoxic T lymphocytes
SEQ ID NO:12: Designed peptide recognized by HLA-A2 restricted cytotoxic T lymphocytes
SEQ ID NO:13: Designed peptide recognized by HLA-A2 restricted cytotoxic T lymphocytes
SEQ ID NO:14: Designed peptide recognized by HLA-A2 restricted cytotoxic T lymphocytes
SEQ ID NO:15: Designed peptide recognized by HLA-A2 restricted cytotoxic T lymphocytes
SEQ ID NO:16: Designed peptide recognized by HLA-A2 restricted cytotoxic T lymphocytes
SEQ ID NO:17: Designed peptide recognized by HLA-A2 restricted cytotoxic T lymphocytes
SEQ ID NO:18: Designed peptide recognized by HLA-A2 restricted cytotoxic T lymphocytes
SEQ ID NO:19: Designed peptide recognized by HLA-A2 restricted cytotoxic T lymphocytes
SEQ ID NO:20: Designed peptide recognized by HLA-A2 restricted cytotoxic T lymphocytes
SEQ ID NO:21: Designed peptide recognized by HLA-A2 restricted cytotoxic T lymphocytes
SEQ ID NO:22: Designed peptide recognized by HLA-A2 restricted cytotoxic T lymphocytes
SEQ ID NO:23: Designed peptide recognized by HLA-A2 restricted cytotoxic T lymphocytes
SEQ ID NO:24: Designed peptide recognized by HLA-A2 restricted cytotoxic T lymphocytes
SEQ ID NO:25: Designed peptide recognized by HLA-A2 restricted cytotoxic T lymphocytes
SEQ ID NO:26: Designed peptide recognized by HLA-A2 restricted cytotoxic T lymphocytes
SEQ ID NO:27: Designed peptide recognized by HLA-A2 restricted cytotoxic T lymphocytes
SEQ ID NO:28: Designed peptide recognized by HLA-A2 restricted cytotoxic T lymphocytes
SEQ ID NO:29: Designed peptide recognized by HLA-A2 restricted cytotoxic T lymphocytes
SEQ ID NO:30: Designed peptide recognized by HLA-A2 restricted cytotoxic T lymphocytes
SEQ ID NO:31: Designed peptide recognized by HLA-A2 restricted cytotoxic T lymphocytes
SEQ ID NO:32: Designed peptide recognized by HLA-A2 restricted cytotoxic T lymphocytes
SEQ ID NO:33: Designed peptide recognized by HLA-A2 restricted cytotoxic T lymphocytes
SEQ ID NO:34: Designed peptide recognized by HLA-A2 restricted cytotoxic T lymphocytes
SEQ ID NO:35: Designed peptide recognized by HLA-A2 restricted cytotoxic T lymphocytes
SEQ ID NO:36: Designed peptide recognized by HLA-A2 restricted cytotoxic T lymphocytes
SEQ ID NO:37: Designed peptide recognized by HLA-A2 restricted cytotoxic T lymphocytes
SEQ ID NO:38: Designed peptide recognized by HLA-A2 restricted cytotoxic T lymphocytes
SEQ ID NO:39: Designed peptide recognized by HLA-A2 restricted cytotoxic T lymphocytes
SEQ ID NO:40: Designed peptide recognized by HLA-A2 restricted cytotoxic T lymphocytes
SEQ ID NO:41: Designed peptide recognized by HLA-A2 restricted cytotoxic T lymphocytes
SEQ ID NO:42: Designed peptide recognized by HLA-A2 restricted cytotoxic T lymphocytes
SEQ ID NO:43: Designed peptide recognized by HLA-A2 restricted cytotoxic T lymphocytes
SEQ ID NO:44: Designed peptide recognized by HLA-A2 restricted cytotoxic T lymphocytes
SEQ ID NO:45: Designed peptide recognized by HLA-A2 restricted cytotoxic T lymphocytes
SEQ ID NO:46: Designed peptide recognized by HLA-A2 restricted cytotoxic T lymphocytes
SEQ ID NO:47: Designed peptide recognized by HLA-A2 restricted cytotoxic T lymphocytes
SEQ ID NO:48: Designed peptide recognized by HLA-A2 restricted cytotoxic T lymphocytes
SEQ ID NO:49: Designed peptide recognized by HLA-A2 restricted cytotoxic T lymphocytes
SEQ ID NO:50: Designed peptide recognized by HLA-A2 restricted cytotoxic T lymphocytes
SEQ ID NO:51: Designed peptide recognized by HLA-A2 restricted cytotoxic T lymphocytes
SEQ ID NO:52: Designed peptide recognized by HLA-A2 restricted cytotoxic T lymphocytes
SEQ ID NO:53: Designed peptide recognized by HLA-A2 restricted cytotoxic T lymphocytes
SEQ ID NO:54: Designed peptide recognized by HLA-A2 restricted cytotoxic T lymphocytes
SEQ ID NO:55: Designed peptide recognized by HLA-A2 restricted cytotoxic T lymphocytes
SEQ ID NO:56: Designed peptide recognized by HLA-A2 restricted cytotoxic T lymphocytes
SEQ ID NO:57: Designed peptide recognized by HLA-A2 restricted cytotoxic T lymphocytes
SEQ ID NO:58: Designed peptide recognized by HLA-A2 restricted cytotoxic T lymphocytes
SEQ ID NO:59: Designed peptide recognized by HLA-A2 restricted cytotoxic T lymphocytes
SEQ ID NO:60: Designed peptide recognized by HLA-A2 restricted cytotoxic T lymphocytes
SEQ ID NO:61: Designed peptide recognized by HLA-A2 restricted cytotoxic T lymphocytes
SEQ ID NO:62: Designed peptide recognized by HLA-A2 restricted cytotoxic T lymphocytes
SEQ ID NO:63: Designed peptide recognized by HLA-A2 restricted cytotoxic T lymphocytes
SEQ ID NO:64: Designed peptide recognized by HLA-A2 restricted cytotoxic T lymphocytes
SEQ ID NO:65: Designed peptide recognized by HLA-A2 restricted cytotoxic T lymphocytes
SEQ ID NO:66: Designed peptide recognized by HLA-A2 restricted cytotoxic T lymphocytes
SEQ ID NO:67: Designed peptide recognized by HLA-A2 restricted cytotoxic T lymphocytes
SEQ ID NO:68: Designed peptide recognized by HLA-A2 restricted cytotoxic T lymphocytes
SEQ ID NO:69: Designed peptide recognized by HLA-A2 restricted cytotoxic T lymphocytes
SEQ ID NO:70: Designed peptide recognized by HLA-A2 restricted cytotoxic T lymphocytes
SEQ ID NO:71: Designed peptide recognized by HLA-A2 restricted cytotoxic T lymphocytes
SEQ ID NO:72: Designed peptide recognized by HLA-A2 restricted cytotoxic T lymphocytes
SEQ ID NO:73: Designed peptide recognized by HLA-A2 restricted cytotoxic T lymphocytes
SEQ ID NO:74: Designed peptide recognized by HLA-A2 restricted cytotoxic T lymphocytes
SEQ ID NO:75: Designed peptide recognized by HLA-A2 restricted cytotoxic T lymphocytes
SEQ ID NO:76: Designed peptide recognized by HLA-A2 restricted cytotoxic T lymphocytes
SEQ ID NO:77: Designed peptide recognized by HLA-A2 restricted cytotoxic T lymphocytes
SEQ ID NO:78: Designed peptide recognized by HLA-A2 restricted cytotoxic T lymphocytes
SEQ ID NO:79: Designed peptide recognized by HLA-A2 restricted cytotoxic T lymphocytes
SEQ ID NO:80: Designed peptide recognized by HLA-A2 restricted cytotoxic T lymphocytes
SEQ ID NO:81: Designed peptide recognized by HLA-A2 restricted cytotoxic T lymphocytes
SEQ ID NO:82: Designed peptide recognized by HLA-A2 restricted cytotoxic T lymphocytes
SEQ ID NO:83: Designed peptide recognized by HLA-A2 restricted cytotoxic T lymphocytes
SEQ ID NO:84: Designed peptide recognized by HLA-A2 restricted cytotoxic T lymphocytes
SEQ ID NO:85: Designed peptide recognized by HLA-A2 restricted cytotoxic T lymphocytes
SEQ ID NO:86: Designed peptide recognized by HLA-A2 restricted cytotoxic T lymphocytes
SEQ ID NO:87: Designed peptide recognized by HLA-A2 restricted cytotoxic T lymphocytes
SEQ ID NO:88: Designed peptide recognized by HLA-A2 restricted cytotoxic T lymphocytes
SEQ ID NO:89: Designed peptide recognized by HLA-A2 restricted cytotoxic T lymphocytes
SEQ ID NO:90: Designed peptide recognized by HLA-A2 restricted cytotoxic T lymphocytes
SEQ ID NO:91: Designed peptide recognized by HLA-A2 restricted cytotoxic T lymphocytes
SEQ ID NO:92: Designed peptide recognized by HLA-A2 restricted cytotoxic T lymphocytes
SEQ ID NO:93: Designed peptide recognized by HLA-A2 restricted cytotoxic T lymphocytes
SEQ ID NO:94: Designed peptide recognized by HLA-A2 restricted cytotoxic T lymphocytes
SEQ ID NO:95: Designed peptide recognized by HLA-A2 restricted cytotoxic T lymphocytes
SEQ ID NO:96: Designed peptide recognized by HLA-A2 restricted cytotoxic T lymphocytes
SEQ ID NO:97: Designed peptide recognized by HLA-A2 restricted cytotoxic T lymphocytes
SEQ ID NO:98: Designed peptide recognized by HLA-A2 restricted cytotoxic T lymphocytes
SEQ ID NO:99: Designed peptide recognized by HLA-A2 restricted cytotoxic T lymphocytes
SEQ ID NO:100: Designed peptide recognized by HLA-A2 restricted cytotoxic T lymphocytes
SEQ ID NO:101: Designed peptide recognized by HLA-A2 restricted cytotoxic T lymphocytes
SEQ ID NO:102: Designed peptide recognized by HLA-A2 restricted cytotoxic T lymphocytes
SEQ ID NO:103: Designed peptide recognized by HLA-A2 restricted cytotoxic T lymphocytes
SEQ ID NO:104: Designed peptide recognized by HLA-A2 restricted cytotoxic T lymphocytes
SEQ ID NO:105: Designed peptide recognized by HLA-A2 restricted cytotoxic T lymphocytes
SEQ ID NO:106: Designed peptide recognized by HLA-A2 restricted cytotoxic T lymphocytes
SEQ ID NO:107: Designed peptide recognized by HLA-A2 restricted cytotoxic T lymphocytes
SEQ ID NO:108: Designed peptide recognized by HLA-A2 restricted cytotoxic T lymphocytes
SEQ ID NO:109: Designed peptide recognized by HLA-A2 restricted cytotoxic T lymphocytes
SEQ ID NO:110: Designed peptide recognized by HLA-A2 restricted cytotoxic T lymphocytes
SEQ ID NO:111: Designed peptide recognized by HLA-A2 restricted cytotoxic T lymphocytes
SEQ ID NO:112: Designed peptide recognized by HLA-A2 restricted cytotoxic T lymphocytes
SEQ ID NO:113: Designed peptide recognized by HLA-A2 restricted cytotoxic T lymphocytes
SEQ ID NO:114: Designed peptide recognized by HLA-A2 restricted cytotoxic T lymphocytes
SEQ ID NO:115: Designed peptide recognized by HLA-A2 restricted cytotoxic T lymphocytes
SEQ ID NO:116: Designed peptide recognized by HLA-A2 restricted cytotoxic T lymphocytes
SEQ ID NO:117: Designed peptide recognized by HLA-A2 restricted cytotoxic T lymphocytes
SEQ ID NO:118: Designed peptide recognized by HLA-A2 restricted cytotoxic T lymphocytes
SEQ ID NO:119: Designed peptide recognized by HLA-A2 restricted cytotoxic T lymphocytes
SEQ ID NO:120: Designed peptide recognized by HLA-A2 restricted cytotoxic T lymphocytes
SEQ ID NO:121: Designed peptide recognized by HLA-A2 restricted cytotoxic T lymphocytes
SEQ ID NO:122: Designed peptide recognized by HLA-A2 restricted cytotoxic T lymphocytes
SEQ ID NO:123: Designed peptide recognized by HLA-A2 restricted cytotoxic T lymphocytes
SEQ ID NO:124: Designed peptide recognized by HLA-A2 restricted cytotoxic T lymphocytes
SEQ ID NO:125: Designed peptide recognized by HLA-A2 restricted cytotoxic T lymphocytes
SEQ ID NO:126: Designed peptide recognized by HLA-A2 restricted cytotoxic T lymphocytes
SEQ ID NO:127: Designed peptide recognized by HLA-A2 restricted cytotoxic T lymphocytes
SEQ ID NO:128: Designed peptide recognized by HLA-A2 restricted cytotoxic T lymphocytes
SEQ ID NO:129: Designed peptide recognized by HLA-A2 restricted cytotoxic T lymphocytes
SEQ ID NO:130: Designed peptide recognized by HLA-A2 restricted cytotoxic T lymphocytes
SEQ ID NO:131: Designed peptide recognized by HLA-A2 restricted cytotoxic T lymphocytes
SEQ ID NO:132: Designed peptide recognized by HLA-A2 restricted cytotoxic T lymphocytes
SEQ ID NO:133: Designed peptide recognized by HLA-A2 restricted cytotoxic T lymphocytes
SEQ ID NO:134: Designed peptide recognized by HLA-A2 restricted cytotoxic T lymphocytes
SEQ ID NO:135: Designed peptide recognized by HLA-A2 restricted cytotoxic T lymphocytes
SEQ ID NO:136: Designed peptide recognized by HLA-A2 restricted cytotoxic T lymphocytes
SEQ ID NO:137: Designed peptide recognized by HLA-A2 restricted cytotoxic T lymphocytes
SEQ ID NO:138: Designed peptide recognized by HLA-A2 restricted cytotoxic T lymphocytes
SEQ ID NO:139: Designed peptide recognized by HLA-A2 restricted cytotoxic T lymphocytes
SEQ ID NO:140: Designed peptide recognized by HLA-A2 restricted cytotoxic T lymphocytes
SEQ ID NO:141: Designed peptide recognized by HLA-A2 restricted cytotoxic T lymphocytes
SEQ ID NO:142: Designed peptide recognized by HLA-A2 restricted cytotoxic T lymphocytes
SEQ ID NO:143: Designed peptide recognized by HLA-A2 restricted cytotoxic T lymphocytes
SEQ ID NO:144: Designed peptide recognized by HLA-A2 restricted cytotoxic T lymphocytes
SEQ ID NO:145: Designed peptide recognized by HLA-A2 restricted cytotoxic T lymphocytes
SEQ ID NO:146: Designed peptide recognized by HLA-A2 restricted cytotoxic T lymphocytes
SEQ ID NO:147: Designed peptide recognized by HLA-A2 restricted cytotoxic T lymphocytes
SEQ ID NO:148: Designed peptide recognized by HLA-A2 restricted cytotoxic T lymphocytes
SEQ ID NO:149: Designed peptide recognized by HLA-A2 restricted cytotoxic T lymphocytes
SEQ ID NO:150: Designed peptide recognized by HLA-A2 restricted cytotoxic T lymphocytes
SEQ ID NO:151: Designed peptide recognized by HLA-A2 restricted cytotoxic T lymphocytes
SEQ ID NO:152: Designed peptide recognized by HLA-A2 restricted cytotoxic T lymphocytes
SEQ ID NO:153: Designed peptide recognized by HLA-A2 restricted cytotoxic T lymphocytes
SEQ ID NO:154: Designed peptide recognized by HLA-A2 restricted cytotoxic T lymphocytes
SEQ ID NO:155: Designed peptide recognized by HLA-A2 restricted cytotoxic T lymphocytes
SEQ ID NO:156: Designed peptide recognized by HLA-A2 restricted cytotoxic T lymphocytes
SEQ ID NO:157: Designed peptide recognized by HLA-A2 restricted cytotoxic T lymphocytes
SEQ ID NO:158: Designed peptide recognized by HLA-A2 restricted cytotoxic T lymphocytes
SEQ ID NO:159: Designed peptide recognized by HLA-A2 restricted cytotoxic T lymphocytes
SEQ ID NO:160: Designed peptide recognized by HLA-A2 restricted cytotoxic T lymphocytes
SEQ ID NO:161: Designed peptide recognized by HLA-A2 restricted cytotoxic T lymphocytes
SEQ ID NO:162: Designed peptide recognized by HLA-A2 restricted cytotoxic T lymphocytes
SEQ ID NO:163: Designed peptide recognized by HLA-A2 restricted cytotoxic T lymphocytes
SEQ ID NO:164: Designed peptide recognized by HLA-A2 restricted cytotoxic T lymphocytes
SEQ ID NO:165: Designed peptide recognized by HLA-A2 restricted cytotoxic T lymphocytes
SEQ ID NO:166: Designed peptide recognized by HLA-A2 restricted cytotoxic T lymphocytes
SEQ ID NO:167: Designed peptide recognized by HLA-A2 restricted cytotoxic T lymphocytes
SEQ ID NO:168: Designed peptide recognized by HLA-A2 restricted cytotoxic T lymphocytes
SEQ ID NO:169: Designed peptide recognized by HLA-A2 restricted cytotoxic T lymphocytes
SEQ ID NO:170: Designed peptide recognized by HLA-A2 restricted cytotoxic T lymphocytes
SEQ ID NO:171: Designed peptide recognized by HLA-A2 restricted cytotoxic T lymphocytes
SEQ ID NO:172: Designed peptide recognized by HLA-A2 restricted cytotoxic T lymphocytes
SEQ ID NO:173: Designed peptide recognized by HLA-A2 restricted cytotoxic T lymphocytes
SEQ ID NO:174: Designed peptide recognized by HLA-A2 restricted cytotoxic T lymphocytes
SEQ ID NO:175: Designed peptide recognized by HLA-A2 restricted cytotoxic T lymphocytes
SEQ ID NO:176: Designed peptide recognized by HLA-A2 restricted cytotoxic T lymphocytes
SEQ ID NO:177: Designed peptide recognized by HLA-A2 restricted cytotoxic T lymphocytes
SEQ ID NO:178: Designed peptide recognized by HLA-A2 restricted cytotoxic T lymphocytes
SEQ ID NO:179: Designed peptide recognized by HLA-A2 restricted cytotoxic T lymphocytes
SEQ ID NO:180: Designed peptide recognized by HLA-A2 restricted cytotoxic T lymphocytes
SEQ ID NO:181: Designed peptide recognized by HLA-A2 restricted cytotoxic T lymphocytes
SEQ ID NO:182: Designed peptide recognized by HLA-A2 restricted cytotoxic T lymphocytes
SEQ ID NO:183: Designed peptide recognized by HLA-A2 restricted cytotoxic T lymphocytes
SEQ ID NO:184: Designed peptide recognized by HLA-A2 restricted cytotoxic T lymphocytes
SEQ ID NO:185: Designed peptide recognized by HLA-A2 restricted cytotoxic T lymphocytes
SEQ ID NO:186: Designed peptide recognized by HLA-A2 restricted cytotoxic T lymphocytes
SEQ ID NO:187: Designed peptide recognized by HLA-A2 restricted cytotoxic T lymphocytes
SEQ ID NO:188: Designed peptide recognized by HLA-A2 restricted cytotoxic T lymphocytes
SEQ ID NO:189: Designed peptide recognized by HLA-A2 restricted cytotoxic T lymphocytes
SEQ ID NO:190: Designed peptide recognized by HLA-A2 restricted cytotoxic T lymphocytes
SEQ ID NO:191: Designed peptide recognized by HLA-A2 restricted cytotoxic T lymphocytes
SEQ ID NO:192: Designed peptide recognized by HLA-A2 restricted cytotoxic T lymphocytes
SEQ ID NO:193: Designed peptide recognized by HLA-A2 restricted cytotoxic T lymphocytes
SEQ ID NO:194: Designed peptide recognized by HLA-A2 restricted cytotoxic T lymphocytes
SEQ ID NO:195: Designed peptide recognized by HLA-A2 restricted cytotoxic T lymphocytes
SEQ ID NO:196: Designed peptide recognized by HLA-A2 restricted cytotoxic T lymphocytes
SEQ ID NO:197: Designed peptide recognized by HLA-A2 restricted cytotoxic T lymphocytes
SEQ ID NO:198: Designed peptide recognized by HLA-A2 restricted cytotoxic T lymphocytes
SEQ ID NO:199: Designed peptide recognized by HLA-A2 restricted cytotoxic T lymphocytes
SEQ ID NO:200: Designed peptide recognized by HLA-A2 restricted cytotoxic T lymphocytes
SEQ ID NO:201: Designed peptide recognized by HLA-A2 restricted cytotoxic T lymphocytes
SEQ ID NO:202: Designed peptide recognized by HLA-A2 restricted cytotoxic T lymphocytes
SEQ ID NO:203: Designed peptide recognized by HLA-A2 restricted cytotoxic T lymphocytes
SEQ ID NO:204: Designed peptide recognized by HLA-A2 restricted cytotoxic T lymphocytes
SEQ ID NO:205: Designed peptide recognized by HLA-A2 restricted cytotoxic T lymphocytes
SEQ ID NO:206: Designed peptide recognized by HLA-A2 restricted cytotoxic T lymphocytes
SEQ ID NO:207: Designed peptide recognized by HLA-A2 restricted cytotoxic T lymphocytes
SEQ ID NO:208: Designed peptide recognized by HLA-A2 restricted cytotoxic T lymphocytes
SEQ ID NO:209: Designed peptide recognized by HLA-A2 restricted cytotoxic T lymphocytes
SEQ ID NO:210: Designed peptide recognized by HLA-A2 restricted cytotoxic T lymphocytes
SEQ ID NO:211: Designed peptide recognized by HLA-A2 restricted cytotoxic T lymphocytes
SEQ ID NO:212: Designed peptide recognized by HLA-A2 restricted cytotoxic T lymphocytes
SEQ ID NO:213: Designed peptide recognized by HLA-A2 restricted cytotoxic T lymphocytes
SEQ ID NO:284: “Xaa” may be “Asp” or “Glu”.
SEQ ID NO:352: “n” may be “a”, “c”, “g” or “t”.
SEQ ID NO:358: Designed peptide recognized by HLA-A2 restricted cytotoxic T lymphocytes
SEQ ID NO:359: Designed peptide recognized by HLA-A2 restricted cytotoxic T lymphocytes
SEQ ID NO:360: Designed peptide recognized by HLA-A2 restricted cytotoxic T lymphocytes
SEQ ID NO:361: Designed peptide recognized by HLA-A2 restricted cytotoxic T lymphocytes
SEQ ID NO:362: Designed peptide recognized by HLA-A2 restricted cytotoxic T lymphocytes
SEQ ID NO:363: Designed peptide recognized by HLA-A2 restricted cytotoxic T lymphocytes
SEQ ID NO:364: Designed peptide recognized by HLA-A2 restricted cytotoxic T lymphocytes
SEQ ID NO:365: Designed peptide recognized by HLA-A2 restricted cytotoxic T lymphocytes
SEQ ID NO:366: Designed peptide recognized by HLA-A2 restricted cytotoxic T lymphocytes
SEQ ID NO:367: Designed peptide recognized by HLA-A2 restricted cytotoxic T lymphocytes
SEQ ID NO:368: Designed peptide recognized by HLA-A2 restricted cytotoxic T lymphocytes
SEQ ID NO:369: Designed peptide recognized by HLA-A2 restricted cytotoxic T lymphocytes
SEQ ID NO:370: Designed peptide recognized by HLA-A2 restricted cytotoxic T lymphocytes
SEQ ID NO:371: Designed peptide recognized by HLA-A2 restricted cytotoxic T lymphocytes
SEQ ID NO:372: Designed peptide recognized by HLA-A2 restricted cytotoxic T lymphocytes
SEQ ID NO:373: Designed peptide recognized by HLA-A2 restricted cytotoxic T lymphocytes
SEQ ID NO:374: Designed peptide recognized by HLA-A2 restricted cytotoxic T lymphocytes
SEQ ID NO:375: Designed peptide recognized by HLA-A2 restricted cytotoxic T lymphocytes
SEQ ID NO:376: Designed peptide recognized by HLA-A2 restricted cytotoxic T lymphocytes
SEQ ID NO:377: Designed peptide recognized by HLA-A2 restricted cytotoxic T lymphocytes
SEQ ID NO:378: Designed peptide recognized by HLA-A2 restricted cytotoxic T lymphocytes
SEQ ID NO:379: Designed peptide recognized by HLA-A2 restricted cytotoxic T lymphocytes
SEQ ID NO:380: Designed peptide recognized by HLA-A2 restricted cytotoxic T lymphocytes
SEQ ID NO:381: Designed peptide recognized by HLA-A2 restricted cytotoxic T lymphocytes
SEQ ID NO:388: Designed peptide recognized by HLA-A26 restricted cytotoxic T lymphocytes
SEQ ID NO:389: Designed peptide recognized by HLA-A26 restricted cytotoxic T lymphocytes
SEQ ID NO:390: Designed peptide recognized by HLA-A26 restricted cytotoxic T lymphocytes
SEQ ID NO:391: Designed peptide recognized by HLA-A26 restricted cytotoxic T lymphocytes
SEQ ID NO:392: Designed peptide recognized by HLA-A26 restricted cytotoxic T lymphocytes
SEQ ID NO:393: Designed peptide recognized by HLA-A26 restricted cytotoxic T lymphocytes
SEQ ID NO:394: Designed peptide recognized by HLA-A26 restricted cytotoxic T lymphocytes
SEQ ID NO:395: Designed peptide recognized by HLA-A26 restricted cytotoxic T lymphocytes
SEQ ID NO:396: Designed peptide recognized by HLA-A26 restricted cytotoxic T lymphocytes
SEQ ID NO:397: Designed peptide recognized by HLA-A26 restricted cytotoxic T lymphocytes
SEQ ID NO:398: Designed peptide recognized by HLA-A26 restricted cytotoxic T lymphocytes
SEQ ID NO:399: Designed peptide recognized by HLA-A26 restricted cytotoxic T lymphocytes
SEQ ID NO:400: Designed peptide recognized by HLA-A26 restricted cytotoxic T lymphocytes
SEQ ID NO:401: Designed peptide recognized by HLA-A26 restricted cytotoxic T lymphocytes
SEQ ID NO:402: Designed peptide recognized by HLA-A26 restricted cytotoxic T lymphocytes
SEQ ID NO:403: Designed peptide recognized by HLA-A26 restricted cytotoxic T lymphocytes
SEQ ID NO:404: Designed peptide recognized by HLA-A26 restricted cytotoxic T lymphocytes
SEQ ID NO:405: Designed peptide recognized by HLA-A26 restricted cytotoxic T lymphocytes
SEQ ID NO:406: Designed peptide recognized by HLA-A26 restricted cytotoxic T lymphocytes
SEQ ID NO:407: Designed peptide recognized by HLA-A26 restricted cytotoxic T lymphocytes
SEQ ID NO:408: Designed peptide recognized by HLA-A26 restricted cytotoxic T lymphocytes

1. An isolated peptide consisting of an amino acid sequence selected from the group consisting of any one of SEQ ID NO:1 to SEQ ID NO:213, SEQ ID NO:358 to SEQ ID NO:381, and SEQ ID NO:388 to SEQ ID NO:408.

2. An isolated polypeptide consisting of an amino acid sequence selected from the group consisting of any one of SEQ ID NO:215 to SEQ ID NO:288 (excluding SEQ ID NO:228, SEQ ID NO:236, SEQ ID NO:261, and SEQ ID NO:269), SEQ ID NO:356, SEQ ID NO:357, SEQ ID NO:385, and SEQ ID NO:386.

3. An isolated peptide consisting of an amino acid sequence selected from the group consisting of any one of SEQ ID NO:1 to SEQ ID NO:213, SEQ ID NO:358 to SEQ ID NO:381, and SEQ ID NO:388 to SEQ ID NO:408, wherein the peptide is recognized by a cytotoxic T lymphocyte and/or induces a cytotoxic T lymphocyte.

4. The peptide according to claim 3, wherein said peptide is recognized by a cytotoxic T lymphocyte in an HLA-A2-restricted manner or HLA-A26-restricted manner and/or induces a cytotoxic T lymphocyte in an HLA-A2-restricted manner or HLA-A26-restricted manner.

5. An isolated polypeptide consisting of an amino acid sequence selected from the group consisting of any one of SEQ ID NO:214 to SEQ ID NO:288, SEQ ID NO:356, SEQ ID NO:357, and SEQ ID NO:385 to SEQ ID NO:387, wherein the polypeptide is recognized by a cytotoxic T lymphocyte and/or induces a cytotoxic T lymphocyte.

6. The polypeptide according to claim 5, wherein said polypeptide is recognized by the cytotoxic T lymphocyte in an HLA-A2-restricted manner or HLA-A26-restricted manner and/or induces the cytotoxic T lymphocyte in an HLA-A2-restricted manner or HLA-A26-restricted manner.

7. A pharmaceutical composition comprising one or more of peptides that consist of an amino acid sequence selected from the group consisting of any of one of SEQ ID NO:1 to SEQ ID NO:213, SEQ ID NO:358 to SEQ ID NO:381, and SEQ ID NO:388 to SEQ ID NO:408, and/or, one or more of polypeptides that consist of an amino acid sequence selected from the group consisting of any one of SEQ ID NO:214 to SEQ ID NO:288, SEQ ID NO:356, SEQ ID NO:357, and SEQ ID NO:385 to SEQ ID NO:387; and a pharmaceutically acceptable carrier.

8. A cancer vaccine comprising an immunoprotective effective amount of one or more of peptides that consist of an amino acid sequence selected from the group consisting of any one of SEQ ID NO:1 to SEQ ID NO:213, SEQ ID NO:358 to SEQ ID NO:381, and SEQ ID NO:388 to SEQ ID NO:408, and/or, one or more of polypeptides that consist of an amino acid sequence selected from the group consisting of any one of SEQ ID NO:214 to SEQ ID NO:288, SEQ ID NO:356, SEQ ID NO:357, and SEQ ID NO:385 to SEQ ID NO:387; and a pharmaceutically acceptable carrier.

9. The cancer vaccine according to claim 8, wherein the vaccine is used for treating one or more of cancers selected from the group consisting of colon cancer, esophageal cancer, oral squamous cell cancer, renal cancer, pulmonary cancer, gynecological cancer, and prostate cancer.

10. A method for inducing a cytotoxic T lymphocyte, wherein the method comprises contacting peripheral blood mononuclear cells with one or more of peptides that consist of an amino acid sequence selected from the group consisting of any one of SEQ ID NO:1 to SEQ ID NO:213, SEQ ID NO:358 to SEQ ID NO:381, and SEQ ID NO:388 to SEQ ID NO:408, and/or, one or more of polypeptides that consist of an amino acid sequence selected from the group consisting of any one of SEQ ID NO:214 to SEQ ID NO:288, SEQ ID NO:356, SEQ ID NO:357, and SEQ ID NO:385 to SEQ ID NO:387.

11. An isolated polynucleotide encoding a peptide or polypeptide consisting of an amino acid sequence selected from the group consisting of any one of SEQ ID NO:1 to SEQ ID NO:288, SEQ ID NO:356 to SEQ ID NO:381, and SEQ ID NO:385 to SEQ ID NO:408, or a complementary strand thereof.

12. An isolated polynucleotide consisting of a nucleotide sequence selected from the group consisting of any one of SEQ ID NO:290 to SEQ ID NO:355 (excluding SEQ ID NO:299 and SEQ ID NO:332) and SEQ ID NO:382 to SEQ ID NO:384, or a complementary strand thereof.

13. An isolated polynucleotide consisting of a nucleotide sequence selected from the group consisting of any one of SEQ ID NO:289 to SEQ ID NO:355 and SEQ ID NO:382 to SEQ ID NO:384, or a complementary strand thereof, wherein a polypeptide encoded by the polynucleotide induces a cytotoxic T lymphocyte and/or is recognized by a cytotoxic T lymphocyte.

14. The polynucleotide or the complementary strand thereof according to claim 13, wherein said polypeptide induces a cytotoxic T lymphocyte in an HLA-A2-restrcited manner or HLA-A26-restricted manner and/or is recognized by a cytotoxic T lymphocyte in an HLA-A2-restrcited manner or HLA-A26-restricted manner.

15. An isolated polynucleotide that hybridizes to the polynucleotide or the complementary strand thereof according to any one of claims 11 to 14 under stringent conditions.

16. A recombinant vector comprising the polynucleotide or the complementary strand thereof according to any one of claims 11 to 14, or a polynucleotide that hybridizes to said polynucleotide or the complementary strand thereof under stringent conditions.

17. The recombinant vector according to claim 16, wherein the recombinant vector is a recombinant expression vector.

18. A transformant transformed with a recombinant vector or a recombinant expression vector, wherein the recombinant vector or the recombinant expression vector comprises the polynucleotide or the complementary strand thereof according to any one of claims 11 to 14, or a polynucleotide that hybridizes to said polynucleotide or the complementary strand thereof under stringent conditions.

19. A method for producing a polypeptide, wherein the method comprises culturing a transformant transformed with the recombinant vector according to claim 17.

20. An antibody immunologically recognizing the peptide according to claim 1, 3, or 4, or the polypeptide according to claim 2, 5, or 6.

21. A method for screening for a compound that enhances recognition of the peptide according to claim 4 or the polypeptide according to claim 6 at least by an HLA-A2-restricted or HLA-A26-restricted cytotoxic T lymphocyte, wherein said method comprises contacting said peptide or said polypeptide, with a compound; and determining whether said compound enhances said recognition by measuring IFN-γ production from said cytotoxic T lymphocytes.

22. A method for screening for a compound that enhances recognition of the peptide according to claim 4 at least by an HLA-A2-restricted or HLA-A26-restricted cytotoxic T lymphocyte, wherein said method comprises contacting HLA-A2⁺ cells or HLA-A26⁺ cells which have been pulsed with said peptide, with said cytotoxic T lymphocytes which recognize a complex of the peptide and HLA-A2 molecule or a complex of the peptide and HLA-A26 molecule in the presence or absence of a compound; and determining whether said compound enhances said recognition by measuring IFN-γ production from said cytotoxic T lymphocytes.

23. A method for screening for a compound that enhances recognition of the peptide according to claim 4 at least by an HLA-A2-restricted or HLA-A26-restricted cytotoxic T lymphocyte, wherein said method comprises contacting HLA-A2⁺ cells or HLA-A26⁺ cells into which the polynucleotide according to claim 14 have been transfected, with said cytotoxic T lymphocytes which recognize a complex of the peptide and HLA-A2 molecule or a complex of the peptide and HLA-A26 molecule in the presence or absence of a compound; and determining whether said compound enhances said recognition by measuring IFN-γ production from said cytotoxic T lymphocytes.

24. A compound that is obtained by the screening method according to claim 21.

25. A compound that enhances recognition of at least one of the peptide according to claim 4 and/or the polypeptide according to claim 6 by an HLA-A2-restricted or HLA-A26-restricted cytotoxic T lymphocyte through interaction with the same.

26. A compound that enhances the expression of the polynucleotide or the complementary strand thereof according to any one of claims 11 to 14 through interaction with the same.

27. A pharmaceutical composition used for treating cancer, the composition comprising at least one member selected from the group consisting of the peptides according to claim 4, the polypeptides according to claim 6, the polynucleotide or the complementary strand thereof according to any of claims 11 to 14, or a polynucleotide that hybridizes to said polynucleotide or the complementary strand thereof under stringent conditions, a recombinant vector or recombinant expression vector comprising said polynucleotide or the complementary strand thereof, a transformant comprising said recombinant vector or recombinant expression vector, and an antibody that immunologically recognizes said peptide or polypeptide; and a pharmaceutically acceptable carrier.

28. A method for measuring quantitatively or qualitatively the peptide according to claim 4, or the polypeptide according to claim 6, or the polynucleotide or the complementary strand thereof according to any one of claims 11 to 14, or a polynucleotide that hybridizes to said polynucleotide or the complementary strand thereof under stringent conditions, wherein said method comprises detecting the presence of or determining the amount of said peptide or said polypeptide or said polynucleotide in a sample.

29. The measuring method according to claim 28, wherein the method is used in an examination for a cancer disease.

30. A reagent kit comprising at least one member selected from a group consisting of the peptide according to claim 4, the polypeptide according to claim 6, an antibody that immunologically recognizes said peptide or polypeptide, the polynucleotide or the complementary strand thereof according to claims 11 to 14, or a polynucleotide that hybridizes to said polynucleotide or the complementary strand thereof under stringent conditions, a recombinant vector or recombinant expression vector comprising said polynucleotide or the complementary strand thereof, a transformant comprising said recombinant vector or recombinant expression vector; and a buffered solution.

31. The reagent kit according to claim 30, wherein the reagent kit is used in diagnosing cancer disease.

32. A method for treating cancer comprising in vivo administering the cancer vaccine according to claim 8 or 9 to a patient afflicted with cancer.

33. A method for treating cancer comprising in vivo administering the cancer vaccine according to claim 8 or 9 to a patient afflicted with cancer, in an amount sufficient to induce cytotoxic T lymphocytes in said patient which recognize a complex of the peptide according to claim 4 and HLA-A2 molecule or a complex of said peptide and HLA-A26 molecule, and thereby to lyse cancer cells in said patient.

34. A method for treating a cancer patient comprising treating peripheral blood mononuclear cells which have been isolated from said patient with the cancer vaccine according to claim 8 or 9; and administering the thus treated peripheral blood mononuclear cells to said patient.

35. A method for treating a cancer patient comprising treating peripheral blood mononuclear cells which have been isolated from said patient with the cancer vaccine according to claim 8 or 9 in an amount sufficient to induce cytotoxic T lymphocytes in said patient which recognize a complex of the peptide according to claim 4 and HLA-A2 molecule or a complex of said peptide and HLA-A26 molecule, and thereby lyse cancer cells in said patient; and administering the thus treated peripheral blood mononuclear cells to said patient.

36. A method for diagnosing cancer comprising measuring quantitatively or qualitatively the peptide according to claim 4, or the polypeptide according to claim 6, or the polynucleotide or the complementary strand thereof according to any one of claims 11 to 14, or a polynucleotide that hybridizes to said polynucleotide or the complementary strand thereof under stringent conditions.


SEQUENCE LISTING (408 sequences)

SEQ ID NO: 1 - protein, 9 aa, Artificial
  Designed peptide recognized by HLA-A2 restricted cytotoxic T lymphocytes
KLTGMAFRV

SEQ ID NO: 2 - protein, 10 aa, Artificial
  Designed peptide recognized by HLA-A2 restricted cytotoxic T lymphocytes
ALNDHFVKLI

SEQ ID NO: 3 - protein, 9 aa, Artificial
  Designed peptide recognized by HLA-A2 restricted cytotoxic T lymphocytes
ILGYTEHQV

SEQ ID NO: 4 - protein, 10 aa, Artificial
  Designed peptide recognized by HLA-A2 restricted cytotoxic T lymphocytes
GIVEGLMTTV

SEQ ID NO: 5 - protein, 10 aa, Artificial
  Designed peptide recognized by HLA-A2 restricted cytotoxic T lymphocytes
TLLAGMNKFL

SEQ ID NO: 6 - protein, 10 aa, Artificial
  Designed peptide recognized by HLA-A2 restricted cytotoxic T lymphocytes
TLSGGELQRV

SEQ ID NO: 7 - protein, 9 aa, Artificial
  Designed peptide recognized by HLA-A2 restricted cytotoxic T lymphocytes
IVVEHDLSV

SEQ ID NO: 8 - protein, 10 aa, Artificial
  Designed peptide recognized by HLA-A2 restricted cytotoxic T lymphocytes
ILTYFRGSEL

SEQ ID NO: 9 - protein, 10 aa, Artificial
  Designed peptide recognized by HLA-A2 restricted cytotoxic T lymphocytes
ILAGKQKPNL

SEQ ID NO: 10 - protein, 10 aa, Artificial
  Designed peptide recognized by HLA-A2 restricted cytotoxic T lymphocytes
LEGEYIKLKV

SEQ ID NO: 11 - protein, 10 aa, Artificial
  Designed peptide recognized by HLA-A2 restricted cytotoxic T lymphocytes
ALGEEWKGYV

SEQ ID NO: 12 - protein, 10 aa, Artificial
  Designed peptide recognized by HLA-A2 restricted cytotoxic T lymphocytes
NLSVLNLVIV

SEQ ID NO: 13 - protein, 9 aa, Artificial
  Designed peptide recognized by HLA-A2 restricted cytotoxic T lymphocytes
VLTHGRVRL

SEQ ID NO: 14 - protein, 10 aa, Artificial
  Designed peptide recognized by HLA-A2 restricted cytotoxic T lymphocytes
KIQRLVTPRV

SEQ ID NO: 15 - protein, 9 aa, Artificial
  Designed peptide recognized by HLA-A2 restricted cytotoxic T lymphocytes
VQFVQGIFV

SEQ ID NO: 16 - protein, 9 aa, Artificial
  Designed peptide recognized by HLA-A2 restricted cytotoxic T lymphocytes
KSALTVQFV

SEQ ID NO: 17 - protein, 10 aa, Artificial
  Designed peptide recognized by HLA-A2 restricted cytotoxic T lymphocytes
IMNQEKLAKL

SEQ ID NO: 18 - protein, 10 aa, Artificial
  Designed peptide recognized by HLA-A2 restricted cytotoxic T lymphocytes
LQFSLKKLGV

SEQ ID NO: 19 - protein, 9 aa, Artificial
  Designed peptide recognized by HLA-A2 restricted cytotoxic T lymphocytes
NMFTNQGTV

SEQ ID NO: 20 - protein, 10 aa, Artificial
  Designed peptide recognized by HLA-A2 restricted cytotoxic T lymphocytes
RLHNDRVYYV

SEQ ID NO: 21 - protein, 10 aa, Artificial
  Designed peptide recognized by HLA-A2 restricted cytotoxic T lymphocytes
TIGENLQLLV

SEQ ID NO: 22 - protein, 10 aa, Artificial
  Designed peptide recognized by HLA-A2 restricted cytotoxic T lymphocytes
SLGTCFGKFT

SEQ ID NO: 23 - protein, 10 aa, Artificial
  Designed peptide recognized by HLA-A2 restricted cytotoxic T lymphocytes
RLHVTALDYL

SEQ ID NO: 24 - protein, 10 aa, Artificial
  Designed peptide recognized by HLA-A2 restricted cytotoxic T lymphocytes
LLGKTPVTQV

SEQ ID NO: 25 - protein, 10 aa, Artificial
  Designed peptide recognized by HLA-A2 restricted cytotoxic T lymphocytes
ALQKDVEDFL

SEQ ID NO: 26 - protein, 10 aa, Artificial
  Designed peptide recognized by HLA-A2 restricted cytotoxic T lymphocytes
VESVDGYQFV

SEQ ID NO: 27 - protein, 10 aa, Artificial
  Designed peptide recognized by HLA-A2 restricted cytotoxic T lymphocytes
ILGDKFPCTL

SEQ ID NO: 28 - protein, 10 aa, Artificial
  Designed peptide recognized by HLA-A2 restricted cytotoxic T lymphocytes
ALGGLPGPYI

SEQ ID NO: 29 - protein, 9 aa, Artificial
  Designed peptide recognized by HLA-A2 restricted cytotoxic T lymphocytes
VLVEDTCLC

SEQ ID NO: 30 - protein, 9 aa, Artificial
  Designed peptide recognized by HLA-A2 restricted cytotoxic T lymphocytes
ALCTFALST

SEQ ID NO: 31 - protein, 10 aa, Artificial
  Designed peptide recognized by HLA-A2 restricted cytotoxic T lymphocytes
YQGEPDEISI

SEQ ID NO: 32 - protein, 9 aa, Artificial
  Designed peptide recognized by HLA-A2 restricted cytotoxic T lymphocytes
ILALFMPPT

SEQ ID NO: 33 - protein, 10 aa, Artificial
  Designed peptide recognized by HLA-A2 restricted cytotoxic T lymphocytes
VLIEILCTRT

SEQ ID NO: 34 - protein, 9 aa, Artificial
  Designed peptide recognized by HLA-A2 restricted cytotoxic T lymphocytes
RLYYAMKGA

SEQ ID NO: 35 - protein, 10 aa, Artificial
  Designed peptide recognized by HLA-A2 restricted cytotoxic T lymphocytes
GVPPGQGFGV

SEQ ID NO: 36 - protein, 9 aa, Artificial
  Designed peptide recognized by HLA-A2 restricted cytotoxic T lymphocytes
YQSEFGRDL

SEQ ID NO: 37 - protein, 9 aa, Artificial
  Designed peptide recognized by HLA-A2 restricted cytotoxic T lymphocytes
GVISPRFDV

SEQ ID NO: 38 - protein, 9 aa, Artificial
  Designed peptide recognized by HLA-A2 restricted cytotoxic T lymphocytes
TLMMKHGYI

SEQ ID NO: 39 - protein, 10 aa, Artificial
  Designed peptide recognized by HLA-A2 restricted cytotoxic T lymphocytes
LLPSRQFGFI

SEQ ID NO: 40 - protein, 10 aa, Artificial
  Designed peptide recognized by HLA-A2 restricted cytotoxic T lymphocytes
SVYAHFPINV

SEQ ID NO: 41 - protein, 9 aa, Artificial
  Designed peptide recognized by HLA-A2 restricted cytotoxic T lymphocytes
VIQENGSLV

SEQ ID NO: 42 - protein, 9 aa, Artificial
  Designed peptide recognized by HLA-A2 restricted cytotoxic T lymphocytes
ILSNQTVDI

SEQ ID NO: 43 - protein, 10 aa, Artificial
  Designed peptide recognized by HLA-A2 restricted cytotoxic T lymphocytes
LIFLIFFFFL

SEQ ID NO: 44 - protein, 9 aa, Artificial
  Designed peptide recognized by HLA-A2 restricted cytotoxic T lymphocytes
VVIFKIFLI

SEQ ID NO: 45 - protein, 10 aa, Artificial
  Designed peptide recognized by HLA-A2 restricted cytotoxic T lymphocytes
GMADSQNMLV

SEQ ID NO: 46 - protein, 10 aa, Artificial
  Designed peptide recognized by HLA-A2 restricted cytotoxic T lymphocytes
IISEKYQVFI

SEQ ID NO: 47 - protein, 9 aa, Artificial
  Designed peptide recognized by HLA-A2 restricted cytotoxic T lymphocytes
KLIEEKGAV

SEQ ID NO: 48 - protein, 9 aa, Artificial
  Designed peptide recognized by HLA-A2 restricted cytotoxic T lymphocytes
FLFPIQAKT

SEQ ID NO: 49 - protein, 10 aa, Artificial
  Designed peptide recognized by HLA-A2 restricted cytotoxic T lymphocytes
SLINSNVGFV

SEQ ID NO: 50 - protein, 10 aa, Artificial
  Designed peptide recognized by HLA-A2 restricted cytotoxic T lymphocytes
KLGVCFDVPT

SEQ ID NO: 51 - protein, 10 aa, Artificial
  Designed peptide recognized by HLA-A2 restricted cytotoxic T lymphocytes
YQHKEEYQLV

SEQ ID NO: 52 - protein, 10 aa, Artificial
  Designed peptide recognzied by HLA-A2 restricted cytotoxic T lymphocytes
MVFLKGKLGV

SEQ ID NO: 53 - protein, 9 aa, Artificial
  Designed peptide recognized by HLA-A2 restricted cytotoxic T lymphocytes
ALAAALAHI

SEQ ID NO: 54 - protein, 9 aa, Artificial
  Designed peptide recognized by HLA-A2 restricted cytotoxic T lymphocytes
IQAKTFHHV

SEQ ID NO: 55 - protein, 10 aa, Artificial
  Designed peptide recognized by HLA-A2 restricted cytotoxic T lymphocytes
KVVSSKTKKV

SEQ ID NO: 56 - protein, 10 aa, Artificial
  Designed peptide recognized by HLA-A2 restricted cytotoxic T lymphocytes
ATFKSFEDRV

SEQ ID NO: 57 - protein, 10 aa, Artificial
  Designed peptide recognized by HLA-A2 restricted cytotoxic T lymphocytes
KIGPRRIHTV

SEQ ID NO: 58 - protein, 10 aa, Artificial
  Designed peptide recognized by HLA-A2 restricted cytotoxic T lymphocytes
TLVKNCIVLI

SEQ ID NO: 59 - protein, 9 aa, Artificial
  Designed peptide recognized by HLA-A2 restricted cytotoxic T lymphocytes
RQWYESHYA

SEQ ID NO: 60 - protein, 9 aa, Artificial
  Designed peptide recognized by HLA-A 2 restriced cytotoxic T lymphocytes
KLTPEEEEI

SEQ ID NO: 61 - protein, 10 aa, Artificial
  Designed peptide recognized by HLA-A2 restricted cytotoxic T lymphocytes
ILNKKRSKKI

SEQ ID NO: 62 - protein, 9 aa, Artificial
  Designed peptide recognized by HLA-A2 restricted cytotoxic T lymphocytes
YLNEQVKAI

SEQ ID NO: 63 - protein, 10 aa, Artificial
  Designed peptide recognized by HLA-A2 restricted cytotoxic T lymphocytes
SLLELHKLAT

SEQ ID NO: 64 - protein, 10 aa, Artificial
  Designed peptide recognized by HLA-A2 restricted cytotoxic T lymphocytes
FLQDIKKPDC

SEQ ID NO: 65 - protein, 10 aa, Artificial
  Designed peptide recognized by HLA-A2 restricted cytotoxic T lymphocytes
INLELYASYV

SEQ ID NO: 66 - protein, 10 aa, Artificial
  Designed peptide recognized by HLA-A2 restricted cytotoxic T lymphocytes
KIYKIGQGYL

SEQ ID NO: 67 - protein, 9 aa, Artificial
  Designed peptide recognized by HLA-A2 restricted cytotoxic T lymphocytes
KLIKNNAST

SEQ ID NO: 68 - protein, 9 aa, Artificial
  Designed peptide recognized by HLA-A2 restricted cytotoxic T lymphocytes
GMTHIVREV

SEQ ID NO: 69 - protein, 10 aa, Artificial
  Designed peptide recognized by HLA-A2 restricted cytotoxic T lymphocytes
RLLPLRQKKA

SEQ ID NO: 70 - protein, 10 aa, Artificial
  Designed peptide recognized by HLA-A2 restricted cytotoxic T lymphocytes
FLIFEDRKFA

SEQ ID NO: 71 - protein, 9 aa, Artificial
  Designed peptide recognized by HLA-A2 restricted cytotoxic T lymphocytes
ALGPSICML

SEQ ID NO: 72 - protein, 10 aa, Artificial
  Designed peptide recognized by HLA-A2 restricted cytotoxic T lymphocytes
QLADALGPSI

SEQ ID NO: 73 - protein, 10 aa, Artificial
  Designed peptide recognized by HLA-A2 restricted cytotoxic T lymphocytes
GLPLHRGCLL

SEQ ID NO: 74 - protein, 9 aa, Artificial
  Designed peptide recognized by HLA-A2 restricted cytotoxic T lymphocytes
YLYLISSCI

SEQ ID NO: 75 - protein, 10 aa, Artificial
  Designed peptide recognized by HLA-A2 restricted cytotoxic T lymphocytes
YLISSCIKPI

SEQ ID NO: 76 - protein, 9 aa, Artificial
  Designed peptide recognized by HLA-A2 restricted cytotoxic T lymphocytes
VISCYICKV

SEQ ID NO: 77 - protein, 10 aa, Artificial
  Designed peptide recognized by HLA-A2 restricted cytotoxic T lymphocytes
WLSDQLQNNC

SEQ ID NO: 78 - protein, 9 aa, Artificial
  Designed peptide recognized by HLA-A2 restricted cytotoxic T lymphocytes
MLCGNIYPI

SEQ ID NO: 79 - protein, 10 aa, Artificial
  Designed peptide recognized by HLA-A2 restricted cytotoxic T lymphocytes
YLPSGSSAHL

SEQ ID NO: 80 - protein, 9 aa, Artificial
  Designed peptide recognized by HLA-A2 restricted cytotoxic T lymphocytes
SMQDDAFPA

SEQ ID NO: 81 - protein, 9 aa, Artificial
  Designed peptide recognized by HLA-A2 restricted cytotoxic T lymphocytes
TLIPTFDSV

SEQ ID NO: 82 - protein, 10 aa, Artificial
  Designed peptide recognized by HLA-A2 restricted cytotoxic T lymphocytes
FQRVRALCYV

SEQ ID NO: 83 - protein, 10 aa, Artificial
  Designed peptide recognized by HLA-A2 restricted cytotoxic T lymphocytes
VLGSNGMVSM

SEQ ID NO: 84 - protein, 10 aa, Artificial
  Designed peptide recognized by HLA-A2 restricted cytotoxic T lymphocytes
FLTKIFHPNV

SEQ ID NO: 85 - protein, 10 aa, Artificial
  Designed peptide recognzied by HLA-A2 restricted cytotoxic T lymphocytes
LLLENYEEYA

SEQ ID NO: 86 - protein, 9 aa, Artificial
  Designed peptide recognized by HLA-A2 restricted cytotoxic T lymphocytes
VLLTIKCLL

SEQ ID NO: 87 - protein, 9 aa, Artificial
  Designed peptide recognized by HLA-A2 restricted cytotoxic T lymphocytes
GLFRMKLLL

SEQ ID NO: 88 - protein, 9 aa, Artificial
  Designed peptide recognized by HLA-A2 restricted cytotoxic T lymphocytes
NLPPHIIRL

SEQ ID NO: 89 - protein, 9 aa, Artificial
  Designed peptide recognized by HLA-A2 restricted cytokine T lymphocytes
KLTNTYCLV

SEQ ID NO: 90 - protein, 9 aa, Artificial
  Designed peptide recognized by HLA-A2 restricted cytotoxic T lymphocytes
GLLVPNNTT

SEQ ID NO: 91 - protein, 9 aa, Artificial
  Designed peptide recognized by HLA-A2 restricted cytotoxic T lymphocytes
GMVVNDWCA

SEQ ID NO: 92 - protein, 10 aa, Artificial
  Designed peptide recognized by HLA-A2 restricted cytotoxic T lymphocytes
VLLRQGVLGI

SEQ ID NO: 93 - protein, 10 aa, Artificial
  Designed peptide recognized by HLA-A2 restricted cytotoxic T lymphocytes
GLMIHSGDPV

SEQ ID NO: 94 - protein, 9 aa, Artificial
  Designed peptide recognized by HLA-A2 restricted cytotoxic T lymphocytes
ILATRTQNV

SEQ ID NO: 95 - protein, 8 aa, Artificial
  Designed peptide recognized by HLA-A2 restricted cytotoxic T lymphocytes
FVADIFLA

SEQ ID NO: 96 - protein, 10 aa, Artificial
  Designed peptide recognized by HLA-A2 restricted cytotoxic T lymphocytes
FIMESGAKGC

SEQ ID NO: 97 - protein, 10 aa, Artificial
  Designed peptide recognized by HLA-A2 restricted cytotoxic T lymphocytes
WIPNNVLTAV

SEQ ID NO: 98 - protein, 9 aa, Artificial
  Designed peptide recognized by HLA-A2 restricted cytotoxic T lymphocytes
RIMNTFSVV

SEQ ID NO: 99 - protein, 9 aa, Artificial
  Designed peptide recognized by HLA-A2 restricted cytotoxic T lymphocytes
LVSATMSGV

SEQ ID NO: 100 - protein, 9 aa, Artificial
  Designed peptide recognized by HLA-A2 restricted cytotoxic T lymphocytes
SLNRRIQLV

SEQ ID NO: 101 - protein, 9 aa, Artificial
  Designed peptide recognized by HLA-A2 restricted cytotoxic T lymphocytes
RLATALQKL

SEQ ID NO: 102 - protein, 10 aa, Artificial
  Designed peptide recognized by HLA-A2 restricted cytotoxic T lymphocytes
QLVEEELDRA

SEQ ID NO: 103 - protein, 10 aa, Artificial
  Designed peptide recognized by HLA-A2 restricted cytotoxic T lymphocytes
GISLANQQYV

SEQ ID NO: 104 - protein, 9 aa, Artificial
  Designed peptide recognized by HLA-A2 restricted cytotoxic T lymphocytes
FLHSGHLHA

SEQ ID NO: 105 - protein, 9 aa, Artificial
  Designed peptide recognized by HLA-A2 restricted cytotoxic T lymphocytes
ELVRFRQKV

SEQ ID NO: 106 - protein, 9 aa, Artificial
  Designed peptide recognized by HLA-A2 restricted cytotoxic T lymphocytes
KLSEAAGRV

SEQ ID NO: 107 - protein, 9 aa, Artificial
  Designed peptide recognized by HLA-A2 restricted cytotoxic T lymphocytes
MVLDLMQQL

SEQ ID NO: 108 - protein, 10 aa, Artificial
  Designed peptide recognized by HLA-A2 restricted cytotoxic T lymphocytes
IMQNLLSKDV

SEQ ID NO: 109 - protein, 10 aa, Artificial
  Designed peptide recognized by HLA-A2 restricted cytotoxic T lymphocytes
ELAEEEPHLV

SEQ ID NO: 110 - protein, 9 aa, Artificial
  Designed peptide recognized by HLA-A2 restricted cytotoxic T lymphocytes
GLADSGWFL

SEQ ID NO: 111 - protein, 9 aa, Artificial
  Designed peptide recognized by HLA-A2 restricted cytotoxic T lymphocytes
KQYRHTDCV

SEQ ID NO: 112 - protein, 10 aa, Artificial
  Designed peptide recognized by HLA-A2 restricted cytotoxic T lymphocytes
VQWLFDEAQL

SEQ ID NO: 113 - protein, 10 aa, Artificial
  Designed peptide recognized by HLA-A2 restricted cytotoxic T lymphocytes
IIIASHWTDV

SEQ ID NO: 114 - protein, 10 aa, Artificial
  Designed peptide recognized by HLA-A2 restricted cytotoxic T lymphocytes
NLGRELRHTL

SEQ ID NO: 115 - protein, 9 aa, Artificial
  Designed peptide recognized by HLA-A2 restricted cytotoxic T lymphocytes
LLGRGLSGA

SEQ ID NO: 116 - protein, 9 aa, Artificial
  Designed peptide recognized by HLA-A2 restricted cytotoxic T lymphocytes
VLYLFYQDM

SEQ ID NO: 117 - protein, 9 aa, Artificial
  Designed peptide recognized by HLA-A2 restricted cytotoxic T lymphocytes
YVARNAKDV

SEQ ID NO: 118 - protein, 10 aa, Artificial
  Designed peptide recognized by HLA-A2 restricted cytotoxic T lymphocytes
LIQDTSRPPL

SEQ ID NO: 119 - protein, 10 aa, Artificial
  Designed peptide recognized by HLA-A2 restricted cytotoxic T lymphocytes
GLFIFSIVFL

SEQ ID NO: 120 - protein, 10 aa, Artificial
  Designed peptide recognized by HLA-A2 restricted cytotoxic T lymphocytes
WLLLPLLGAV

SEQ ID NO: 121 - protein, 9 aa, Artificial
  Designed peptide recognized by HLA-A2 restricted cytotoxic T lymphocytes
ILFRGVGMV

SEQ ID NO: 122 - protein, 10 aa, Artificial
  Designed peptide recognized by HLA-A2 restricted cytotoxic T lymphocytes
GLQARNNARV

SEQ ID NO: 123 - protein, 10 aa, Artificial
  Designed peptide recognized by HLA-A2 restricted cytotoxic T lymphocytes
DVYGVFQFKV

SEQ ID NO: 124 - protein, 10 aa, Artificial
  Designed peptide recognized by HLA-A2 restricted cytotoxic T lymphocytes
SLNPILFRGV

SEQ ID NO: 125 - protein, 9 aa, Artificial
  Designed peptide recognized by HLA-A2 restricted cytotoxic T lymphocytes
TLHTWGSKV

SEQ ID NO: 126 - protein, 9 aa, Artificial
  Designed peptide recognized by HLA-A2 restricted cytotoxic T lymphocytes
CLPSGFPGL

SEQ ID NO: 127 - protein, 9 aa, Artificial
  Designed peptide recognized by HLA-A2 restricted cytotoxic T lymphocytes
NLVKCIKRL

SEQ ID NO: 128 - protein, 9 aa, Artificial
  Designed peptide recognized by HLA-A2 restricted cytotoxic T lymphocytes
TVFLEGNLV

SEQ ID NO: 129 - protein, 9 aa, Artificial
  Designed peptide recognized by HLA-A2 restricted cytotoxic T lymphocytes
FLLLLLFET

SEQ ID NO: 130 - protein, 9 aa, Artificial
  Designed peptide recognized by HLA-A2 restricted cytotoxic T lymphocytes
YIFFCVLFL

SEQ ID NO: 131 - protein, 9 aa, Artificial
  Designed peptide recognized by HLA-A2 restricted cytotoxic T lymphocytes
FLLLFGFWK

SEQ ID NO: 132 - protein, 9 aa, Artificial
  Designed peptide recognized by HLA-A2 restricted cytotoxic T lymphocytes
SVHPRLFLL

SEQ ID NO: 133 - protein, 9 aa, Artificial
  Designed peptide recognized by HLA-A2 restricted cytotoxic T lymphocytes
ILFPRKPSA

SEQ ID NO: 134 - protein, 10 aa, Artificial
  Designed peptide recognized by HLA-A2 restricted cytotoxic T lymphocytes
KVARTIGISV

SEQ ID NO: 135 - protein, 10 aa, Artificial
  Designed peptide recognized by HLA-A2 restricted cytotoxic T lymphocytes
FLAILGGAKV

SEQ ID NO: 136 - protein, 9 aa, Artificial
  Designed peptide recognized by HLA-A2 restricted cytotoxic T lymphocytes
VVMRVDFNV

SEQ ID NO: 137 - protein, 9 aa, Artificial
  Designed peptide recognized by HLA-A2 restricted cytotoxic T lymphocytes
LITLPVDFV

SEQ ID NO: 138 - protein, 10 aa, Artificial
  Designed peptide recognized by HLA-A2 restricted cytotoxic T lymphocytes
SLFDEEGAKI

SEQ ID NO: 139 - protein, 10 aa, Artificial
  Designed peptide recognized by HLA-A2 restricted cytotoxic T lymphocytes
QLINNMLDKV

SEQ ID NO: 140 - protein, 10 aa, Artificial
  Designed peptide recognized by HLA-A2 restricted cytotoxic T lymphocytes
FCLDNGAKSV

SEQ ID NO: 141 - protein, 9 aa, Artificial
  Designed peptide recognized by HLA-A2 restricted cytotoxic T lymphocytes
IIGGGMAFT

SEQ ID NO: 142 - protein, 10 aa, Artificial
  Designed peptide recognized by HLA-A2 restricted cytotoxic T lymphocytes
ALFVSFIINV

SEQ ID NO: 143 - protein, 10 aa, Artificial
  Designed peptide recognized by HLA-A2 restricted cytotoxic T lymphocytes
VLITIADTFV

SEQ ID NO: 144 - protein, 9 aa, Artificial
  Designed peptide recognized by HLA-A2 restricted cytotoxic T lymphocytes
FLFLDKYGL

SEQ ID NO: 145 - protein, 9 aa, Artificial
  Designed peptide recognized by HLA-A2 restricted cytotoxic T lymphocytes
ALTFGYEYV

SEQ ID NO: 146 - protein, 10 aa, Artificial
  Designed peptide recognized by HLA-A2 restricted cytotoxic T lymphocytes
YLGWQCLIAL

SEQ ID NO: 147 - protein, 10 aa, Artificial
  Designed peptide recognized by HLA-A2 restricted cytotoxic T lymphocytes
KLLWILLLAT

SEQ ID NO: 148 - protein, 9 aa, Artificial
  Designed peptide recognized by HLA-A2 restricted cytotoxic T lymphocytes
MLFIHAEVI

SEQ ID NO: 149 - protein, 9 aa, Artificial
  Designed peptide recognized by HLA-A2 restricted cytotoxic T lymphocytes
KLIKRSGYI

SEQ ID NO: 150 - protein, 10 aa, Artificial
  Designed peptide recognized by HLA-A2 restricted cytotoxic T lymphocytes
SLPVCSLKLI

SEQ ID NO: 151 - protein, 10 aa, Artificial
  Designed peptide recognized by HLA-A2 restricted cytotoxic T lymphocytes
FVISLPVCSL

SEQ ID NO: 152 - protein, 10 aa, Artificial
  Designed peptide recognized by HLA-A2 restricted cytotoxic T lymphocytes
LQFDENTNWL

SEQ ID NO: 153 - protein, 9 aa, Artificial
  Designed peptide recognized by HLA-A2 restricted cytotoxic T lymphocytes
FLNGYNCTV

SEQ ID NO: 154 - protein, 10 aa, Artificial
  Designed peptide recognized by HLA-A2 restricted cytotoxic T lymphocytes
AMLKTRRSYL

SEQ ID NO: 155 - protein, 10 aa, Artificial
  Designed peptide recognized by HLA-A2 restricted cytotoxic T lymphocytes
TLMKPSSFTT

SEQ ID NO: 156 - protein, 10 aa, Artificial
  Designed peptide recognized by HLA-A2 restricted cytotoxic T lymphocytes
LLVNSGPLAV

SEQ ID NO: 157 - protein, 10 aa, Artificial
  Designed peptide recognized by HLA-A2 restricted cytotoxic T lymphocytes
MLGSADEPGV

SEQ ID NO: 158 - protein, 10 aa, Artificial
  Designed peptide recognized by HLA-A2 restricted cytotoxic T lymphocytes
KQNDLPGISV

SEQ ID NO: 159 - protein, 10 aa, Artificial
  Designed peptide recognized by HLA-A2 restricted cytotoxic T lymphocytes
YLTMLHLYLC

SEQ ID NO: 160 - protein, 10 aa, Artificial
  Designed peptide recognized by HLA-A2 restricted cytotoxic T lymphocytes
ITGEAFVQFA

SEQ ID NO: 161 - protein, 10 aa, Artificial
  Designed peptide recognized by HLA-A2 restricted cytotoxic T lymphocytes
VVACNLYPFV

SEQ ID NO: 162 - protein, 9 aa, Artificial
  Designed peptide recognized by HLA-A2 restricted cytotoxic T lymphocytes
MLGGRVKTL

SEQ ID NO: 163 - protein, 9 aa, Artificial
  Designed peptide recognized by HLA-A2 restricted cytotoxic T lymphocytes
QLYTLQPKL

SEQ ID NO: 164 - protein, 9 aa, Artificial
  Designed peptide recognized by HLA-A2 restricted cytotoxic T lymphocytes
GLVEFARNL

SEQ ID NO: 165 - protein, 10 aa, Artificial
  Designed peptide recognized by HLA-A2 restricted cytotoxic T lymphocytes
FVALSDVCDV

SEQ ID NO: 166 - protein, 9 aa, Artificial
  Designed peptide recognized by HLA-A2 restricted cytotoxic T lymphocytes
RLDFNLIRV

SEQ ID NO: 167 - protein, 9 aa, Artificial
  Designed peptide recognized by HLA-A2 restricted cytotoxic T lymphocytes
ILAHTNLRL

SEQ ID NO: 168 - protein, 10 aa, Artificial
  Designed peptide recognized by HLA-A2 restricted cytotoxic T lymphocytes
CMVYDLYKTL

SEQ ID NO: 169 - protein, 10 aa, Artificial
  Designed peptide recognized by HLA-A2 restricted cytotoxic T lymphocytes
WQLVKELKEA

SEQ ID NO: 170 - protein, 9 aa, Artificial
  Designed peptide recognized by HLA-A2 restricted cytotoxic T lymphocytes
LLLTAPNLL

SEQ ID NO: 171 - protein, 10 aa, Artificial
  Designed peptide recognized by HLA-A2 restricted cytotoxic T lymphocytes
ALFPGLAPET

SEQ ID NO: 172 - protein, 9 aa, Artificial
  Designed peptide recognized by HLA-A2 restricted cytotoxic T lymphocytes
WLLGGHVEL

SEQ ID NO: 173 - protein, 10 aa, Artificial
  Designed peptide recognized by HLA-A2 restricted cytotoxic T lymphocytes
FLHLLQADNV

SEQ ID NO: 174 - protein, 10 aa, Artificial
  Designed peptide recognized by HLA-A2 restricted cytotoxic T lymphocytes
LQSDHFLHLL

SEQ ID NO: 175 - protein, 10 aa, Artificial
  Designed peptide recognized by HLA-A2 restricted cytotoxic T lymphocytes
MMMLQNILQI

SEQ ID NO: 176 - protein, 10 aa, Artificial
  Designed peptide recognized by HLA-A2 restricted cytotoxic T lymphocytes
QLVGLLSPMV

SEQ ID NO: 177 - protein, 10 aa, Artificial
  Designed peptide recognized by HLA-A2 restricted cytotoxic T lymphocytes
LLMAESHQEI

SEQ ID NO: 178 - protein, 9 aa, Artificial
  Designed peptide recognized by HLA-A2 restricted cytotoxic T lymphocytes
KLHQAACLI

SEQ ID NO: 179 - protein, 9 aa, Artificial
  Designed peptide recognized by HLA-A2 restricted cytotoxic T lymphocytes
ILSHCCVGL

SEQ ID NO: 180 - protein, 10 aa, Artificial
  Designed peptide recognized by HLA-A2 restricted cytotoxic T lymphocytes
SLFWLLGGHV

SEQ ID NO: 181 - protein, 9 aa, Artificial
  Designed peptide recognized by HLA-A2 restricted cytotoxic T lymphocytes
KLFAPWRGL

SEQ ID NO: 182 - protein, 10 aa, Artificial
  Designed peptide recognized by HLA-A2 restricted cytotoxic T lymphocytes
KLGEESGDEI

SEQ ID NO: 183 - protein, 10 aa, Artificial
  Designed peptide recognized by HLA-A2 restricted cytotoxic T lymphocytes
YDYDGYRLRV

SEQ ID NO: 184 - protein, 9 aa, Artificial
  Designed peptide recognized by HLA-A2 restricted cytotoxic T lymphocytes
RGGPPFAFV

SEQ ID NO: 185 - protein, 9 aa, Artificial
  Designed peptide recognized by HLA-A2 restricted cytotoxic T lymphocytes
TLGDAHIYL

SEQ ID NO: 186 - protein, 9 aa, Artificial
  Designed peptide recognized by HLA-A2 restricted cytotoxic T lymphocytes
TMIAHITGL

SEQ ID NO: 187 - protein, 10 aa, Artificial
  Designed peptide recognized by HLA-A2 restricted cytotoxic T lymphocytes
TLNHIEPLKI

SEQ ID NO: 188 - protein, 10 aa, Artificial
  Designed peptide recognized by HLA-A2 restricted cytotoxic T lymphocytes
LMALPPCHAL

SEQ ID NO: 189 - protein, 9 aa, Artificial
  Designed peptide recognized by HLA-A2 restricted cytotoxic T lymphocytes
KLLWTTSRV

SEQ ID NO: 190 - protein, 10 aa, Artificial
  Designed peptide recognized by HLA-A2 restricted cytotoxic T lymphocytes
RLVQNCLWTL

SEQ ID NO: 191 - protein, 9 aa, Artificial
  Designed peptide recognized by HLA-A2 restricted cytotoxic T lymphocytes
VLFYAITTL

SEQ ID NO: 192 - protein, 9 aa, Artificial
  Designed peptide recognized by HLA-A2 restricted cytotoxic T lymphocytes
IMFDVTSRV

SEQ ID NO: 193 - protein, 10 aa, Artificial
  Designed peptide recognized by HLA-A2 restricted cytotoxic T lymphocytes
LTGEFEKKYV

SEQ ID NO: 194 - protein, 10 aa, Artificial
  Designed peptide recognized by HLA-A2 restricted cytotoxic T lymphocytes
ALYEKDNTYL

SEQ ID NO: 195 - protein, 10 aa, Artificial
  Designed peptide recognized by HLA-A2 restricted cytotoxic T lymphocytes
FMILASPRYV

SEQ ID NO: 196 - protein, 10 aa, Artificial
  Designed peptide recognized by HLA-A2 restricted cytotoxic T lymphocytes
KLTSLQLQHL

SEQ ID NO: 197 - protein, 10 aa, Artificial
  Designed peptide recognized by HLA-A2 restricted cytotoxic T lymphocytes
SLQLQHLFMI

SEQ ID NO: 198 - protein, 9 aa, Artificial
  Designed peptide recognized by HLA-A2 restricted cytotoxic T lymphocytes
QVLPMLRFV

SEQ ID NO: 199 - protein, 9 aa, Artificial
  Designed peptide recognized by HLA-A2 restricted cytotoxic T lymphocytes
KMVTMVSVL

SEQ ID NO: 200 - protein, 9 aa, Artificial
  Designed peptide recognized by HLA-A2 restricted cytotoxic T lymphocytes
ALFKCYMFL

SEQ ID NO: 201 - protein, 9 aa, Artificial
  Designed peptide recognized by HLA-A2 restricted cytotoxic T lymphocytes
FLALPLEDV

SEQ ID NO: 202 - protein, 10 aa, Artificial
  Designed peptide recognized by HLA-A2 restricted cytotoxic T lymphocytes
RLPLCRPQFL

SEQ ID NO: 203 - protein, 9 aa, Artificial
  Designed peptide recognized by HLA-A2 restricted cytotoxic T lymphocytes
LMPERRPHL

SEQ ID NO: 204 - protein, 10 aa, Artificial
  Designed peptide recognized by HLA-A2 restricted cytotoxic T lymphocytes
FLQLQSIKDA

SEQ ID NO: 205 - protein, 9 aa, Artificial
  Designed peptide recognized by HLA-A2 restricted cytotoxic T lymphocytes
KILFKTWHL

SEQ ID NO: 206 - protein, 9 aa, Artificial
  Designed peptide recognized by HLA-A2 restricted cytotoxic T lymphocytes
ILFKTWHLI

SEQ ID NO: 207 - protein, 9 aa, Artificial
  Designed peptide recognized by HLA-A2 restricted cytotoxic T lymphocytes
FLPPFSLSL

SEQ ID NO: 208 - protein, 10 aa, Artificial
  Designed peptide recognized by HLA-A2 restricted cytotoxic T lymphocytes
SLPLFLPPFL

SEQ ID NO: 209 - protein, 10 aa, Artificial
  Designed peptide recognized by HLA-A2 restricted cytotoxic T lymphocytes
GLYFLYSMPV

SEQ ID NO: 210 - protein, 10 aa, Artificial
  Designed peptide recognized by HLA-A2 restricted cytotoxic T lymphocytes
FVGGHVGWPT

SEQ ID NO: 211 - protein, 10 aa, Artificial
  Designed peptide recognized by HLA-A2 restricted cytotoxic T lymphocytes
RLHNDRVYYV

SEQ ID NO: 212 - protein, 10 aa, Artificial
  Designed peptide recognized by HLA-A2 restricted cytotoxic T lymphocytes
YIGENLQLLV

SEQ ID NO: 213 - protein, 9 aa, Artificial
  Designed peptide recognized by HLA-A2 restricted cytotoxic T lymphocytes
YVSEKIMKL

SEQ ID NO: 214 - protein, 335 aa, Homo sapiens
MGKVKVGVNGFGRIGRLVTRAAFNSGKVDIVAINDPFIDLNYMVYMFQYDSTHGKFHGTVKAENGKLVINGNPITIFQERDPSKIKWGDAGAEYVVESTGVFTTMEKAGAHLQGGAKRVIISAPSADAPMFVMGVNHEKYDNSLKIISNASCTTNCLAPLAKVIHDNFGIVEGLMTTVHAITATQKTVDGPSGKLWRDGRGALQNIIPASTGAAKAVGKVIPELNGKLTGMAFRVPTANVSVVDLTCRLEKPAKYDDIKKVVKQASEGPLKGILGYTEHQVVSSDFNSDTHSSTFDAGAGIALNDHFVKLISWYDNEFGYSNRVVDLMAHMASKE

SEQ ID NO: 215 - protein, 599 aa, Homo sapiens
MADKLTRIAIVNHDKCKPKKCRQECKKSCPVVRMGKLCIEVTPQSKIAWISETLCIGCGICIKKCPFGALSIVNLPSNLEKETTHRYCANAFKLHRLPIPRPGEVLGLVGTNGIGKSTALKILAGKQKPNLGKYDDPPDWQEILTYFRGSELQNYFTKILEDDLKAIIKPQYVDQIPKAAKGTVGSILDRKDETKTQAIVCQQLDLTHLKERNVEDLSGGELQRFACAVVCIQKADIFMFDEPSSYLDVKQRLKAAITIRSLINPDRYIIVVEHDLSVLDYLSDFICCLYGVPSAYGVVTMPFSVREGINIFLDGYVPTENLRFRDASLVFKVAETANEEEVKKMCMYKYPGMKKKMGEFELAIVAGEFTDSEIMVMLGENGTGKTTFIRMLAGRLKPDEGGEVPVLNVSYKPQKISPKSTGSVRQLLHEKIRDAYTHPQFVTDVMKPLQIENIIDQEVQTLSGGELQRVALALCLGKPADVYLIDEPSAYLDSEQRLMAARVVKRFILHAKKTAFVVEHDFIMATYLADRVIVFDGVPSKNTVANSPQTLLAGMNKFLSQLEITFRRDPNNYRPRINKLNSIKDVEQKKSGNYFFLDD

SEQ ID NO: 216 - protein, 101 aa, Homo sapiens
MSDQEAKPSTEDLGDKKEGEYIKLKVIGQDSSEIHFKVKMTTHLKKLKESYCQRQGVPMDSLRFLFEGQRIADDHTPKELGMEEEDVIEVYQEQTGGHSTV

SEQ ID NO: 217 - protein, 249 aa, Homo sapiens
MKLNISFPATGCQKLIEVDDERKLRTFYEKRMATEVAADALGEEWKGYVVRISGGNDKQGFPMKQGVLTHGRVRLLLSKGHSCYRPRRTGERKRKSVRGCIVDANLSVLNLVIVKKGEKDIPGLTDTTVPRRLGPKRASRIRKLFNLSKEDDVRQYVVRKPLNKEGKKPRTKAPKIQRLVTPRVLQHKRRRIALKKQRTKKNKEEAAEYAKLLAKRMKEAKEKRQEQIAKRRRLSSLRASTSKSESSQK

SEQ ID NO: 218 - protein, 184 aa, Homo sapiens
MREYKLVVLGSGGVGKSALTVQFVQGIFVEKYDPTIEDSYRKQVEVDAQQCMLEILDTAGTEQFTAMRDLYMKNGQGFALVYSITAQSTFNDLQDLREQILRVKDTDDVPMILVGNKCDLEDERVVGKEQGQNLARQWNNCAFLESSAKSKINVNEIFYDLVRQINRKTPVPGKARKKSSCQLL

SEQ ID NO: 219 - protein, 162 aa, Homo sapiens
MKETIMNQEKLAKLQAQVRIGGKGTARRKKKVVHRTATADDKKLQFSLKKLGVNNISGIEEVNMFTNQGTVIHFNNPKVQASLAANTFTITGHAETKQLTEMLPSILNQLGADSLTSLRRLAEALPKQSVDGKAPLATGEDDDDEVPDLVENFDEASKNEAN

SEQ ID NO: 220 - protein, 180 aa, Homo sapiens
MRPLTEEETRVMFEKIAKYIGENLQLLVDRPDGTYCFRLHNDRVYYVSEKIMKLAANISGDKLVSLGTCFGKFTKTHKFRLHVTALDYLAPYAKYKVWIKPGAEQSFLYGNHVLKSGLGRITENTSQYQGVVVYSMADIPLGFGVAAKSTQDCRKVDPMAIVVFHQADIGEYVRHEETLT

SEQ ID NO: 221 - protein, 166 aa, Homo sapiens
MAATMFRATLRGWRTGVQRGCGLRLLSQTQGPPDYPRFVESVDEYQFVERLLPATRIPDPPKHEHYPTPSGWQPPRDPPPNLPYFVRRSRMHNIPVYKDITHGNRQMTVIRKVEGDIWALQKDVEDFLSPLLGKTPVTQVNEVTGTLRIKGYFDQELKAWLLEKGF

SEQ ID NO: 222 - protein, 194 aa, Homo sapiens
MAASLVGKKIVFVTGNAKKLEEVVQILGDKFPCTLVAQKIDLPEYQGEPDEISIQKCQEAVRQVQGPVLVEDTCLCFNALGGLPGPYIKWFLEKLKPEGLHQLLAGFEDKSAYALCTFALSTGDPSQPVRLFRGRTSGRIVAPRGCQDFGWDPCFQPDGYEQTYAEMPKAEKNAVSHRFRALLELQEYFGSLAA

SEQ ID NO: 223 - protein, 466 aa, Homo sapiens
MSYPGYPPTGYPPFPGYPPAGQESSFPPSGQYPYPSGFPPMGGGAYPQVPSSGYPGAGGYPAPGGYPAPGGYPGAPQPGGAPSYPGVPPGQGFGVPPGGAGFSGYPQPPSQSYGGGPAQVPLPGGFPGGQMPSQYPGGQPTYPSQPATVTQVTQGTIRPAANFDAIRDAEILRKAMKGFGTDEQAIVDVVANRSNDQRQKIKAAFKTSYGKDLIKDLKSELSGNMEELILALFMPPTYYDAWSLRLAMQGAGTQERVLIEILCTRTNQEIREIVRCYQSEFGRDLELDIRSDTSGHFERLLVSMCQGNRDENQSINHQMAQEDAQRLYQAGEGRLGTDESCFNMILATRSFPQLRATMEAYSRMANRDLLSSVSREFSGYVESGLKTILQCALNRPAFFAERLYYAMKGAGTDDSTLVRIVVTRSEIDLVQIKQMFAQMYQKTLGTMIAGDTSGDYRRLLLAIVGQ

SEQ ID NO: 224 - protein, 130 aa, Homo sapiens
MVRMNVLADALKSINNAEKRGKRQVLIRPCSKVIVRFLTVMMKHGYIGEFEIIDDHRAGKIVVNLTGRLNKCGVISPRFDVQLKDLEKWQNNLLPSRQFGFIVLTTSAGIMDHEEARRKHTGGLILGFFF

SEQ ID NO: 225 - protein, 192 aa, Homo sapiens
MLTILSNQTVDIPENVDITLKGRTVIVKGPRGTLRRDFNHINVELSLLGKKKKRLRVDKWWGNRLELATVRTICSHVQNMIKGVTLGFRYKMRSVYAHFPINVVIQENGSLVEIRNFLGELYIRRVRMRPGVACSVSQAQKDELILEGNDIELVSNSAALIQQATTVKNKDIRKFLDGIYVSEKGTVQQADE

SEQ ID NO: 226 - protein, 67 aa, Homo sapiens
MLLYINRARPEGGRGAGAEGRSNQISNFLLIINPLFTAVSVVIFLIFLIFFFFLLLLFTSCVYVGNL

SEQ ID NO: 227 - protein, 66 aa, Homo sapiens
MHFHNICLLERSIISEKYQVFIKFLGMADSQNMLVSLQYSSRRANQGRAGMRSDICVTKSIFLISL

SEQ ID NO: 228 - protein, 145 aa, Homo sapiens
MILQCSIEMPNISYAWKELKEQLGEEIDSKVKGMVFLKGKLGVCFDVPTASVTEIQEKWHDSRRWQLSVATEQPELEGPREGYGGFRGQREGSRGFRGQRDGNRRFRGQREGSRGPRGQRSGGGNKSNRSQNKGQKRSFSKAFGQ

SEQ ID NO: 229 - protein, 49 aa, Homo sapiens
MRNSATFKSFEDRVGTIKSKVVGDRENGSDNLPSSAGSGDKPLSDPAPF

SEQ ID NO: 230 - protein, 208 aa, Homo sapiens [sequence omitted]

SEQ ID NO: 231 - protein, 183 aa, Homo sapiens
MTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 232 - protein, 403 aa, Homo sapiens
MSHRKFSAPRHGSLGFLPRKRSSRHRGKVKSFPKDDPSKPVHLTAFLGYKAGMTHIVREVDRPGSKVNKKEVVEAVTIVETPPMVVVGIVGYVETPRGLRTFKTVFAEHISDECKRRFYKNWHKSKKKAFTKYCKKWQDEDGKKQLEKDFSSMKKYCQVIRVIAHTQMRLLPLRQKKAHLMEIQVNGGTVAEKLDWARERLEQQVPVNQVFGQDEMIDVIGVTKGKGYKGVTSRWHTKKLPRKTHRGLRKVACIGAWHPARVAFSVARAGQKGYHHRTEINKKIYKIGQGYLIKDGKLIKNNASTDYDLSDKSINPLGGFVHYGEVTNDFVMLKGCVVGTKKRVLTLRKSLLVQTKRRALEKIDLKFIDTTSKFGHGRFQTMEEKKAFMGPLKKDRIAKEEGA

SEQ ID NO: 233 - protein, 480 aa, Homo sapiens
MAVARAALGPLVTGLYDVQAFKFGDFVLKSGLSSPIYIDLRGIVSRPRLLSQVADILFQTAQNAGISFDTVCGVPYTALPLATVICSTNQIPMLIRRKETKDYGTKRLVEGTINPGETCLIIEDVVTSGSSVLETVEVLQKEGLKVTDAIVLLDREQGGKDKLQAHGIRLHSVCTLSKMLEILEQQKKVDAETVGRVKRFIQENVFVAANHNGSPLSIKEAPKELSFGARAELPRIHPVASKLLRLMQKKETNLCLSADVSLARELLQLADALGPSICMLKTHVDILNDFTLDVMKELITLAKCHEFLIFEDRKFADIGNTVKKQYEGGIFKIASWADLVNAHVVPGSGVVKGLQEVGLPLHRGCLLIAEMSSTGSLATGDYTRAAVRMAEEHSEFVVGFISGSRVSMKPEFLHLTPGVQLEAGGDNLGQQYNSPQEVIGKRGSDIIIVGRGIISAADRLEAAEMYRKAAWEAYLSRLGV

SEQ ID NO: 234 - protein, 86 aa, Homo sapiens [sequence omitted]

SEQ ID NO: 235 - protein, 54 aa, Homo sapiens
MLCGNIYPIDHPILMCLWLSDQLQNNCVVILCPKLLINFYLQIEKEGPCKENGK

SEQ ID NO: 236 - protein, 672 aa, Homo sapiens
MGVGRLDMYVLHPPSAGAERTLASVCALLVWHPAGPGEKVVRVLFPGCTPPACLLDGLVRLQHLRFLREPVVTPQDLEGPGRAESKESVGSRDSSKREGLLATHPRPGQERPGVARKEPARAEAPRKTEKEAKAPRELKKDPKPSVSRTQPREVRRAASSVPDLKKTDAQAAPKPRKAPSTSHSGFPPVADGPRSPPSLRCGEASPPSAACGSPASQLVATPSLELGPIPAGEEKALELPLAASSIPRPRTPSPESHRSPAEGSERLSLSPLRGGEAGPDASPTVTTPTVTTPSLPAEVGSPHSTEVDESLSVSFEQVLPPSAPTSEAGLSLPLRGPRARRSASPHDVDLCLVSPCEFEHRKAVPMAPAPASPGSSDDSSARSQERAGGLGAEETPPTSVSESLPTLSDSDPVPLAPGAADSDEDTEGFGVPRHDPLPDPLKVPPPLPDPSSICMVDPEMLPPKTARQTEDVSRTRKPLARPDSRAAAPKATPVAAAKTKGLAGGDRASRPLSARSEPSEKGGRAPLSRKSSTPKTATRGPSGSASSRPGVSATPPKSPVYLDLAYLPSGSSAHLVDEEFFQRVRALCYVISGQDQRKEEGMRAVLDALLASKQHWDRDLQVTLIPTFDSVAMHTWYAETHARHQALGITVLGSDSMVSMQDDAFPACKVEF

SEQ ID NO: 237 - protein, 222 aa, Homo sapiens
MNSNVENLPPHIIRLVYKEVTTLTADPPDGIKVFPNEEDLTDLQVTIEGPEGTPYAGGLFRMKLLLGKDFPASPPKGYFLTKIFHPNVGANGEICVNVLKRDWTAELGIRHVLLTIKCLLIHPNPESALNEEAGRLLLENYEEYAARARLLTEIHGGAGGPSGRAEAGRALASGTEASSTDPGAPGGPGGAEGTMAKKHAGERDKKLAAKKKTDKKRALRRL

SEQ ID NO: 238 - protein, 245 aa, Homo sapiens
MAVRASFENNCEIGCFAKLTNTYCLVAIGGSENFYSVFEGELSDTIPVVHASIAGCRIIGRMCVGNRHGLLVPNNTTDQELQHIRNSLPDTVQIRRVEERLSALGNVTTCNDYVALVHPDLDRETEEILADVLKVEVFRQTVADQVLVGSYCVFSNQGGLVHPKTSIEDQDELSSLLQVPLVAGTVNRGSEVIAAGMVVNDWCAFCGLDTTSTELSVVESVFKLNEAQPSTIATSMRDSLIDSLT

SEQ ID NO: 239 - protein, 117 aa, Homo sapiens
MESGAKGCEVVVSGKLRGQRAKSMKFVDGLMIHSGDPVNYYVDTAVRHVLLRQGVLGIKVKIMLPWDPTGKIGPKKPLPDHVSIVEPKDEILPTTPISEQKGGKPEPPAMPQPVPTA

SEQ ID NO: 240 - protein, 444 aa, Homo sapiens
MREIVHIQAGQCGNQIGAKFWEVISDEHGIDPTGTYHGDSDLQLDRISVYYNEATGGKYVPRAILVDLEPGTMDSVRSGPFGQIFRPDNFVFGQSGAGNNWAKGHYTEGAELVDSVLDVVRKEAESCDCLQGFQLTHSLGGGTGSGMGTLLISKIREEYPDRIMNTFSVVPSPKVSDTVVEPYNATLSVHQLVENTDETYCIDNEALYDICFRTLKLTTPTYGDLNHLVSATMSGVTTCLRFPGQLNADLRKLAVNMVPFPRLHFFMPGFAPLTSRGSQQYRALTVPELTQQVFDAKNMMAACDPRHGRYLTVAAVFRGRMSMKEVDEQMLNVQNKNSSYFVEWIPNNVKTAVCDIPPRGLKMAVTFIGNSTAIQELFKRISEQFTAMFRRKAFLHWYTGEGMDEMEFTEAESNMNDLVSEYQQYQDATAEEEEDFGEEAEEEA

SEQ ID NO: 241 - protein, 92 aa, Homo sapiens
MDEQIRLMDQNLKCLSAAEEKYSQKEDKYEEEIKILTDKLKEAETRAEFAERSVAKLEKTIDDLEDKLKCTKEEHLCTQRMLDQTLLDLNEM

SEQ ID NO: 242 - protein, 453 aa, Homo sapiens [sequence omitted]

SEQ ID NO: 243 - protein, 209 aa, Homo sapiens
MKELAEEEPHLVEQFQKLSEAAGRVGSDMTSQQEFTSCLKETLSGLAKNATDLQNSSMSEEELTKAMEGLGMDEGDGEGNILPIMQSIMQNLLSKDVLYPSLKEITEKYPEWLQSHRESLPPEQFEKYQEQHSVMCKICEQFEAETPTDSETTQKARFEMVLDLMQQLQDLGHPPKELAGEMPPGLNFDLDALNLSGPPGASGEQCLIM

SEQ ID NO: 244 - protein, 354 aa, Homo sapiens
MRRLMSSRDWPRTRTGTGILSSQPEENPYWWNANMVFIPYCSSDVWSGASSKSEKNEYAFMGALIIQEVVRELLGRGLSGAKVLLLAGSSAGGTGVLLNVDRVAEQLEKLGYPAIQVRGLADSGWFLDNKQYRHTDCVDTITCAPTEAIRRGIRYWNGVVPERCRRQFQEGEEWNCFFGYKVYPTLRCPVFVVQWLFDEAQLTVDNVHLTGQPVQEGLRLYIQNLGRELRHTLKDVPASFAPACLSHEIIIRSHWTDVQVKGTSLPRALHCWDRSLHDSHKASKTPLKGCPVHLVDSCPWPHCNPSCPTVRDQFTGQEMNVAQFLMHMGFDMQTVAQPQGLEPSELLGMLSNGS

SEQ ID NO: 245 - protein, 295 aa, Homo sapiens
MELIQDTSRPPLEYVKGVPLIKYFAEALGPLQSFQARPDDLLISTYPKSGTTWVSQILDMIYQGGDLEKCHRAPIFMRVPFLEFKAPGIPSGMETLKDTPAPRLLKTHLPLALLPQTLLDQKVKVVYVARNAKDVAVSYYHFYHMAKVHPEPGTWDSFLEKFMVGEVSYGSWYQHVQEWWELSRTHPVLYLFYEDMKENPKREIQKILEFVGHSLPEETVDFMVQHTSFKEMKKNPMTNYTTVPQEFMDHSISPFMRKGMAGDWKTTFTVAQNERFDADYAEKMAGCSLSFRSEL

SEQ ID NO: 246 - protein, 439 aa, Homo sapiens
MEPSTAARAWALFWLLLPLLGAVCASGPRTLVLLDNLNVRETHSLFFRSLKDRGFELTFKTADDPSLSLIKYGEFLYDNLIIFSPSVEDFGGNINVETISAFIDGGGSVLVAASSDIGDPLRELGSECGIEFDEEKTAVIDHHNYDISDLGQHTLIVADTENLLKAPTIVGKSSLNPILFRGVGMVADPDNPLVLDILTGSSTSYSFFPDKPITQYPHAVGKNTLLIAGLQARNNARVIFSGSLDFFSDSFFNSAVQKAAPGSQRYSQTGNYELAVALSRWVFKEEGVLRVGPVSHHRVGETAPPNAYTVTDLVEYSIVIQQLSNGKWVPFDGDDIQLEFVRIDPFVRTFLKKKGGKYSVQFKLPDVYGVFQFKVDYNRLGYTHLYSSTQVSVRPLQHTQYERFIPSAYPYYASAFSMMLGLFIFSIVFLHMKEKEKSD

SEQ ID NO: 247 - protein, 56 aa, Homo sapiens
METLHTWGSKVLGYSWIFRTSAYPQVSQASGGEASDPWPTCYPPQGLDLSSREGTE

SEQ ID NO: 248 - protein, 46 aa, Homo sapiens
MGFKGPGVFLDLQDICLPSGFPGLGWGGIRSLANLLSTPGFRPLFP

SEQ ID NO: 249 - protein, 61 aa, Homo sapiens
IGTVFLEGNLVKCIKRLKNTDVLCAGNSTSSNFSLKPYQRCIQRIIYKEGCLIMIVIIINN

SEQ ID NO: 250 - protein, 73 aa, Homo sapiens
MFDSPFYELNYFIRVGNFCFLIKWKLAFLTLFLLLFYRNAFCWPGTVAHPCNPSTVGGRDGWITRSGDRDHPG

SEQ ID NO: 251 - protein, 43 aa, Homo sapiens
MLFVGRAQLLIHVIPALWEAETGGSQGQEIETILANALKLRLC

SEQ ID NO: 252 - protein, 30 aa, Homo sapiens
MYIFFCVLFLLLLLFETGSCSVAQAGVQWH

SEQ ID NO: 253 - protein, 87 aa, Homo sapiens
MNCNTQSQTRALPRPLGGCTPSSSARLRSLRPRLKEGVAGNPGNLSEVTPHPYTPSVHPRLFLLLFGFWKGIHLQAAHPGGACFLKP

SEQ ID NO: 254 - protein, 211 aa, Homo sapiens [sequence omitted]

SEQ ID NO: 255 - protein, 417 aa, Homo sapiens
MSLSNKLTLDKLDVKGKRVVMRVDFNVPMKNNQITNNQRIKAAVPSIKFCLDNGAKSVVLMSHLGRPDGVPMPDKYSLEPVAVELKSLLGKDVLFLKDCVGPEVEKACANPAAGSVILLENLRFHVEEEGKGKDASGNKVKAEPAKIEAFRASLSKLGDVYVNDAFGTAHRAHSSMVGVNLPQKAGGFLMKKELNYFAKALESPERPFLAILGGAKVADKIQLINNMLDKVNEMIIGGGMAFTFLKVLNNMEIGTSLFDEEGAKIVKDLMSKAEKNGVKITLPVDFVTADKFDENAKTGQATVASGIPAGWMGLDCGPESSKKYAEAVTRAKQIVWNGPVGVFEWEAFARGTKALMDEVVKATSRGCITIIGGGDTATCCAKWNTEDKVSHVSTGGGASLELLEGKVLPGVDALSNI

SEQ ID NO: 256 - protein, 568 aa, Homo sapiens
MVLGPEQKMSDDSVSGDHGESASLGNINPAYSNPSLSQSPGDSEEYFATYFNEKISIPEEEYSCFSFRKLWAFTGPGFLMSIAYLDPGNIESDLQSGAVAGFKLLWILLLATLVGLLLQRLAARLGVVTGLHLAEVCHRQYPKVPRVILWLMVELAIIGSDMQEVIGSAIAINLLSVGRIPLWGGVLITIADTFVFLFLDKYGLRKLEAFFGFLITIMALTFGYEYVTVKPSQSQVLKGMFVPSCSGCRTPQIEQAVGIVGAVIMPHNMYLHSALVKSRQVNRNNKQEVREANKYFFIESCIALFVSFIINVFVVSVFAEAFFGKTNEQVVEVCTNTSSPHAGLFPKDNSTLAVDIYKGGVVLGCYFGPAALYIWAVGILAAGQSSTMTGTYSGQFVMEGFLNLKWSRFARVVLTRSIAIIPTLLVAVFQDVEHLTGMNDFLNVLQSLQLPFALIPILTFTSLRPVMSDFANGLGWRIAGGILVLIICSINMYFVVVYVRDLGHVALYVVAAVVSVAYLGFVFYLGWQCLIALGMSFLDCGHTCHLGLTAQPELYLLNTMDADSLVSR

SEQ ID NO: 257 - protein, 46 aa, Homo sapiens
MLFIHAEVIQFPPSYRSILIHPTLEMQHLCGRLFHKPPRLLRLGRY

SEQ ID NO: 258 - protein, 36 aa, Homo sapiens
MASLQFVISLPVCSLKLIKRSGYIELLYRCEGMDKS

SEQ ID NO: 259 - protein, 898 aa, Homo sapiens
MSVTEEDLCHHMKVVVRVRPENTKEKAAGFHKVVHVVDKHILVFDPKQEEVSFFHGKKTTNQNVIKKQNKDLKFVFDAVFDETSTQSEVFEHTTKPILRSFLNGYNCTVLAYGATGAGKTHTMLGSADEPGVMYLTMLHLYKCMDEIKEEKICSTAVSYLEVYNEQIRDLLVNSGPLAVREDTQKGVVVHGLTLHQPKSSEEILHLLDNGNKNRTQHPTDMNATSSRSHAVFQIYLRQQDKTASINQNVRIAKMSLIDLAGSERASTSGAKGTRFVEGTNINRSLLALGNVINALADSKRKNQHIPYRNSKLTRLLKDSLGGNCQTIMIAAVSPSSVFYDDTYNTLKYANRAKDIKSSLKSNVLNVNNHITQYVKICNEQKAEILLLKEKLKAYEEQKAFTNENDQAKLMISNPQEKEIERFQEILNCLFQNREEIRQEYLKLEMLLKENELKSFYQQQCHKQIEMMCSEDKVEKATGKRDHRLAMLKTRRSYLEKRREEELKQFDENTNWLHRVEKEMGLLSQNGHIPKELKKDLHCHHLHLQNKDLKAQIRHMMDLACLQEQQHRQTEAVLNALLPTLRKQYCTLKEAGLSNAAFESDFKEIEHLVERKKVVVWADQTGEQPKQNDLPGISVLMTFSQLGPVQPIPCCSSSGGTNLVKIPTEKRTRRKLMPSPLKGQHTLKSPPSQSVQLNDSLSKELQPIVYTPEDCRKAFQNPSTVTLMKPSSFTTSFQAISSNINSDNCLKMLCEVAIPHNRRKECGQEDLDSTFTICEDIKSSKCKLPEQESLPNDNKDILQRLDPSSFSTKHSMPVPSMVPSYMAMTTAAKRKRKLTSSTSNSSLTADVNSGFAKRVRQDNSSEKHLQENKPTMEHKRNICKINPSMVRKFGRNISKGNLR

SEQ ID NO: 260 - protein, 71 aa, Homo sapiens
MSKDRANMQHRYIELFLNSTTGASNGAYSSQVMQGMGVSAAQATYSGLESQSVSGCYGAGYSGQNSMGGYD

SEQ ID NO: 261 - protein, 592 aa, Homo sapiens
MAPGQLALFSVSDKTGLVEFARNLTALGLNLVASGGTAKALRDAGLAVRDVSELTGFPEMLGGRVKTLHPAVHAGILARNIPEDNADMARLDFNLIRVVACNLYPFVKTVASPGVTVEEAVEQIDIGGVTLLRAAAKNHARVTVVCEPEDYVVVSTEMQSSESKDTSLETRRQLALKAFTHTAQYDEAISDYFRKQYSKGVSQMPLRYGMNPHQTPAQLYTLQPKLPITVLNGAPGFINLCDALNAWQLVKELKEALGIPAAASFKHVSPAGAAVGIPLSEDEAKVCMVYDLYKTLTPISAAYARARGADRMSSFGDFVALSDVCDVPTAKIISREVSDGIIAPGYEEEALTILSKKKNGNYCVLQMDQSYKPDENEVRTLFGLHLSQKRNNGVVDKSLFSNVVTKNKDLPESALRDLIVATIAVKYTQSNSVCYAKNGQVIGIGAGQQSRIHCTRLAGDKANYWWLRHHPQVLSMKFKTGVKRAEISNAIDQYVTGTIGEDEDLIKWKALFEEVPELLTEAEKKEWVEKLTEVSISSDAFFPFRDNVDRAKRSGVAYIAAPSGSAADKVVIEACDELGIILAHTNLRLFHH

SEQ ID NO: 262 - protein, 62 aa, Homo sapiens
MFELLPNCMLFILNSPSDRIPRPREVKKTSPRSITLLLTAPNLLDSKSNGFPGTMMLVDLKK

SEQ ID NO: 263 - protein, 43 aa, Homo sapiens
MTALFPGLAPETEQPDIHTPRRQLEVPPGNQNHPQRRPPDTDI

SEQ ID NO: 264 - protein, 303 aa, Homo sapiens
MKPTGTDPRILSIAAEVAKSPEQNVPVILLKLKEIINITPLGSSELKKIKQDIYCYDLIQYCLLVLSQDYSRIQGGWTTISQLTQILSHCCVGLEPGEDAEEFYNELLPSAAENFLVLGRQLQTCFINAAKAEEKDELLHFFQIVTDSLFWLLGGHVELIQNVLQSDHFLHLLQADNVQIGSAVMMMLQNILQINSGDLLRIGRKALYSILDEVIFKLFSTPSPVIRSTATKLLLLMAESHQEILILLRQSTCYKGLRRLLSKQETGTEFSQELRQLVGLLSPMVYQEVEEQIQTIKDVAGDK

SEQ ID NO: 265 - protein, 264 aa, Homo sapiens [sequence omitted]

SEQ ID NO: 266 - protein, 248 aa, Homo sapiens
MSGGGVIRGPAGNNDCRIYVGNLPPDIRTKDIEDVFYKYGAIRDIDLKNRRGGPPFAFVEFEDPRDAEDAVYGRDGYDYDGYRLRVEFPRSGRGTGRGGGGGGGGGAPRGRYGPPSRRSENRVVVSGLPPSGSWQDLKDHMREAGDVCYADVYRDGTGVVEFVRKEDMTYAVRKLDNTKFRSHEGETAYIRVKVDGPRSPSYGRSRSRSRSRSRSRSRSNSRSRSYSPRRSRGSPRYSPRHSRSRSRT

SEQ ID NO: 267 - protein, 313 aa, Homo sapiens
MPVAGSELPRRPLPPAAQERDAEPRPPHGELQYLGQIQHILRCGVRKDDRTGTGTLSVFGMQARYSLRDEFPLLTTKRVFWKGVLEELLWFIKGSTNAKELSSKGVKIWDANGSRDFLDSLGFSTREEGDLGPVYGFQWRHFGAEYRDMESDYSGQGVDQLQRVIDTIKTNPDDRRIIMCAWNPRDLPLMALPPCHALCQFYVVNSELSCQLYQRSGDMGLGVPFNIASYALLTYMIAHITGLKPGDFIHTLGDAHIYLNHIEPLKIQLQREPRPFPKLRILRKVEKIDDFKAEDFQIEGYNPHPTIKMEMAV

SEQ ID NO: 268 - protein, 511 aa, Homo sapiens
MAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 269 - protein, 128 aa, Homo sapiens
MFDVTSRVTYKNVPNWHRDLVRVCENIPIVLCGNKVDIKDRKVKAKSIVFHRKKNLQYYDISAKSNYNFEKPFLWLARKLIGDPNLEFVAMPALAPPEVVMDPALAAQYEHDLEVAQTTALPDEDDDL

SEQ ID NO: 270 - protein, 506 aa, Homo sapiens
MEDHQHVPIDIQTSKLLDWLVDRRHCSLKWQSLVLTIREKINAAIQDMPESEEIAQLLSGSYIHYFHCLRILDLLKGTEASTKNIFGRYSSQRMKDWQEIIALYEKDNTYLVELSSLLVRNVNYEIPSLKKQIAKCQQLQQEYSRKEEECQAGAAEMREQFYHSCKQYGITGENVRGELLALVKDLPSQLAEIGAAAQQSLGEAIDVYQASVGFVCESPTEQVLPMLRFVQKRGNSTVYEWRTGTEPSVVERPHLEELPEQVAEDAIDWGDFGVEAVSEGTDSGISAEAAGIDWGIFPESDSKDPGGDGIDWGDDAVALQITVLEAGTQAPEGVARGPDALTLLEYTETRNQFLDELMELEIFLAQRAVELSEEADVLSVSQFQLAPAILQGQTKEKMVTMVSVLEDLIGKLTSLQLQHLFMILASPRYVDRVTEFLQQKLKQSQLLALKKELMVQKQQEALEEQAALEPKLDLLLEKTKELQKLIEADISKRYSGRPVNLMGTSL

SEQ ID NO: 271 - protein, 136 aa, Homo sapiens
MTSLCMAMTEEQHKSVVIDCSSSQPQFCNAGSNRFCEDWMQAFLNGAKGGNPFLFRQVLENFKLKAIQDTNNLKRFIRQAEMNHYALFKCYMFLKNCGSGDILLKIVKVEHEEMPEAKNVIAVLEEFMKEALDQSF

SEQ ID NO: 272 - protein, 509 aa, Homo sapiens
MFTNDMMECKQDEIVMQGMDPSALEALINFAYNGNLAIDQQNVQSLLMGASFLQLQSIKDACCTFLRERLHPKNCLGVRQFAETMMCAVLYDAANSFIHQHFVEVSMSEEFLALPLEDVLELVSRDELNVKSEEQVFEAALAWVRYDREQRGPYLPELLSNIRLPLCRPQFLSDRVQQDDLVRCCHKCRDLVDEAKDYHLMPERRPHLPAFRTRPRCCTSIAGLIYAVGGLNSAGDSLNVVEVFDPIANCWERCRPMTTARSRVGVAVVNGLLYAIGGYDGQLRLSTVEAYNPETDTWTRVGSMNSKRSAMGTVVLDGQIYVCGGYDGNSSLSSVETYSPETDKWTVVTSMSSNRSAAGVTVFEGRIYVSGGHDGLQIFSSVEHYNHHTATWHPAAGMLNKRCRHGAASLGSKMFVCGGYDGSGFLSIAEMYSSVADQWCLIVPMHTRRSRVSLVASCGRLYAVGGYDGQSNLSSVEMYDPETDCWTFMAPMACHEGGVGVGCIPLLTI

SEQ ID NO: 273 - protein, 49 aa, Homo sapiens
MSFSAILSPFSSLSVNVRNLRQRGKGRQNSRILTLIVKILFKTWHLIFL

SEQ ID NO: 274 - protein, 109 aa, Homo sapiens
MESHSVTQAGVQWHDLGSLHSPLLGSSDSPTSASRVAGITGMQHHTQLIFLFLVEMGFHHVGQAGLKLLTSGDPPASASQSAGITGVGHHTWPIMEDFLMVMFELGFGE

SEQ ID NO: 275 - protein, 54 aa, Homo sapiens
MESNIIYTPSLPLFLPPFLPPSLPPFLPPFSLSLSLPASLPFFLLCLLPCDWGK

SEQ ID NO: 276 - protein, 66 aa, Homo sapiens
MLLYRLAQLGLYFLYSMPVEHQMLNTSTCCDFAIPAHITHLISFVGGHVGWPTHWQVNSLIWTMSH

SEQ ID NO: 277 - protein, 180 aa, Homo sapiens; identical to SEQ ID NO: 220

SEQ ID NO: 278 - protein, 34 aa, Homo sapiens
MGLERGFDPRSLCAFAAEPHNLSFQKHFQNANIF

SEQ ID NO: 279 - protein, 168 aa, Homo sapiens
MLRVNFFFFFFFFFSFSLRLGLALLPRLEWSGVILAYCSLCLPGSSSPASASGVAGTTGSCHHGQPTFACFVKMGSHSVAQAGLKLLGSGDPPVSASQSAGITIVSHHVQLEGSTSFTFCKHICIFTPPFPSFSLFISHFYIDLLFYNKTLLPKKKKKKKKKKKKKKK

SEQ ID NO: 280 - protein, 158 aa, Homo sapiens [sequence omitted]

SEQ ID NO: 281 - protein, 43 aa, Homo sapiens
MPLGPVQVYLSLISESCSSCLTLPHGSSVHLSITVLNPFSISV

SEQ ID NO: 282 - protein, 61 aa, Homo sapiens
MKKLTLPMGLPPFLPLFSLWYPSRVFPSPLQSPISHLFFFSPSSFSYCVLPATSHRLVVYK

SEQ ID NO: 283 - protein, 207 aa, Homo sapiens
MQKMLPEIDQNKDRMLEILEGKGLSFLFPLLKLEKELLKQIKLDPSPQTIYKWIKDNISPKLHVDKGFVNILMTSFLQYISSEVNPPSDETDSSSAPSKEQLEQEKQLLLSFKPVMQKFLHDHVDLQVSALYALQVHCYNSNFPKGMLLRFFVHFYDMEIIEEEAFLAWKEDITQEFPGKGKALFQVNQWLTWLETAEEEESEEEAD

SEQ ID NO: 284 - protein, 105 aa, Homo sapiens
  UNSURE (80)..(80): X at position 80 may be “Asp” or “Glu”
FSCLSFLSSWDYRHAPPCLANFAFLVETGFHHVGQAGLKLPTSGDLPTSASQSAGITGMSYRAWPVYFWRQSLALLPRLXGSGATLNSASRVQAILVRHLPSSWG

SEQ ID NO: 285 - protein, 91 aa, Homo sapiens
LTAVFFSFIHFAFFLYFRFNSTFKKSYLYICIFIFIFQDLICLFFIMGYYCSMVQNLLFFPKLLVIFKIFVNFLPLASSQVPAFSQSAGFP

SEQ ID NO: 286 - protein, 75 aa, Homo sapiens
PKSLPGHPLAYSLTGHAPAVHTGSYQSSSWAPFQTSEESFQHEEGVQNKQREREREREREREREKRNINNAGSKR

SEQ ID NO: 287 - protein, 83 aa, Homo sapiens
MYCVFNRNEDACRYGSAIGVLASLAYQRYKAGVDDFIQNYVDPTPDPNTAYASYPGASVDNYQQPPFTQNAETTEGYQPPPVY

SEQ ID NO: 288 - protein, 117 aa, Homo sapiens
MVRATAMPTSLSRCTACSTATRMPAAMAVPSGCWPPWPTSATRLAWTTSSRITLTPLRTPTLPTPPTQVHLWTTTNSHPSPRTRRPPRATSRPLCTERRLAWEGGQRGPSPLPWTFP

SEQ ID NO: 289 - DNA, 1280 bp, Homo sapiens
gtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaaccaaaaaaaaaaaaaaaaaaaa

SEQ ID NO: 290 - DNA, 2978 bp, Homo sapiens
gccgtgagaacacgctgtgtggctgaaaagtgaaggcaagagctgatttggcctctgtgctcccctccgcaaggggatcgttttctccagaagagctggatattctttcgcccagttatggcagacaagttaacgagaattgctattgtcaaccatgacaaatgtaaacctaagaaatgtcgacaggaatgcaaaaagagttgtcctgtagttcgaatgggaaaattatgcatagaggttacaccccagagcaaaatagcatggatttccgaaactctttgtattggttgtggtatctgtattaagaaatgcccctttggcgccttatcaattgtcaatctaccaagcaacttggaaaaagaaaccacacatcgatattgtgccaatgccttcaaacttcacaggttgcctatccctcgtccaggtgaagttttgggattagttggaactaatggtattggaaagtcaactgctttaaaaattttagcaggaaaacaaaagccaaaccttggaaagtacgatgatcctcctgactggcaggagattttgacttatttccgtggatctgaattacaaaattactttacaaagattctagaagatgacctaaaagccatcatcaaacctcaatatgtagaccagattcctaaggctgcaaaggggacagtgggatctattttggaccgaaaagatgaaacaaagacacaggcaattgtatgtcagcagcttgatttaacccacctaaaagaacgaaatgttgaagatctttcaggaggagagttgcagagatttgcttgtgctgtcgtttgcatacagaaagctgatattttcatgtttgatgagccttctagttacctagatgtcaagcagcgtttaaaggctgctattactatacgatctctaataaatccagatagatatatcattgtggtggaacatgatctaagtgtattagactatctctccgacttcatctgctgtttatatggtgtaccaagcgcctatggagttgtcactatgccttttagtgtaagagaaggcataaacatttttttggatggctatgttccaacagaaaacttgagattcagagatgcatcacttgtttttaaagtggctgagacagcaaatgaagaagaagttaaaaagatgtgtatgtataaatatccaggaatgaagaaaaaaatgggagaatttgagctagcaattgtagctggagagtttacagattctgaaattatggtgatgctgggggaaaatggaacgggtaaaacgacatttatcagaatgcttgctggaagacttaaacctgatgaaggaggagaagtaccagttctaaatgtcagttataagccacagaaaattagtcccaaatcaactggaagtgttcgccagttactacatgaaaagataagagatgcttatactcacccacaatttgtgaccgatgtaatgaagcctctgcaaattgaaaacatcattgatcaagaggtgcagacattatctggtggtgaactacagcgagtagctttagccctttgcttgggcaaacctgctgatgtctatttaattgatgaaccatctgcatatttggattctgagcaaagactgatggcagctcgagttgtcaaacgtttcatactccatgcaaaaaagacagcctttgttgtggaacatgacttcatcatggccacctatctagcggatcgcgtcatcgtttttgatggtgttccatctaagaacacagttgcaaacagtcctcaaacccttttggctggcatgaataaatttttgtctcagcttgaaattacattcagaagagatccaaacaactataggccacgaataaacaaacttaattcaattaaggatgtagaacaaaagaagagtggaaactactttttcttggatgattagactgactctgagaatattgataagccatttattaaaaggagtatttactagaattttttgtcatataaaacttgaatcaggattttatgccccacatactctggaacttgaagtataatatacttaatataacataaaaagccagttgggttctaaattgtagttgaaacacagaaaatgccacttttctgttcctgaagaggctcttttgtgcataatattctaaaatgaagacatttcaagctatacaaattacttccaagttttcatgatgtatgggaagattttcagtaggtgtattatattcacggtaccaaatgctgaccagtgttgctccattttttaaatcttgaaaagggtttctgtacttacctggtttgccaagtatgccagtgtaatgaaactgcccttattttaaaagccagtcaaagattccactgattgacatttgataaataaacatcaggattatgtttattgtttgttttcagtctttgcactatattaccagtatatggtttccgaggaagattatctactgcaaaacaccactgttggaaaaataggtatttttaaattgtttttaatcttttttggtgcttttaaacatgtttagcaaaaaccaattcagttccattccccgcaaaaaacccctaactttactctgaactttttttgtttttgcattccatgaggttctgtattcagtcattctctaggtaatgtcatttttgtacacatatatttatataatcactgattgagatttaggaaaaagcatttctaaagaatatttgcttcccttagaactacagactcgaaatctttaaagatggtgcctaagcatctatgtattttttttaagttccacagatttttctgttgggcagccaaggattataaaccacttccctaaaggcaacattaatgcaaaagcccccaccccatggcttccatcttttgcatcaccaccactcctgaacccccatttctgatttgtcagaatttttttttaacaaaactaaaaatgaaaaaaaaaaaaaaaaaaaaaaaaaaaaaaa

SEQ ID NO: 291 - DNA, 1218 bp, Homo sapiens
gaagttactgcagccgcggtgttgtgctgtggggaagggagaaggatttgtaaaccccggagcgaggttctgcttacccgaggccgctgctgtgcggagacccccgggtgaagccaccgtcatcatgtctgaccaggaggcaaaaccttcaactgaggacttgggggataagaaggaaggtgaatatattaaactcaaagtcattggacaggatagcagtgagattcacttcaaagtgaaaatgacaacacatctcaagaaactcaaagaatcatactgtcaaagacagggtgttccaatgaattcactcaggtttctctttgagggtcagagaattgctgataatcatactccaaaagaactgggaatggaggaagaagatgtgattgaagtttatcaggaacaaacggggggtcattcaacagtttagatattctttttattttttttcttttccctcaatccttttttatttttaaaaatagttcttttgtaatgtggtgttcaaaacggaattgaaaactggcaccccatctctttgaaacatctggtaatttgaattctagtgctcattattcattattgtttgttttcattgtgctgatttttggtgatcaagcctcagtccccttcatattaccctctcctttttaaaaattacgtgtgcacagagaggtcacctttttcaggacattgcattttcaggcttgtggtgataaataagatcgaccaatgcaagtgttcataatgactttccaattggccctgatgttctagcatgtgattacttcactcctggactgtgactttcagtgggagatggaagtttttcagagaactgaactgtggaaaaatgacctttccttaacttgaagctacttttaaaatttgagggtctggaccaaaagaagaggaatatcaggttgaagtcaagatgacagataaggtgagagtaatgactaactccaaagatggcttcactgaagaaaaggcattttaagattttttaaaaatcttgtcagaagatcccagaaaagttctaattttcattagcaattaataaagctatacatgcagaaatgaatacaacagaacactgctctttttgattttatttgtactttttggcctgggatatgggttttaaatggacattgtctgtaccagcttcattaaaataaacaatatttgtaaaaatcaaaaaaaaaaaaaaaaaaa

SEQ ID NO: 292 - DNA, 825 bp, Homo sapiens
cgcctcggaggcgttcagctgcttcaagatgaagctgaacatctccttcccagccactggctgccagaaactcattgaagtggacgatgaacgcaaacttcgtactttctatgagaagcgtatggccacagaagttgctgctgacgctctgggtgaagaatggaagggttatgtggtccgaatcagtggtgggaacgacaaacaaggtttccccatgaagcagggtgtcttgacccatggccgtgtccgcctgctactgagtaaggggcattcctgttacagaccaaggagaactggagaaagaaagagaaaatcagttcgtggttgcattgtggatgcaaatctgagcgttctcaacttggttattgtaaaaaaaggagagaaggatattcctggactgactgatactacagtgcctcgccgcctgggccccaaaagagctagcagaatccgcaaacttttcaatctctctaaagaagatgatgtccgccagtatgttgtaagaaagcccttaaataaagaaggtaagaaacctaggaccaaagcacccaagattcagcgtcttgttactccacgtgtcctgcagcacaaacggcggcgtattgctctgaagaagcagcgtaccaagaaaaataaagaagaggctgcagaatatgctaaacttttggccaagagaatgaaggaggctaaggagaagcgccaggaacaaattgcgaagagacgcagactttcctctctgcgagcttctacttctaagtctgaatccagtcagaaataagattttttgagtaacaaataaataagatcagactctgaaaaaaaaaa

SEQ ID NO: 293 - DNA, 1978 bp, Homo sapiens
ggccggagcggcgcggcagcggcaggaccgccgtggcgcctagagtagcgacccggggggagcgcggggcgacgctggctgcagggacccggtgacagcgtgagaggtactaggttttgacaagcttgcatcatgcgtgagtataagctagtcgttcttggctcaggaggcgttggaaagtctgctttgactgtacaatttgttcaaggaatttttgtagaaaaatacgatcctacgatagaagattcttatagaaagcaagttgaagtagatgcacaacagtgtatgcttgaaatcttggatactgcaggaacggagcaatttacagcaatgagggatttatacatgaaaaatggacaaggatttgcattagtttattccatcacagcacagtccacatttaacgatttacaagacctgagagaacagattcttcgagttaaagacactgatgatgttccaatgattcttgttggtaataagtgtgacttggaagatgaaagagttgtagggaaggaacaaggtcaaaatctagcaagacaatggaacaactgtgcattcttagaatcttctgcaaaatcaaaaataaatgttaatgagatcttttatgacctagtgcggcaaattaacagaaaaactccagtgcctgggaaggctcgcaaaaagtcatcatgtcagctgctttaatatactaaatgcattgtagctctgagccaggtctgaagaactgttgcccaattcaacagtgccagcattccaactttgttaaacctaccaacatcttaaatggactttcctgtggtggtaccctttaagaggcggatgaaagctactatatcagtttgcacattctaatcactttccagtatcacaagagagatttttacttatataatagtcctagagtttgcagctggtaaaaccagaggctacatccagtattactgctaagagacattcttcatccaccaatgttgtacatgtatgaaaatggtgtactgtatactttaacatgccccatactttgtattggagagtacaataatgtaaatcctaaaagcaccactattttagcataataaaagaaagtccaaagagctcctatatagactactccagataacttcgcttctttgatacttgtagcttattgtaattttttttaagaaattcaaggtcattattattgtacaaaataagcgctttgattaacacagctatatagtttttttaatttttaaaaaacctgtggagacggtgatcttgtctttaaaacatgatagtcctttcagtataatgtcttagattaaagacgttgcctttaatatctgttgggaaggaaatgtccagacttttcaaatctcttattatatgtttcctttttttgtttacatagggaacaatgtttatagtcgtgtgtacagtgggggtctacaacaagaagtgtatattttcaaacaattttttaatgatttaacaatttttgtaaatcattttcaggcttctgcagctgtagattctcactgtgaatcccttgcttgctcatgcataagtgtatttgcaataccaaatatacaggtttagtatttttgcctgttagtgattgtttcacatgtgtaacgttttggttgagatgttaaatggtggacgagtactgtggatgtgaatgtgggaagtaattttaatcatatgtaattggtcacaaggcctaatttgcagtaactattgctgttttatttaacaatgccttgttgctttgtatgcattaatgtttggatgtaaagattgtgtgtctatccaacagggagccacagtatttaaattgaccaacctaatgttacaactactttgaggtggccaaatgtaaactaaaagccttaattaaagtggtgcaattttgtataacttagcatcagtagttcaataaatttggattgccatgcaagggcttgccttataaaaaaaaaaaaaaaaaaaa

SEQ ID NO: 294 - DNA, 895 bp, Homo sapiens
gccatcttgcgtccccgcgtgtgtgcgcctaatctcaggtggtccacccgagaccccttgagcaccaaccctagtcccccgcgcggccccttattcgctccgacaagatgaaagaaacaatcatgaaccaggaaaaactcgccaaactgcaggcacaagtgcgcattggtgggaaaggaactgctcgcagaaagaagaaggtggttcatagaacagccacagcagatgacaaaaaacttcagttctccttaaagaagttaggggtaaacaatatctctggtattgaagaggtgaatatgtttacaaaccaaggaacagtgatccactttaacaaccctaaagttcaggcatctctggcagcgaacactttcaccattacaggccatgctgagacaaagcagctgacagaaatgctacccagcatcttaaaccagcttggtgcggatagtctgactagtttaaggagactggccgaagctctgcccaaacaatctgtggatggaaaagcaccacttgctactggagaggatgatgatgatgaagttccagatcttgtggagaattttgatgaggcttccaagaatgaggcaaactgaattgagtcaacttctgaagataaaacctgaagaagttactgggagctgctattttatattatgactgctttttaagaaatttttgtttatggatctgataaaatctagatctctaatatttttaagcccaagccccttggacactgcagctcttttcagtttttgcttatacacaattcattctttgcagctaattaagccgaagaagcctgggaatcaagtttgaaacaaagattaataaagttctttgcctagtaaaaaaaaaaaaaaaaaaaaaaataaaaaaaaaaaaaaaaaaa

SEQ ID NO: 295 - DNA, 1358 bp, Homo sapiens
gttccaacccagggggaaaaatgcggcctttgactgaagaggagacccgtgtcatgtttgagaagatagcgaaatacattggggagaatcttcaactgctggtggaccggcccgatggcacctactgtttccgtctgcacaacgaccgggtgtactatgtgagtgagaagattatgaagctggccgccaatatttccggggacaagctggtgtcgctggggacctgctttggaaaattcactaaaacccacaagtttcggttgcacgtcacagctctggattaccttgcaccttatgccaagtataaagtttggataaagcctggtgcagagcagtccttcctgtatgggaaccatgtgttgaaatctggtctgggtcgaatcactgaaaatacttctcagtaccagggcgtggtggtgtactccatggcagacatccctttgggttttggggtggcagccaaatctacacaagactgcagaaaagtagaccccatggcgattgtggtatttcatcaagcagacattggggaatatgtgcggcatgaagagacgttgacttaaaacgaagccattccaaggacagacggctgtatggaaaggccgagctttgtttcctgtgtttgtgtggactccaccatcatgttgaattttgtcaacactctggcctcttcagggacttcttatttactgtactctctatcactgacaaatgcaggctggattcttattatatacagagatggctcaaaaatggggtttcagatctttgtgacgaaatagaatactgtttcatatttgaatcagagggcttcttgttctgagaaataggttcaaaatcattggaaccaggaacaagaatagcttattgttatctgtgataacactgttttctaaacacaaggattttcttttttattaatatgcaacatagacattgccataacagaataataaaccacatgtggggttttaaaaatgaaatttggctaataggagcaattcagctatttttctatacagtaattggtgtgtggtatagaagaaaaacgggttcaaaccccacttctgccacctaccagctatatggccttgaatgagtcattcagctttaataaggttcattttcttctgtttaaaaagacacaaaacttgaaaatcagctttggccatctacctgagaattagaaagtctgatttttggaattagaaatcatgattgtaggctgggcacagtggctcgcgcctgtaatcccagcactttgggaggccaaggcggacggatcacttgaggttaggagtttgagaccagcctggccaacatggtgaaaccccatctctactaaaaaaaaaaaaaaa

SEQ ID NO: 296 - DNA, 2033 bp, Homo sapiens
ggtaaagatggcagctaccatgttccgggctacgctgcggggatggagaaccggtgtccagcggggctgcgggctacggctgttgagccagacccagggccctccagattaccccaggtttgtggagtctgtggatgaatatcagtttgtggagcgcctgttaccggctaccaggatcccagatcccccaaagcatgaacattatcctacccctagtggctggcagcctcccagagaccccccacccaacctgccttactttgtacgacgctctcggatgcacaacatccccgtctacaaggacatcacgcatggcaaccggcagatgactgtgatccggaaagtggaaggggacatctgggccctgcagaaagacgtggaagattttctgagcccgctgctggggaagacacctgtcacccaggtcaatgaggtgacaggtaccctacggatcaagggctactttgaccaggagcttaaagcctggctcttggagaaaggcttctgaggcccagcccgagcagcctgcttgtcagcatgccctgtggatcaagtctagggggcctcaggaggagggaggtgggtgttggagcccctgagacaggggatacagaaactagggctaaaggactttggggtcaggccttgcttgcataaaggagaaaacaactctatgtacatgctgggggagagtgcctaatgtgggagaccaaatagggatcaccaggctaatggggggcgtcagcagctttctctccctcctatcttggcctgttcttttttgttttttgagacggagtctcactctgttgcccagggtggagtgcagtggcatgatcttggctcactgcaacttccacctctgggatcaagggattctcctgcctcagcctcttgagtagctgggattacaggcgcccaccaccacagcctgctaatttttgtatttttagtagagatggggtttcaccatgttggccaggctggtctcaaactcctgactcgaagtgatccgcccaccttggcctcccaaagcgttgggattataggcatgagccatgtgcctggtccaccttggcctgttttgtttttctttccttgggctcagcaattcaaattctagttgttatttggtggaagcagtagcccaaccccagtttaggggaaggtagcacagggcagagccactgggcactttgtttccttggccctccgaagctcactgttgcaaatacccccaagcctttgctctaggccagatcttgtttggtgcaggtgatggagaacacagatgactcgggcatgggtcttggagatcttctgttcaaagtacagtgctggcactggggcacagagtgcccacgttagccccgggctctgatagagaggtaggaggcacgttcttggtcactgttccattgcagaccagacttgctggcctgaccacaagggagtggctgggaactcacagccagcatagggacatccccctgcagccttctgacctgcaatcaaggctggggaggggtttgcaggcaggaatatgctgacctttcaccctgccatcccatcccaaccccagctcactagccttcatatatgccttatacttggagtcacaggggccaaaggcctgagaccccaccctgcccccaaactggctaagacagctttcagttcctgactccccaacttggtctctgccctgaagcagggcactgaactctgggctgcttctctgtgtgtaaaatgggcacatcttcctaatctgttaatggtcagtggtgtccccaaggatagtgctggcttccatggaaaccctcactcctggagattccattccattttcaagtgtacagccacagcaaggagcccgacactgatttgatcgattctgtgacacaaaccccaccaattgttaatgcaagtttttatttggctgtatatacaatttaagctattaaaatttgtacaatatttacaaattaaataatcatctgaaactgtcaaaaaaaaaaaaaaaaaaaaaaaaa

SEQ ID NO: 297 - DNA, 1059 bp, Homo sapiens
gttttctgtcactggacgccaaggagttttcggtggctcagctgggtaaccggggatcaccatggcggcctcattggtggggaagaagatcgtgtttgtaacggggaacgccaagaagctggaggaggtcgttcagattctaggagataagtttccatgcactttggtggcacagaaaattgacctgccggagtaccaaggggagccggatgagatttccatacagaaatgtcaggaggcagttcgccaggtacaggggcccgtgctggttgaggacacttgtctgtgcttcaatgcccttggagggctccccggcccctacataaagtggtttctggagaagttaaagcctgaaggtctccaccagctcctggccgggttcgaggacaagtcagcctatgcgctctgcacgtttgcactcagcaccggggacccaagccagcccgtgcgcctgttcaggggccggacctcgggccggatcgtggcacccagaggctgccaggactttggctgggacccctgctttcagcctgatggatatgagcagacgtacgcagagatgcctaaggcggagaagaacgctgtctcccatcgcttccgggccctgctggagctgcaggaatactttggcagtttggcagcttgacttctgcagctggaggaggcccctcaggccggggatctggggagggctagcccaaaacctcccgcatcgggcaggcaccccctgaagtacttccttcagggtttcccctttgtgagggtgtcgagtagcctcaccggcctgtctggaggagcagctggctctgctctgagaaactctggcaagtggacgccattctcttgcccttaggattcactgctctctcctacagccgccaggcctggggtcctgaaaggaccttgggtggtaaagctgtacttggtgggagtgagggcgtggggaggaaccatgcaaatcgccttccatggtttttaaatgcagtaaataacatttctggatgagacttgtttccaaaataaaccagctatatctgttttgaaaaaaaaaaaaaaaaa

SEQ ID NO: 298 - DNA, 1769 bp, Homo sapiens
gggttgggctgtgacgctgctgctggggtcagaatgtcatacccaggctatcccccaacaggctacccacctttccctggatatcctcctgcaggtcaggagtcatcttttcccccttctggtcagtatccttatcctagtggctttcctccaatgggaggaggtgcctacccacaagtgccaagtagtggctacccaggagctggaggctaccctgcgcctggaggttatccagcccctggaggctatcctggtgccccacagccagggggagctccatcctatcccggagttcctccaggccaaggatttggagtcccaccaggtggagcaggcttttctgggtatccacagccaccttcacagtcttatggaggtggtccagcacaggttccactacctggtggctttcctggaggacagatgccttctcagtatcctggaggacaacctacttaccctagtcagcctgccacagtgactcaggtcactcaaggaactatccgaccagctgccaacttcgatgctataagagatgcagaaattcttcgtaaggcaatgaagggttttgggacagatgagcaggcaattgtggatgtggtggccaaccgttccaatgatcagaggcaaaaaattaaagcagcatttaagacctcctatggcaaggatttaatcaaagatctcaaatcagagttaagtggaaatatggaagaactgatcctggccctcttcatgcctcctacgtattacgatgcctggagcttacggaaagcaatgcagggagcaggaactcaggaacgtgtattgattgagattttgtgcacaagaacaaatcaggaaatccgagaaattgtcagatgttatcagtcagaatttggacgagaccttgaaaaggacattaggtcagatacatcaggacattttgaacgtttacttgtgtccatgtgccagggaaatcgtgatgagaaccagagtataaaccaccaaatggctcaggaagatgctcagcgtctctatcaagctggtgaggggagactagggaccgatgaatcttgctttaacatgatccttgccacaagaagctttcctcagctgagagctaccatggaggcttattctaggatggctaatcgagacttgttaagcagtgtgagccgtgagttttccggatatgtagaaagtggtttgaagaccatcttgcagtgtgccctgaaccgccctgccttctttgctgagaggctctactatgctatgaaaggtgctggcacagatgactccaccctggtccggattgtggtcactcgaagtgagattgaccttgtacaaataaaacagatgttcgctcagatgtatcagaagactctgggcacaatgattgcaggtgacacgagtggagattaccgaagacttcttctggctattgtgggccagtaggagggatttttttttttttaatgaaaaaaaaatttctattcatagcttatccttcagagcaatgacctgcatgcagcaatatcaaacatcagctaaccgaaagagctttctgtcaaggaccgtatcagggtaatgtgcttggtttgcacatgttgttattgccttaattctaattttattttgttctctacatacaatcaatgtaaagccatatcacaatgatacagtaatattgcaatgtttgtaaaccttcattcttactagtttcattctaatcaagatgtcaaattgaataaaaatcacagcaatctttgaaaaaaaaaaaaaaaaaaaaa

SEQ ID NO: 299 - DNA, 463 bp, Homo sapiens
cgcgccgccacaatggtgcgcatgaatgtcctggcagatgctctcaagagtatcaacaatgccgaaaagagaggcaaacgccaggtgcttattaggccgtgctccaaagtcatcgtccggtttctcactgtgatgatgaagcatggttacattggcgaatttgaaatcattgatgaccacagagctgggaaaattgttgtgaacctcacaggcaggctaaacaagtgtggggtgatcagccccagatttgacgtgcaactcaaagacctggaaaaatggcagaataatctgcttccatcccgccagtttggtttcattgtactgacaacctcagctggcatcatggaccatgaagaagcaagacgaaaacacacaggagggaaaatcctgggattctttttctagggatgtaatacatatatttacaaataaaatgcctcatggacaaaaaaaaaaaaaaaaa

SEQ ID NO: 300 - DNA, 703 bp, Homo sapiens
gcgagaatgaagactattctcagcaatcagactgtcgacattccagaaaatgtcgacattactctgaagggacgcacagttatcgtgaagggccccagaggaaccctgcggagggacttcaatcacatcaatgtagaactcagccttcttggaaagaaaaaaaagaggctccgggttgacaaatggtggggtaacagaaaggaactggctaccgttcggactatttgtagtcatgtacagaacatgatcaagggtgttacactgggcttccgttacaagatgaggtctgtgtatgctcacttccccatcaacgttgttatccaggagaatgggtctcttgttgaaatccgaaatttcttgggtgaaaaatacatccgcagggttcggatgagaccaggtgttgcttgttcagtatctcaagcccagaaagatgaattaatccttgaaggaaatgacattgagcttgtttcaaattcagcggctttgattcagcaagccacaacagttaaaaacaaggatatcaggaaatttttggatggtatctatgtctctgaaaaaggaactgttcagcaggctgatgaataagatctaagagttacctggctacagaaagaagatgccagatgacacttaagacctacttgtgatatttaaatgatgcaataaaagacctattgatttgggaaaaaaaaaaaaaaaaaaa

SEQ ID NO: 301 - DNA, 887 bp, Homo sapiens
gcgggggccaggggtgggcgcgggagacggggcggtacccggggtgggagagggacggggagggggcgaggggcggaggccgagggggcagggggggtgggcggcggccagtgtttacagatgagctttaactgccgcctcaggcgtggagacggagaccccgcagcccggcggcgcctcagcccttcaacgacagtattgagtggtcaggttacaataaaccggagagaaaaggtccgcttgcactttttttagttttcttatttttagacacccctcccctccagggtgatctttaaaaaagcaaaacaaaaaacacgacttttccagccgctcagccgttttttcctttcgtccgaagccgttttctgatttgacttttctcgccggccggtctcaggccgcacagacgttccagaggaggagggtgacatttttactccctttttggggctaaccatttatgcttttgtacatcaaccgtgcgcggccggaggggggcaggggggcgggggccgaggggcgttccaatcaaatttctaactttctgttaattattaatcccctttttactgcggtttctgttgtcatttttaaaatttttttaattttttttttttttttacttttactttttacctcttgtgtatatgtagggaatttatagggaaatatgtactttatggaataaattttaagaactaaaatatattttattttaaataaagtaatggacctttaatcttacgcagctaaattactgattatatatttgctgagctgatttaagggttaaaaaaattgtatcaagagttttattttttgacttcaaagccttgttaataaagcctcttttatacatgtgaaaaaaaaaaaaaaaaaaaaaa

SEQ ID NO: 302 - DNA, 905 bp, Homo sapiens
gttctagatcgcgagtggccgccctttttttttttttttttgcaggaaggaattccatttattgtggatgcattttcacaatatatgtttattggagcgatccattatcagtgaaaagtatcaagtgtttataaaatttttaggaatggcagattcacagaacatgctagtcagcctgcagtattcctccagaagagctaaccagggcagggctggcatgagaagtgacatctgcgttacaaagtctatcttcctcataagtctgtaaagagcaattgaatcttctagctttagcaaacctaagccaaaggaaggaaagccacgaagaatgcagaagtcaaaccctcatgacaaagtaggcacaagtctacaataagctaaatcagaatttacaaatacaagtgtcccaggtagcattgactcccgtcattggagtgaaatggatcaaagtttgaattaaggcctatggtaaggtaacattgctttgttgtacttttgaacaagagctcctcctgatcactattacatatttttctagaaaatctaaagttcagaagagaatgtatcactgctgacttttattccaatatttggatggagtaagttttagggtagaattttgttcagtttggatttaatcttttgaaaagtaaattccttgtttactggtttgactataattctctgttatctttacgaggtaaaactgcaagctgactagcatgttctgtgaatctgccattcctaaaaattttataaacacttgatacttttcactgataatggatcgctccaataaacatatattgtgaaaatgcatccacaataaatggaattccttcctgcaaaaaaaaaaaaaaaaaaaaaaaaaaaaaaaaaaaaaaaaaaaaaaaaaaaaaaaaaaa

SEQ ID NO: 303 - DNA, 1832 bp, Homo sapiens
tcagcaaagggaaatcaccctgaaaggttttagaaatggtagttttggagttttggtggcaaccaatgttgctgcacgtgggttagacatccctgaggttgatttggttatacaaagctctccaccaaaggatgtagagtcctacattcatcgatccgggcggacaggcagagctggaaggacgggggtgtgcatctgcttttatcagcacaaggaagaatatcagttagtacaagtggagcaaaaagcgggaattaagttcaaacgaataggtgttccttctgcaacagaaataataaaagcttccagcaaagatgccatcaggcttttggattccgtgcctcccactgccattagtcacttcaaacaatcagctgagaagctgatagaggagaagggagctgtggaagctctggcagcagcactggcccatatttcaggtgccacgtccgtagaccagcgctccttgatcaactcaaatgtgggttttgtgaccatgatcttgcagtgctcaattgaaatgccaaatattagttatgcttggaaagaacttaaagagcagctgggcgaggagattgattccaaagtgaagggaatggtttttctcaaaggaaagctgggtgtttgctttgatgtacctaccgcatcagtaacagaaatacaggagaaatggcatgattcacgacgctggcagctctctgtggccacagagcaaccagaactggaaggaccacgggaaggatatggaggcttcaggggacagcgggaaggcagtcgaggcttcaggggacagcgggacggaaacagaagattcagaggacagcgggaaggcagtagaggcccgagaggacagcgatcaggaggtggcaacaaaagtaacagatcccaaaacaaaggccagaagcggagtttcagtaaagcatttggtcaataattagaaatagaagatttatatagcaaaaagagaatgatgtttggcaatatagaactgaacattatttttcatgcaaagttaaaagcacattgtgcctccttttgaccacttgccaagtccctgtctctttcagacacagacaagcttcatttaaattatttcatctgatcattatcatttataactttattgttacttcatcagtttttccttttgaaaggtgtatgaattcattacatttttattctaatgtattatctgtagattagaagataaaatcaagcatgtatctgcctatactttgtgagttcacctgtctttatactcaaaagtgtcccttaatagtgtccttccctgaaataaatacctaagggagtgtaacagtctctggaggaccactttgagcctttggaagttaaggtttcctcagccacctgccgaacagtttctcatgtggtcctattatttgtctactgagacttaatactgagcaatgttttgaaacaagatttcaaactaatctgggttgtaatacagtttataccagtgtatgctctagacttggaagatgtagtatgtttgatgtggattacctatacttatgttcgttttgatacatttttagcttctcattataaggtgattcatgctttagtgaattcttcatagatagtatatataaaagtacattttaatagaaagccagggttttaaggaatttcacatgtataaggtggctccatagctttatttgtaagtaggctggataaatggtgcttaaatggtaatgtactccacttcttcctattggaagattaacattatttaccaagaaggacttaagggagtaaggggcgcagattagcattgctcaagagtatgtaaaaaaaaaaaaaaa

SEQ ID NO: 304 - DNA, 1824 bp, Homo sapiens
ggcaggacagaagacttcagctgccctgtccacagtgggctctgccatcagcaggaagcttggagacatgaggaactctgcgaccttcaagtcgtttgaggaccgagttgggaccataaagtctaaggttgtgggtgacagagagaacggcagtgacaacctcccttcctcagcggggagtggtgacaagcccctgtcggatcccgcacctttctaagcctgtggttgcttcacccgctgcagagcacacgcaacccagcctcagcatcacagccgcagctctgttcagcggagcagccagccagggcggatgagcagagccggccctgaggacagtcctgcccatccacgcggagatgtggctgccgcgtttgcatgaatttggagaaaacaggcttgtacacagatgttttacactcacgtttgtagatgaaacagatcactgtgctgtccttcctaggggtgcaggaagtggacagggcggagggtttgaaagaatattgagccaaagcccaggctccctttgggaatcatgttagcccatcagaatgttgaaggattgaagagttctaagcgtaaaataagtggcattttctgacttcttcctcctcctccttccctgactcacagaaggaatgcaatcacccagcaagtcctacctgttacgcaattttttatctcaaaatgccgaacgagaaaactgtccattttctgagacccccagaaaggaaactgaccctcagcagctgcctgattgttacgcgaatctagctttaacggaagcaaattcattattttttaaatgcagtggacttttcaaaaagtttaaattaggcaaagcagctttagcctcatagaatattatttctttggactcaagctgaaatacaagccttacattgccttatgctttatttctttctaatttttatatgtatatagatgagggttccttaatggttgtgagcattgtgtggaattttacacctggcctgcgtggcagcctcttccagttgaggtgttttatgtcacgcacactccatcccagtgtacaaaacctgcttctcttctcaaccgtggcagctcccgctggctcctatgccctgccctaaagggctcttgagcctctgggaatgggaggggccaagagaaggaaaaccctgtctttagcaccctttaaaagaactgtgccccccttctcagtgctgcctttgcatgggcctggcccggctcacattcgtcagtgactccaaccctcctgcttgctgtacttgggatgaaacgaccccacaggtcaagtggagggtggggcgtgggcatcagccaggattgccgttacagtctttttctcaggagctacaaagatctcttcctgttactaaatggtcgcaccccagcagcctctctcgcacaccggggccctgcatgtcagatggcgtggtctgcagggggagctctgtgccttagtggctcttggcaggacactgagggcctgcctgtggtgtgcccggctctgccactcccgggaggggaagggctgctcagctcaaggtgtcctgttcggtagagcaagtgtcctctgacagccgtgtccccggacagttcagacacccttggggatggcactccacacacgacagagatgcaggggccagggaagcccagcgctcggtgcccttcgtccagggttaaaatcggcctgtggggtgtggtgagaaggcaggttgtgcgggtgttgaccgatgtatcttttccttaaagttattataataatgggtaatttgtcaataaagcattcctttgggggaaaaaaaaaaaaaaaaa

SEQ ID NO: 305 - DNA, 759 bp, Homo sapiens
cgatgggcatctctcgggacaactggcacaagcgccgcaaaaccgggggcaagagaaagccctaccacaagaagcggaagtatgagttggggcgcccagctgccaacaccaagattggcccccgccgcatccacacagtccgtgtgcggggaggtaacaagaaataccgtgccctgaggttggacgtggggaatttctcctggggctcagagtgttgtactcgtaaaacaaggatcatcgatgttgtctacaatgcatctaataacgagctggttcgtaccaagaccctggtgaagaattgcatcgtgctcatcgacagcacaccgtaccgacagtggtacgagtcccactatgcgctgcccctgggccgcaagaagggagccaagctgactcctgaggaagaagagattttaaacaaaaaacgatctaaaaaaattcagaagaaatatgatgaaaggaaaaagaatgccaaaatcagcagtctcctggaggagcagttccagcagggcaagcttcttgcgtgcatcgcttcaaggccgggacagtgtggccgagcagatggctatgtgctagagggcaaagagttggagttctatcttaggaaaatcaaggcccgcaaaggcaaataaatccttgttttgtcttcacccatgtaataaaggtgtttattgttttgttcccaaaaaaaaaaaaaaaaaaaaaaaaaaaaaaaaaaaaaaaaaaaaaaaaaaaaaaaaaaaaaaaaaaaaaaaaaaaaaa

SEQ ID NO: 306 - DNA, 938 bp, Homo sapiens
gtcgtcggggtttcctgcttcaacagtgcttggacggaacccggcgctcgttccccaccccggccggccgcccatagccagccctccgtcacctcttcaccgcaccctcggactgccccaaggcccccgccgccgctccagcgccgcgcagccaccgccgccgccgccgcctctccttagtcgccgccatgacgaccgcgtccacctcgcaggtgcgccagaactaccaccaggactcagaggccgccatcaaccgccagatcaacctggagctctacgcctcctacgtttacctgtccatgtcttactactttgaccgcgatgatgtggctttgaagaactttgccaaatactttcttcaccaatctcatgaggagagggaacatgctgagaaactgatgaagctgcagaaccaacgaggtggccgaatctttcttcaggatatcaagaaaccagactgtgatgactgggagagcgggctgaatgcaatggagtgtgcattacatttggaaaaaaatgtgaatcagtcactactggaactgcacaaactggccactgacaaaaatgacccccatttgtgtgacttcattgagacacattacctgaatgagcaggtgaaagccatcaaagaattgggtgaccacgtgaccaacttgcgcaagatgggagcgcccgaatctggcttggcggaatatctctttgacaagcacaccctgggagacagtgataatgaaagctaagcctcgggctaatttccccatagccgtggggtgacttccctggtcaccaaggcagtgcatgcatgttggggtttcctttaccttttctataagttgtaccaaaacatccacttaagttctttgatttgtaccattccttcaaataaagaaatttggtaaaaaaaaaaaaaaaaaaaaaaaaaaaaaaaaaaaaaaaaa

SEQ ID NO: 307 - DNA, 1281 bp, Homo sapiens
gcgtgatgtctcacagaaagttctccgctcccagacatgggtccctcggcttcctgcctcggaagcgcagcagcaggcatcgtgggaaggtgaagagcttccctaaggatgacccatccaagccggtccacctcacagccttcctgggatacaaggctggcatgactcacatcgtgcgggaagtcgacaggccgggatccaaggtgaacaagaaggaggtggtggaggctgtgaccattgtagagacaccacccatggtggttgtgggcattgtgggctacgtggaaacccctcgaggcctccggaccttcaagactgtctttgctgagcacatcagtgatgaatgcaagaggcgtttctataagaattggcataaatctaagaagaaggcctttaccaagtactgcaagaaatggcaggatgaggatggcaagaagcagctggagaaggacttcagcagcatgaagaagtactgccaagtcatccgtgtcattgcccacacccagatgcgcctgcttcctctgcgccagaagaaggcccacctgatggagatccaggtgaacggaggcactgtggccgagaagctggactgggcccgcgagaggcttgagcagcaggtacctgtgaaccaagtgtttgggcaggatgagatgatcgacgtcatcggggtgaccaagggcaaaggctacaaaggggtcaccagtcgttggcacaccaagaagctgccccgcaagacccaccgaggcctgcgcaaggtggcctgtattggggcatggcatcctgctcgtgtagccttctctgtggcacgcgctgggcagaaaggctaccatcaccgcactgagatcaacaagaagatttataagattggccagggctaccttatcaaggacggcaagctgatcaagaacaatgcctccactgactatgacctatctgacaagagcatcaaccctctgggtggctttgtccactatggtgaagtgaccaatgactttgtcatgctgaaaggctgtgtggtgggaaccaagaagcgggtgctcaccctccgcaagtccttgctggtgcagacgaagcggcgggctctggagaagattgaccttaagttcattgacaccacctccaagtttggccatggccgcttccagaccatggaggagaagaaagcattcatgggaccactgaagaaagaccgaattgcaaaggaagaaggagcttaatgccaggaacagattttgcagttggtggggtctcaataaaattattttccactgaaaaaaaaaa

SEQ ID NO: 308 - DNA, 1698 bp, Homo sapiens
gtttgaagcaaacaggcagcgcgcgacaatggcggtcgctcgtgcagctttggggccattggtgacgggtctgtacgacgtgcaggctttcaagtttggggacttcgtgctgaagagcgggctttcctcccccatctacatcgatctgcggggcatcgtgtctcgaccgcgtcttctgagtcaggttgcagatattttattccaaactgcccaaaatgcaggcatcagttttgacaccgtgtgtggagtgccttatacagctttgccattggctacagttatctgttcaaccaatcaaattccaatgcttattagaaggaaagaaacaaaggattatggaactaagcgtcttgtagaaggaactattaatccaggagaaacctgtttaatcattgaagatgttgtcaccagtggatctagtgttttggaaactgttgaggttcttcagaaggagggcttgaaggtcactgatgccatagtgctgttggacagagagcagggaggcaaggacaagttgcaggcgcacgggatccgcctccactcagtgtgtacattgtccaaaatgctggagattctcgagcagcagaaaaaagttgatgctgagacagttgggagagtgaagaggtttattcaggagaatgtctttgtggcagcgaatcataatggttctcccctttctataaaggaagcacccaaagaactcagcttcggtgcacgtgcagagctgcccaggatccacccagttgcatcgaagcttctcaggcttatgcaaaagaaggagaccaatctgtgtctatctgctgatgtttcactggccagagagctgttgcagctagcagatgctttaggacctagtatctgcatgctgaagactcatgtagatattttgaatgattttactctggatgtgatgaaggagttgataactctggcaaaatgccatgagttcttgatatttgaagaccggaagtttgcagatataggaaacacagtgaaaaagcagtatgaaggaggtatctttaaaatagcttcctgggcagatctagtaaatgctcacgtggtgccaggctcaggagttgtgaaaggcctgcaagaagtgggcctgcctttgcatcgggggtgcctccttattgcggaaatgagctccaccggctccctggccactggggactacactagagcagcggttagaatggctgaggagcactctgaatttgttgttggttttatttctggctcccgagtaagcatgaaaccagaatttcttcacttgactccaggagttcagttggaagcaggaggagataatcttggccaacagtacaatagcccacaagaagttattggcaaacgaggttccgatatcatcattgtaggtcgtggcataatctcagcagctgatcgtctggaagcagcagagatgtacagaaaagctgcttgggaagcgtatttgagtagacttggtgtttgagtgcttcagatacatttttcagatacaatgtgaagacattgaagatatgtggtcccctgaaagtcactggctggaaataatccaattattcctgcttggattcttccacagggcctgtgtaagaatgggttctggagttctcatggtctttaggaaatattgagtaatttgtaatcaccgcattgatactataataagttcattcttaaaaaaaaaaaaaaaaaaaa

SEQ ID NO: 309 - DNA, 1102 bp, Homo sapiens
gttataaatggttataaagctcctgttactcatattagttatttacatcaaaaagcttttagaaaatggtacgaggtaaccaattcttgtcatggtgaaatctgattgagtaaccaagcagttttactattctggtgctgcttcataacaaaaatgaaaagctgcatgcatctacagcaggcatggattgtttatgtcgtatgatatcctttattaagtaagttcacttatagtatttctataatttgattcattgccgtaatagagccatgtaggaaatgcactgattgcatgttattgtggcaagaatatcctaaatgtcattaaaatcctccaacatgatggatctacttatggtcttgtttgttgacatgacaaattaacattcttatagttacatctggaaatgagcatttgaaatagataatcctttaagccttgtggcaaaatttttgtggcttttgtttaactttgaaaggttattatgcactaaccttttttggtggctaattagggtttaaatacagaaacaagatttcaaataaaactgtctttggcagtgagtaaatagcatattttgaagtagagttgtatactttttcataagatgtttgggaatttttttcctgaagtaataatttattccacatctacatcagtgaaagctatctacctatcctgagtctatcttaaaggaaaaaaagaaaaaaaccttatctcttgcccttattttgaattttccactctttcattaatttgttttaagctccgtgttggaaaaaaggggtagtgcattttaaattgaccttcatacgcttttaaaataagacaaatctacttgataatgtacctttatttgatctcaagttgtataaaaccaataaatttgtgttactgcagtagtaatcttatgcacacggtgatttcatgttatatatgcaaagtaggcaactgttttcttagttacagaagtttcaagcttcacttttgtgcagtagaaacaaaagtaggctacagtctgtgccatgttgatgtacagtttctgaaattgttttacaagactttgataataaaacccttaaacttaaaaaaaaaaaaaaaa

SEQ ID NO: 310 - DNA, 519 bp, Homo sapiens
tcaaaagtcaaggcatcatttaaaataatctgatttcagacaaatgctgtgtggaaacatctatcctatagatcatcctattcttatgtgtctttggttatcagatcaattacagaataattgtgttgtgatattgtgtcctaaattgctcattaatttttatttacagattgaaaaagagggaccgtgtaaagaaaatggaaaataaatatctttcaaagactcttttagataaacacgatgaggcaaaatcaggttcattcattcaacgatagtttctaaacagtacttaaatagcggttggaaaacgtagccttcattttatgattttttcatatgtggaaatctattacatgtaatacaaaacaaacatgtagtttgaaggcggtcagatttctttgagaaatctttgtagagttaattttatggaaattaaaatcagaattaaatgctaaaaaaaaaaaaaaaaaaaaaaaaaaataaaaaaaaaaaaaaataaaaaaaaaaaaaaaaaaaaaa

SEQ ID NO: 311 - DNA, 2335 bp, Homo sapiens
ccgaggtggctgctggtgggggctcctgggacgacaggctgcgcaggctcatctcccccaacctgggggtcgtgttcttcaacgcctgcgaggccgcgtcgcggctggcgcgcggcgaggatgaggcggagctggcgctgagcctcctggcgcagctgggcatcacgcctctgccactcagccgcggccccgtgccagccaaacccaccgtgctcttcgagaagatgggcgtgggccggctggacatgtatgtgctgcacccgccctccgccggcgccgagcgcacgctggcctctgtgtgcgccctgctggtgtggcaccccgccggccccggcgagaaggtggtgcgcgtgctgttccccggttgcaccccgcccgcctgcctcctggacggcctggtccgcctgcagcacttgaggttcctgcgagagcccgtggtgacgccccaggacctggaggggccggggcgagccgagagcaaagagagcgtgggctcccgggacagctcgaagagagagggcctcctggccacccaccctagacctggccaggagcgccctggggtggcccgcaaggagccagcacgggctgaggccccacgcaagactgagaaagaagccaaggccccccgggagttgaagaaagaccccaaaccgagtgtctcccggacccagccgcgggaggtgcgccgggcagcctcttctgtgcccaacctcaagaagacgaatgcccaggcggcacccaagccccgcaaagcgcccagcacgtcccactctggcttcccgccggtggcaaatggaccccgcagcccgcccagcctccgatgtggagaagccagcccccccagtgcagcctgcggctctccggcctcccagctggtggccacgcccagcctggagctggggccgatcccagccggggaggagaaggcactggagctgcctttggccgccagctcaatcccaaggccacgcacaccctcccctgagtcccaccggagccccgcagagggcagcgagcggctgtcgctgagcccactgcggggcggggaggccgggccagacgcctcacccacagtgaccacacccacggtgaccacgccctcactacccgcagaggtgggctccccgcactcgaccgaggtggacgagtccctgtcggtgtcctttgagcaggtgctgccgccatccgcccccaccagtgaggctgggctgagcctcccgctgcgtggcccccgggcgcggcgctcggcttccccacacgatgtggacctgtgcctggtgtcaccctgtgaatttgagcatcgcaaggcggtgccaatggcaccggcacctgcgtcccccggcagctcgaatgacagcagtgcccggtcacaggaacgggcaggtgggctgggggccgaggagacgccacccacatcggtcagcgagtccctgcccaccctgtctgactcggatcccgtgcccctggcccccggtgcggcagactcagacgaagacacagagggctttggagtccctcgccacgaccctttgcctgaccccctcaaggtccccccaccactgcctgacccatccagcatctgcatggtggaccccgagatgctgccccccaagacagcacggcaaacggagaacgtcagccgcacccggaagcccctggcccgccccaactcacgcgctgccgcccccaaagccactccagtggctgctgccaaaaccaaggggcttgctggtggggaccgtgccagccgaccactcagtgcccggagtgagcccagtgagaagggaggccgggcacccctgtccagaaagtcctcaacccccaagactgccactcgaggcccgtcggggtcagccagcagccggcccggggtgtcagccaccccacccaagtccccggtctacctggacctggcctacctgcccagcgggagcagcgcccacctggtggatgaggagttcttccagcgcgtgcgcgcgctctgctacgtcatcagtggccaggaccagcgcaaggaggaaggcatgcgggccgtcctggacgcgctactggccagcaagcagcattgggaccgtgacctgcaggtgaccctgatccccactttcgactcggtggccatgcatacgtggtacgcagagacgcacgcccggcaccaggcgctgggcatcacggtgttgggcagcaacagcatggtgtccatgcaggatgacgccttcccggcctgcaaggtggagttctagccccatcgccgacacgccccccactcagcccagcccgcctgtccctagattcagccacatcagaaataaactgtgacttccaaaaaaaaaaa

SEQ ID NO: 312 - DNA, 1027 bp, Homo sapiens
ggggcggcgggttggtctacgctgtgcgcggcggacgtcggaggcagcggggagcggagcggggccgccggggcctctccagggccgcagcggcagcagttgggccccccgccccggccggcggaccgaagaacgcaggaagggggccggggggacccgcccccggccggccgcagccatgaactccaacgtggagaacctacccccgcacatcatccgcctggtgtacaaggaggtgacgacactgaccgcagacccacccgatggcatcaaggtctttcccaacgaggaggacctcaccgacctccaggtcaccatcgagggccctgaggggaccccatatgctggaggtctgttccgcatgaaactcctgctggggaaggacttccctgcctccccacccaagggctacttcctgaccaagatcttccacccgaacgtgggcgccaatggcgagatctgcgtcaacgtgctcaagagggactggacggctgagctgggcatccgacacgtactgctgaccatcaagtgcctgctgatccaccctaaccccgagtctgcactcaacgaggaggcgggccgcctgctcttggagaactacgaggagtatgcagctcgggcccgtctgctcacagagatccacgggggcgccggcgggcccagcggcagggccgaagccggtcgggccctggccagtggcactgaagcttcctccaccgaccctggggccccagggggcccgggaggggctgagggtaccatggccaagaagcatgctggcgagcgcgataagaagctggcggccaagaaaaagacggacaagaagcgggcgctgcggcggctgtagtgggctctcttcctccttccaccgtgaccccaacctctcctgtcccctccctccaactctgtctctaagttatttaaattatggctggggtcggggagggtacagggggcactgggacctggatttgtttttctaaataaagttggaaaagcaaaaaaaaaaaaaaaaaaaaaaaaaaaa

SEQ ID NO: 313 - DNA, 1068 bp, Homo sapiens
gcggagcttgttactggttacttggcctcatggcggtccgagcttcgttcgagaacaactgtgagatcggctgctttgccaagctcaccaacacctactgtctggtagcgatcggaggctcagagaacttctacagtgtgttcgagggcgagctctccgataccatccccgtggtgcacgcgtctatcgccggctgccgcatcatcgggcgcatgtgtgtggggaacaggcacggtctcctggtacccaacaataccaccgaccaggagctgcaacacattcgcaacagcctcccagacacagtgcagattaggcgggtggaggagcggctctcagccttgggcaatgtcaccacctgcaatgactacgtggccttggtccacccagacttggacagggagacagaagaaattctggcagatgtgctcaaggtggaagtcttcagacagacagtggccgaccaggtgctagtaggaagctactgtgtcttcagcaatcagggagggctggtgcatcccaagacttcaattgaagaccaggatgagctgtcctctcttcttcaagtcccccttgtggcggggactgtgaaccgaggcagtgaggtgattgctgctgggatggtggtgaatgactggtgtgccttctgtggcctggacacaaccagcacagagctgtcagtggtggagagtgtcttcaagctgaatgaagcccagcctagcaccattgccaccagcatgcgggattccctcattgacagcctcacctgagtcaccttccaagttgttccatgggctcctggctctggactgtggccaaccttctccacattccgcccaatctgtaccggatgctggcagggaggtggcagagagctcactgggactgaggggctgggcacccaacccttttccacctgtgcttatcgcctggatctatcattactgcaaaaacctgctctgttgtgctggctggcaggccctgtggctgctggctgagggttctgctgtcctgtgccaccccattaaagtgcagttccctccggaaaaaaaaaaaaaaaaaaaaaaaaaa

SEQ ID NO: 314 - DNA, 810 bp, Homo sapiens
tgcaaatatccaagaagaggaagtttgtcgctgatggcatcttcaaagctgaactgaatgagtttcttactcgggagctggctgaagatggctactctggagttgaggtgcgagttacaccaaccaggacagaaatcattatcttagccaccagaacacagaatgttcttggtgagaagggccggcggattcgggaactgactgctgtagttcagaagaggtttggctttccagagggcagtgtagagctttatgctgaaaaggtggccactagaggtctgtgtgccattgcccaggcagagtctctgcgttacaaactcctaggagggcttgctgtgcggagggcctgctatggtgtgctgcggttcatcatggagagtggggccaaaggctgcgaggttgtggtgtctgggaaactccgaggacagagggctaaatccatgaagtttgtggatggcctgatgatccacagcggagaccctgttaactactacgttgacactgctgtgcgccacgtgttgctcagacagggtgtgctgggcatcaaggtgaagatcatgctgccctgggacccaactggtaagattggccctaagaagcccctgcctgaccacgtgagcattgtggaacccaaagatgagatactgcccaccacccccatctcagaacagaagggtgggaagccagagccgcctgccatgccccagccagtccccacagcataacagggtctccttggcagctgtattctggagtctggatgttgctctctaaagacctttaataaaattttgtacaaagaaaaaaaaa

SEQ ID NO: 315 - DNA, 2505 bp, Homo sapiens
cgtttgcacctcgctgctccagcctctggggcgcattccaaccttccagcctgcgacctgcggagaaaaaaaaattacttattttcttgccccatacataccttgaggcgagcaaaaaattaaattttaaccatgagggaaatcgtgcacatccaggctggtcagtgtggcaaccagatcggtgccaagttctgggaggtgatcagtgatgaacatggcatcgaccccaccggcacctaccacggggacagcgacctgcagctggaccgcatctctgtgtactacaatgaagccacaggtggcaaatatgttcctcgtgccatcctggtggatctagaacctgggaccatggactctgttcgctcaggtccttttggccagatctttagaccagacaactttgtatttggtcagtctggggcaggtaacaactgggccaaaggccactacacagagggcgccgagctggttgattctgtcctggatgtggtacggaaggaggcagagagctgtgactgcctgcagggcttccagctgacccactcactgggcgggggcacaggctctggaatgggcactctccttatcagcaagatccgagaagaataccctgatcgcatcatgaataccttcagtgtggtgccttcacccaaagtgtctgacaccgtggtcgagccctacaatgccaccctctccgtccatcagttggtagagaatactgatgagacctattgcattgacaacgaggccctctatgatatctgcttccgcactctgaagctgaccacaccaacctacggggatctgaaccaccttgtctcagccaccatgagtggtgtcaccacctgcctccgtttccctggccagctcaatgctgacctccgcaagttggcagtcaacatggtccccttcccacgtctccatttctttatgcctggctttgcccctctcaccagccgtggaagccagcagtatcgagctctcacagtgccggaactcacccagcaggtcttcgatgccaagaacatgatggctgcctgtgacccccgccacggccgatacctcaccgtggctgctgtcttccgtggtcggatgtccatgaaggaggtcgatgagcagatgcttaacgtgcagaacaagaacagcagctactttgtggaatggatccccaacaatgtcaagacagccgtctgtgacatcccacctcgtggcctcaagatggcagtcaccttcattggcaatagcacagccatccaggagctcttcaagcgcatctcggagcagttcactgccatgttccgccggaaggccttcctccactggtacacaggcgagggcatggacgagatggagttcaccgaggctgagagcaacatgaacgacctcgtctctgagtatcagcagtaccaggatgccaccgcagaagaggaggaggatttcggtgaggaggccgaagaggaggcctaaggcagagcccccatcacctcaggcttctcagttcccttagccgtcttactcaactgcccctttcctctccctcagaatttgtgtttgctgcctctatcttgttttttgttttttcttctggggggggggtctagaacagtgcctggcacatagtaggcgctcaataaatacttgtttgttgaatgtctcctctctctttccactctgggaaacctaggtttctgccattctgggtgaccctgtatttctttctggtgcccattccatttgtccagttaatacttcctcttaaaaatctccaagaagctgggtctccagatcccatttagaaccaaccaggtgctgaaaacacatgtagataatggccatcatcctaagcccaaagtagaaaatggtagaaggtagtgggtagaagtcactatataaggaaggggatgggattttccattctaaaagttttggagagggaaatccaggctattaaagtcactaaatttctaagtatgtccatttcccatctcagcttcaagggaggtgtcagcagtattatctccactttcaatctccctccaagctctactctggaggagtctgtcccactctgtcaagtggaatccttccctttccaactctacctccctcactcagctcctttcccctgatcagagaaagggatcaagggggttgggaggggggaaagagaccagccttggtccctaagcctccagaaacgtcttcttaatccccaccttttcttactcccaaaaaagaatgaacacccctgactctggagtggtgtatactgccacatcagtgtttgagtcagtccccagaggagaggggaaccctcctccatcttttttgcaacatctcatttcttccttttgctgttgcttcccccctcacacacttggttttgttctatcctacatttgagatttctattttatgttgaacttgctgctttttttcatattgaaaagatgacatcgccccaagagccaaaaataaatgggaattgaaaaaaaaaaaaaaaaa

SEQ ID NO: 316 - DNA, 1588 bp, Homo sapiens
gttgccgagagatggatgagcagattagactgatggaccagaacctgaagtgtctgagtgctgctgaagaaaagtactctcaaaaagaagataaatatgaggaagaaatcaagattcttactgataaactcaaggaggcagagacccgtgctgagtttgctgagagatcggtagccaagctggaaaagacaattgatgacctggaagataaactgaaatgcaccaaagaggagcacctctgtacacaaaggatgctggaccagaccctgcttgacctgaatgagatgtagaacgccccagtcccaccctgctgctgctcctccctctgacccagactccgcctgaggccagcctgcgggaagctgacctttaactgagggctgatctttaactggaaggctgctttctcctttcaccaccccctccttccctgtgtctttttcgccaaactgtctctgcctcttcccggagaatccagctgggctagaggctgagcacctttggaaacaacatttaagggaatgtgagcacaatgcataatgtctttaaaaagcatgttgtgatgtacacattttgtaattaccttttttgttgttttgtagcaaccatttgtaaaacattccaaataattccacagtcctgaagcagcaatcgaatccctttctcacttttggaaggtgacttttcaccttaatgcatattcccctctccatagaggagaggaaaaggtgtaggcctgccttaccgagagccaaacagagcccagggagactccgctgtgggaaacctcattgttctgtacaaagtactagctaaaccagaaaggtgattccaggaggagttagccaaacaacaacaaaaacaaaaaatgtgctgttcaagttttcagctttaagatatctttggataatgttatttctattttttatttttttcattagaagttaccaaattaagatggtaagacctctgagaccaaaattttgtcccatctctaccccctcacaactgcttacagaatggatcatgtcccccttatgttgaggtgaccacttaattgctttcctgcctccttgaaagaaagaaagaaagaagactgtgtttttgccactgatttagccatgtgaaactcatctcatcacccttttctgggtttgaagctgctgtctctagaagtgccatctcaattgtgctttgtatcagtcagtgctggagaaatcttgaatagcttatgtacaaaactttttaaattttatattattttgaaactttgctttgggtttgtggcaccctggccaccccatctggctgtgacagcctctgcagtccgtgggctggcagtttgttgatcttttaagtttccttccctacccagtccccattttctggtaaggtttctaggaggtctgttaggtgtacatcctgcagcttattggcttaaaatgtactctccttttatgtggtctctttggggccgattgggagaaagagaaatcaatagtgcaactgttttgatactgaatattgacaagtgtctttttgaaataaagaaccagtccctccaaccctcaaaaaaaaaaaaaaaaaa

SEQ ID NO: 317 - DNA, 1831 bp, Homo sapiens
gttgaggactacgcgcggcccaggcctgggccccccgctgctccaggccgcgctgggccttgggcgggctgggtggcactggcctgcgggccgggcggcgagcggggggcgcgggcgggcctggctgcagcccacgggccgggagacgggtgtgcaggtgtacaacagcctcaccgggaggaaggaacccctaatcgtggcgcacgccgaagccgcctcctggtatagctgtggaccaactgtatatgatcatgcgcaccttggccatgcttgctcatatgttagatttgatatcattcgaaggatcctaaccaaggtttttggatgcagcatagtcatggtgatgggtattacagatgtagatgataaaatcatcaaaagagccaatgagatgaatatttcccccgcttccctcgccagtctttatgaggaagacttcaagcaggacatggcagccctgaaggttctcccacccacggtgtacctgagggtaaccgaaaatattcctcagataatttctttcattgaaggaatcattgcttcgtgggaacgcttattcaacggcaaaaggcaatgtctacttcgatctgaaagtctagaggagacaaagtatggcaaaattggtcggcgtggtccctggtccagtccggagaccagcggacttctgacaagccgtcatgccaatgacttcgccctgtggaaggcggccaaaccccaggaggtgttctgggcctctccctggggacccgggaggccgggctggcacatcgagtgctctgccatcgctagtatggtatttggaagtcaactggatatccattcaggtgggatagatttagcttttccacatcatgagaacgaaattgcacagtgcgaagtctttcatcagtgcgagcagtggggaaattattttctgcattctgggcatttgcacgccaaaggcaaagaagaaaaaatgtccaaatcattaaagaactacattactattaaggactttctgaagaccttttcccccgatgtcttccggttcttctgcctgcggagcagctaccgctcagccatcgactacagtgacagcgccatgctccaagctcagcagctgctcctggggctgggctctttcctggaggacgcacgtgcctacatgaaggggcagctggcctgcggctccgtcagggaagcgatgctgtgggagaggctctccagcaccaagagggccgtgaaggcggccttggcagatgattttgacacacccagggtggttgatgccatcctgggccttgcacaccacgggaatggacagctcagggcgtccctgaaggaacctgaagggccgagaagtcctgctgtgtttggtgccatcatctcttactttgaacagttttttgaaactgttggaatttctctggcaaatcaacagtacgtttcaggagacggcagcgaggctaccttgcatggtgtggtggacgagctggtgcggttccggcagaaggtccggcagtttgcgctggccatgcccgaggccacgggggacgcccggcggcagcagctcctagaaaggcagcccctgctggaagcatgcgacaccctgcgccggggcctgactgcccacggcatcaacatcaaggacagaagcagtacaacatccacgtgggaactgctggatcaaaggacaaaagaccaaaaatcagcgggctgaggatggagcacagccatgaacctgctcacgacaagacgcacccatgcttctcagggtcaaggctttatgttaaagcttcctgtcggggctgctaggtcagcattaaagtaaggcaaccaaaaaaaaaaaaaa

SEQ ID NO: 318 - DNA, 3476 bp, Homo sapiens
gccgccgctgaggaaggctgtagtgtcggggccgaagcggacagggaattggaggagcttctggaaagatgccctcttcgcttcccaagagaagtttttccaggaactattcgacagtgaactggcttcccaagccactgcggagttcgagaaggcaatgaaggagttggctgaggaagaaccccacctggtggagcagttccaaaagctctcagaggctgcagggagagtgggcagtgatatgacctcccaacaagaattcacttcttgcctaaaggaaacactaagtggattagccaaaaatgccactgaccttcagaactccagcatgtcggaagaagagctgaccaaggccatggaggggctaggcatggacgaaggggatggggaagggaacatcctccccatcatgcagagtattatgcagaacctactctccaaggatgtgctgtacccatcactgaaggagatcacagaaaagtatccagaatggttgcagagtcatcgggaatctctacctccagagcagtttgaaaaatatcaggagcagcacagcgtcatgtgcaaaatatgtgagcagtttgaggcagagacccccacagacagtgaaaccactcaaaaggctcgttttgagatggtgctggatcttatgcagcagctacaagatttaggccatcctccaaaagagctggctggagagatgcctcctggcctcaactttgacctggatgccctcaatctttcgggcccaccaggtgccagtggtgaacagtgtctgatcatgtgaaacacaacacgttttcctctctgagtcccagctatggggaacatctggagtcagcagaaccattgggacctgaggcaggagtgtcacctgcgggagaagtctgcccgctgccctctgtcatcccattcaagattgtgccataccagctgaggtttttcctctgtctctctaggaatagggtctgtttcacaggccatttctgtgaaccctactccattgtggtttctgccactatcaaagttccagctacctgcaaggtgaaggaaggcatcccttttggggcatgcactttctttcctttctcaaaataatgttatatgtggccacactgatgttcacctttacgtccagggtctttgtgccttgtctctactccctctcttggatctggggaggaggggcagagacctgggactctgtatttctatagttctcctggcagagcctttgagaatggggagaaacagcctgggctggggctacaggtctgtcactatgctctcttgccttcagacagaccattctgaattctctaaagggaaagggcttttgcatctaatcacaatagagttgaaagagaggccttaggattctcctctctctaggtgctgagccctcacctccctgttccaggctgagaactcaaatggttaccctgcttcttcctacaatgctgtgtgatatgggtgaacccagcccctgaccttcctctatcccctgcccatcctcccttttacctcctctcttttttaaacacctgtttatcccaacctttttgagctcaagctgtgataaagaagggcccatcctatttcccctcatctagtccatttacgattctcactgactccccgtcttcctggcagacacaaataaacccagtgtcaggtctaggaaattaatggctattcttccccagatacattctggcttatttgagatacatgattctcttagaatcctgtcccttggttcaggaaagtagcttggaaaaggagtaggggtatagcttgggtcccttttcctgcaaggccccatggggcagaatataataaatattctgagtgaggagtgtggtctttttctgatcttcctcagcttccgtaagttgcagagtgaggtatattaggagactagttctacacaatattgtaatgctgggttccatcaacacccaccttccacaactcagtctgcacctcagttggcaaaggagactggatggccatctttcctcatgttcccttgagtatttcaatgtagaaagcccttcaagtggtattatattttaaccttttacattattgttattaatgttagtaatatattgttatgttttctaaattatttttctttaagctgacgtggctttttttctgtggctcccagtgggtctacggaccttggctgacatatgttggtaggtactctggtcagctcagctggctgtcctggttcactcagaagataagtctctccaaagcaaattcacatgcattatgagtcgctttgagcttctgacatgtcacttgccccgaggttaaaacttttcaccccttgaagaccttacatgttttatggtattggtgaggaaggaaatgttctcaaggtctcaggctatttgggaaattccaactcctataccttaccagagcatggaagagcccagatctgaatgtaaaacgtctctgttctgccagagatggaaaaaatacaggtatacttgtgatatagtcatggggcttcagtgtcactattttctccttaaagctccagccaaaaactggacaaggatagagaggaggagggaagaacaaaagagcccttctctatgaaccttgtgccttctgtcctaccagttttcttttacagattctcacttctgctagcctagccagggcttactccaggaatctaaatagatgccctagtccactttatctttgttcccaaggcactcatttttattttgattttgattgaatgtgagcaggttgacctcaggtcacactttgttccaaaaacttttggaattattccaggacttgtggtggagttatggtactctagggcagtctttctcaaactatgtatggtaaaggaccaggttttttgttttccagtccttcacttatcaatatgcattcctattgccgatgacaggtatggagttcacactgtgtgctgccgacccggcaagtttgacagcacccaaactggccagactgttctgtaggttaagtccattgatcatgtacttggatatcacagcaacattgaaatgctaaaaagtttttaaacactctcaatttctaattcaccatgtcacagactggtgaaaaaaaaaaaaaggtgttcactgaccagcacaagtctgcagatcatctttgagtagcactgttttggggccctcggtctctctgaagaccctagcagaactgatacctacctgtatctcttgttctctcctatttgagtttcacttccagagaacttgttcttcagcaagaatgtgtcactagtaaggacatctctagcatttctctagccttccttttctgctgctcaaaaataatcgttacaaagcttaggtttaagctgtatatgaaatatttatgcgactctcaaactttaaaggagttgctcctttgttccaaaattaaatgtgttagataaaaaaaaaaaaaaa

SEQ ID NO: 319 - DNA, 1665 bp, Homo sapiens
caacgacggcagccccgccggctactacctgaaggagtccaggggcagccggcggtggctcctcttcctggaaggcggctggtactgcttcaaccgcgagaactgcgactccagatacgacaccatgcggcgcctcatgagctcccgggactggccgcgcactcgcacaggcacagggatcctgtcctcacagccggaggagaacccctactggtggaacgcaaacatggtcttcatcccctactgctccagtgatgtttggagcggggcttcatccaagtctgagaagaacgagtacgccttcatgggcgccctcatcatccaggaggtggtgcgggagcttctgggcagagggctgagcggggccaaggtgctgctgctggccgggagcagcgcggggggcaccggggtgctcctgaatgtggaccgtgtggctgagcagctggagaagctgggctacccagccatccaggtgcgaggcctggctgactccggctggttcctggacaacaagcagtatcgccacacagactgcgtcgacacgatcacgtgcgcgcccacggaggccatccgccgtggcatcaggtactggaacggggtggtcccggagcgctgccgacgccagttccaggagggcgaggagtggaactgcttctttggctacaaggtctacccgaccctgcgctgccctgtgttcgtggtgcagtggctgtttgacgaggcacagctgacggtggacaacgtgcacctgacggggcagccggtgcaggagggcctgcggctgtacatccagaacctcggccgcgagctgcgccacacactcaaggacgtgccggccagctttgcccccgcctgcctctcccatgagatcatcatccggagccactggacggatgtccaggtgaaggggacgtcgctgccccgagcactgcactgctgggacaggagcctccatgacagccacaaggccagcaagacccccctcaagggctgccccgtccacctggtggacagctgcccctggccccactgcaacccctcatgccccaccgtccgagaccagttcacggggcaagagatgaacgtggcccagttcctcatgcacatgggcttcgacatgcagacggtggcccagccgcagggactggagcccagtgagctgctggggatgctgagcaacggaagctaggcagactgtctggaggaggagccggcactgaggggcccagacacccgctgccccagtgccacctcaccccccaccagcaggccctcccgtctcttcgggacagggccccagccgtcccccctgtctgggtctgcccactgccctcctgccccggctttccctgcccctctcccacagcccagccagagacaagggacctgctgtcatccccatctgtggcctgggggtccttcctgacaacgagggggtagccagaagagaagcactggattcctcagtccaccagctcagacagcacccaccggccccacccatcaagcccttttatattattttataaagtgacttttttattactttaattttttaaaaaaaggaaaataagaatatatgatgaatgatattgttttgtaactttttaaaaatgattttaaagagacaaaaaagaaaaaaaaaaaaaaaaaaaaaaaaaaaaa

SEQ ID NO: 320 - DNA, 1571 bp, Homo sapiens
tgctgttgtgatggtggtaagggaacgggcctggctctggcccctgacgcaggaacatggagctgatccaggacacctcccgcccgccactggagtacgtgaagggggtcccgctcatcaagtactttgcagaggcactggggcccctgcagagcttccaggcccggcctgatgacctgctcatcagcacctaccccaagtccggcaccacctgggtgagccagattctggacatgatctaccagggtggtgacctggagaagtgtcaccgagctcccatcttcatgcgggtgcccttccttgagttcaaagccccagggattccctcagggatggagactctgaaagacacaccggccccacgactcctgaagacacacctgcccctggctctgctcccccagactctgttggatcagaaggtcaaggtggtctatgttgcccgcaacgcaaaggatgtggcagtttcctactaccacttctaccacatggccaaggtgcaccctgagcctgggacctgggacagcttcctggagaagttcatggtcggagaagtgtcctacggatcctggtaccagcacgtgcaggagtggtgggagctgagccgcacccaccctgttctctacctcttctatgaagacatgaaggagaaccccaaaagggagattcaaaagatcctggagtttgtggggcactccctgccagaggagaccgtggacttcatggttcagcacacgtcgttcaaggagatgaagaagaaccctatgaccaactacaccaccgtcccccaggagttcatggaccacagcatctcccccttcatgaggaaaggcatggctggggactggaagaccaccttcaccgtggcgcagaatgagcgcttcgatgcggactatgcggagaagatggcaggctgcagcctcagcttccgctctgagctgtgagaggggctcctggggtcactgcagagggagtgtgcgaatcaaacctgaccaagcggctcaagaataaaatatgaattgagggcctgggacggtaggtcatgtctgtaatcccagcaatttggaggctgaggtgggaggatcatttgagcccaggagttcgagaccaacctgggcaacatagtgagattctgttaaaaaaataaaataaaataaaaccaatttttaaaaagagaataaaatatgattgtgggccaggcagagtggctcatgcctgtaatcccagcaatttgagaagttgaggctagaggatcactggaggacaggagtttgggaccagcctgttcaacattacaagacatcatccctacaaaaatttgagaaaattatctgtacgtgatggtgggcacctgtagtcccaactacttgacaagtgaaggcaggaggatcgcctgagccagggaggttatggctgcagttggctgactgggctaatccactcaagcctgagggacagagcaaatcttgcttgagaaataaataaaatacaatttacttaacataaattatgattcaggaccagtctggccaacatggtgaaaccccgtctctactaaaaaaaaaaaaaaa

SEQ ID NO: 321 - DNA, 1549 bp, Homo sapiens
gctgggtccttcggcaggaggaggaagatggagcccagcaccgcggcccgggcttgggccctcttttggttgctgctgcccttgcttggcgcggtttgcgccagcggaccccgcaccttagtgctgctggacaacctcaacgtgcgggagactcattcgcttttcttccggagcctgaaggaccggggctttgagctcacattcaagaccgctgatgaccccagcctgtctctcataaagtatggggaattcctctatgacaatctcatcattttctccccttcggtagaagattttggaggcaacatcaacgtggagaccatcagtgcctttattgacggcggaggcagtgtgctggtagctgccagctccgacattggtgaccctcttcgagagctgggcagtgagtgcgggattgagtttgacgaggagaaaacggctgtcattgaccatcacaactatgacatctcagaccttggccagcatacgctcatcgtggctgacactgagaacctgctgaaggccccaaccatcgttgggaaatcatctctaaatcccatcctctttcgaggtgttgggatggtggccgatcctgataaccctttggtgctggacatcctgacgggctcttccacctcttactccttcttcccggacaagcctatcacccagtatccacatgcggtggggaagaacaccctcctcattgctgggctccaggccaggaacaatgcccgcgtcatcttcagcggctccctcgacttcttcagcgactccttcttcaactcagcagtgcagaaggcggcgcccggctcccagaggtattcccagacaggcaactatgaactagctgtggccctctcccgctgggtgttcaaggaggagggtgtcctccgtgtggggcctgtgtcccatcatcgggtgggcgagacagccccacccaatgcctacactgtcactgacctagtggagtatagcatcgtgatccagcagctctcaaatggcaaatgggtcccctttgatggcgatgacattcagctggagtttgtccgcattgatccttttgtgaggaccttcctgaagaagaaaggtggcaaatacagtgttcagttcaagttgcccgacgtgtatggtgtattccagtttaaagtggattacaaccggctaggctacacacacctgtactcttccactcaggtatccgtgcggccactccagcacacgcagtatgagcgcttcatcccctcggcctacccctactacgccagcgccttctccatgatgctggggctcttcatcttcagcatcgtcttcttgcacatgaaggagaaggagaagtccgactgaggggctagagccctctccgcacagcgtggagacggggcagggaggggggttattaggattggtggttttgttttgctttgtttaaagccgtgggaaaatggcacaactttacctctgtgggagatgcaacactgagagccaaggggtgggagttgggataatttttatataaaagaagtttttccactttgaattgctaaaa

SEQ ID NO: 322 - DNA, 2064 bp, Homo sapiens
ctttgctcccctgccgccttctagtgggccgcctggtggtggacttgtcctgtgggatggagaccctccatacatggggttcaaaggtcctggggtattcctggatcttcaggacatctgcctaccctcaggtttcccaggcctcgggtggggaggcatcagatccctggccaacctgctatccaccccagggtttagacctctcttcccgtgagggaacagagtagtagcttcatttgtggcagattatctgccttgtgataaactttagtggcctccctttctgttttgccataatctgcctcctcccccactcccatacccatctacaaagcagccttgaaaataaccacccctgtggttcagtgtgggtcatgatctgtcaatccaggcttagtacatttccaaatggaattaccagaaggatgggcatcaaactgggaaaatgatttggtggcttgctagccgtgttcctttttctcccaaggcattcgttcagatgtgcagtgttggcagacccaggggatgccatttacctggcccactggtcgagatttggtcatcccaggaacttgggcttcagaaaagatgtttctgggctttggaccagattctggctggtctgtggtttgcccctcctgccacacccagtggctttcattgggcttttttcctctagttattgtcaggaacagtggggaatggcaggtattgcatttggtgaacacactggataactggaatcagagccgtgtgactgagggaagaggccttcacagcttcaatgcagaatgggatgattatctgccctgaaacgaaggatgcttctttgaggggagttgggaccaatactgggaacagcaccgctcctggcttctctggctttgcttctctctgaagtaatttagacttcgtgaaccttacatactggctccatctgtatattgggagagcctgtccctctcagcctcacagtagtgaagttaggaagcatggagtccacagtgttctttgaaagaatcaaggcaccaagtccatgcatttacatttctctcttgagatcagtccttttgttagcagcctgcaattctgaccacggcaaccaaaaatttctgaccgtggcaaccaaaaatcaagcgcgaggaaacctgggggtttatttggaggtgggagaggatgtcccttcttctggtaggactatgtctggctacattgtctttccatttttttctgattgtccccttgcccccctcccttttctgggggcacagagggcatggcaattttgtttcctagctttccatattcctactgcagtgctttcttcagaatacttgttcagatcggattcttggctgtgagggcaaccagctgctttcctgtttctttaaaagtggatcccacactgccgtgtcctgcacagtgagttctgcgttgtagaaaccatctagcagtggtgactcagggagtggcctctgctcggtatggggcctctgcaacatgaggttatggggcttctacgctgtggggtcaaaggagacatcatgatccctactggcagaaagagcagagccccagagtgggttccacttacggggtcagtgtcttttctgagatattcctcggaaccacatttaaattctttttcatattgtttgcataataattgccttctagtgcctactttataggactgagaggatttaaatgagataatccatgttatggatttaacacagcctctggcacaagtctaaatgttgcatgtaagtgttaactattatactggaaagaaggctcagttccttgatttaggtgtgggagaaaaatatatatatattttgagaccagccctggccaacatggtgaaactcatgtctacgaaaaatacaaaagttagcctggtgttggtggcgcatgcctatagttgcagctactcgggaggctgagacacaggaatcacttgaacccgggaggcggaggttgcagggagccgagatcacaccactgcactccagcctgggtaacagagtgagatactgtctcaaaaaaaaaaaaaaaaaa

SEQ ID NO: 323 - DNA, 1317 bp, Homo sapiens
gggtaatctctactttcctatactgccaaagaatgtgaggaagaaatgggactctttggttatttattgatgcgactgtaaattggtacagtatttctggagggcaatttggtaaaatgcatcaaaagacttaaaaatacggacgtactttgtgctgggaactctacatctagcaatttctctttaaaaccatatcagagatgcatacaaagaattatatataaagaagggtgtttaataatgatagttataataataaataattgaaacaatctgaatcccttgcaattggaggtaaattatgtcttagttataattagattgtgaatcagccaactgaaaatcctttttgcatatttcaatgtcctaaaaagacacggttgctctatatatgaagtgaaaaaaggatatggtagcattttatagtactagttttgctttaaaatgctatgtaaatatacaaaaaaactagaaagaaatatatataaccttgttattgtatttgggggagggatactgggataatttttattttctttgaatctttctgtgtcttcacatttttctacagtgaatttaatcaaatagtaaagttgttgtaaaaataaaagtggatttagaaagatccagttcttgaaaacactgtttctggtaatgaagcagaatttaagttggtaatattaaggtgaatgtcatttaagggagttacatctttattctgctaaagaagaggatcattgatttctgtacagtcagaacagtacttgggtttgcaacagctttctgagaaaagctaggtgtttaatagtttaactgaaagtttaactatttaaaagactaaatgcacattttatggtatctgatattttaaaaagtaatgtttgattctcctttttatgagttaaattattttatacgagttggtaatttttgctttttaataaagtggaagcttgcttttttaactctttttttattgttattttatagaaatgctttttgttggccgggcacagttgctcatccatgtaatcccagcactgtgggaggccgagacgggtggatcacaaggtcaggagatcgagaccatcctggctaatgcgttgaaactccgtctctgctaagaatacaaaaaattagctgggcgtggtggtgggcacctgtagtcccagctactcaggaggctgaggcaggagaatggtgtgaacctgggaggtggagcttgcagtgagcagagcttgcagtgagacgagcttgtgccactgcactccagcctgggcaacagagtaagactcagtctcaaaaaaaaaaaaaaaa

SEQ ID NO: 324 - DNA, 1483 bp, Homo sapiens
ttttcaatctttttgatctcacgaccactttttaaaaaatgtatatctttttttgtgtgttgttcttgttgttgttgttgtttgagacagggtcttgctctgtcgcccaggctggagttcagtggcactaacgtggctcatgccgcctcaacctcctagactcaagtgaccctccctcctcagcctcccaagtatctgggatcaaaggtgtgaaccaccaggcctggctaatttttttttattttttgtagagatgacatttctccatgttgcccaggctggtttcaaactcctgggctcaagcaagcctcaccctcccaaagtggtgggattacaagtatgagccaccacacctggccaggaccactggacactcttaaaaatgatgaaagacttcatggagaagcttgtattcatgtggattatagctatcaattgttactgtattagacatttaaaagaaaatagaaatgaggaaaatgaatttatttaaaaatagtaaacccaaatacatgtgaacataaatcacatttttatgaaaagatcactattttccacaattaaaaattgaggtttatgtagtttttgagatctctttaatgtctggcataatagaatacagctggattctcatgtatgcttctgcacaatcttttgtgatttttttttctttttttttgagacggagtcttgctctgtcacccaggctggagtgcagtggtgccatctcagctcagtgcaacctccactgccggggttcaagcgattatcctgcctcccgcatgaaatggggttttgccatgttggccaggcttgtctcgaactcctgacctcaggtgagccactgtgtccggccctgttgtggtatgttgaagtatataaagaaaatccaggttcacacggatacatatttttaaatggagaaatattggaatagccttttcagataattatggatattcttctttgatactgtacaaaatttggcaagtgatagttccttcaaagatagctgcagtgtggaatctgaaatcctatcaaggaagtttttgtactctgttaaactaaaaatccactgatccgttttacactttatttatgtgaagagtggtaacttttacccatgcatgattttgtaatatcctgtattggtcatttggaaaatactggtttactgaattatgcagttcttccaaatattgacacatttctttatataaatatcaagtcacatttgttaatatcacaactgatatcagaaaagtcccatggtaaatgcaaatttcacaaaattctaatgtttccttggaatcttgaattttatcattggcaacaaatacagttttccttgaaatgacagcctcactttttttaatttttgagtgagaagatacctgccaaatattcaagtctgaatggcgattgcttaccagtcattctttcatgttaaaatggtcttcctccatttaaaaaaaaaaaaaaa

SEQ ID NO: 325 - DNA, 1067 bp, Homo sapiens
gacggtcatcgattacaacggggaacgcacgctggatggttttaagaaattcctggagagcggtggccaggatggggcaggggatgatgacgatctcgaggacctggaagaagcagaggagccagacatggaggaagacgatgatcagaaagctgtgaaagatgaactgtaatacgcaaagccagacccgggcgctgccgagacccctcgggggctgcacacccagcagcagcgcacgcctccgaagcctgcggcctcgcttgaaggagggcgtcgccggaaacccagggaacctctctgaagtgacacctcacccctacacaccgtccgttcacccccgtctcttccttctgcttttcggtttttggaaagggatccatctccaggcagcccaccctggtggggcttgtttcctgaaaccatgatgtactttttcatacatgagtctgtccagagtgcttgctaccgtgttcggagtctcgctgcctccctcccgcgggaggtttctcctctttttgaaaattccgtctgtgggatttttagacatttttcgacatcagggtatttgttccaccttggccaggcctcctcggagaagcttgtcccccgtgtgggagggacggagccggactggacatggtcactcagtaccgcctgcagtgtcgccatgactgatcatggctcttgcatttttgggtaaatggagacttccggatcctgtcagggtgtcccccatgcctggaagaggagctggtggctgccagccctgggtcccggcacaggcctgggccttccccttccctcaagccagggctcctcctcctgtcgtgggctcattgtgaccactggcctctctacagcacggcctgtggcctgttcaaggcagaaccacgacccttgactcccgggtggggaggtggccaaggatgctggagctgaatcagacgctgacagttcttcaggcatttctatttcacaatcgaattgaacacattggccaaataaagttgaaattttcccccccaaaaaaaaaaaaaaaaaaaaaaaaaaaaaaaaaaaaaaaaaaa

SEQ ID NO: 326 - DNA, 915 bp, Homo sapiens
aggagccgcagggccgtaggcagccatggcgcccagccggaatggcatggtcttgaagccccacttccacaaggactggcagcggcgcgtggccacgtggttcaaccagccggcccgtaagatccgcagacgtaaggcccggcaagccaaggcgcgccgcatcgccccgcgccccgcgtcgggtcccatccggcccatcgtgcgctgccccacggttcggtaccacacgaaggtgcgcgccggccgcggcttcagcctggaggagctcagggtggccggcattcacaagaaggtggcccggaccatcggcatttctgtggatccgaggaggcggaacaagtccacggagtccctgcaggccaacgtgcagcggctgaaggagtaccgctccaaactcatcctcttccccaggaagccctcggcccccaagaagggagacagttctgctgaagaactgaaactggccacccagctgaccggaccggtcatgcccgtccggaacgtctataagaaggagaaagctcgagtcatcactgaggaagagaagaatttcaaagccttcgctagtctccgtatggcccgtgccaacgcccggctcttcggcatacgggcaaaaagagccaaggaagccgcagaacaggatgttgaaaagaaaaaataaagccctcctggggacttggaatcagtcggcagtcatgctgggtctccacgtggtgtgtttcgtgggaacaactgggcctgggatggggcttcactgctgtgacttcctcctgccaggggatttggggctttcttgaaagacagtccaagccctggataatgctttactttctgtgttgaagcactgttggttgtttggttagtgactgatgtaaaacggttttcttgtggggaaaaaaaaaaaaaaaaaaaaaa

SEQ ID NO: 327 - DNA, 2338 bp, Homo sapiens
agcgcacgtcggcagtcggctccctcgttgaccgaatcaccgacctctctccccagctgtatttccaaaatgtcgctttctaacaagctgacgctggacaagctggacgttaaagggaagcgggtcgttatgagagtcgacttcaatgttcctatgaagaacaaccagataacaaacaaccagaggattaaggctgctgtcccaagcatcaaattctgcttggacaatggagccaagtcggtagtccttatgagccacctaggccggcctgatggtgtgcccatgcctgacaagtactccttagagccagttgctgtagaactcaaatctctgctgggcaaggatgttctgttcttgaaggactgtgtaggcccagaagtggagaaagcctgtgccaacccagctgctgggtctgtcatcctgctggagaacctccgctttcatgtggaggaagaagggaagggaaaagatgcttctgggaacaaggttaaagccgagccagccaaaatagaagctttccgagcttcactttccaagctaggggatgtctatgtcaatgatgcttttggcactgctcacagagcccacagctccatggtaggagtcaatctgccacagaaggctggtgggtttttgatgaagaaggagctgaactactttgcaaaggccttggagagcccagagcgacccttcctggccatcctgggcggagctaaagttgcagacaagatccagctcatcaataatatgctggacaaagtcaatgagatgattattggtggtggaatggcttttaccttccttaaggtgctcaacaacatggagattggcacttctctgtttgatgaagagggagccaagattgtcaaagacctaatgtccaaagctgagaagaatggtgtgaagattaccttgcctgttgactttgtcactgctgacaagtttgatgagaatgccaagactggccaagccactgtggcttctggcatacctgctggctggatgggcttggactgtggtcctgaaagcagcaagaagtatgctgaggctgtcactcgggctaagcagattgtgtggaatggtcctgtgggggtatttgaatgggaagcttttgcccggggaaccaaagctctcatggatgaggtggtgaaagccacttctaggggctgcatcaccatcataggtggtggagacactgccacttgctgtgccaaatggaacacggaggataaagtcagccatgtgagcactgggggtggtgccagtttggagctcctggaaggtaaagtccttcctggggtggatgctctcagcaatatttagtactttcctgccttttagttcctgtgcacagcccctaagtcaacttagcattttctgcatctccacttggcattagctaaaaccttccatgtcaagattcagctagtggccaagagatgcagtgccaggaacccttaaacagttgcacagcatctcagctcatcttcactgcaccctggatttgcatacattcttcaagatcccatttgaattttttagtgactaaaccattgtgcattctagagtgcatatatttatattttgcctgttaaaaagaaagtgagcagtgttagcttagttctcttttgatgtaggttattatgattagctttgtcactgtttcactactcagcatggaaacaagatgaaattccatttgtaggtagtgagacaaaattgatgatccattaagtaaacaataaaagtgtccattgaaaccgtgatttttttttttttcctgtcatactttgttaggaagggtgagaatagaatcttgaggaacggatcagatgtctatattgctgaatgcaagaagtggggcagcagcagtggagagatgggacaattagataaatgtccattctttatcaagggcctactttatggcagacattgtgctagtgcttttattctaacttttatttttatcagttacacatgatcataatttaaaaagtcaaggcttataacaaaaaagccccagcccattcctcccattcaagattcccactccccagaggtgaccactttcaactcttgagtttttcaggtatatacctccatgtttctaagtaatatgcttatattgttcacttcctttttttttattttttaaagaaatctatttcataccatggaggaaggctctgttccacatatatttccacttcttcattctctcggtatagttttgtcacaattatagattagatcaaaagtctacataactaatacagctgagctatgtagtatgctatgattaaatttacttatgtaaaaaaaaaaaaaaaaaaaa

SEQ ID NO: 328 - DNA, 2519 bp, Homo sapiens
gcgtgtcaggtggttgcggagctgaatcatattctaagaactcagccactcaggtatccaccatggtgctgggtcctgaacagaagatgtcagatgacagtgtttctggagatcatggggagtctgccagtcttggtaacatcaaccctgcctatagtaatccctctctttcacagtcccctggggactcagaggagtacttcgccacttactttaatgagaagatctccattcctgaggaggagtactcttgttttagctttcgtaaactctgggctttcaccggaccaggttttcttatgagcattgcctacctggatccaggaaatattgaatccgatttgcagtctggagcagtggctggatttaagttgctctggatccttctgttggccacccttgtggggctgctgctccagcggcttgcagctagactgggagtggttactgggctgcatcttgctgaagtatgtcaccgtcagtatcccaaggtcccacgagtcatcctgtggctgatggtggagttggctatcatcggctcagacatgcaagaagtcattggctcagccattgctatcaatcttctgtctgtaggaagaattcctctgtggggtggcgttctcatcaccattgcagatacttttgtatttctcttcttggacaaatatggcttgcggaagctagaagcattttttggctttctcatcactattatggccctcacatttggatatgagtatgttacagtgaaacccagccagagccaggtactcaagggcatgttcgtaccatcctgttcaggctgtcgcactccacagattgaacaggctgtgggcatcgtgggagctgtcatcatgccacacaacatgtacctgcattctgccttagtcaagtctagacaggtaaaccggaacaataagcaggaagttcgagaagccaataagtactttttcattgaatcctgcattgcactctttgtttccttcatcatcaatgtctttgttgtctcagtctttgctgaagcattttttgggaaaaccaacgagcaggtggttgaagtctgtacaaataccagcagtcctcatgctggcctctttcctaaagataactcgacactggctgtggacatctacaaagggggtgttgtgctgggatgttactttgggcctgctgcactctacatttgggcagtggggatcctggctgcaggacagagctccaccatgacaggaacctattctggccagtttgtcatggagggattcctgaacctaaagtggtcacgctttgcccgagtggttctgactcgctctattgccatcatccccactctgcttgttgctgtcttccaagatgtagagcatctaacagggatgaatgactttctgaatgttctacagagcttacagcttccctttgctctcatacccatcctcacatttacgagcttgcggccagtaatgagtgactttgccaatggactaggctggcggattgcaggaggaatcttggtccttatcatctgttccatcaatatgtactttgtagtggtttatgtccgggacctagggcatgtggcattatatgtggtggctgctgtggtcagcgtggcttatctgggctttgtgttctacttgggttggcaatgtttgattgcactgggcatgtccttcctggactgtgggcatacgtgccatctgggattgacagctcagcctgaactctatcttctgaacaccatggacgctgactcacttgtgtctagatgactgacagcctgagagactctataagaacatgtttttctaagccctttttgtgccaggtgtcccgttaacgtctctgttagttcaaaggtgagttttgttcagacgttttgaacaaaaggcaaagatttcctcatgggaagggtgttcaaaactgacagctataaatgtaggtcagagacccacccacctcataacagtcatacactcccagagttaaacgattggcctctgatggccacacacccctatgggcttgtgtcttggactctgggatttaaaagatatatatgtatatatatttatgtaaacctgagcatctcagtttgggtagagttttaaggttatatgaaactgatagaacttttgtattttttttttaaatattgttttgatatatcagatatttgttttgtctggatcacaagtgagaaagaaaagagaataatgctctttttcacaatgaactggtcaaattgactatcttgtaaagtgatactttactatgtcagtgaaatttcagtttgattttagtcaaccagattatatcctttgtatctactgatattaacacatcatattaacacatcatttcttaaaaaaacatttcttctgtttggcaatcataattaactctggttatcagcctattttgtaattattgtctttgtcgtcacttttcttgaattgttttctagtcattaaacagatatgaaggcaaaaaaaaaaaaaaaaaaaaaaaaaaaaaaaaaa

SEQ ID NO: 329 - DNA, 1623 bp, Homo sapiens
cccccagaccattatggcctccctgcagtttgtgatctctttgcctgtatgcagcttaaagttgataaagagaagtggttatatcgaactcctctacagatgtgaaggaatggacaagagttgagcagcctttctgctgattatcacacatcatgagctgagtgactgcagcttgccaaatctttgtgtttctgggtctgaccaattagcttagttcttctcctgcctaattttgaactagtaaagcaaagtgagtcatcagattatgagttactgtttaaaagaaaaatgctgtttattcatgctgaggtgattcagttccctccttcttacagaagtattttaattcaccccacactagaaatgcagcatctttgtggacgtctttttcacaagcctccaaggctccttagattgggtcgttactaaaagtacattaaaacactcttgtttatcgaagtatattgatgtattctaaagctagtaaacttccctaacgtttaattgccctacagatgcttctcttgctgtgggttttcttttgttagtggtctgaaataattattttcctgttctattaatacatagtgtattttgcacaaaaaaattaacctggtcaatagtgattaccaaaatatatattaataatcttggcaatttttgacattaattatgaaacattttagcccacgttagttctacattattcttcacttaaactcagctactgcaaattttgtctttctgtaaatgttattaaaatatccagtgagctctttagaaggactcagtattatttcaagactatttttgaggtaattctagccttttaaaatattctacagacctacggggcttaaaagaaccccagtaccgactaagcaaataggcaaaagacatgttggaaatgtagtatagtacttgaaacagtcactatcatagggataattggtgcatcctgtgtaaatggaagctgagcttgacacctggtgcttttaagtagggataaagtcatcctctcactgcaagcacagcatacctgtacctccaaaagtgacgttttagtgaacaggccgttttcaacacttgtgccttggggtgttcattgaagctttgtgaaaactactgatgttttctcagtctccttaaagttacgtccatgctttaaaatgtctgtgtaggagagaagtggggtttataatgttttctctaagatatctttgctgctttccagactttgaaactattaagcttcttaactgcctcttaccggaaatacttctggggaaacttcatggtcccaaaatgtcattgccatacagcttcactagagttctttgaaccacagctgaaaagagctttgtattattttttaattccctccccagatatcatttaggagtattatataaaggtggtgggcaaaaacaatgtaaggagcctttccagttatcttgagttgcagctctgtagtttcttgaggccaaacacactgtattttacaagtcaaaatataatttacattaatcactatgttaatgagtatgtaaaacattcttttgcattgatgaattttgtatctgcttccattaaaagcataacagccataaaaaaaaaaaaaaa

SEQ ID NO: 330 - DNA, 3379 bp, Homo sapiens
ctggcctgggcctgaagtgagtgagaggcacatgaagagaagtattcaagtatttatacagataggaatcaagataatcaacaatgtctgtcactgaggaagacctgtgccaccatatgaaagtagtagttcgtgtacgtccggaaaacactaaagaaaaagcagctggatttcataaagtggttcatgttgtggataaacatatcctagtttttgatcccaaacaagaagaagtcagttttttccatggaaagaaaactacaaatcaaaatgttataaagaaacaaaataaggatcttaaatttgtatttgatgctgtttttgatgaaacgtcaactcagtcagaagtttttgaacacactactaagccaattcttcgtagttttttgaatggatataattgcacagtacttgcctatggtgccactggtgctgggaagacccacactatgctaggatcagctgatgaacctggagtgatgtatctaacaatgttacacctttacaaatgcatggatgagattaaagaagagaaaatatgtagtactgcagtttcatatctggaggtatataatgaacagattcgtgatctcttagtaaattcagggccacttgctgtccgggaagatacccaaaaaggggtggtcgttcatggacttactttacaccagcccaaatcctcagaagaaattttacatttattggataatggaaacaaaaacaggacacaacatcccactgatatgaatgccacatcttctcgttctcatgctgttttccaaatttacttgcgacaacaagacaaaacagcaagtatcaatcaaaatgtccgtattgccaagatgtcactcattgacctggcaggatctgagcgagcaagtacttccggtgctaaggggacccgatttgtagaaggcacaaatattaatagatcacttttagctcttgggaatgtcatcaatgccttagcagattcaaagagaaagaatcagcatatcccttacagaaatagtaagcttactcgcttgttaaaggattctcttggaggaaactgtcaaactataatgatagctgctgttagtccttcctctgtattctacgatgacacatataacactcttaagtatgctaaccgggcaaaggacattaaatcttctttgaagagcaatgttcttaatgtcaataatcatataactcaatatgtaaagatctgtaatgagcagaaggcagagattttattgttaaaagaaaaactaaaagcctatgaagaacagaaagccttcactaatgaaaatgaccaagcaaagttaatgatttcaaaccctcaggaaaaagaaatcgaaaggtttcaagaaatcctgaactgcttgttccagaatcgagaagaaattagacaagaatatctgaagttggaaatgttacttaaagaaaatgaacttaaatcattctaccaacaacagtgccataaacaaatagaaatgatgtgttctgaagacaaagtagaaaaggccactggaaaacgagatcatagacttgcaatgttgaaaactcgtcgctcctacctggagaaaaggagggaggaggaattgaagcaatttgatgagaatactaattggctccatcgtgtcgaaaaagaaatgggactcttaagtcaaaacggtcatattccaaaggaactcaagaaagatcttcattgtcaccatttgcacctccagaacaaagatttgaaagcacaaattagacatatgatggatctagcttgtcttcaggaacagcaacacaggcagactgaagcagtattgaatgctttacttccaaccctaagaaaacaatattgcacattaaaagaagccggcctgtcaaatgctgcttttgaatctgacttcaaagagatcgaacatttggtagagaggaaaaaagtggtagtttgggctgaccaaactggcgaacaaccaaagcaaaacgatctacccgggatttctgttcttatgaccttttcacaacttggaccagttcagcctattccttgttgctcatcttcaggtggaactaatctggttaagattcctacagaaaaaagaactcggagaaaactaatgccatctcccttgaaaggacagcatactctaaagtctccaccatctcaaagtgtgcagctcaatgattctcttagcaaagaacttcagcctattgtatatacaccagaagactgtagaaaagcttttcaaaatccgtctacagtaaccttaatgaaaccatcatcatttactacaagttttcaggctatcagctcaaacataaacagtgataattgtctgaaaatgttgtgtgaagtagctatccctcataatagaagaaaagaatgtggacaggaggacttggactctacatttactatatgtgaagacatcaagagctcgaagtgtaaattacccgaacaagaatcactaccaaatgataacaaagacattttacaacggcttgatccttcttcattctcaactaagcattctatgcctgtaccaagcatggtgccatcctacatggcaatgactactgctgccaaaaggaaacggaaattaacaagttctacatcaaacagttcgttaactgcagacgtaaattctggatttgccaaacgtgttcgacaagataattcaagtgagaagcacttacaagaaaacaaaccaacaatggaacataaaagaaacatctgtaaaataaatccaagcatggttagaaaatttggaagaaatatttcaaaaggaaatctaagataaatcacttcaaaaccaagcaaaatgaagttgatcaaatctgcttttcaaagtttatccaataccctttcaaaaatatatttaaaatctttgaaagaagacccatcttaaagctaagtttacccaagtactttcagcaagcagaaaaatgaaactctttgttttcttcttttgtgttctaaaaaaataaaatttcaaaagaaaaggttgtcttttaagttttttaaatatttgttgccttttaaaatccctgagtgtaagttaccatggtggcagcttagttttactatgccacaacaagttgactaggacattttagtaaatggtagtgagttaaattatctttattattttttaaaaataagaatttagaagtggtaaaattatggcccaagatgtatttggttctctattatgttttgatacattattttaatcatatatatgactttccttttcaaaaatactttaatgtacaagtgtaaatatatgtgcccataaaatcattgtaaatattatttagtcatcacaaataaaatattgtcccttgctacttgatatattaaagatgtagattttaaagtgaaaaaaaaaaaaaaaaa

SEQ ID NO: 331 - DNA, 964 bp, Homo sapiens
tctcaaccctgtgagagtccatattgagattggcccagatggaagagtgacgggtgaagcagatgttgagtttgctactcatgaagaagctgtggcagctatgtccaaagacagggccaatatgcagcacagatatatagaactcttcttgaattcaacaacaggggccagcaatggggcgtatagcagccaggtgatgcaaggcatgggggtgtctgctgcccaggccacttacagtggcctggagagccagtcagtgagtggctgttacggggccggctacagtgggcagaacagcatgggtggctatgactagttttgttaggaacatttgagttacttcaatcattttcacaggcagccaacaagcaattaagagcagttataatagaggaagctgggggacccattttgcaccatgagtttgtgaaaaatctggattaaaaaattacctcttcagtgttttctcatgcaaaattttcttctagcatgtgataatgagtaaactaaaactattttcagcttttctcaattaacattttggtagtatacttcagagtgatgttatctaagtttaagtagtttaagtatgttaaatgtggatcttttacaccacatcacagtgaacacactggggagacgtgcttttttggaaaactcaaaggtgctagctccctgattcaaagaaatatttctcatgtttgttcattctagtttatattttcatttaaaatcctttaggttaagtttaagctttttaaaagttagttttgagaattgagacacaatactaatactgtaggaattggtgaggccttgacttaaaactttctttgtactgtgatttccttttgggtgtattttgctaagtgaaacttgttaaattttttgttaactaaatttttttcttaaaataaagactttttcacaaaaaaaaaaaaaaaaaaaaaaaaaaaaaaaaaaa

SEQ ID NO: 332 - DNA, 1937 bp, Homo sapiens
gcctcccgctcgccctgaacccagtgcctgcagccatggctcccggccagctcgccttatttagtgtctctgacaaaaccggccttgtggaatttgcaagaaacctgaccgctcttggtttgaatctggtcgcttccggagggactgcaaaagctctcagggatgctggtctggcagtcagagatgtctctgagttgacgggatttcctgaaatgttggggggacgtgtgaaaactttgcatcctgcagtccatgctggaatcctagctcgtaatattccagaagataatgctgacatggccagacttgatttcaatcttataagagttgttgcctgcaatctctatccctttgtaaagacagtggcttctccaggtgtaactgttgaggaggctgtggagcaaattgacattggtggagtaaccttactgagagctgcagccaaaaaccacgctcgagtgacagtggtgtgtgaaccagaggactatgtggtggtgtccacggagatgcagagctccgagagtaaggacacctccttggagactagacgccagttagccttgaaggcattcactcatacggcacaatatgatgaagcaatttcagattatttcaggaaacagtacagcaaaggcgtatctcagatgcccttgagatatggaatgaacccacatcagacccctgcccagctgtacacactgcagcccaagcttcccatcacagttctaaatggagcccctggatttataaacttgtgcgatgctttgaacgcctggcagctggtgaaggaactcaaggaggctttaggtattccagccgctgcctctttcaaacatgtcagcccagcaggtgctgctgttggaattccactcagtgaagatgaggccaaagtctgcatggtttatgatctctataaaaccctcacacccatctcagcggcatatgcaagagcaagaggggctgataggatgtcttcatttggtgattttgttgcattgtccgatgtttgtgatgtaccaactgcaaaaattatttccagagaagtatctgatggtataattgccccaggatatgaagaagaagccttgacaatactttccaaaaagaaaaatggaaactattgtgtccttcagatggaccaatcttacaaaccagatgaaaatgaagttcgaactctctttggtcttcatttaagccagaagagaaataatggtgtcgtcgacaagtcattatttagcaatgttgttaccaaaaataaagatttgccagagtctgccctccgagacctcatcgtagccaccattgctgtcaagtacactcagtctaactctgtgtgctacgccaagaacgggcaggttatcggcattggagcaggacagcagtctcgtatacactgcactcgccttgcaggagataaggcaaactattggtggcttagacaccatccacaagtgctttcgatgaagtttaaaacaggagtgaagagagcagaaatctccaatgccatcgatcaatatgtgactggaaccattggcgaggatgaagatttgataaagtggaaggcactgtttgaggaagtccctgagttactcactgaggcagagaagaaggaatgggttgagaaactgactgaagtttctatcagctctgatgccttcttccctttccgagataacgtagacagagctaaaaggagtggtgtggcgtacattgcggctccctccggttctgctgctgacaaagttgtgattgaggcctgcgacgaactgggaatcatcctcgctcatacgaaccttcggctcttccaccactgattttaccacacactgttttttggcttgcttatgtgtaggtgaacagtcacgcctgaaactttgaggataactttttaaaaaaataaaacagtatctcttaatcactggaaaaaaaaaaaaaaa

SEQ ID NO: 333 - DNA, 2029 bp, Homo sapiens
cggacgcgtgggctgaagtgtattgaaacaaaatgattatttcataatgtttgaattgcttccaaactgcatgttatttattctaaactctccctctgataggataccgagacctagagaagttaagaaaacaagccccagatcaatcacattgctcctgacagcacctaaccttctagattccaagtccaatggttttcctggtactatgatgctagtagatcttaaaaaataataatctagatctacactatccaatatggtagccactggccacatgtgattttagctcttgatatgtgtttaattcgaatttagatgtgccgtaagtgtaaaatgcacactgaatttcaaagacttagtatgaaaaaaaaaagtatctcaatttttattacatgaagtgataacctagaaacactggacgaagtaaatattataaaaattaattttacatatttccttttatttaatgtggctactagaaacttatgtatggctcacatatttctactgaacagcactgatatatttggcaatatacagggtatacattatatatatatatatatattctatgtaaatagttcattaaataattcctatagtaaaatatgagaaacaggaaactcatatccaacttggaaaagtttcaaatataatacttattaagaaatacaggttctattcttcaggtactcttacctagaatgaattaaggatatgtagaaactcttatgttacttataaaataataaagagatactgtaggtctctacagagatgctggagtgtccctatagtacctctagagtatagtatagagatactataggatctctttactatatagatatatggtatagactatagaggtatctctagactatatattatttatactctactatcctatatagtatctctatatagagagataatatatatactccatatagtatactctagagatgctataagtttctgtagtatctttatagtagagataatagggagggataatagtatagggacgtactatatacctatagtatctctataagtacacaagttcccccattaatcatctgttttcagttgttaaccaaataaggtattatgcaggctcatctttttatataagaagaaaagactgaatgctccctatgacttcattttctccctgttcccactaccatgtccccaaacatactgacacacaaagaaaatataaatacccctgattcttttagccttaaacttagggcctcatcctaaacatcttaagctcagcatctattccaaaggaactctttaatgataacgtttctttgtaaagagacgaatatcatctggggtttcctctgaaatgtagatgaacctcacttcagttctttgtgtactaaaaagacccacccttctcacccatgtggcttttgtctttccttgtgttatgtaataagtgatggggtagaaggatggggggtggaggaagctacatcatggagagaggaggtagaagacagctgtggaaaatttggatggtggaagactagaatctactaccatttacatgctaatgaaaaccagaaatggtcttttttcccccgtttgatctgatttgaaagtttgtgacttttgtctagtatgtcattttaccccctgtgtttttctcattgttgttagctataaaaactctttgaaattctcaaaataaaggaaggtttctttcaggaataggagggctgaggtcaagctgtgaaactgcattcttcatagtgggtctattggggtggaaaacccgcctttgaagtaatagaaggtagaagcccggaaggaaacaggtctggccctggatccaggaaaataaagaaagaaataattggagatgtagcttagtacttagaattatcccagaagtgaacaacatcagtaaggtctgggcttccccctaacgtaacctcttatgagggtttcaacctcacatccatatatcttttaaaaaaaaaaaaaaaaaaaaaaaaaaaaaaaa

SEQ ID NO: 334 - DNA, 2923 bp, Homo sapiens
ccagcctggacgacagagcagactccatctcaaaaaaaaaaaaaaaatggtcaccccttttgctcctaaatcaccctcaaagtaaaagagaacaagaaacagaagcagaaatccatatttagtgaaataagacaacacctgtagctccaaacctgtagaagcagatcccagagaaaagcaggccagttctctcctggaatcccagaaagtcccaggaattggaggcttcagtgctgcagtagggaggggactaaaaacaaagtctgtatatggagcagtaagacctccgggttctcatcccccatcctgtgtgctctcgggtgactagccctctccctctccaggtttagcttctggagaaattaaatcaaagaggctctagaactggggatggcaggcattgagtgcagggagtgagttgccagaggctgagagcagagagatttagtggcagtgggcagagcaaacagcaaaatgactgccctcttccctggccttgctccagaaactgagcagccagacatacacactcccagaaggcagttggaggtccccccgggcaatcagaaccacccacagagaagacctccagatactgacatttagaagcctctgaccaaagagctgcctggccacccactggcttactcgctgacaggccatgctcctgccgtacacaccgatcctatcagtcatcctcatgggctcctttccagacttctgaggaaagcttccaacatgaagagggagttcaaaacaaacagagagagagagagagagagagagagagagagagagagagagagaaaagaaatataaacaatgcagggagcaaaagataacttcaaataaactcaaaatattcttctgggagagatgagacaatattgcatacataaaacaagaagaggatgctatcactaaattttaaaaaaaagtaacagaaagtaagaaagacctctaagaaatgtttctgaaatgaggtataatttttttttttttgagggggagtcttgctctgtcacccaggctggagcgcagtggcgtgatctcggctcactgcaacctccgtctcccgggttcaagtgattctcctgcctcagcttcctgagtagctgggattacaggcatgcgccaccatgcctggctaattttgcatttttagtagagacggggtttcaccatgttggccaggctggtctgaaactcccgacctcaggtgatctgcccacctcagcctcccaaagtgctgggattacaggcatgagctaccgtgcctggcctgtgtatttttggagacagagtcttgctctgttgcccaggctggagggcagtggtgcaaactctgcctcccgggttcaagcgattctcgtgcgtcacctcccaagtagctggggctagaggcatgtgccaccatgtctggttactttttatataaaccttcagtttaatcttttagcttactgctgtgtttttcagctttattcactttgctttttttctatattttcgtttcaactcaacttttaaaaaatcatacttgtatatttgtatatttatatttattttccaagacctcatttgcctctttttcataatgggctattattgctccatggtacagaatcttctgttctttcctaaactattagttatttttaaaatatttgttaactttctcccccttgctagttctcaggtacccgctttctcccagagtgctggctttccttaattatttgctgacttgtgctggtgtgtaacccttcatacttaggtatttctatttgtctgactgctgatttgattccaatccagtgtttcttctgattcgtggagaagagacgacaacgctgtgaggctctggttttggtggcttgtctgggtgtgggagctccttgtcatcatgggatttttagctccctgggtcgccatcctacatggccactctcctgccgatgctgcccgctactcagcaggaggggaagtcgagaccacctccttaaccttacagacattgattgtgagcttggagcaccttccgtgactggaccacccatgcagtggtcctttgctttttgcatttttaactgcaattttccccaagagtcttccctaatacagtctcttaggaattgacggtgggattaaacaaggcatgtttctgcactgaatatggtctatgggttgccactttgcaacctcagctttaaggcttatttcctcccaggaaactgatgttaactttttaagttaaaaatgtgtatataagtaatacatgtttattttggaaattagaaaatacaggccaggtgtggtggctcatgcctgtaatcccagcattttgggaggttgaggcgggaggattgcctgggcccgggagtttgagaccagcctgggcaacatagggaaattccatctctacaaaaaaattaaacattggctgggtgtggtggtgcccacctgtggtcacagctactcgggagactgaggtgggaggatagcttgggcaggggaggttgaggctacagtgagccacgatcacaccaatgtgacacagtgagaccctgtctcaaaaaaaaaaaaaaagaaagaaaatataggtaaacacaaagacaaaaaagcagggcatggtggctcacacctgtaattctagcaccttgggaggctgaggcaggaggatcacttgaggtcaggaactcgagacgagaccaggctgggcagcatggcaggaccccatctctataaaaagtacaaaaattagcagggcacggtggtgtgcacctgtggtcctgctacttgggaagttgaggtgggaggatcacctgagccctggaggttgagggtgctgtgagccatgatggcaccactccactccagtccaggtgaaagagccagatcctgtctcaaaaaaaaaaaaaaa

SEQ ID NO: 335 - DNA, 2283 bp, Homo sapiens
cgcctccaggccccttcccgcgccgcgacgcacgctgccccggaaggccgcggcgctgtagtgcggcgccccaggttctttagtggaagaacgcgaagcgaggatgagtgatccgtggaggcagtaacaggcgcggcgagggagaagtgattcccgaagaatcaaggctgggccggacccggtggcctggcaacaggtaataagagaaatgaagccaacaggtacagacccaaggatcttatctatagctgctgaagttgcaaaaagccctgagcagaatgtccctgttatactgttgaagttaaaagaaataataaacatcacacctttaggaagctcagagttgaagaaaatcaaacaagatatatattgttatgatctcattcaatattgcctcttggtcctcagtcaagattattctcgaatccagggtggttggactacaatttcccagcttacacagatattaagccattgctgtgtgggcttggagccaggagaagatgcagaggaattttacaatgaattacttccatcagctgcagaaaattttctagttttggggagacaattacaaacatgttttatcaatgcagctaaggctgaagaaaaagatgaattactacactttttccaaattgtgactgattctctcttctggcttttgggaggccatgttgaacttattcagaatgtactacaaagtgatcatttcttacatttactgcaagctgacaatgtccaaataggatctgcagtcatgatgatgctacagaatatattacagatcaacagtggtgatttactcagaataggaagaaaagccctgtattcaattttagatgaagttattttcaagcttttttcaactcctagtccagttataagaagtactgctacaaaactcctactgttgatggctgaatcccatcaggaaattttgattttactgagacaaagtacctgctacaaaggactcagacgtctactaagtaaacaggaaactgggactgaattcagtcaagaacttagacagcttgttggccttttaagcccaatggtctatcaggaagtagaagagcagatccaaacgatcaaagatgttgctggagataaataggcagaaggaagaagaggacctcaaattacaattgcaacttcaaagacagagagccatgagactttcccgagaattgcagctgagtatgctcgaaatagttcatccaggtcaggtggagaaacactatcgggaaatggaagagaaatcagcactgattatccagaaacattggagagggtacagggaaaggaaaaattttcaccaacagaggcagtctctcatagagtataaagcagctgtcacacttcaaagagcagcgcttaaattcctagcgaagtaccgtaagaaaaagaaactatttgctccttggcgaggactccaagaactcactgatgcacgccgagttgaactgaagaaacgagtggatgactatgtcagaagacatttgggctctccaatgtcagatgtggtcagtagggagctccatgcccaagctcaagaacgactgcaacactactttatgggcagggccctagaagagcgagcccagcagcacagagaagctctgatagcacagatcagcaccaacgttgaacagctaatgaaggcaccaagtctgaaggaggcagaagggaaagaacctgagctcttcctaagtagatccaggcctgtggcagccaaggccaagcaggcccatctcacaaccctgaagcacatacaagcaccctggtggaagaagcttggagaagaatctggagatgagattgatgttccaaaggatgagcttagtatagaattagaaaatttattcattggtggaaccaaaccaccttagtgagtaaccctaagaattgacacaaatctcatattttaggagattatattggttctgcctctggcatgctggtagactagggccatcctaacttattattttccagaggttctcctccagacaagacctgcagtaagcaaagagttatattctacctctctctcaattttctttttcttttctctgtatcctcatccttagccacacacagatttgtgtggcttttattgtagaactaaacttagcatagtgttctgttgtttacatgaagtgtgtttttctttggtttcttctgttttccaactaaatatttttttctaaataaatattttcaacaattgatttgaaaaaaaaaaaaaaaaaaaaaaaaaaaaaaaaaaaaaaaaaa

SEQ ID NO: 336 - DNA, 2765 bp, Homo sapiens
gggagacgtggtgccgctgcgggctcgctctgccgtgcgctaggcttggtgggaaggcctgttctcgagtccgcgcttttcgtcaccgccatgtcgggaggtggtgtgattcgtggccccgcagggaacaacgattgccgcatctacgtgggtaacttacctccagacatccgaaccaaggacattgaggacgtgttctacaaatacggcgctatccgcgacatcgacctcaagaatcgccgcgggggaccgcccttcgccttcgttgagttcgaggacccgcgagacgcggaagacgcggtgtatggtcgcgacggctatgattacgatgggtaccgtctgcgggtggagtttcctcgaagcggccgtggaacaggccgaggcggcggcgggggtggaggtggccggagctccccgaggtcgctatggcccccatccaggcggtctgaaaacagagtggttgtctctggactgcctccaagtggaagttggcaggatttaaaggatcacatgcgtgaagcaggtgatgtatgttatgctgatgtttaccgagatggcactggtgtcgtggagtttgtacggaaagaagatatgacctatgcagttcgaaaactggataacactaagtttagatctcatgagggagaaactgcctacatccgggttaaagttgatgggcccagaagtccaagttatggaagatctcgatctcgaagccgtagtcgtagcagaagccgtagcagaagcaacagcaggagtcgcagttactccccaaggagaagcagaggatcaccacgctattctccccgtcatagcagatctcgctctcgtacataagatgattggtgacactttttgtagaacccatgttgtatacagttttcctttattcagtacaatcttttcattttttaattcaaactgttttgttcagaatgggctaaagtgttgaattgcattcttgtaatatccccttgctcctaacatctacattcccttcgtgtctttgataaattgtattttaagtgatgtcatagacaggattgtttaaatttagttaactccatactcttcagactgtgatattgtgtaaatgtctatctgccctggtttgtgtgaactgggatgttgggggtgtttgtggttatcttacctggggaagttcttatgtttatcttgcttttcatgtgtctttctgtagacatatctgaagagatggattaagaatgctttggattaaggattgtggagcacatttcaatcattttaggattgtcaaaaggaggattgaggaggatcagatcaataatggaggcaatggtatgactccaagtgctattgtcacagatgaaattggcagtattgaccttatactaaaaggcaggggttaaaaatgattatatacattttccttaaaacacttgcaaacattttattcagttgtctttagctacaattgctttgctttttaaaccttggcaattgtggcaaaattatattgcccattttgtagcaacttattttgctcccttccccccatttttgttttaatagggactaatgtgggaagaactggctaatttgtcacagtgcttagttacaactgttaatgtgtgacctgctgttggtgtacatgtgggtacagggtgtttttaaatccaacaagatagagtataatatcaatactgctaaatctgcatgtcctctgtgtgactgatagagcgttgctatttcatttttttaagacaaaatgaaagcaaaatatagagttccaatgtattggtgtagataatctagttgggaatacttttaagtctcaccttcccctttaaactaatattcataattggttcatatgtttaaaagactttaatttacaaattaaattgcaaatgggagcattagatttagttttagacttaggtgggtagcaatgccagtaaacttaaattacgtaacttcttgcaaccacgaaacctgtaatacgctgtacagtaacaagtgttggcattatcagttgaactgtaaatacaaaatgcttcttccaattagtctctatgatgattaagtttctaaaatttatctgaacaccattcagaaacttgttttggggaatttgatagttattgatgtgcatctgttaaactgatgacagacataactcatcattccccagaaaccttttttgattacagtatctaacattttgcctcctcttttttggttttgctggttataaaggtttggattggagagggctcactggatcccaatccttggagctggatcattggattcaaatcataatgtggataggatagggaggatgaattacccaggattcatggagcgggatcagattaccaggaacataggagtggattcctgccccaaccaaaccgcattcgtgtggatttttttattcaacttaattggctattccaaagattttttttttcctatttttgacgattggagcccttaagatgcacgatggaattgtgttttgcgttttttggtaaaaggagcaaagcgaggacctggagataaacgctggagcaatctccttggaaggattcagcacgagtagatggtaaacatttaaaggggaaagggggggtttgtttaaaatagtaaatcagtaagtcacttctaaatttaaagaaaacaaaattggagttgaagaataagtaggtttccaattggctattgccgttttctttgaaaaaataaacattttttaaaaaactaaaaaaaaaaaaaaa

SEQ ID NO: 337 - DNA, 1567 bp, Homo sapiens
ggggtcctgccaccgcgccacttggcctgcctccgtcccgccgcgccacttcgcctgcctccgtcccccgcccgccgcgccatgcctgtggccggctcggagctgccgcgccggcccttgccccccgccgcacaggagcgggacgccgagccgcgtccgccgcacggggagctgcagtacctggggcagatccaacacatcctccgctgcggcgtcaggaaggacgaccgcacgggcaccggcaccctgtcggtattcggcatgcaggcgcgctacagcctgagagatgaattccctctgctgacaaccaaacgtgtgttctggaagggtgttttggaggagttgctgtggtttatcaagggatccacaaatgctaaagagctgtcttccaagggagtgaaaatctgggatgccaatggatcccgagactttttggacagcctgggattctccaccagagaagaaggggacttgggcccagtttatggcttccagtggaggcattttggggcagaatacagagatatggaatcagattattcaggacagggagttgaccaactgcaaagagtgattgacaccatcaaaaccaaccctgacgacagaagaatcatcatgtgcgcttggaatccaagagatcttcctctgatggcgctgcctccatgccatgccctctgccagttctatgtggtgaacagtgagctgtcctgccagctgtaccagagatcgggagacatgggcctcggtgtgcctttcaacatcgccagctacgccctgctcacgtacatgattgcgcacatcacgggcctgaagccaggtgactttatacacactttgggagatgcacatatttacctgaatcacatcgagccactgaaaattcagcttcagcgagaacccagacctttcccaaagctcaggattcttcgaaaagttgagaaaattgatgacttcaaagctgaagactttcagattgaagggtacaatccgcatccaactattaaaatggaaatggctgtttagggtgctttcaaaggagctcgaaggatattgtcagtctttaggggttgggctggatgccgaggtaaaagttctttttgctctaaaagaaaaaggaactaggtcaaaaatctgtccgtgacctatcagttattaatttttaaggatgttgccactggcaaatgtaactgtgccagttctttccataataaaaggctttgagttaactccctgagggtatctgacaatgctgaggttatgaacaaagtgaggagaatgaaatgtatgtgctcttagcaaaaacatgtatgtgcatttcaatcccacgtacttataaagaaggttggtgaatttcccaagctatttttggaatatttttagaatattttaagaatttcccaagctattccctcaaatttgagggagctgagtaaccccatcgatcatgatgtagagtgtggttatgaactttaaagttatagttgttttatatgttgctataataaagaagtgttttgcattcgtcaaaaaaaaaaaaaaaaaaaaaaaaaaaaaaaaaaaaaaaaaa

SEQ ID NO: 338 - DNA, 2224 bp, Homo sapiens
caactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaagctcatcatactggctagtggtggaccccaagctttagtaaatataatgaggacctatacttacgaaaaactactgtggaccacaagcagagtgctgaaggtgctatctgtctgctctagtaataagccggctattgtagaagctggtggaatgcaagctttaggacttcacctgacagatccaagtcaacgtcttgttcagaactgtctttggactctcaggaatctttcagatgctgcaactaaacaggaagggatggaaggtctccttgggactcttgttcagcttctgggttcagatgatataaatgtggtcacctgtgcagctggaattctttctaacctcacttgcaataattataagaacaagatgatggtctgccaagtgggtggtatagaggctcttgtgcgtactgtccttcgggctggtgacagggaagacatcactgagcctgccatctgtgctcttcgtcatctgaccagccgacaccaagaagcagagatggcccagaatgcagttcgccttcactatggactaccagttgtggttaagctcttacacccaccatcccactggcctctgataaaggctactgttggattgattcgaaatcttgccctttgtcccgcaaatcatgcacctttgcgtgagcagggtgccattccacgactagttcagttgcttgttcgtgcacatcaggatacccagcgccgtacgtccatgggtgggacacagcagcaatttgtggagggggtccgcatggaagaaatagttgaaggttgtaccggagcccttcacatcctagctcgggatgttcacaaccgaattgttatcagaggactaaataccattccattgtttgtgcagctgctttattctcccattgaaaacatccaaagagtagctgcaggggtcctctgtgaacttgctcaggacaaggaagctgcagaagctattgaagctgagggagccacagctcctctgacagagttacttcactctaggaatgaaggtgtggcgacatatgcagctgctgttttgttccgaatgtctgaggacaagccacaagattacaagaaacggctttcagttgagctgaccagctctctcttcagaacagagccaatggcttggaatgagactgctgatcttggacttgatattggtgcccagggagaaccccttggatatcgccaggatgatcctagctatcgttcttttcactctggtggatatggccaggatgccttgggtatggaccccatgatggaacatgagatgggtggccaccaccctggtgctgactatccagttgatgggctgccagatctggggcatgcccaggacctcatggatgggctgcctccaggtgacagcaatcagctggcctggtttgatactgacctgtaaatcatcctttaggtaagaagttttaaaaagccagtttgggtaaaatacttttactctgcctacagaacaaagacttggttggtagggtgggagtggtttaggctatttgtaaatctgccacaaaaacaggtatatactttgaaaggagatgtcttggaacattggaatgttctcagatttctggttgttatgtgatcatgtgtggaagttattaactttaatgttttttgccacagcttttgcaacttaatactcaaatgagtaacatttgctgttttaaacattaatagcagcctttctctctttatacagctgtattgtctgaacttgcattgtgattggcctgtagagttgctgagagggctcgaggggtgggctggtatctcagaaagtgcctgacacactaaccaagctgagtttcctatgggaacaattgaagtaaactttttgttctggtcccttttggtcgaggagtaacaatacaaatggattttgggagtgactcaagaagtgaagaatgcacaagaatggatcacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 339 - DNA, 854 bp, Homo sapiens
gggacacagccggccaggagaaattcggtggactgagagatggctattatatccaagcccagtgtgccatcataatgtttgatgtaacatcgagagttacttacaagaatgtgcctaactggcatagagatctggtacgagtgtgtgaaaacatccccattgtgttgtgtggcaacaaagtggatattaaggacaggaaagtgaaggcgaaatccattgtcttccaccgaaagaagaatcttcagtactacgacatttctgccaaaagtaactacaactttgaaaagcccttcctctggcttgctaggaagctcattggagaccctaacttggaatttgttgccatgcctgctctcgccccaccagaagttgtcatggacccagctttggcagcacagtatgagcacgacttagaggttgctcagacaactgctctcccggatgaggatgatgacctgtgagaatgaagctggagcccagcgtcagaagtctagttttataggcagctgtcctgtgatgtcagcggtgcagcgtgtgtgccacctcattattatctagctaagcggaacatgtgcttcatctgtgggatgctgaaggagatgagtgggctttcggagtgaatgtggcagtttaaaaaataacttcattgtttggacctgcatatttagctgttttggaacgcagttgattccttgagtttcatatataagactgctgcagtcacatcccaatattcagtggtgaaatcttgtttgttactgtcattcccattccttttcgtttagaatcagaataaagttgtatttcaaatatttaaaaaaaaaaaaaaaaaaaaaaaaaaaaaaaaaaaaaaa

SEQ ID NO: 340 - DNA, 1816 bp, Homo sapiens
gaaagatggaggaccatcagcacgtgcccatcgacatccagaccagcaagctgctcgattggctggtggacagaaggcactgcagcctgaaatggcagagtctggtgctgacgatccgcgagaagatcaatgctgccatccaggacatgccagagagcgaagagatcgcccagctgctgtctgggtcctacattcactactttcactgcctaagaatcctggaccttctcaaaggcacagaggcctccacgaagaatatttttggccgatactcttcacagcggatgaaggattggcaggagattatagctctgtatgagaaggacaacacctacttagtggaactctctagcctcctggttcggaatgtcaactatgagatcccctcactgaagaagcagattgccaagtgccagcagctgcagcaagaatacagccgcaaggaggaggagtgccaggcaggggctgccgagatgcgggagcagttctaccactcctgcaagcagtatggcatcacgggcgaaaatgtccgaggagaactgctggccctggtgaaggacctgccgagtcagctggctgagattggggcagcggctcagcagtccctgggggaagccattgacgtgtaccaggcgtctgtggggtttgtgtgtgagagccccacagagcaggtgttgccaatgctgcggttcgtgcagaagcggggaaactcaacggtgtacgagtggaggacagggacagagccctctgtggtggaacgaccccacctcgaggagcttcctgagcaggtggcagaagatgcgattgactggggcgactttggggtagaggcagtgtctgaggggactgactctggcatctctgccgaggctgctggaatcgactggggcatcttcccggaatcagattcaaaggatcctggaggtgatgggatagactggggagacgatgctgttgctttgcagatcacagtgctggaagcaggaacccaggctccagaaggtgttgccaggggcccagatgccctgacactgcttgaatacactgagacccggaatcagttccttgatgagctcatggagcttgagatcttcttagcccagagagcagtggagttgagtgaggaggcagatgtcctgtctgtgagccagttccagctggctccagccatcctgcagggccagaccaaagagaagatggttaccatggtgtcagtgctggaggatctgattggcaagcttaccagtcttcagctgcaacacctgtttatgatcctggcctcaccaaggtatgtggaccgagtgactgaattcctccagcaaaagctgaagcagtcccagctgctggctttgaagaaagagctgatggtgcagaagcagcaggaggcacttgaggagcaggcggctctggagcctaagctggacctgctactggagaagaccaaggagctgcagaagctgattgaagctgacatctccaagaggtacagcgggcgccctgtgaacctgatgggaacctctctgtgacaccctccgtgttcttgcctgcccatcttctccgcttttgggatgaagatgatagccagggctgttgttttggggcccttcaaggcaaaagaccaggctgactggaagatggaaagccacaggaaggaagcggcacctgatggtgatcttggcactctccatgttctctacaagaagctgtggtgattggccctgtggtctatcaggcgaaaaccacagattctccttctagttagtatagcggacttaataaaagaggaaaaaactcttgcttcaaaaaaaaaaaaaaa

SEQ ID NO: 341 - DNA, 696 bp, Homo sapiens
gaaaaaacaaagagagacattaacaggtttctgagtgtggccagtcttcaaggacttattcatgaaggcaccatgacttctttgtgcatggccatgacagaggagcagcataagtctgtggtcatcgattgcagcagctcccagcctcagttctgcaatgcaggaagtaaccggttttgtgaggattggatgcaagcttttttaaatggtgccaaaggaggtaacccttttcttttccgacaagtactggagaactttaaactaaaggccatacaagacacaaacaatttgaagagatttatccgacaggcagaaatgaatcattatgctttgtttaaatgttacatgttcctaaagaactgtggtagtggagatatacttttgaagattgttaaagtggaacatgaagaaatgcctgaagccaaaaatgtgatagctgtccttgaagaattcatgaaagaagctcttgaccaaagtttttgatcatatgttttgagataattgtatgatcaagttgtatatttaagtcttagtgtttgaaattgcagttataattgttcataggattgctatttaagatgatttgaaactcaatccagattttctttttgtattttaccaatttaacttaaataaaaatctgaagaacaaaaaaaaaaaaaaaaaaaaaaaaaaaaaaaaaaaaaaaaaaaaaaa

SEQ ID NO: 342 - DNA, 4912 bp, Homo sapiens
cttttttgtttttgagactccatcccaggcaggagtgcagtggcacgatcttggctcactgcagcctctgcctcccaggttcaagcgactctcctgccccagtgacccaagtagctgggattacaggcatgcgcccactaatttttgtatttattgtagagacagggtttcatcatgttgcccaggctgctctcaaactcctgatccaccctcaagcaatccaccttccttggcctcccaaagtgctgggattacaggcatgagccaccctgcgtccggcccaaaatttagtgacttaaaacaagtatttattatctcacagtttctgctggccaggagttcagaagcagcttagacatttctcacaaagttgctgtcatctgaaggcttggctggagctggaggatgctcttccaagctcactcagattggggaccacaaattcagtgcccaccggattgtcttagcagcctcgatcccgtatttccatgctatgtttacaaatgacatgatggagtgcaagcaggatgagattgtaatgcaaggaatggacccaagtgccctggaggctctgatcaactttgcctacaacggcaaccttgccattgaccagcaaaatgtccagtcattgctgatgggggcgagcttcctgcagctgcagagcatcaaagacgcctgctgcacattccttcgagaacggcttcacccaaaaaactgcctgggtgtgcgccagtttgctgagacaatgatgtgtgctgtgctgtacgacgctgccaacagcttcatccaccagcactttgtggaggtgtccatgtcagaagagttcctggccctgcccttggaagacgtgcttgagctggtgtctcgggatgagctgaatgtcaaatctgaggagcaggtctttgaagctgcattggcctgggtcagatacgaccgggagcagaggggtccctacctgcctgagctgctgtccaatatccgcctgcccctctgtcggccccagttcctttcagacagagtacagcaggatgacctggtgcgttgctgccacaaatgcagggacctggtagacgaagcaaaggactaccacctcatgccagagcgccggccccacctgccagctttcagaacccggccacgctgctgcacatccatcgctggacttatctacgctgtagggggcctcaactcagcaggtgattccctgaatgtggtggaagtgttcgaccccattgccaattgctgggagagatgccgtcccatgacaacagcccgcagccgcgttggcgtggctgtggtgaacgggcttctctatgccatcggaggatatgacggccagctacggctgagcactgtggaggcctacaacccggagacagacacatggaccagagtggggagcatgaatagcaagagaagtgccatggggacagtcgtgctggatgggcagatctacgtctgtgggggctacgatggcaactcttccctcagctccgtggagacctactcacctgagacggacaaatggacagtggtgacctcgatgagctcgaatcgcagtgctgctggggttacagtctttgagggcaggatatatgtgtcaggcggccatgatggtttgcagatcttcagcagtgtggaacactacaaccaccacacagccacctggcaccctgcagctggcatgctcaacaagcgctgccggcacggagccgcctccctggggagcaagatgtttgtctgcgggggctacgatggctctggcttcctcagcattgccgagatgtacagctctgtggcagaccagtggtgcctgattgtccccatgcacacgcgcaggagccgggtctccctggtggccagctgtgggcgcctctacgctgttgggggctacgacggacagtcaaacctaagctcagtggagatgtatgacccagagacagactgctggacattcatggcccccatggcgtgccatgagggaggggtcggtgtgggctgcatccctctcctcaccatctaaggcagaggatgggatgtggtggggcagggatctggtacagacataggcgcttccttccaggaacagtccctcaggagaggcagtggaccagaagagatggcgaaacgtgagctcgccggaggtacagtttttccaggtgcttaagccctccccaactgtgccacccttgtgaccttcaggcttgggtcatcaagatgcacagcatggaacacaagctcctctggatcctgcagctggtgacatggaactgttttctggtccacatgaacacaggctccatccaggcccagctcctacccaccgcctctctgtgggccagctgttcacagaaggccttccatctgatgctccccatcgcctgcttgctctccagccgagtctggccaatttgccatggggaggctgcagtgtccaagcctgctggaaactgggatgtagctggggacgaaaggacagacccaagcgttctccctgcctgagatggtgtggccacagcagtggaaggctgcacacaggcacattccttcttccacagtggggcaccaaggattctgtcctcattgctgggtaagcagggagaagagaagttttccccatgtctaattttgggatttcagtgaggccttttccatctgtccaggagaacagaagggaaaaaaagatacttgaaagaaactgaaggaaatttaaacaaagaaacacttgaaagaaactggaaagaaaaataatttttttatgtgaacaaattttgcaagaagaaaaaagcataaaagacactaacggcaaatctatgtttaaatggaaaatcgtctaactggagaagggcggtatccaccccacattcggatcccagggtcctgaggcctcgcattgagctgggggttccctctgagccccagtgtgtgtggaatcagtgcactcttgactgggcctgtagtaaggtgctcatggggtttgtcttctcacccaccatcagaggacttttaaaatcataggcgtagagagttagctatctgctgaattactgccactcttcttggtgggggctcctagctgtggctgggggctccaggcgcccctgtgattacctcctactgccaccatggcgctcattcagattccccactctcactaacattgcttccttttttgaccagcaggaaacagcaggtctggccagattctcacttgcccatcaatctcgttcttggatgatttccctcattgtgatgcttctggggcacgttgaccatatgcacctctagaacctaaccagggcttccttctaccagctgtgggcgggcttggtctggtaaccttgtctgctctgccattccactgctcctccatccactcgccaatcccaagagtctggcctccctccagccctgggcagactgaccagcaaggtggacctttacattcaagcacagctggcttttatgacataaagaactaaaggccgaaagaatctcttgctgctgcaaagaacagattttatatttcttcctctaatcttggcaaatgacctttaccttttggaaagatttcatattgcttcctcctccctggataggacctaatgtagcacagcgggactcaaagaggaggacattttctcttgccagtgcactgggcagtggggctgtccttcaactgctgctgccaaaattggttttctaaaattcttccagtagagactaaaagaagattcaattcctgtaacccaagactgagtcttagggctccagtctccacctgcttggtttcctatcctttgctgcctgcctggggtggcctggaagcctgttcagaaaggcacaatgtggagcctggggtgtctcccccaccccaggaccgtcaggtttaccagtgtgtgcaatcgccatgtattcagagggaagtacctttgttacctacaacttaggagctaggcctctgctacaagcacttgaaaatgatatttttatttttaacgtctcaacaatctgatatcggatgtcgtttaacctgggctcgtggtagggctccagcatttctccctccttcctggtttgcctgtaggggtagactcggaaggtgggtggggtgtgcatttcctgttaggagtgtatcagtgcttgtcttattataagcccctttcttttgtgaatttgaagtagcaccaacaagcctggattgtgaaggtattaagaatcggtctgtgggctactgagtgggtccttaggatactggcccagattttgccactgggtatggcagatcattttctaccatggcctgctgctcttgtagtggacttcctgagtccaatcccacctcctggtgtagaatttacactgctgcacctgaggtcgatgtttcaaagtaagatcaagccagtgttttgatctgggctctgagcacaagtcaggaaacaccaacatattcacactctcccagtaggttcctcagtccgatggtgaatggctattcgtaaatggctggtctggctctttggtgttggagcctttccaatagccccatgaaaagaagcatcacccaaggatattgtaaaaaggatgtaacaaggagatagggtagacattgtactcagtgggccttggggcctagcccagctctgagcagaggactgtggcattcactgtccttgagtgtttcaccttcttggataacacacgggccttctcttctggatttcatcagagattacagccagatgggggctgaagaccatcctcttgaccacagaggtgtgactgtgggaattcctcccaatttatggtttcccagaaaatcttagttccttttatttatagaatgcatgtcttttgtgttaagaaaccaaagagaaataaagagaacactcctaataaaaaaaaaaaaaaa

SEQ ID NO: 343 - DNA, 2731 bp, Homo sapiens
  misc_feature (2578)..(2578): n at position 2578 is a or g or c or t
gacacaagaccaagtttgaaacagatgcaaataaactaaaatacagctctaatgtctttttctgctattctgtctcccttttcctctttgtctgtaaatgtaaggaatttgagacaaagaggaaaagggagacagaatagcagaatcttgactttgatagtcaagatcttgttcaagacttggcacttgatctttttgtaaacctagtattatgttatggtcaagtgggaagcatgaatttaaatgtagtatttaaaagattacttggtcttggctttccaatctgtgggagtgtttggatcattttatacacacacacacacacacacacacacacacacacctttttttttttttgatatgaggtcttaccctgtcacccagactagagtgcagtggcgcaatcttagctcactgcaacctctgcctacctggctcaagggatcctcccacctcagcctccagagtagctgggactacaggcacatgccaccaagcccagctaatttttcatatttttggtagagatggggtttcactctgttgcccaggctggtctcaaactcctgagctcaagtgatccacccaccttggccttccaaagtgctgggattacaggtgtgagctactgcacctggtacattattataatttaataatagtgacaggaaaaggtaaaaattgcaagcaacaataataaccaataatgcagaaatacatataaaatgttaactgtaaaggcagatattgttgcctatatcagccaatgtagatcttctggttttatacttactggtatactataaatgaaatttagtgacagcatactgtataccaaactagagtttacccattgttatttcttatttagatttttagatacattgtcttttgattctaacattggcattatcttaatttaatctctgtatatttttcttctttttggaaatatgcgtttgtaaaagcttaccttataagctactatggtgaccttggcctatcagatgggtgttctcttaaaccgttttatactgctttataccagactcaaaattgccaacatagttatctgctaattctcaataacttaaagccacattatttttgctagagtaattagtactacattgtttatttctagatgcactttaatattcatgtcaccagagtaatttagttttactgttttattaatctgattgattaagtctatcccaatatgtctctgcagttgggtgacccaatatgtctctgcagttgggtgacccaatatgtctctgcagttgggtgactacagaaataattcatgcagattacctcgctatttgtatcatattatatacaaatgacatactctagtgcatttgaaatgtaatttaatggaatcttgtagtttacaaaattaagattgtctttaaagtaaatcattattcctttaatttactcctctcatgacccctgtaactgtttttcacttcagttcaacatacatttatgccttgcctcctgtataccagccagtaggtgctggggttgcaaaataactaagatcaacccatacatgctggaagctctcaatctagtaggagaggatagacacgaaagctcataatttggttggtttggttttgagacagggcagtctctgtcgccagctggaggcagcggcatgatctcagctcactgcagctttgacctcccaggctgaagtgaccctttcacctcagtacccaaaagtagctggaattacaggtgcccacctgccagaaaactcagttttacttaaattatttattttcaacgtgtagctcatgaagatactaactttaaatggcagggagtagattgagaatcatcatggaagacctttttttttttttcttgagatggagtctcactccgtcacccaagctggagtgcaatggcatgatcttggctcactgcattctccacttctgggttcaagtgattctcctacctcagcctcccgagtagctgggattacaggcatgcagcaccacacccagctaatttttttatttttagtagagatggggtttcaccatgttggccaggctggtctaaaactcctgacctcaggtgatccacctgcctcagcctcccaaagtgctgggattacaggtgtgggccaccacacctggcccatcatggaagactttctgatggtgatgtttgaattgggttttggagagtgaattagaatgttttgaactaatacagtaaaggacagtagaaagaaggaacaacatggacagagaccttaaggcatgaaatgtcattctgtattcagttagacgtttagtcttgatagaaggattttttccttagaacagattacacctatatgataagattttattttgtttttatttaataatagttcagttaaaatataagcccaaaattgctccataaaatttggcagcagttatgctattgacagcatataaaaagcactcaatcgagctaggtgcagtggctcatggctgtaattccagcactttgggaggccccagcaaaaggatcacttgattcaaggaatttgagaccagtntgggcaacatggcaagactctatctgtatgaaaaaaaaaattttttaattagctgggaatagtgatgtgtgcccttgcagtgagctgtgatttgctctactgcactccaccttgagtagcagagtgaaactgtctttgaaaaaaaaaaaaaaa

SEQ ID NO: 344 - DNA, 561 bp, Homo sapiens
gcttaaacttcagatacccaagctgttcataaattagtgactacataagatagttctggttttacgactatagtgtgacactggttgagtcagtcagcttctctgggcctcatctgtaaaaggaaatggagagcaacattatctatactccctccctccctctatttctccctcccttccttcctccttcccttcctccctttcttcctcccttctccctttctctctccctccctgcctccctccctttcttccttctctgcctccttccctgtgattggggaaaataatagctcaagtaattttaaaaaaattcaatttagttatttctttagcatattactaaagacttagactcctaagactttccgtaagggaaactagatgtgctttgtagtgcagtcctaatttttaaaatttaaggagcccaggcatggtggctcacgcctatagtctcagaactttggatggctcaggcggaaggattgcttgagcccaggaatttgagaccagcctgggcaacatagcaaaacctcatctttaccaaaaaaaaaaaaaaaa

SEQ ID NO: 345 - DNA, 3443 bp, Homo sapiens
ccttttttttttttttttaccaaaaaaggggggaaaaaggatattagttttgttatcttcctcaattacaaaatgcctgttttttactctctgtcacacaaaccctgataaacatgcttctttatcgattagcacagttgggcttatactttttgtacagtatgccagtggaacatcagatgctgaacactagcacatgctgtgattttgcaatccctgcccacatcacacacttgatatcatttgtgggaggtcatgtgggttggcctacacactggcaagtaaactcattgatttggacaatgagtcactgacaatgaggatgttattttattttagcaggtttgtgccaacgtcattgcagaggcaagaagaagaagggatatgaatgatacactgacagataccaacttaataaataaaaagcttgccaatcttgaaaaaaaaggaacatggacttaacaccttgcaaagattttaagtattttacatattctaccaagcatgttatatagccaaagcctgtaatctctttctggcattaatctggctttttaaaaaaattctgttttgtttttgttttattaaaatcatggcctctggaattcattatcagtttctctccacttaaaccaagagagtactgctggttgcaaaaggatgttagaaatcatcccccattgtgctgttttctctaataatttgaagaagggtgttatttgctatgtgtatacactgtctccaaaatacattaactccttgtaaaggcctgacgccagttgtattagtctgttctcacattgttataaagaaatacctgagattgggtaatttataagaaaagaggtttaattgtctcacgggtctgcaggttgtacaggaagcatggcagcatctgcttctggggaggcctggggaaacttacaatcatggcagaaggcaaaagtggggacaggcacaccacatggccagagcaggagcaagagagagagacggaggggaggtaccacacgcttaaataactacatcttaggagaactcattcactgtcatgaggacagtaccatggggatggtgctgaaccattcataagaacctacccctgtgatctaaccacctccctgcaggccccacctccaacactggggattatatttcaacatgcgattttgagcaggaggacacatccaaactgtatcaccagtattcctccaagttgctgtcctctgcatagagtgctttttcccagatacttaaaggtaaaggggccaaagtatcccacccacaaaatctgtcctcagtatcccctgcatcctcatgcttcagagtcactcttatttctcaggtgaaatgagcaggtacgtatgactgtgatgtcacttaaatacaaaaaaatacaaaaaaaaaattcaaacaaaaataaataagaaatgagcaggtacggctgcctgggcaccacaccagagaagctcagaggcaaggtccacccaccccagggagaaacgctgaggatcaaactggtgaagagcaggccacattgcactttcctcttgctcttgttcacaagctgttcctcagaaatcacaggagatgtaaaggtctactggtttgctccctgagggaccttctggaaacttcatgggcctctaggcaatcaaaaggtcttctttttcaccagccccaatctttggaggaaacagagctttgaggaagaaaaagcaggaaaaccaggcacctggtctagaaaagtcagtgaagggggtccacagtggagctttccaccatactattctgaggctgcttcacaaagaccaaagctcctgcctaaaggttttgcctgctccccaggcatccagacatcactggctttttcctactccaacccaagaaaaggattctgtctaatcacaagtccaattggctgtctctcttgtctgaccaaaatcctgttctcgccaaacagaagcactgctatctacttccatctccccagcctactgtgtacaaagcatgccttgccacctgcccccctcatctttttgttcatgtgagaatgcatgggtcccaggagcttcggtgagaggacagagaaaaatgggaaattccattaccaaaacattttagagaaagaagaaaaggcaaaggagaatggaacgattgttgctacttgctggcagattaaaagaactgatgaagaactttcctgctttctccgggtgcactggctcacgcttgcgatcccacactttggggggccgaggcaagcagattgcctgagctcaggagttcaagaccagccagggcaacacagtgaaaccccatctctactaaaatacaaaaaattagccaggcgtggcagcatgcgcctgtaatcccagctactcgggaggctgaggcaggagaatcgcttgaacctgggaagcaggggttgcagtgagcagagactgggtcactgcactctagcctgggtgacagagcaagactctaactccaaaaaacaaaaaaaaaatctttcctgctttctctgcctatatactaagtaatccacatgagatagtcccatgattataaacactgcctaaagaaggatttaaaaataaacagacatttaaaatttttatagagaactttttacaatcaagatgaccaggcactgttaagaaaatgaaaagagctttgttatataacttatacctctcccaaaattctaaactatttatcctacttggaaaatatctcatataatctacctgctagtctggtccagttttctaatcattttttgctaacaagaagtgttttcttatatacaatataattcttttatttccatttaagccaatctttttgttccatctcttaagaatttgagaaaatgtgggtttgcttcctctttccaacttagttaaccaacttaatatgagtcatcccttggtttttctctaaattcagtaatcagccttcacagatctcacttaacagattcacagatctcttaaccatttttcaggcttcagaatttcctctttgccacccttaatatgaacagttaacctgggtttatctacgcttttacatcaaatccaaactgctttgtttttagggcctccagaacccaatcttgctttcccacaaccttatttctatgttttgtcccttctataaactaaatatactcccattgctaattaagctattccctcatccctccagtttagacagtcttctcactttatttaatcccaatccatctccacatggccgcttcaacaccagtatatccaccaaaaaacatttgccaatgtcccccatccagaatgatctcctttcatattgcaggcagaaaccagggtgagtggttcagtcatggctgtgtggttgaatgtgctggttctgttagcactgcagcatctttttaggttctgactagttctagatcgcga

SEQ ID NO: 346 - DNA, 1358 bp, Homo sapiens; identical to SEQ ID NO: 295

SEQ ID NO: 347 - DNA, 1047 bp, Homo sapiens
ctggataccatttgttgaaagatgttattactctagctgaacttacaagagactttagaccagggatctaaattacagtggccttagtgaccttgtccttatcttcttaggacagctgagaagccactgggacttagagcctttaaaaggagattaactgtcccaaaaggatctttgctactgaccagcagacacttcttccttcagtagcctttcatactgtgttgagtaacaccctagggtgtccattaaagttttgagttttacctagggcccagagccatgaatcaggattctgtctacatgattcgtgttttcattggtgtcaaaatacaaaagccaaagttctggctatgaattgttaacttggaagaaatactaactgccaccacttattaagtgcctactgtgtgccaggctctgaactaggtgcttcatatacattatcctaaattatctcaacatatgaggtaggtgttttaatttttattttatagaacttggtgtgtttgactgttaagctatggggctagagagagggtttgatcccaggtccctctgtgcttttgctgctgagccacacaacctctcatttcaaaaacactttcaaaatgctaacatattctaattcactctaggccaccaaaaactttaatactaatatctgatttgtaaatgacttaatgtatccttgaccctatcagctgaatttaatgaaatattcctctctgctgtgaaattttaccagtatagtatttggtctagtgacagagcgagactccgtctacacacacacacacacacacacacacatccttcctcctctaaccccaaactaagatcacagaaggtgatccagtcagagaacagagggaaatcttaccaggaagggcttaagtacacttttttttaaaacagctttattgtttttaaagcctacaatttgataagccttgacatatgtatacctgtgaaagcatcaccacaatcaagacactggacatatctatcacaccccatcctaaaaaaaaaaaaaaaaaaaaaaaa

SEQ ID NO: 348 - DNA, 1306 bp, Homo sapiens
tccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttctttttttttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtaaagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgccaaaaaaaaaaaaaaaaaaaaaaaaaaaaaaaaaaaaaaaaaaaaaaa

SEQ ID NO: 349 - DNA, 341 bp, Homo sapiens
agaaaataagccattcctcataccaatataggatcagctccttgacctctgaggggcaggagtgcttcctggtgtgtgtattagaatcccttcctgccttgtttcatggcagtgaaatgcctcttggtcctgtccaagtgtatctttcactgatttctgaatcatgttctagttgcttgaccctgccacatgggtccagtgttcatctgagcataactgtactaaatcctttttccatatcagtataataaaggagtgatgtgcaataaaaaaaaaaaaaaaaaaaaaaaaaaaaaaaaaaaaaaaaaaaaaaaaaaaaaaaaaaaaaaaaaaaaaaaaaa

SEQ ID NO: 350 - DNA, 791 bp, Homo sapiens
ggcacctgtagtcccagctactcgggaggctgaggcaggaaaatctcttgaacctgggaggcagaggttgcagtgagccgagattgtgccactgcactctagcctagctgacagagtgaggctccgtcctaaaaaaaaaaaaaagtaaatatctgttgatgaaaaaactgacacttcctatgggtttacctcctttccttccgttgttttctctttggtatccctcacgcgttttcccctctccgctgcagtcacctatttcccacttgtttttcttctctccttcttctttttcttattgtgtcctccctgccaccagtcacaggcttgtggtctacaaataatgctggtttgggtttattttaaaacatctaacatgagatcagtgcctgctttttaaagaagcattacattatgtattagttatacaaattattagacaatgtcttatctttattttattgttttacacatagaacagagactatttggagcctttggaataacattccagcgtataaatataaatgaaatagtttggcaaattaactctctccaggggtcatctagaaatatgattctgtcatcagatagaaattctattgctagagtcctttagccagcaaatagattttctatgcttggtgagcaaattcatcacaaatttgaagctagttacaaataaaataaaataaaataaattaattaaaaagaaattttaaaaatcccaacttacagtttaaaaagaagaaaagtggaaaaaaaaaacaatgaacaaaaaaaaaaaaaaa

SEQ ID NO: 351 - DNA, 1474 bp, Homo sapiens
ttcagcagttagctaaattacaagatcgagaatggttaacagaactttttcaacaaagcaaggtcaatatgcagaaaatgctcccagaaattgatcagaataaggaccgcatgttggagattttggaaggaaagggactgagtttcttattcccactcctcaaattggagaaggaactgttgaagcaaataaagttggatccatcccctcaaaccatatataaatggattaaagataacatctctcccaaacttcatgtagataaaggatttgtgaacatcttaatgactagcttcttacagtacatttctagtgaagtaaacccccccagcgatgaaacagattcatcctctgctccttccaaagaacagttagagcaggaaaaacaactactactatctttcaagccagtaatgcagaaatttcttcatgatcacgttgatctacaagtcagtgccctgtatgctctccaggtgcactgctataacagcaacttcccaaaaggcatgttacttcgcttttttgtgcacttctatgacatggaaattattgaagaagaagctttcttggcttggaaagaagatataacccaagagtttccgggaaaaggcaaggctttgttccaggtgaatcagtggctaacctggttagaaactgctgaagaagaagaatcagaggaagaagctgactaaagaaccagccaaagccttaaattgtgcaaaacatactgttgctatgatgtaactgcatttgacctaaccactgcgaaaattcattccgctgtaatgttttcacaatatttaaagcagaagcacgtcagttaggatttccttctgcataaggtttttttgtagtgtaatgtcttaatcatagtctaccatcaaatattttaggagtatctttaatgtttagatagtatattagcagcatgcaataattacatcataagttctcaagcagaggcagtctattgcaaggaccttctttgctgccagttatcataggctgttttaagttagaaaactgaatagcaacactgaatactgtagaaatgcactttgctcagtaatacttgagttgttgcaatatttgattatccatttggttgttacagaaaaattcttaactgtaattgatggttgttgccgtaatagtatattgcctgtatttctacctctagtaatgggctttatgtgctagattttaatatccttgagcctgggcaagtgcacaagtctttttaaaagaaacatggtttacttgcacaaaactgatcagttttgagagatcgttaatgcccttgaagtggtttttgtgggtgtgaaacaaatggtgagaatttgaattggtccctcctattatagtattgaaattaagtctacttaatttatcaagtcatgttcatgccctgattttatatacttgtatctatcaataaacattgtgatacttgaaaaaaaaaaaaaaaaaaaaa

SEQ ID NO: 352 - DNA, 2932 bp, Homo sapiens
  misc_feature (1332)..(1332): n at position 1332 is a or c or g or t
ccagcctggacgacagagcagactccatctcaaaaaaaaaaaaaaaatggtcaccccttttgctcctaaatcaccctcaaagtaaaagagaacaagaaacagaagcagaaatccatatttagtgaaataagacaacacctgtagctccaaacctgtagaagcagatcccagagaaaagcaggccagttctctcctggaatcccagaaagtcccaggaattggaggcttcagtgctgcagtagggaggggactaaaaacaaagtctgtatatggagcagtaagacctccgggttctcatcccccatcctgtgtgctctcgggtgactagccctctccctctccaggtttagcttctggagaaattaaatcaaagaggctctagaactggggatggcaggcattgagtgcagggagtgagttgccagaggctgagagcagagagatttagtggcagtgggcagagcaaacagcaaaatgactgccctcttccctggccttgctccagaaactgagcagccagacatacacactcccagaaggcagttggaggtccccccgggcaatcagaaccacccacagagaagacctccagatactgacatttagaagccttctgaccaaagagcttgcctggccacccactggcttactcgctgacaggccatgctcctgccgtacacaccggatcctatcagtcatcctcatgggctcctttccagacttctgaggaaagcttccaacatgaagagggagttcaaaacaaacagagagagagagagagagagagagagagagagagagagagagagaaaagaaatataaacaatgcagggagcaaaagataacttcaaataaactcaaaatattcttctgggagagatgagacaatattgcatacataaaacaagaagaggatgctatcactaaattttaaaaaaaagtaacagaaagtaagaaagacctctaagaaatgtttctgaaatgaggtataatttttttttttttgagggggagtcttgctctgtcacccaggctggagcgcagtggcgtgatctcggctcactgcaacctccgtctcccgggttcaagtgattctcctgcctcagcttcctgagtagctgggattacaggcatgcgccaccatgcctggctaattttgcatttttagtagagacggggtttcaccatgttggccaggctggtctgaaactcccgacctcaggtgatctgcccacctcagcctcccaaagtgctgggattacaggcatgagctaccgtgcctggcctgtgtatttttggagacagagtcttgctctgttgcccaggctgganggcagtggtgcaactctaaactctgcctcccgggttcaagcgattctcgtgcgtcacctcccaagtagctggggctagaggcatgtgccaccatgtctggttactttttatataaaccttcagtttaatcttttagcttactgctgtgtttttcagctttattcactttgctttttttctatattttcgtttcaactcaacttttaaaaaatcatacttgtatatttgtatatttatatttattttccaagacctcatttgcctctttttcataatgggctattattgctccatggtacagaatcttctgttctttcctaaactattagttatttttaaaatatttgttaactttctcccccttgctagttctcaggtacccgctttctcccagagtgctggctttccttaattatttgctgacttgtgctggtgtgtaacccttcatacttaggtatttctatttgtctgactgctgatttgattccaatccagtgtttcttctgattcgtggagaagagacgacaacgctgtgaggctctggttttggtggcttgtctgggtgtgggagctccttgtcatcatgggatttttagctccctgggtcgccatcctacatggccactctcctgccgatgctgcccgctactcagcaggaggggaagtcgagaccacctccttaaccttacagacattgattgtgagcttggagcaccttccgtgactggaccacccatgcagtggtcctttgctttttgcatttttaactgcaattttccccaagagtcttccctaatacagtctcttaggaattgacggtgggattaaacaaggcatgtttctgcactgaatatggtctatgggttgccactttgcaacctcagctttaaggcttatttcctcccaggaaactgatgttaactttttaagttaaaaatgtgtatataagtaatacatgtttattttggaaattagaaaatacaggccaggtgtggtggctcatgcctgtaatcccaacattttgggaggttgaggcgggaggattgcctgggcccgggagtttgagaccagcctgggcaacatagggaaattccatctctacaaaaaaattaaacattggctgggtgtggtggtgcccacctgtggtcacagctactcgggagactgaggtgggaggatagcttgggcaggggaggttgaggctacagtgagccacgatcacaccaatgtgacacagtgagaccctgtctcaaaaaaaaaaaaaaagaaagaaaatataggtaaacacaaagacaaaaaagcagggcatggtggctcacacctgtaattctagcaccttgggaggctgaggcaggaggatcacttgaggtcaggaactcgagacgagaccaggctgggcagcatggcaggaccccatctctataaaaagtacaaaaattagcagggcacggtggtgtgcacctgtggtcctgctacttgggaagttgaggtgggaggatcacctgagccctggaggttgagggtgctgtgagccatgatggcaccactccactccagtccaggtgaaagagccagatcctgtctcaaaaaaaaaaaaaaa

SEQ ID NO: 353 - DNA, 1254 bp, Homo sapiens
gagagcggggcctacggcgcggccaaggcgggcggctccttcgacctgcggcgcttcctgacgcagccgcaggtggtggcgcgcgccgtgtgcttggtcttcgccttgatcgtgttctcctgcatctatggtgagggctacagcaatgcccacgagtctaagcagatgtactgcgtgttcaaccgcaacgaggatgcctgccgctatggcagtgccatcggggtgctggcctccctggcctaccagcgctacaaggctggcgtggacgacttcatccagaattacgttgaccccactccggaccccaacactgcctacgcctcctacccaggtgcatctgtggacaactaccaacagccacccttcacccagaacgcggagaccaccgagggctaccagccgccccctgtgtactgagcggcggttagcgtgggaagggggacagagagggccctcccctctgccctggactttcccatgagcctcctggaactgccagcccctctctttcacctgttccatcctgtgcagctgacacacagctaaggagcctcatagcctggcgggggctggcagagccacaccccaagtgcctgtgcccagagggcttcagtcagccgctcactcctccagggcacttttaggaaagggtttttagctagtgtttttcctcgcttttaatgacctcagccccgcctgcagtggctagaagccagcaggtgcccatgtgctactgacaagtgcctcagcttccccccggcccgggtcaggccgtgggagccgctattatctgcgttctctgccaaagactcgtgggggccatcacacctgccctgtgcagcggagccggaccaggctcttgtgtcctcactcaggtttgcttcccctgtgcccactgctgtatgatctgggggccaccaccctgtgccggtggcctctgggctgcctcccgtggtgtgagggcggggctggtgctcatggcacttcctccttgctcccacccctggcagcagggaagggctttgcctgacaacacccagctttatgtaaatattctgcagttgttacttaggaagcctggggagggcaggggtgccccatggctcccagactctgtctgtgccgagtgtattataaaatcgtgggggagatgcccggcctgggatgctgtttggagacggaataaatgttttctcattcaaaaaaaaaaaaaaaaaaaaaaaaaaaaaaaaaaaaaaaaaaaaaaaaaaaaaa

SEQ ID NO: 354 - DNA, 1324 bp, Homo sapiens
cctttccaagggagtggttgtgtgatcgccatcttagggaaaagatgttctcgtccgtggcgcacctggcgcgggcgaaccccttcaacacgccacatctgcagctggtgcacgatggtctcggggacctccgcagcagctccccagggcccacgggccagccccgccgccctcgcaacctggcagccgccgccgtggaagagtacagttgtgaatttggctccgcgaagtattatgcactgtgtggctttggtggggtcttaagttgtggtctgacacacactgctgtggttcccctggatttagtgaaatgccgtatgcaggtggacccccaaaagtacaagggcatatttaacggattctcagttacacttaaagaggatggtgttcgtggtttggctaaaggatgggctccgactttccttggctactccatgcagggactctgcaagtttggcttttatgaagtctttaaagtcttgtatagcaatatgcttggagaggagaatacttatctctggcgcacatcactatatttggctgcctctgccagtgctgaattctttgctgacattgccctggctcctatggaagctgctaaggttcgaattcaaacccagccaggttatgccaacactttgagggatgcagctcccaaaatgtataaggaagaaggcctaaaagcattctacaagggggttgctcctctctggatgagacagataccatacaccatgatgaagttcgcctgctttgaacgtactgttgaagcactgtacaagtttgtggttcctaagccccgcagtgaatgttcaaagccagagcagctggttgtaacatttgtagcaggttacatagctggagtcttttgtgcaattgtttctcaccctgctgattctgtggtatctgtgttgaataaagaaaaaggtagcagtgcttctctggtcctcaagagacttggatttaaaggtgtatggaagggactgtttgcccgtatcatcatgattggtaccctgactgcactacagtggtttatctatgactccgtgaaggtctacttcagacttcctcgccctcctccacccgagatgccagagtctctgaagaagaagcttgggttaactcagtagttagatcaaagcaaatgtggactgaatctgcttgttgatcagtgttgaagaaagtgcaaaaggaacttttatatatttgacagtgtaggaaattgtctattcctgatataattactgtagtactcttgcttaaggcaagagtttcagatttactgttgaaataaacccaactcttcatgaaaaaaaaaaaaaaa

SEQ ID NO: 355 - DNA, 2303 bp, Homo sapiens
cggacgcgtgggcggcgatcgcggcctgaggctgctcccggacaagggcaacgagcgtttcgtttggacttctcgacttgagtgcccgcctccttcgccgccgcctctgcagtcctcagcgcagtctttccacaggagccagcatacttcctgaacatggagagtgttgttcgccgctgcccattcttatcccgagtcccccaggcctttctgcagaaagcaggcaaatctctgttgttctatgcccaaaactgccccaagatgatggaagttggggccaagccagcccctcgggcattgtccactgcagcagtacactaccaacagatcaaagaaacccctccggccagtgagaaagacaaaactgctaaggccaaggtccaacagactcctgatggatcccagcagagtccagatggcacacagcttccgtctggacaccccttgcctgccacaagccagggcactgcaagcaaatgccctttcctggcagcacagatgaatcagagaggcagcagtgtcttctgcaaagccagtcttgagcttcaggaggatgtgcaggaaatgaatgccgtgaggaaagaggttgctgaaacctcagcaggccccagtgtggttagtgtgaaaaccgatggaggggatcccagtggactgctgaagaacttccaggacattatgcaaaagcaaagaccagaaagagtgtctcatcttcttcaagataacttgccaaaatctgtttccacttttcagtatgatcgtttctttgagaaaaaaattgatgagaaaaagaatgaccacacctatcgagtttttaaaactgtgaaccggcgagcacacatcttccccatggcagatgactattcagactccctcatcaccaaaaagcaagtgtcagtctggtgcagtaatgactacctaggaatgagtcgccacccacgggtgtgtggggcagttatggacactttgaaacaacatggtgctggggcaggtggtactagaaatatttctggaactagtaaattccatgtggacttagagcgggagctggcagacctccatgggaaagatgccgcactcttgttttcctcgtgctttgtggccaatgactcaaccctcttcaccctggctaagatgatgccaggctgtgagatttactctgattctgggaaccatgcctccatgatccaagggattcgaaacagccgagtgccaaagtacatcttccgccacaatgatgtcagccacctcagagaactgctgcaaagatctgacccctcagtccccaagattgtggcatttgaaactgtccattcaatggatggggcggtgtgcccactggaagagctgtgtgatgtggcccatgagtttggagcaatcaccttcgtggatgaggtccacgcagtggggctttatggggctcgaggcggagggattggggatcgggatggagtcatgccaaaaatggacatcatttctggaacacttggcaaagcctttggttgtgttggagggtacatcgccagcacgagttctctgattgacaccgtacggtcctatgctgctggcttcatcttcaccacctctctgccacccatgctgctggctggagccctggagtctgtgcggatcctgaagagcgctgagggacgggtgcttcgccgccagcaccagcgcaacgtcaaactcatgagacagatgctaatggatgccggcctccctgttgtccactgccccagccacatcatccctgtgcgggttgcagatgctgctaaaaacacagaagtctgtgatgaactaatgagcagacataacatctacgtgcaagcaatcaattaccctacggtgccccggggagaagagctcctacggattgcccccacccctcaccacacaccccagatgatgaactacttccttgagaatctgctagtcacatggaagcaagtggggctggaactgaagcctcattcctcagctgagtgcaacttctgcaggaggccactgcattttgaagtgatgagtgaaagagagaagtcctatttctcaggcttgagcaagttggtatctgctcaggcctgagcatgacctcaattatttcacttaaccccaggccattatcatatccagatggtcttcagagttgtctttatatgtgaattaagttatattaaattttaatctatagtaaaaacatagtcctggaaataaattcttgcttaaatggtgaaaaaaaaaaaaaaaaaaaaaaaaaaaaaaaaaaaaaaaaaaaaaaaaaaaaaaaaaaaaaaaaaaaaaaaaaaaaa

SEQ ID NO: 356 - protein, 361 aa, Homo sapiens
MFSSVAHLARANPFNTPHLQLVHDGLGDLRSSSPGPTGQPRRPRNLAAAAVEEYSCEFGSAKYYALCGFGGVLSCGLTHTAVVPLDLVKCRMQVDPQKYKGIFNGFSVTLKEDGVRGLAKGWAPTFLGYSMQGLCKFGFYEVFKVLYSNMLGEENTYLWRTSLYLAASASAEFFADIALAPMEAAKVRIQTQPGYANTLRDAAPKMYLEEGLKAFYKGVAPLWMRQIPYTMMKFACFERTVEALYKFVVPKPRSECSKPEQLVVTFVAGYIAGVFCAIVSHPADSVVSVLNKEKGSSASLVLKRLGFKGVWKGLFARIIMIGTLTALQWFIYDSVKVYFRLPRPPPPEMPESLKKKLGLTQ

SEQ ID NO: 357 - protein, 640 aa, Homo sapiens
MESVVRRCPFLSRVPQAFLQKAGKSLLFYAQNCPKMMEVGAKPAPRALSTAAVHYQQIKETPPASEKDKTAKAKVQQTPDGSQQSPDGTQLPSGHPLPATSQGTASKCPFLAAQMNQRGSSVFCKASLELQEDVQEMNAVRKEVAETSAGPSVVSVKTDGGDPSGLLKNFQDIMQKQRPERVSHLLQDNLPKSVSTFQYDRFFEKLIDEKKNDHTYRVFKTVNRRAHIFPMADDYSDSLITKKQVSVWCSNDYLGMSRHPRVCGAVMDTLKQHGAGAGGTRNISGTSKFHVDLERELADLHGKDAALLFSSCFVANDSTLFTLAKMMPGCEIYSDSGNHASMIQGIRNSRVPKYIFRHNDVSHLRELLQRSDPSVPKIVAFETVHSMDGAVCPLEELCDVAHEFGAITFVDEVHAVGLYGARGGGIGDRDGVMPKMDIISGTLGKAFGCVGGYIASTSSLIDTVRSYAAGFIFTTSLPPMLLAGALESVRILKSAEGRVLRRQHQRNVKLMRQMLMDAGLPVVHCPSHIIPVRVADAAKNTEVCDELMSRHNIYVQAINYPTVPRGEELLRIAPTPHHTPQMMNYFLENLLVTWKQVGLELKPHSSAECNFCRRPLHFEVMSEREKSYFSGLSKLVSAQA

SEQ ID NO: 358 - protein, 9 aa, Artificial
  Designed peptide recognized by HLA-A2 restricted cytotoxic T lymphocytes
QIGAKFWEV

SEQ ID NO: 359 - protein, 9 aa, Artificial
  Designed peptide recognized by HLA-A2 restricted cytotoxic T lymphocytes
FMPGFAPLT

SEQ ID NO: 360 - protein, 10 aa, Artificial
  Designed peptide recognized by HLA-A2 restricted cytotoxic T lymphocytes
TLLVAVFQDV

SEQ ID NO: 361 - protein, 10 aa, Artificial
  Designed peptide recognized by HLA-A2 restricted cytotoxic T lymphocytes
VAYLGFVFYL

SEQ ID NO: 362 - protein, 10 aa, Artificial
  Designed peptide recognized by HLA-A2 restricted cytotoxic T lymphocytes
LLPTLRKQYC

SEQ ID NO: 363 - protein, 9 aa, Artificial
  Designed peptide recognized by HLA-A2 restricted cytotoxic T lymphocytes
MVYDLYKTL

SEQ ID NO: 364 - protein, 10 aa, Artificial
  Designed peptide recognized by HLA-A2 restricted cytotoxic T lymphocytes
GLCKFGFYEV

SEQ ID NO: 365 - protein, 9 aa, Artificial
  Designed peptide recognized by HLA-A2 restricted cytotoxic T lymphocytes
FGFYEVFKV

SEQ ID NO: 366 - protein, 9 aa, Artificial
  Designed peptide recognized by HLA-A2 restricted cytotoxic T lymphocytes
LQWFIYDSV

SEQ ID NO: 367 - protein, 10 aa, Artificial
  Designed peptide recognized by HLA-A2 restricted cytotoxic T lymphocytes
ALAPMEAAKV

SEQ ID NO: 368 - protein, 10 aa, Artificial
  Designed peptide recognized by HLA-A2 restricted cytotoxic T lymphocytes
RTVEALYKFV

SEQ ID NO: 369 - protein, 9 aa, Artificial
  Designed peptide recognized by HLA-A2 restricted cytotoxic T lymphocytes
VLSCGLTHT

SEQ ID NO: 370 - protein, 9 aa, Artificial
  Designed peptide recognized by HLA-A2 restricted cytotoxic T lymphocytes
ALLFSSCFV

SEQ ID NO: 371 - protein, 10 aa, Artificial
  Designed peptide recognized by HLA-A2 restricted cytotoxic T lymphocytes
FLSRVPQAFL

SEQ ID NO: 372 - protein, 10 aa, Artificial
  Designed peptide recognized by HLA-A2 restricted cytotoxic T lymphocytes
MLLAGALESV

SEQ ID NO: 373 - protein, 10 aa, Artificial
  Designed peptide recognized by HLA-A2 restricted cytotoxic T lymphocytes
LLQDNLPKSV

SEQ ID NO: 374 - protein, 9 aa, Artificial
  Designed peptide recognized by HLA-A2 restricted cytotoxic T lymphocytes
LMSRHNIYV

SEQ ID NO: 375 - protein, 10 aa, Artificial
  Designed peptide recognized by HLA-A2 restricted cytotoxic T lymphocytes
SLIDTVRSYA

SEQ ID NO: 376 - protein, 10 aa, Artificial
  Designed peptide recognized by HLA-A2 restricted cytotoxic T lymphocytes
FLQKAGKSLL

SEQ ID NO: 377 - protein, 9 aa, Artificial
  Designed peptide recognized by HLA-A2 restricted cytotoxic T lymphocytes
LLFSSCFVA

SEQ ID NO: 378 - protein, 9 aa, Artificial
  Designed peptide recognized by HLA-A2 restricted cytotoxic T lymphocytes
GLLKNFQDI

SEQ ID NO: 379 - protein, 9 aa, Artificial
  Designed peptide recognized by HLA-A2 restricted cytotoxic T lymphocytes
SVWCSNDYL

SEQ ID NO: 380 - protein, 10 aa, Artificial
  Designed peptide recognized by HLA-A2 restricted cytotoxic T lymphocytes
LLVTWKQVGL

SEQ ID NO: 381 - protein, 10 aa, Artificial
  Designed peptide recognized by HLA-A2 restricted cytotoxic T lymphocytes
VANDSTLFTL

SEQ ID NO: 382 - DNA, 974 bp, Homo sapiens
gaaacaagttgaagtacctggctttcctccgcaagcggatgaacaccaacccttcccgaggcccctaccacttccgggcccccagccgcatcttctggcggaccgtgcgaggtatgctgccccacaaaaccaagcgaggccaggccgctctggaccgtctcaaggtgtttgacggcatcccacctccctacgacaagaaaaagcggatggtggttcctgctgccctcaaggtcgtgcgtctgaagcctacaagaaagtttgcctatctggggcgcctggctcacgaggttggctggaagtaccaggcagtgacagccaccctggaggagaagaggaaagagaaagccaagatccactaccggaagaagaaacagctcatgaggctacggaaacaggccgagaagaacgtggagaagaaaattgacaaatacacagaggtcctcaagacccacggactcctggtctgagcccaataaagactgttaattcctcatgcgttgcctgcccttcctccattgttgccctggaatgtacgggacccaggggcagcagcagtccaggtgccacaggcagccctgggacataggaagctgggagcaaggaaagggtcttagtcactgcctcccgaagttgcttgaaagcactcggagaattgtgcaggtgtcatttatctatgaccaataggaagagcaaccagttactatgagtgaaagggagccagaagactgattggagggccctatcttgtgagtggggcatctgttggacttcccacctggtcatatactctgcagctgttagaatgtgcaagcacttggggacagcatgagcttgctgttgtacacagggtatttctagaagcagaaatagactgggaagatgcacaaccaaggggttacaggcatcgcccatgctcctcacctgtattttgtaatcagaaataaattgcttttaaagaaaaaaaaaaaaaaaaa

SEQ ID NO: 383 - DNA, 821 bp, Homo sapiens
ggggccggggccgaggccgcggagctcgcggaggcaaggccgaggataaggagtggatgcccgtcaccaagttgggccgcttggtcaaggacatgaagatcaagtccctggaggagatctatctcttctccctgcccattaaggaatcagagatcattgatttcttcctgggggcctctctcaaggatgaggttttgaagattatgccagtgcagaagcagacccgtgccggccagcgcaccaggttcaaggcatttgttgctatcggggactacaatggccacgtcggtctgggtgttaagtgctccaaggaggtggccaccgccatccgtggggccatcatcctggccaagctctccatcgtccccgtgcgcagaggctactgggggaacaagatcggcaagccccacactgtcccttgcaaggtgacaggccgctgcggctctgtgctggtacgcctcatccctgcacccaggggcactggcatcgtctccgcacctgtgcctaagaagctgctcatgatggctggtatcgatgactgctacacctcagcccggggctgcactgccaccctgggcaacttcgccaaggccacctttgatgccatttctaagacctacagctacctgacccccgacctctggaaggagactgtattcaccaagtctccctatcaggagttcactgaccacctcgtcaagacccacaccagagtctccgtgcagcggactcaggctccagctgtggctacaacatagggtttttatacaagaaaaataaagtgaatttagcgtgaaaaaaaaaaaaaaaaaaaaaaaaaa

SEQ ID NO: 384 - DNA, 741 bp, Homo sapiens
gcggcgtgagaagccatgagcagcaaagtctctcgcgacaccctgtacgaggcggtgcgggaagtcctgcacgggaaccagcgcaagcgccgcaagttcctggagacggtggagttgcagatcagcttgaagaactatgatccccagaaggacaagcgcttctcgggcaccgtcaggcttaagtccactccccgccctaagttctctgtgtgtgtcctgggggaccagcagcactgtgacgaggctaaggccgtggatatcccccacatggacatcgaggcgctgaaaaaactcaacaagaataaaaaactggtcaagaagctggccaagaagtatgatgcgtttttggcctcagagtctctgatcaagcagattccacgaatcctcggcccaggtttaaataaggcaggaaagttcccttccctgctcacacacaacgaaaacatggtggccaaagtggatgaggtgaagtccacaatcaagttccaaatgaagaaggtgttatgtctggctgtagctgttggtcacgtgaagatgacagacgatgagcttgtgtataacattcacctggctgtcaacttcttggtgtcattgctcaagaaaaactggcagaatgtccgggccttatatatcaagagccccatgggcaagccccagcgcctatattaaggcccatttgaataaattctatttcccgtttaaaaaaaaaaaaaaaaaaaaaaaaaaaaaaaaaaaaaaaaa

SEQ ID NO: 385 - protein, 142 aa, Homo sapiens
MNTNPSRGPYHFRAPSRIFWRTVRGMLPHKTKRGQAALDRLKVFDGIPPPYDKKKRMVVPAALKVVRLKPTRKFAYLGRLAHEVGWKYQAVTATLEEKRKEKAKIHYRKKKQLMRLRKQAEKNVEKKIDKYTEVLKTHGLLV

SEQ ID NO: 386 - protein, 233 aa, Homo sapiens
MPVTKLGRLVKDMKIKSLEEIYLFSLPIKESEIIDFFLGASLKDEVLKIMPVQKQTRAGQRTRFKAFVAIGDYNGHVGLGVKCSKEVATAIRGAIILAKLSIVPVRRGYWGNKIGKPHTVPCKVTGRCGSVLVRLIPAPRGTGIVSAPVPKKLLMMAGIDDCYTSARGCTATLGNFAKATFDAISKTYSYLTPDLWKETVFTKSPYQEFTDHLVKTHTRVSVQRTQAPAVATT

SEQ ID NO: 387 - protein, 217 aa, Homo sapiens [sequence omitted]

SEQ ID NO: 388 - protein, 9 aa, Artificial
  Designed peptide recognized by HLA-A26 restricted cytotoxic T lymphocytes
LVLDGRGHL

SEQ ID NO: 389 - protein, 9 aa, Artificial
  Designed peptide recognized by HLA-A26 restricted cytotoxic T lymphocytes
HLLGRLAAI

SEQ ID NO: 390 - protein, 9 aa, Artificial
  Designed peptide recognized by HLA-A26 restricted cytotoxic T lymphocytes
AIVAKQVLL

SEQ ID NO: 391 - protein, 9 aa, Artificial
  Designed peptide recognized by HLA-A26 restricted cytotoxic T lymphocytes
VLLGRKVVV

SEQ ID NO: 392 - protein, 9 aa, Artificial
  Designed peptide recognized by HLA-A26 restricted cytotoxic T lymphocytes
AFLRKRMNT

SEQ ID NO: 393 - protein, 9 aa, Artificial
  Designed peptide recognized by HLA-A26 restricted cytotoxic T lymphocytes
HFRAPSRIF

SEQ ID NO: 394 - protein, 9 aa, Artificial
  Designed peptide recognized by HLA-A26 restricted cytotoxic T lymphocytes
VLKTHGLLV

SEQ ID NO: 395 - protein, 9 aa, Artificial
  Designed peptide recognized by HLA-A26 restricted cytotoxic T lymphocytes
PVTKLGRLV

SEQ ID NO: 396 - protein, 9 aa, Artificial
  Designed peptide recognized by HLA-A26 restricted cytotoxic T lymphocytes
KIMPVQKQT

SEQ ID NO: 397 - protein, 9 aa, Artificial
  Designed peptide recognized by HLA-A26 restricted cytotoxic T lymphocytes
VTGRCGSVL

SEQ ID NO: 398 - protein, 9 aa, Artificial
  Designed peptide recognized by HLA-A26 restricted cytotoxic T lymphocytes
RLIPAPRGT

SEQ ID NO: 399 - protein, 9 aa, Artificial
  Designed peptide recognized by HLA-A26 restricted cytotoxic T lymphocytes
DLWKETVFT

SEQ ID NO: 400 - protein, 9 aa, Artificial
  Designed peptide recognized by HLA-A26 restricted cytotoxic T lymphocytes
HLVKTHTRV

SEQ ID NO: 401 - protein, 9 aa, Artificial
  Designed peptide recognized by HLA-A26 restricted cytotoxic T lymphocytes
HTRVSVQRT

SEQ ID NO: 402 - protein, 9 aa, Artificial
  Designed peptide recognized by HLA-A26 restricted cytotoxic T lymphocytes
RTQAPAVAT

SEQ ID NO: 403 - protein, 9 aa, Artificial
  Designed peptide recognized by HLA-A26 restricted cytotoxic T lymphocytes
TLYEAVREV

SEQ ID NO: 404 - protein, 9 aa, Artificial
  Designed peptide recognized by HLA-A26 restricted cytotoxic T lymphocytes
ETVELQISL

SEQ ID NO: 405 - protein, 9 aa, Artificial
  Designed peptide recognized by HLA-A26 restricted cytotoxic T lymphocytes
KVDEVKSTI

SEQ ID NO: 406 - protein, 9 aa, Artificial
  Designed peptide recognized by HLA-A26 restricted cytotoxic T lymphocytes
TIKFQMKVL

SEQ ID NO: 407 - protein, 9 aa, Artificial
  Designed peptide recognized by HLA-A26 restricted cytotoxic T lymphocytes
KVLCLAVAV

SEQ ID NO: 408 - protein, 9 aa, Artificial
  Designed peptide recognized by HLA-A26 restricted cytotoxic T lymphocytes
STMGKPQRL